(19) United States
(12) Patent Application Publication
(10) Pub. No.: US 2025/0042897 A1
(43) Pub. Date: Feb. 6, 2025

(54) HETEROCYCLIC COMPOUNDS AS TRIGGERING RECEPTOR EXPRESSED ON MYELOID CELLS 2 AGONISTS AND METHODS OF USE

(71) Applicant: Vigil Neuroscience, Inc., Watertown, MA (US)

(72) Inventors: Jonathan B. HOUZE, Watertown, MA (US); Bhaumik PANDYA, Watertown, MA (US); Alan P. KAPLAN, Watertown, MA (US); Maxence BOS, Saint-Laurent (CA); John MANCUSO, Saint-Laurent (CA); Ivan FRANZONI, Saint-Laurent (CA)

(73) Assignee: Vigil Neuroscience, Inc., Watertown, MA (US)

(21) Appl. No.: 18/707,494

(22) PCT Filed: Nov. 9, 2022
(86) PCT No.: PCT/US2022/079516

§ 371 Date: May 3, 2024

Related U.S. Application Data
(60) Provisional application No. 63/263,813, filed on Nov. 9, 2021.
(60) Provisional application No. 63/375,137, filed on Sep. 9, 2022.

(51) Int. Cl. C07D 471/04; A61K 31/4375; A61K 31/5025; A61K 31/519; A61K 31/5377

(52) U.S. Cl. CPC C07D 471/04; A61K 31/4375; A61K 31/5025; A61K 31/519; A61K 31/5377

(57) ABSTRACT

The present disclosure provides compounds of Formula (I), useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula (I).

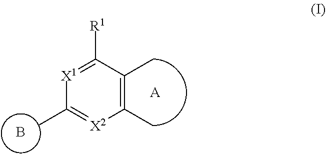

### CROSS REFERENCE TO RELATED APPLICATION

This application claims the benefit of U.S. Provisional Application No. 63/263,813, filed Nov. 9, 2021, and U.S. Provisional Application No. 63/375,137, filed Sep. 9, 2022, the entirety of each of which is incorporated herein by reference.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neruopathology. 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016: Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”). Toll-like receptors (“TLR”). Fc receptors, nucleotide-binding oligomerization domain (“NOD”) and purinergic G protein-coupled receptors. Doens and Fernandez 2014. Madry and Attwell 2015. Hickman and El Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med. 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010: Jay et al. Mol Neurodegener. 2017). TREM2/DAP12 complexes in particular act as a signaling unit that can be characterized as pro-activation on microglial phenotypes in addition to peripheral macrophages and osteoclasts (Otero et al. J Immunol. 2012: Kobayashi et al. J Neurosci. 2016; Jaitin et al., Cell. 2019. In the CNS, signaling through TREM2 has been studied in the context of ligands such as phospholipids, cellular debris, apolipoproteins, and myelin (Wang et al. Cell. 2015: Kober and Brett. J Mol Biol. 2017; Shirotani et al., Sci Rep. 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol. 2015. Wu et al., Mol Brain. 2017).

Coding variants in the TREM2 locus has been associated with late onset Alzheimer's disease (“LOAD”) in human genome-wide association studies, linking a loss-of-receptor function to a gain in disease risk (Jonsson et al. N Engl J Med 2013. Sims et al. Nat Genet 2017). Genetic variation of other genes selectively expressed by microglia in the CNS, for example, CD33. PLCg2 and MS4A4A/6A have reached genome-wide significance for their association with LOAD risk (Hollingworth et al. Nat Genet 2011. Sims et al. Nat Genet 2017. Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM2 in LOAD, homozygous loss-of-function mutations in TREM2 are causal for an early onset dementia syndrome known as Polycystic lipomembranous osteodysplasia with sclerosing leukoencephalopathy (“PLOSL”) or Nasu-Hakola disease (“NHD”) (Golde et al. Alzheimers Res Ther 2013. Dardiotis et al. Neurobiol Aging 2017). This progressive neurodegenerative disease typically manifests in the 3rd decade of life and is pathologically characterized by loss of myelin in the brain concomitant with gliosis, unresolved neuroinflammation, and cerebral atrophy. Typical neuropsychiatric presentations are often preceded by osscous abnormalities, such as bone cysts and loss of peripheral bone density (Bianchin et al. Cell Mol Neurobiol 2004: Madry et al. Clin Orthop Relat Res 2007. Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003. Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R47H loss-of-function mutant transgenic mice (Ulland et al. Cell. 2017. Kang et al. Hum Mol Genet 2018). Although unable to recapitulate the neurological manifestations of PLOSL. TREM2 KO mice show abnormalities in bone ultrastructure (Otero et al. J Immunol 2012). When the TREM2 KO or mutant mice have been crossed onto familial Alzheimer's disease transgenic mouse background such as the 5×FAD amyloidogenic mutation lines, marked phenotypes have been observed (Ulrich et al. Neuron. 2017). These in vivo phenotypes of TREM2 loss-of-function in the CNS include elevated the plaque burden and lower levels of secreted microglial factors SPPI and Osteopontin that are characteristic of the microglial response to amyloid pathology (Ulland et al. Cell. 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios. 5×FAD familial Alzheimer's disease model mice, and Amyotrophic Lateral Sclerosis SOD1 mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5×FAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM2 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases, such as Alzheimer's disease.

### SUMMARY

Provided herein is a compound of Formula I

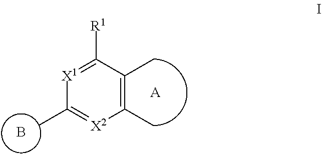

or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

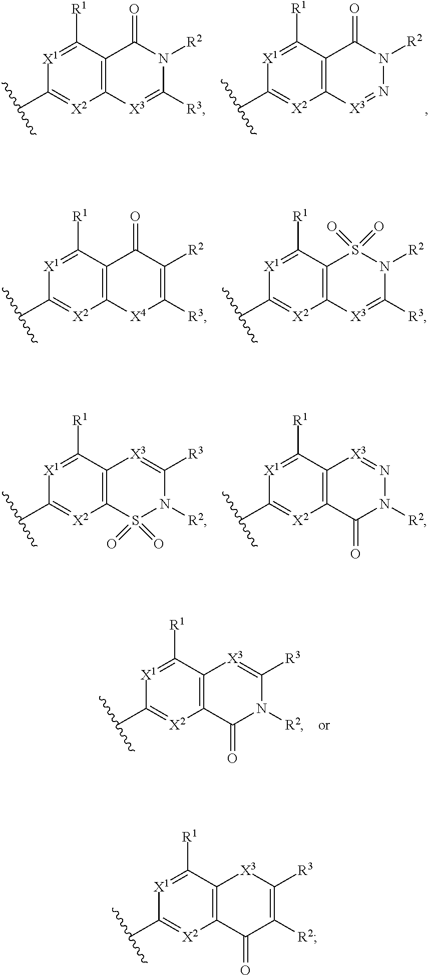

X³ is CR¹⁵, CH or N;
X⁴ is O, NR⁴, C(R⁴)₂, CHR⁴, SO₂, or C═O;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
each R⁴ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
Ring B is

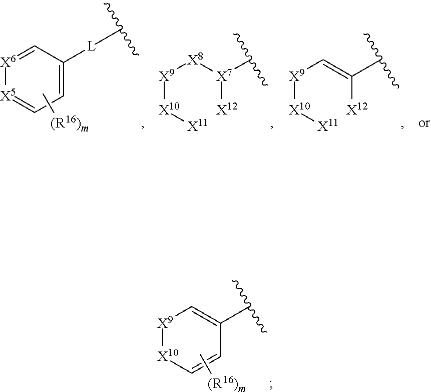

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³, R¹⁴ and R¹⁵ are each independently hydrogen, an optionally substituted C₁₋₆ aliphatic group, halogen, —OR, —CN, —NR₂, —C(═O)R, —C(═O)OR, —C(═O)NR², —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Also provided herein is a pharmaceutical composition comprising a compound of Formula I, or a tautomer thereof, or a pharmaceutically acceptable salt of said compound or said tautomer, and a pharmaceutically acceptable excipient.

Also provided herein is a compound of Formula I, or a tautomer thereof, or a pharmaceutically acceptable salt of said compound or said tautomer, or a pharmaceutical composition as described hereinabove, for use in treating or preventing a condition associated with a loss of function of human TREM2.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

In one aspect, provided herein is a compound of Formula I

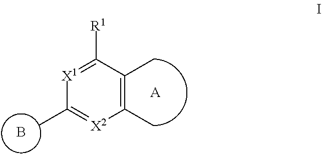

or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

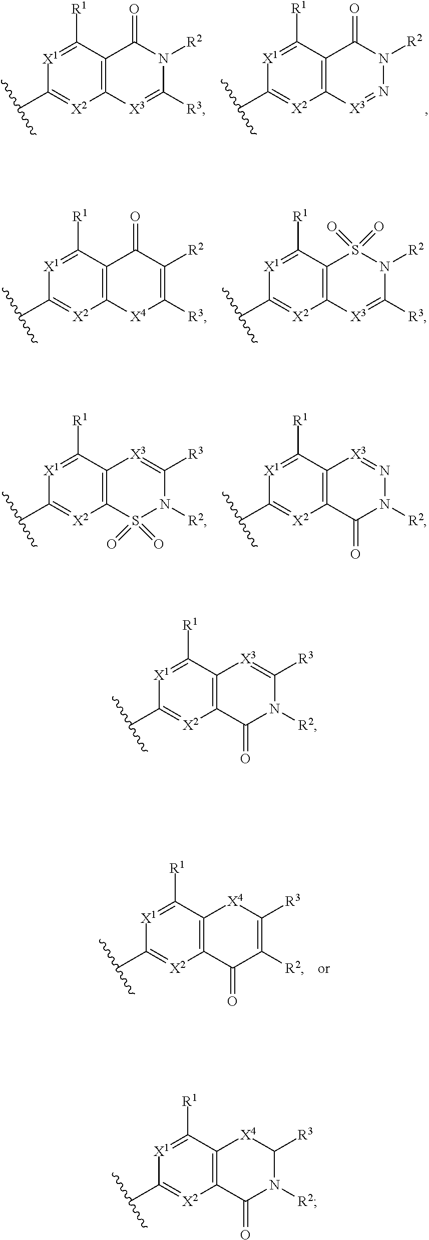

X³ is CR¹⁵, CH or N;
X⁴ is O, NR⁴, C(R⁴)₂, CHR⁴, SO₂, or C═O;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
each R⁴ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
Ring B is

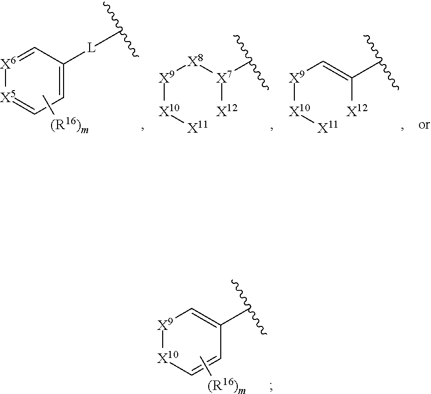

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³, R¹⁴ and R¹⁵ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Provided herein is a compound of Formula I

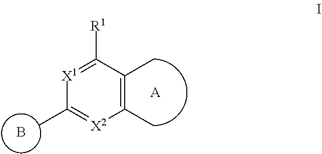

or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula

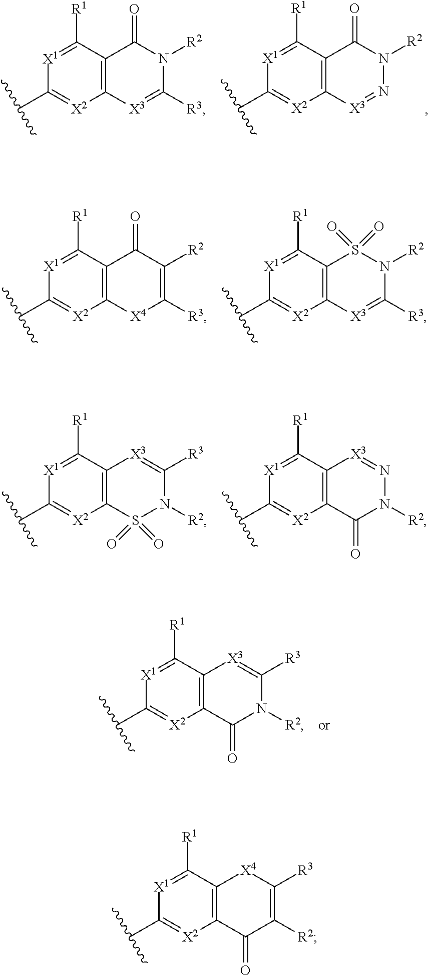

X³ is CR¹⁵, CH or N;
X⁴ is O, NR⁴, C(R⁴)₂, CHR⁴, SO₂, or C═O;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
each R⁴ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
Ring B is

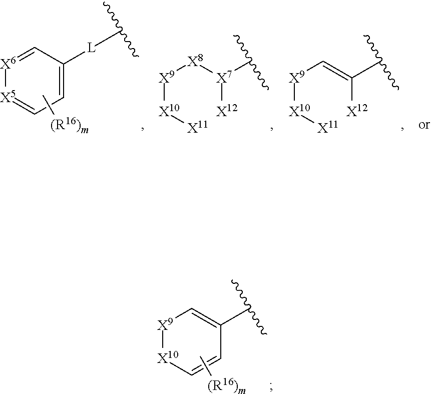

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹º, SO₂, or C═O;
X¹¹ is O, NR¹¹. C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸. R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³, R¹⁴ and R¹⁵ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

In some embodiments, the compound is a compound of Formula IIa:

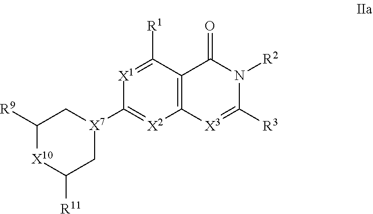

or a tautomer thereof, or a pharmaceutically acceptable salt of said compound or said tautomer, wherein each variable is as defined above and described in embodiments herein both singly and in combination.

In some embodiments, the compound is a compound of Formula IIb:

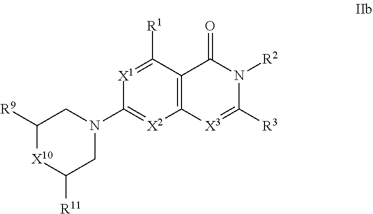

or a tautomer thereof, or a pharmaceutically acceptable salt of said compound or said tautomer, wherein each variable is as defined above and described in embodiments herein both singly and in combination.

In some embodiments, the compound is a compound of Formula IIb′:

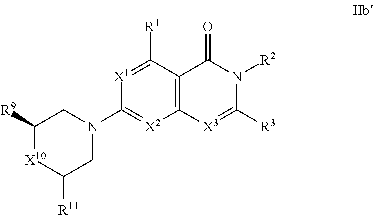

or a tautomer thereof, or a pharmaceutically acceptable salt of said compound or said tautomer, wherein each variable is as defined above and described in embodiments herein both singly and in combination.

In some embodiments, the compound is a compound of Formula IIb″:

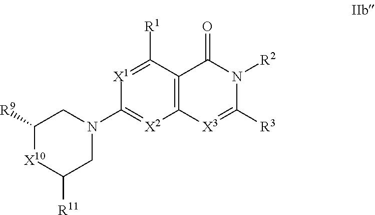

or a tautomer thereof, or a pharmaceutically acceptable salt of said compound or said tautomer, wherein each variable is as defined above and described in embodiments herein both singly and in combination.

In some embodiments, the compound is a compound of Formula IIc:

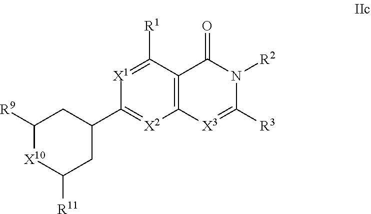

or a tautomer thereof, or a pharmaceutically acceptable salt of said compound or said tautomer, wherein each variable is as defined above and described in embodiments herein both singly and in combination.

In some embodiments, the compound is a compound of Formula IIc′:

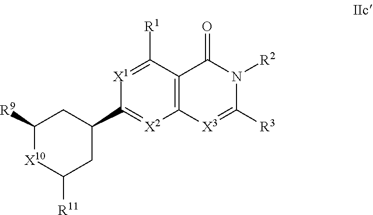

or a tautomer thereof, or a pharmaceutically acceptable salt of said compound or said tautomer, wherein each variable is as defined above and described in embodiments herein both singly and in combination.

In some embodiments, the compound is a compound of Formula IIc″:

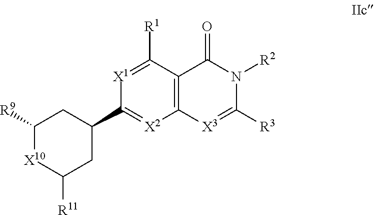

or a tautomer thereof, or a pharmaceutically acceptable salt of said compound or said tautomer, wherein each variable is as defined above and described in embodiments herein both singly and in combination.

In some embodiments, the compound is a compound of Formula IIc″′:

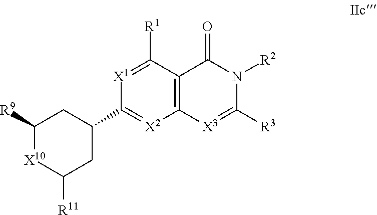

or a tautomer thereof, or a pharmaceutically acceptable salt of said compound or said tautomer, wherein each variable is as defined above and described in embodiments herein both singly and in combination.

In some embodiments, the compound is a compound of Formula IIc″′:

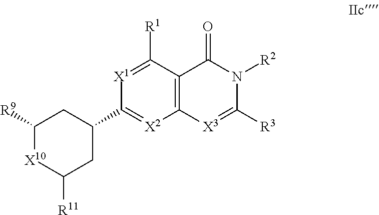

or a tautomer thereof, or a pharmaceutically acceptable salt of said compound or said tautomer, wherein each variable is as defined above and described in embodiments herein both singly and in combination.

In some embodiments, the compound is a compound of Formula IIIa:

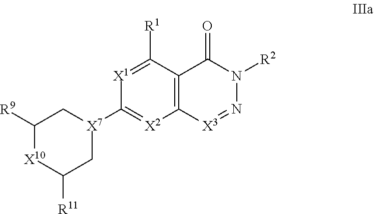

or a tautomer thereof, or a pharmaceutically acceptable salt of said compound or said tautomer, wherein each variable is as defined above and described in embodiments herein both singly and in combination.

In some embodiments, the compound is a compound of Formula IVa:

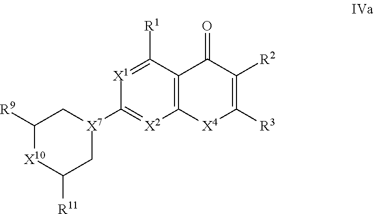

or a tautomer thereof, or a pharmaceutically acceptable salt of said compound or said tautomer, wherein each variable is as defined above and described in embodiments herein both singly and in combination.

In some embodiments, the compound is a compound of Formula Va:

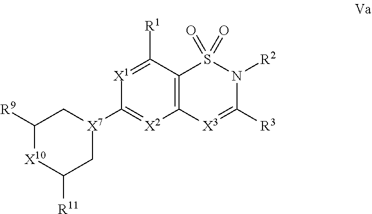

or a tautomer thereof, or a pharmaceutically acceptable salt of said compound or said tautomer, wherein each variable is as defined above and described in embodiments herein both singly and in combination.

In some embodiments, the compound is a compound of Formula VIa:

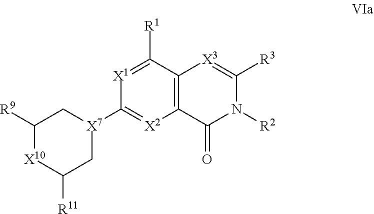

or a tautomer thereof, or a pharmaceutically acceptable salt of said compound or said tautomer, wherein each variable is as defined above and described in embodiments herein both singly and in combination.

In some embodiments, the compound is a compound of Formula VIIa:

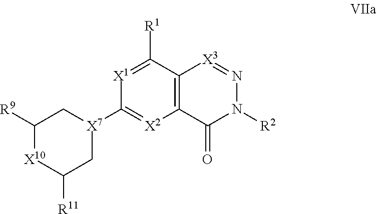

or a tautomer thereof, or a pharmaceutically acceptable salt of said compound or said tautomer, wherein each variable is as defined above and described in embodiments herein both singly and in combination.

In some embodiments, the compound is a compound of Formula VIIIa:

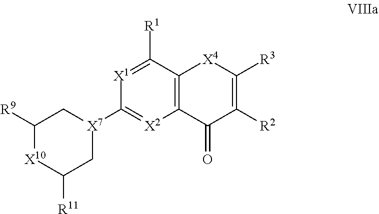

or a tautomer thereof, or a pharmaceutically acceptable salt of said compound or said tautomer, wherein each variable is as defined above and described in embodiments herein both singly and in combination.

In some embodiments, the compound is a compound of Formula IXa:

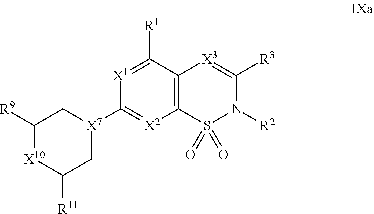

or a tautomer thereof, or a pharmaceutically acceptable salt of said compound or said tautomer, wherein each variable is as defined above and described in embodiments herein both singly and in combination.

In some embodiments, the compound is a compound of Formula VIIa-1 to VIIa-7:

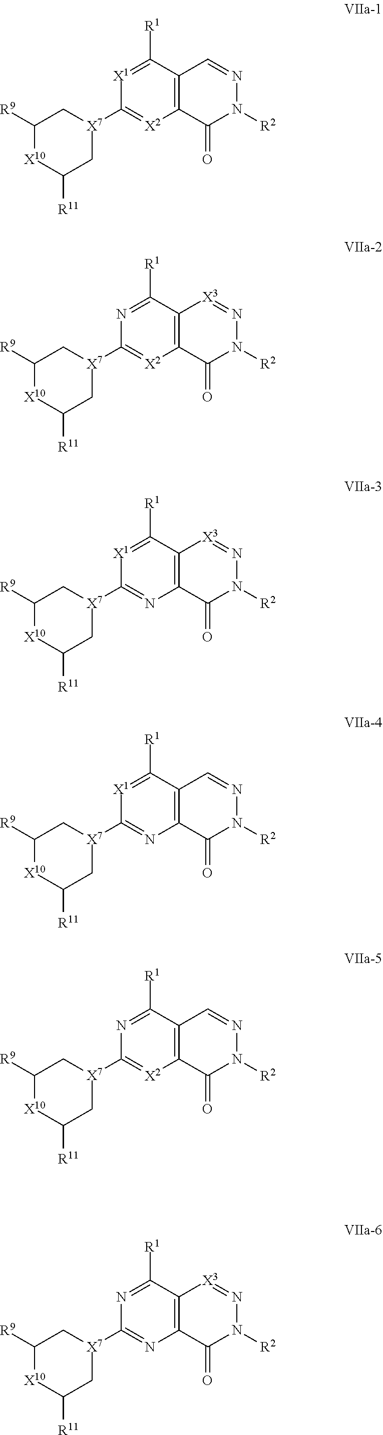

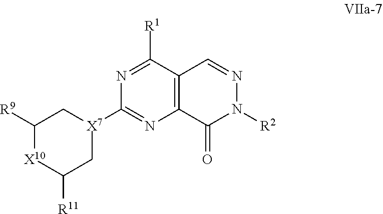

or a tautomer thereof, or a pharmaceutically acceptable salt of said compound or said tautomer, wherein each variable is as defined above and described in embodiments herein both singly and in combination.

In some embodiments, the compound is a compound of Formula VIIb:

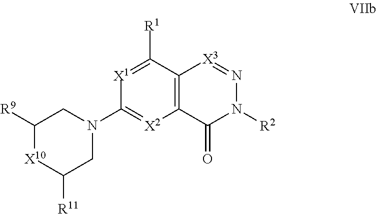

or a tautomer thereof, or a pharmaceutically acceptable salt of said compound or said tautomer, wherein each variable is as defined above and described in embodiments herein both singly and in combination.

In some embodiments, the compound is a compound of Formula VIIb-1 to VIIb-7:

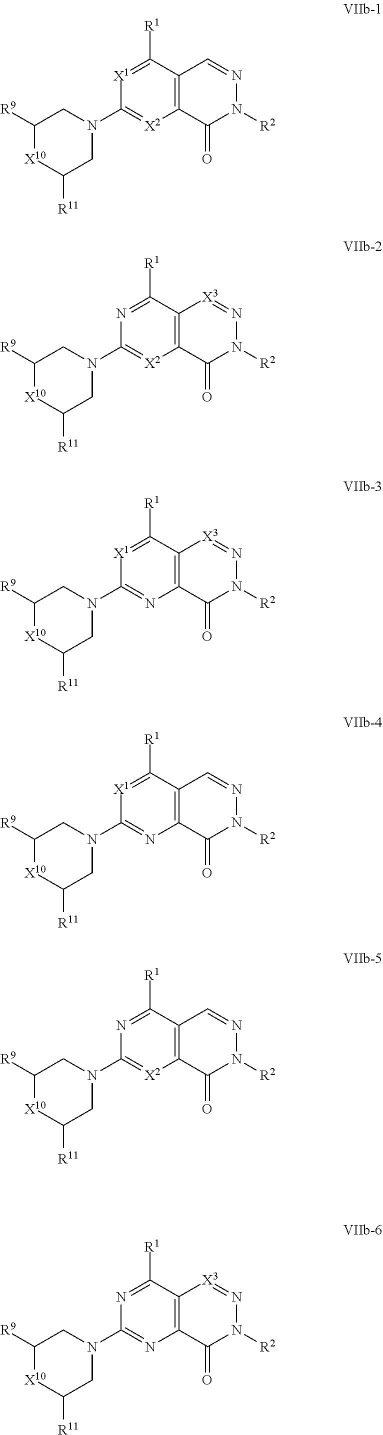

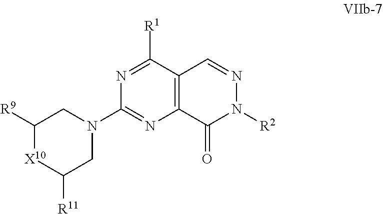

or a tautomer thereof, or a pharmaceutically acceptable salt of said compound or said tautomer, wherein each variable is as defined above and described in embodiments herein both singly and in combination.

In some embodiments, the compound is a compound of Formula VIIc:

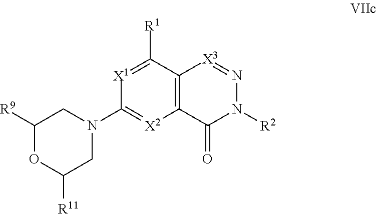

or a tautomer thereof, or a pharmaceutically acceptable salt of said compound or said tautomer, wherein each variable is as defined above and described in embodiments herein both singly and in combination.

In some embodiments, the compound is a compound of Formula VIIc-1 to VIIc-7:

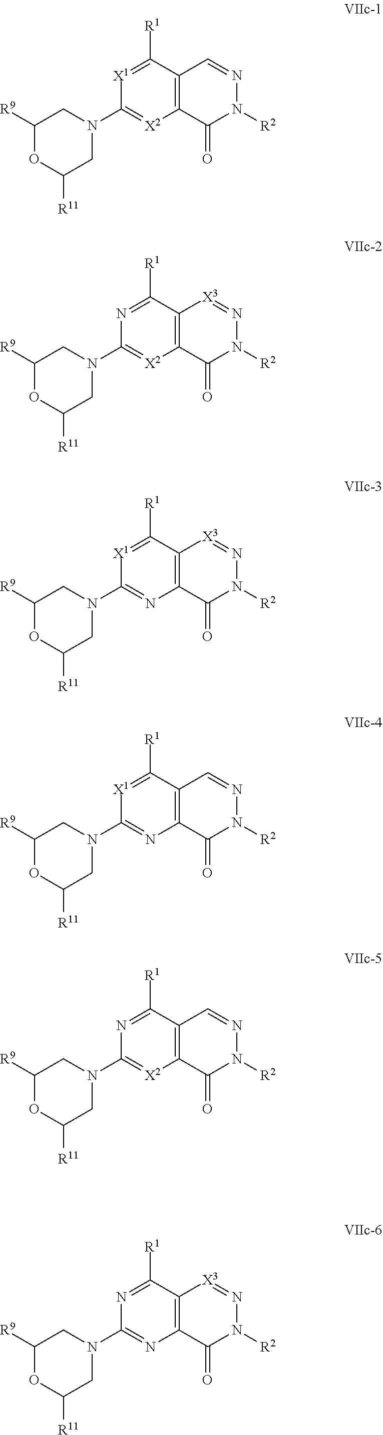

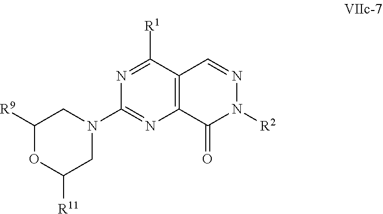

or a tautomer thereof, or a pharmaceutically acceptable salt of said compound or said tautomer, wherein each variable is as defined above and described in embodiments herein both singly and in combination.

In some embodiments, the compound is a compound of Formula VIId:

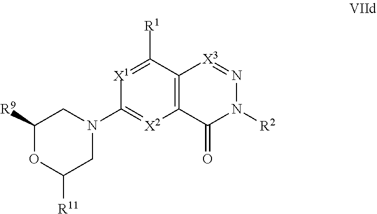

or a tautomer thereof, or a pharmaceutically acceptable salt of said compound or said tautomer, wherein each variable is as defined above and described in embodiments herein both singly and in combination.

In some embodiments, the compound is a compound of Formula VIId-1 to VIId-7:

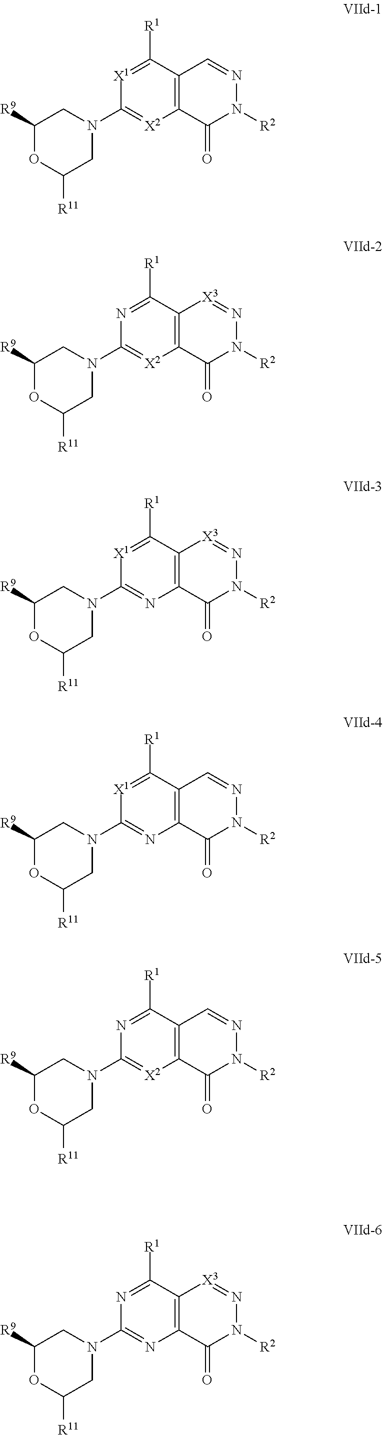

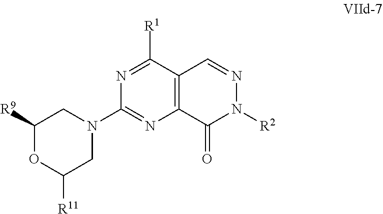

or a tautomer thereof, or a pharmaceutically acceptable salt of said compound or said tautomer, wherein each variable is as defined above and described in embodiments herein both singly and in combination.

In some embodiments, the compound is not
5-(5-chloro-3-methyl-2-pyridinyl)-2,3-dimethyl-7-(2-(1-methyl-1H-pyrazol-4-yl)-4-morpholinyl)pyrido[4,3-d]pyrimidin-4 (3H)-one;
5-(4-chloro-2-fluorophenyl)-2,3-dimethyl-7-(3-methyl-3-phenyl-1-piperidinyl)pyrido[4,3-d]pyrimidin-4 (3H)-one; or
5-(4-chloro-2-fluorophenyl)-2,3-dimethyl-7-(3-(1-methyl-1H-imidazol-2-yl)-1-pyrrolidinyl)pyrido[4,3-d]pyrimidin-4 (3H)-one

As defined generally above, R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹ is an optionally substituted C₁₋₆ aliphatic group. In some embodiments. R¹ is —OR. In some embodiments, R¹ is —NR². In some embodiments, R¹ is —C(═O)R. In some embodiments. R¹ is —C(═O)OR. In some embodiments, R¹ is —C(═O)NR₂. In some embodiments, R² is-SO₂R. In some embodiments, R¹ is-SO₂NR₂. In some embodiments, R¹ is C₁₋₆haloalkyl. In some embodiments. R¹ is an optionally substituted OCH₂—(C₃₋₆cycloalkyl). In some embodiments, R¹ is an optionally substituted 3-8 membered saturated or partially unsaturated monocyclic carbocyclic ring. In some embodiments. R¹ is an optionally substituted 5-12 membered saturated or partially unsaturated bridged carbocyclic ring. In some embodiments, R¹ is an optionally substituted 7-12 membered saturated or partially unsaturated bicyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 6-12 membered saturated or partially unsaturated bridged heterocyclic ring (having 1-4 heteroatoms independently selected from nitrogen, oxygen, and sulfur). In some embodiments, R¹ is an optionally substituted 7-12 membered saturated or partially unsaturated bicyclic heterocyclic ring (having 1-4 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur).

In some embodiments, R¹ is a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹ is phenyl, optionally substituted with 1-3 substituents independently selected from halogen, C₁₋₆ aliphatic, —ORº, or C₁₋₆haloalkyl. In some embodiments, R¹ is phenyl, optionally substituted with 1-3 halogen. In some embodiments, R¹ is a 5-12 membered saturated or partially unsaturated bridged carbocyclic ring, optionally substituted with 1-3 substituents independently selected from halogen, C₁₋₆ aliphatic, —ORº, or C₁₋₆haloalkyl. In some embodiments, R¹ is a C₅₋₈tricycloalkyl ring, optionally substituted with 1-3 substituents independently selected from halogen, C₁₋₆ aliphatic, —ORº, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 substituents independently selected from halogen, C₁₋₆ aliphatic, —ORº, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 halogen.

In some embodiments, R¹ is optionally substituted C₃₋₆cycloalkyl, optionally substituted spiro[3.3]heptanyl, optionally substituted spiro[5.2]octanyl, optionally substituted

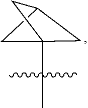 [SMILES: *C12CC(C1)C2]

optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted-OCH₂—(C₃₋₄cycloalkyl). In some embodiments, R¹ is optionally substituted C₃₋₆cycloalkyl. In some embodiments, R¹ is optionally substituted spiro[3.3]heptanyl. In some embodiments, R¹ is optionally substituted spiro[5.2]octanyl. In some embodiments, R¹ is optionally substituted

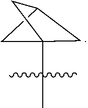 [SMILES: *C12CC(C1)C2]

In some embodiments, R¹ is optionally substituted cyclopent-1-en-1-yl. In some embodiments, R¹ is optionally substituted cyclohex-1-en-1-yl. In some embodiments, R¹ is optionally substituted phenyl. In some embodiments, R¹ is optionally substituted pyridinyl. In some embodiments, R¹ is optionally substituted aziridine-1-yl. In some embodiments, R¹ is optionally substituted pyrrolidine-1-yl. In some embodiments, R¹ is optionally substituted azabicyclo[3.1.0]hexan-3-yl. In some embodiments, R¹ is optionally substituted piperidine-1-yl. In some embodiments, R¹ is optionally substituted-OCH₂—(C₃₋₄cycloalkyl).

In some embodiments, R¹ is optionally substituted with 1-3 groups that are independently halogen; —(CH₂)₀₋₆Rº; —(CH₂)₀₋₆ORº; —O(CH₂)₀₋₆Rº, —O—(CH₂)₀₋₆C(O)ORº; —(CH₂)₀₋₆CH(ORº)₂; —(CH₂)₀₋₆SRº; —(CH₂)₀₋₆Ph, which Ph may be substituted with Rº; —(CH₂)₀₋₆O(CH₂)₀₋₁Ph which Ph may be substituted with Rº; —CH═CHPh, which Ph may be substituted with Rº; —(CH₂)₀₋₆₀ (CH₂)₀₋₁-pyridyl which pyridyl may be substituted with Rº; —NO₂; —CN; —N₃; —(CH₂)₀₋₆N(Rº)₂; —(CH₂)₀₋₆N(Rº)C(O) Rº; —N(Rº)C(S)Rº; —(CH₂)₀₋₆N(Rº)C(O)NRº₂; —N(Rº)C(S)NRº₂; —(CH₂)₀₋₆N(Rº)C(O)ORº; —N(Rº))N(Rº)C(O)Rº; —N(Rº))N(Rº)C(O)NRº₂; —N(Rº))N(Rº)C(O)ORº; —(CH₂)₀₋₆C(O)Rº; —C(S)Rº; —(CH₂)₀₋₆C(O)ORº; —(CH₂)₀₋₆C(O)SRº; —(CH₂)₀₋₆C(O)OSiRº₃; —(CH₂)₀₋₆OC(O)Rº; —OC(O) (CH₂)₀₋₆SRº, —(CH₂)₀₋₆SC(O)Rº; —(CH₂)₀₋₆C(O)NRº₂; —C(S)NRº₂; —C(S)SRº; —SC(S)SRº, —(CH₂)₀₋₆OC(O)NRº₂; —C(O)N(ORº) Rº; —C(O) C(O)Rº; —C(O) CH₂C(O)Rº; —C(NORº) Rº; —(CH₂)₀₋₆SSRº; —(CH₂)₀₋₆S(O)₂Rº; —(CH)₀₋₆S(O)ORº; —(CH)OS(O)₂Rº; —S(O)₂NRº₂; —(CH₂)₀₋₆S(O) Rº; —N(Rº)S(O)₂NRº₂; —N(Rº)S(O)₂Rº; —N(ORº) Rº; —C(NH) NRº₂; —P(O) ¿Rº; —P(O) Rº)₂; —P(O)(ORº)₂; —OP(O) (RºORº; —OP(O) Rº)₂; —OP(O)(ORº)₂; SiRº₃; —(C₁₋₄ straight or branched alkylene)O—N(Rº)₂; or —(C₁₋₄ straight or branched alkylene) C(O)O—N(Rº)₂, wherein each Rº may be substituted as defined elsewhere herein and is independently hydrogen, C₁₋₆ aliphatic, —CH₂Ph, —O(CH₂)₀₋₁Ph, —CH₂—(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of Rº, taken together with their intervening atom(s), form a 3- to 12-membered saturated, partially unsaturated, or aryl mono- or bicyclic ring (having 0 to 4 heteroatoms independently selected from nitrogen, oxygen, and sulfur). In some embodiments, R¹ is optionally substituted with one or more-SFs groups.

In some embodiments, R¹ is —CH₂CH₂CF₃,

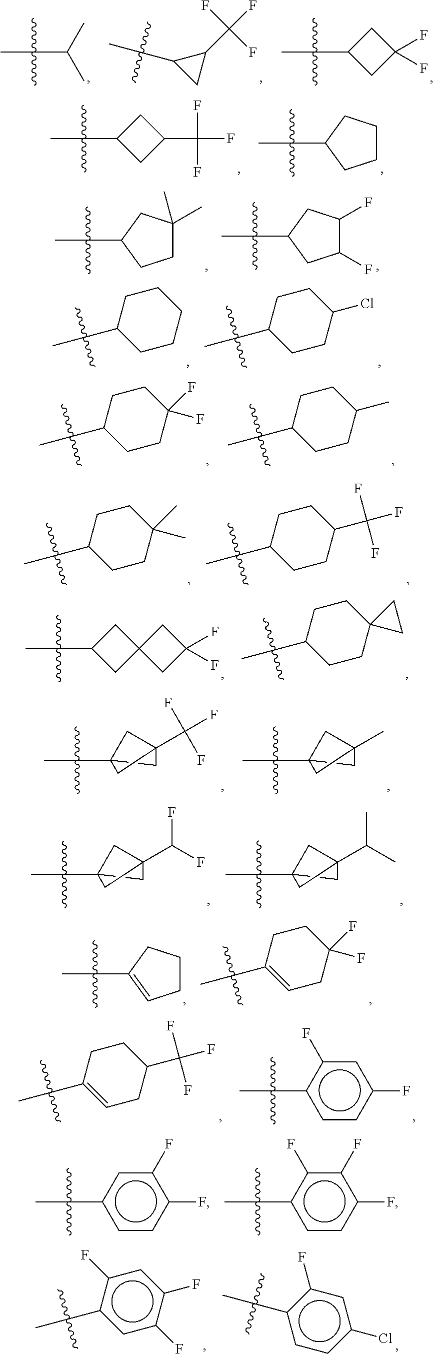

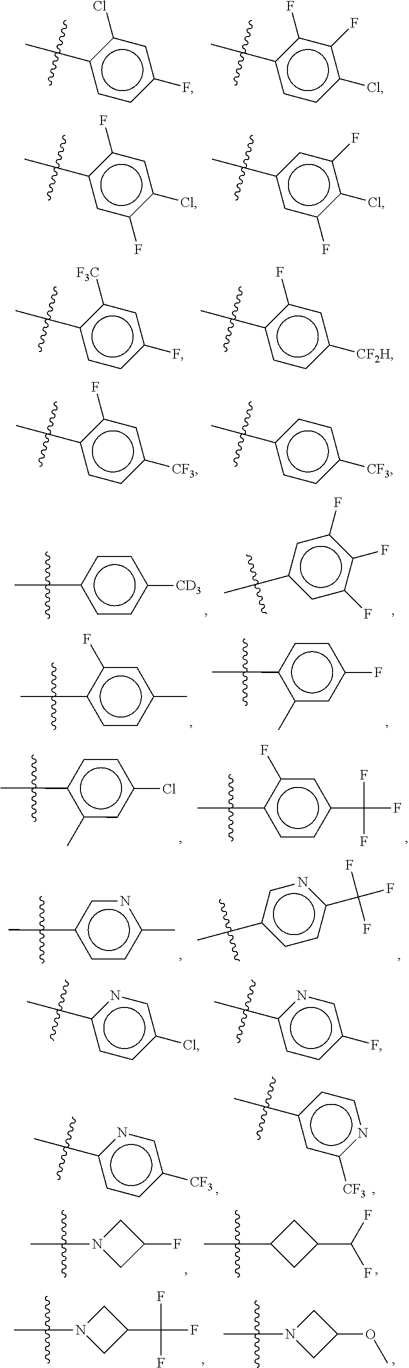

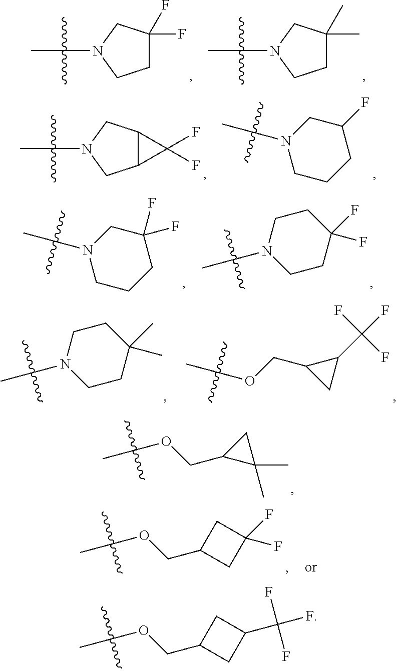

In some embodiments, R¹ is

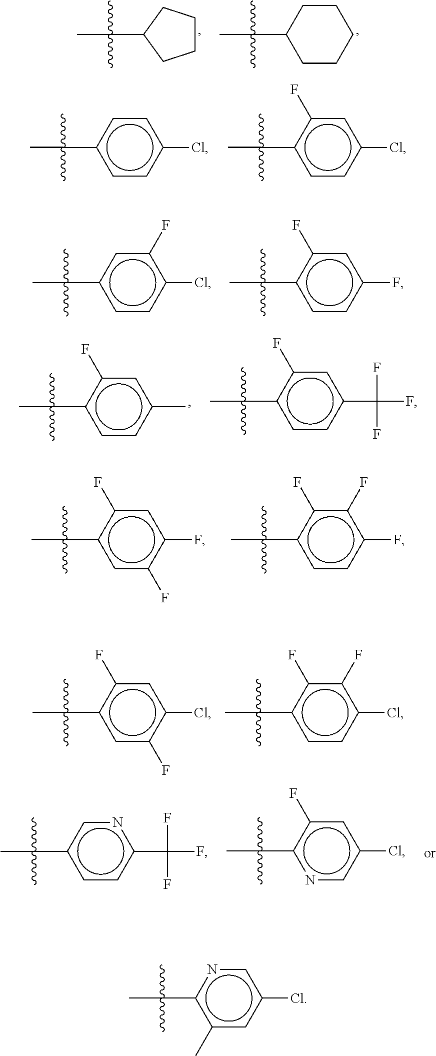

In some embodiments, R¹ is a substituent selected from those shown below:

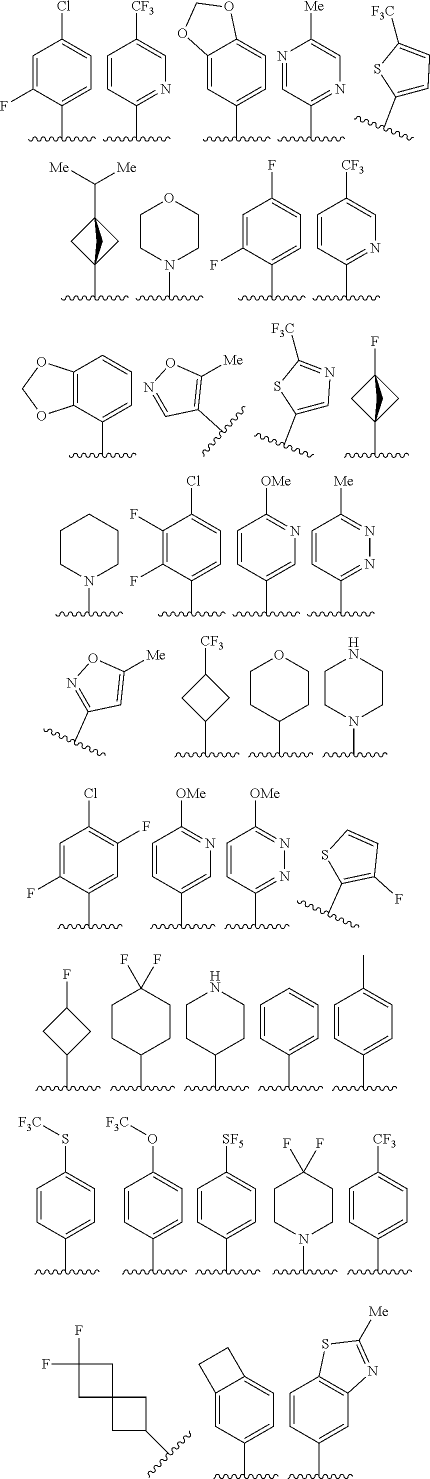

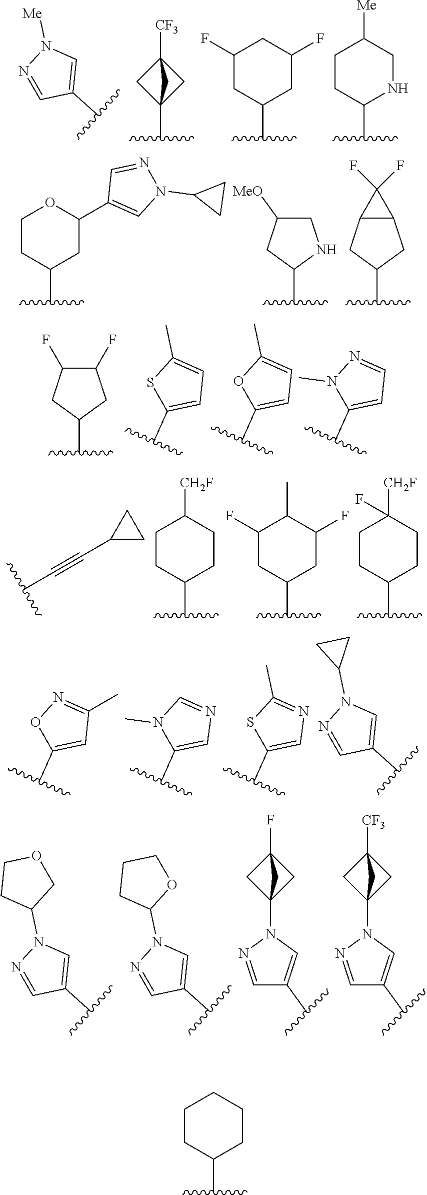

In some embodiments, R¹ is

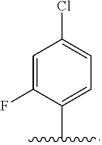 [SMILES: *c1ccc(Cl)cc1F]

In some embodiments, R¹ is

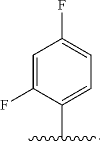 [SMILES: *c1ccc(F)cc1F]

In some embodiments, R¹ is

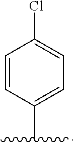 [SMILES: *c1ccc(Cl)cc1]

In some embodiments, R¹ is

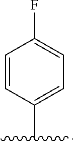 [SMILES: *c1ccc(F)cc1]

In some embodiments, R¹ is selected from those depicted in Table A below. In some embodiments, R¹ is selected from those depicted in Table A-2 below.

As defined generally above, X¹ is CR¹³, CH or N. In some embodiments, X¹ is CH or N. In some embodiments, X¹ is CH. In some embodiments, X¹ is CR¹³. In some embodiments, X¹ is N. In some embodiments, X¹ is selected from those depicted in Table A below. In some embodiments, X¹ is selected from those depicted in Table A-2 below.

As defined generally above, X² is CR¹⁴, CH or N. In some embodiments, X² is CH or N. In some embodiments, X² is CH. In some embodiments, X² is CR¹⁴. In some embodiments, X² is N. In some embodiments, X² is selected from those depicted in Table A below. In some embodiments, X² is selected from those depicted in Table A-2 below.

As defined generally above, Ring A together with the 6-membered ring system to which it is fused forms a bicyclic ring system of formula

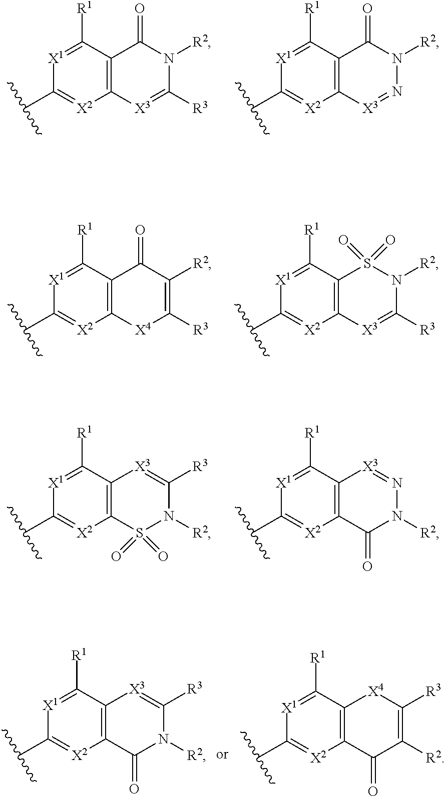

In some embodiments, Ring A together with the 6-membered ring system to which it is fused forms a bicyclic ring system of formula

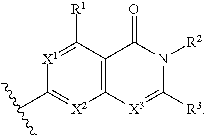

In some embodiments, Ring A together with the 6-membered ring system to which it is fused forms a bicyclic ring system of formula

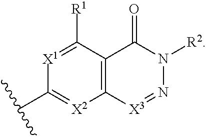

In some embodiments, Ring A together with the 6-membered ring system to which it is fused forms a bicyclic ring system of formula

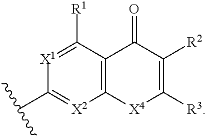

In some embodiments, Ring A together with the 6-membered ring system to which it is fused forms a bicyclic ring system of formula

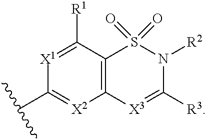

In some embodiments, Ring A together with the 6-membered ring system to which it is fused forms a bicyclic ring system of formula

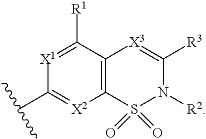

In some embodiments, Ring A together with the 6-membered ring system to which it is fused forms a bicyclic ring system of formula

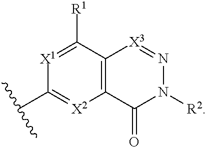

In some embodiments, Ring A together with the 6-membered ring system to which it is fused forms a bicyclic ring system of formula

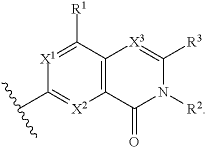

In some embodiments, Ring A together with the 6-membered ring system to which it is fused forms a bicyclic ring system of formula

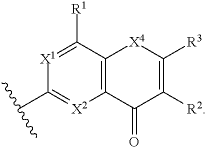

In some embodiments, Ring A together with the 6-membered ring system to which it is fused forms a bicyclic ring system of formula

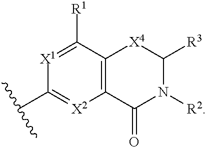

In some embodiments, Ring A together with the 6-membered ring system to which it is fused forms a bicyclic ring system of formula selected from:

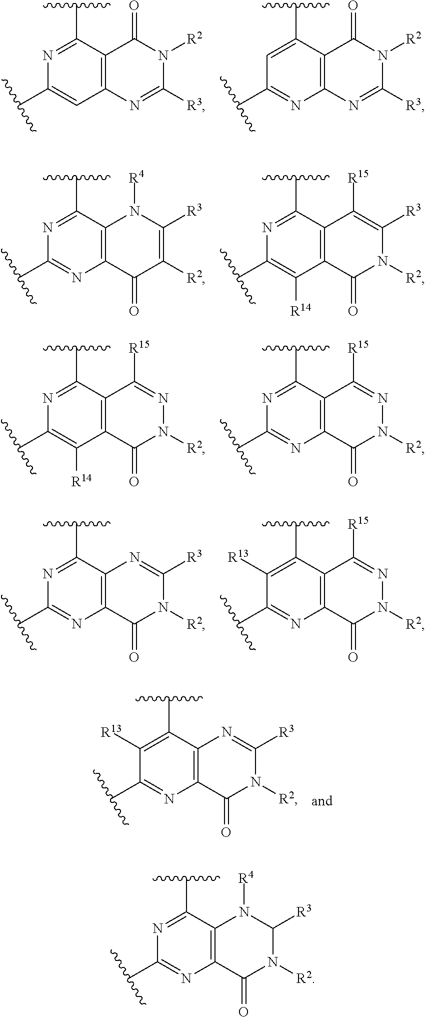

In some embodiments, Ring A together with the 6-membered ring system to which it is fused forms a bicyclic ring system selected from those depicted in Table A below. In some embodiments, Ring A together with the 6-membered ring system to which it is fused forms a bicyclic ring system selected from those depicted in Table A-2 below.

As defined generally above, R² and R³ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted, provided that at least one of R² and R³ is not hydrogen; or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R² is an optionally substituted C₁₋₆ aliphatic group. In some embodiments. R² is halogen. In some embodiments, R² is —OR. In some embodiments, R² is —NR². In some embodiments. R² is —C(═O)R. In some embodiments, R² is —C(═O)OR. In some embodiments, R² is —C(═O)NR₂. In some embodiments, R² is-SO₂R. In some embodiments, R² is-SO₂NR₂. In some embodiments. R² is C₁₋₆haloalkyl. In some embodiments, R² is C₁₋₆haloalkoxy. In some embodiments, R² is an optionally substituted 3-8 membered saturated or partially unsaturated monocyclic carbocyclic ring. In some embodiments, R² is an optionally substituted 6-12 membered saturated or partially unsaturated bridged carbocyclic ring. In some embodiments, R² is an optionally substituted 7-12 membered saturated or partially unsaturated bicyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 3-8 membered saturated or partially unsaturated monocyclic heterocyclic ring (having 1-2 heteroatoms independently selected from nitrogen, oxygen, and sulfur). In some embodiments, R² is an optionally substituted 6-12 membered saturated or partially unsaturated bridged heterocyclic ring (having 1-4 heteroatoms independently selected from nitrogen, oxygen, and sulfur). In some embodiments, R² is an optionally substituted 7-12 membered saturated or partially unsaturated bicyclic heterocyclic ring (having 1-4 heteroatoms independently selected from nitrogen, oxygen, and sulfur). In some embodiments, R² is an optionally substituted 5-6 membered monocyclic heteroaromatic ring (having 1-4 heteroatoms independently selected from nitrogen, oxygen, and sulfur). In some embodiments, R² is an optionally substituted 8-10 membered bicyclic heteroaromatic ring (having 1-5 heteroatoms independently selected from nitrogen, oxygen, and sulfur). In some embodiments. R² is selected from those depicted in Table A below. In some embodiments, R² is selected from those depicted in Table A-2 below.

In some embodiments, R³ is an optionally substituted C₁₋₆ aliphatic group. In some embodiments. R³ is halogen. In some embodiments, R³ is —OR. In some embodiments, R³ is —NR². In some embodiments. R³ is —C(═O)R. In some embodiments, R³ is —C(═O)OR. In some embodiments, R³ is —C(═O)NR₂. In some embodiments, R³ is-SO₂R. In some embodiments, R³ is-SO₂NR₂. In some embodiments. R³ is C₁₋₆haloalkyl. In some embodiments, R³ is C₁₋₆haloalkoxy. In some embodiments, R³ is an optionally substituted 3-8 membered saturated or partially unsaturated monocyclic carbocyclic ring. In some embodiments, R³ is an optionally substituted 6-12 membered saturated or partially unsaturated bridged carbocyclic ring. In some embodiments, R³ is an optionally substituted 7-12 membered saturated or partially unsaturated bicyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 3-8 membered saturated or partially unsaturated monocyclic heterocyclic ring (having 1-2 heteroatoms independently selected from nitrogen, oxygen, and sulfur). In some embodiments, R³ is an optionally substituted 6-12 membered saturated or partially unsaturated bridged heterocyclic ring (having 1-4 heteroatoms independently selected from nitrogen, oxygen, and sulfur). In some embodiments, R³ is an optionally substituted 7-12 membered saturated or partially unsaturated bicyclic heterocyclic ring (having 1-4 heteroatoms independently selected from nitrogen, oxygen, and sulfur). In some embodiments, R³ is an optionally substituted 5-6 membered monocyclic heteroaromatic ring (having 1-4 heteroatoms independently selected from nitrogen, oxygen, and sulfur). In some embodiments, R³ is an optionally substituted 8-10 membered bicyclic heteroaromatic ring (having 1-5 heteroatoms independently selected from nitrogen, oxygen, and sulfur). In some embodiments, R³ is selected from those depicted in Table A below. In some embodiments, R³ is selected from those depicted in Table A-2 below.

In some embodiments, R² is hydrogen. In some embodiments, R² is methyl. In some embodiments, R² is Cl. In some embodiments, R² is a C₁₋₃ haloalkyl. In some embodiments, R² is 3-8 membered saturated monocyclic heterocyclic ring (having 1-2 heteroatoms independently selected from nitrogen, oxygen, and sulfur). In some embodiments, R² is an azetidinyl group. In some embodiments, R² is optionally substituted ethyl. In some embodiments, R² is methoxy. In some embodiments, R² is —CH₂F. In some embodiments, R² is —OCH₂F. In some embodiments, R² is —CD₃.

In some embodiments, R³ is hydrogen. In some embodiments, R³ is methyl. In some embodiments. R³ is Cl. In some embodiments, R³ is —CD₃.

In some embodiments, R² is H and R³ is methyl. In some embodiments, R² is methyl and R³ is methyl. In some embodiments, R² is C₁ and R³ is Cl.

In some embodiments, R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R² and R³ are taken together with their intervening atoms to form an optionally substituted 3-8 membered saturated or partially unsaturated monocyclic carbocyclic ring. In some embodiments, R² and R³ are taken together with their intervening atoms to form an optionally substituted 6-12 membered saturated or partially unsaturated bridged carbocyclic ring. In some embodiments, R² and R³ are taken together with their intervening atoms to form an optionally substituted 7-12 membered saturated or partially unsaturated bicyclic carbocyclic ring. In some embodiments, R² and R³ are taken together with their intervening atoms to form an optionally substituted phenyl. In some embodiments. R² and R³ are taken together with their intervening atoms to form an optionally substituted 8-10 membered bicyclic aromatic carbocyclic ring. In some embodiments, R² and R³ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R² and R³ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R² and R³ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R² and R³ are taken together with their intervening atoms to form an optionally substituted 5-6 membered monocyclic heteroaromatic ring (having 1-4 heteroatoms independently selected from nitrogen, oxygen, and sulfur). In some embodiments, R² and R³ are taken together with their intervening atoms to form an optionally substituted 8-10 membered bicyclic heteroaromatic ring (having 1-5 heteroatoms independently selected from nitrogen, oxygen, and sulfur).

In some embodiments, R² and R³ are taken together with their intervening atoms to form a dioxole ring.

As defined generally above, X³ is CR¹⁵, CH or N. In some embodiments, X³ is CH or N. In some embodiments. X³ is CH. In some embodiments, X³ is CR¹⁵. In some embodiments, X³ is N. In some embodiments. X³ is selected from those depicted in Table A below. In some embodiments, X³ is selected from those depicted in Table A-2 below.

As defined generally above, X⁴ is O, NR⁴, C(R⁴) 2, CHR⁴, SO₂, or C═O. In some embodiments, X⁴ is O. In some embodiments, X⁴ is NR⁴. In some embodiments, X⁴ is NH. In some embodiments, X⁴ is NMe. In some embodiments. X⁴ is C(R⁴)₂. In some embodiments. X⁴ is CHR⁴. In some embodiments, X⁴ is CH₂. In some embodiments, X⁴ is SO₂. In some embodiments, X⁴ is C═O. In some embodiments, X⁴ is CH₂ or NH.

As defined generally above, each R⁴ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R⁴ is hydrogen. In some embodiments, R⁴ is an optionally substituted C₁₋₆ aliphatic group. In some embodiments, R⁴ is halogen. In some embodiments, R⁴ is —OR. In some embodiments, R⁴ is-CN. In some embodiments, R⁴ is —NR². In some embodiments, R⁴ is —C(═O) R. In some embodiments, R⁴ is —C(═O)OR. In some embodiments, R⁴ is —C(═O)NR₂. In some embodiments, R⁴ is-SO₂R. In some embodiments, R⁴ is-SO₂NR₂. In some embodiments, R⁴ is C₁₋₆-haloalkyl. In some embodiments, R⁴ is C₁₋₆haloalkoxy. In some embodiments, R⁴ is methyl.

As defined generally above, each R¹³, R¹⁴, and R¹⁵ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy.

In some embodiments, R¹³ is hydrogen. In some embodiments, R¹³ is an optionally substituted C₁₋₆ aliphatic group. In some embodiments, R¹³ is halogen. In some embodiments, R¹³ is —OR. In some embodiments, R¹³ is-CN. In some embodiments, R¹³ is —NR². In some embodiments, R¹³ is —C(═O)R. In some embodiments, R¹³ is —C(═O)OR. In some embodiments, R¹³ is —C(═O)NR₂. In some embodiments, R¹³ is-SO₂R. In some embodiments, R¹³ is-SO₂NR₂. In some embodiments, R¹³ is C₁₋₆haloalkyl. In some embodiments, R¹³ is C₁₋₆haloalkoxy. In some embodiments, R¹³ is methyl.

In some embodiments, R¹⁴ is hydrogen. In some embodiments, R¹⁴ is an optionally substituted C₁₋₆ aliphatic group. In some embodiments, R¹⁴ is halogen. In some embodiments, R¹⁴ is —OR. In some embodiments, R¹⁴ is-CN. In some embodiments, R¹⁴ is —NR². In some embodiments, R¹⁴ is —C(═O)R. In some embodiments, R¹⁴ is —C(═O)OR. In some embodiments, R¹⁴ is —C(═O)NR₂. In some embodiments, R¹⁴ is-SO₂R. In some embodiments, R¹⁴ is-SO₂NR₂. In some embodiments, R¹⁴ is C₁₋₆haloalkyl. In some embodiments, R¹⁴ is C₁₋₆haloalkoxy. In some embodiments, R¹⁴ is methyl.

In some embodiments, R¹⁵ is hydrogen. In some embodiments, R¹⁵ is an optionally substituted C₁₋₆ aliphatic group. In some embodiments, R¹⁵ is halogen. In some embodiments, R¹⁵ is —OR. In some embodiments, R¹⁵ is-CN. In some embodiments, R¹⁵ is —NR². In some embodiments, R¹⁵ is —C(═O)R. In some embodiments, R¹⁵ is —C(═O)OR. In some embodiments, R¹⁵ is —C(═O)NR₂. In some embodiments, R¹⁵ is-SO₂R. In some embodiments, R¹⁵ is-SO₂NR₂. In some embodiments, R¹⁵ is C₁₋₆haloalkyl. In some embodiments, R¹⁵ is C₁₋₆haloalkoxy. In some embodiments, R¹⁵ is methyl.

As defined generally above, Ring B is

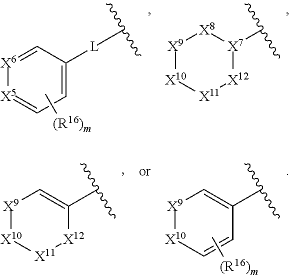

In some embodiments, Ring B is

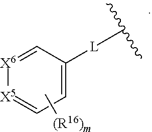

In some embodiments, Ring B is

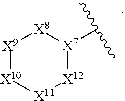

In some embodiments, Ring B is

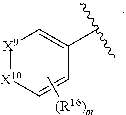

In some embodiments, Ring B is

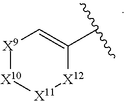

As defined generally above, L is a bond or an optionally substituted straight chain or branched C₁₋₆ alkylene. In some embodiments, L is a bond. In some embodiments, L is an optionally substituted straight chain or branched C₁₋₆ alkylene. In some embodiments, L is optionally substituted ethylene. In some embodiments, L is optionally substituted methylene. In some embodiments, L is selected from those depicted in Table A below. In some embodiments, L is selected from those depicted in Table A-2 below.

As defined generally above, X⁵ is CH, N or CR⁵. In some embodiments, X⁵ is CH. In some embodiments, X⁵ is N. In some embodiments, X⁵ is CR⁵. In some embodiments, X⁵ is selected from those depicted in Table A below. In some embodiments, X⁵ is selected from those depicted in Table A-2 below.

As defined generally above, X⁶ is CH, N or CR⁶. In some embodiments, X⁶ is CH. In some embodiments, X⁶ is N. In some embodiments, X⁶ is CR⁶. In some embodiments, X⁶ is selected from those depicted in Table A below. In some embodiments, X⁶ is selected from those depicted in Table A-2 below.

In some embodiments, X⁵ is N and X⁶ is CH. In some embodiments, X⁵ is N and X⁶ is CR⁶. In some embodiments, X⁵ is CH and X⁶ is N. In some embodiments, X⁵ is CR⁵ and X⁶ is N. In some embodiments, X⁵ is CH and X⁶ is CH. In some embodiments, X⁵ is CH and X⁶ is CR⁶. In some embodiments, X⁵ is CR⁵ and X⁶ is CH.

As defined generally above, R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R¹⁶ is hydrogen. In some embodiments, R¹⁶ is an optionally substituted C₁₋₆ aliphatic group. In some embodiments, R¹⁶ is halogen. In some embodiments, R¹³ is —OR. In some embodiments, R¹⁶ is —CN. In some embodiments, R¹⁶ is —NR². In some embodiments, R¹⁶ is —C(═O)R. In some embodiments, R¹⁶ is —C(═O)OR. In some embodiments, R¹⁶ is —C(═O)NR₂. In some embodiments, R¹⁶ is-SO₂R. In some embodiments, R¹⁶ is-SO₂NR₂. In some embodiments, R¹⁶ is C₁₋₆haloalkyl. In some embodiments, R¹⁶ is C₁₋₆haloalkoxy. In some embodiments, R¹⁶ is —CD₃. In some embodiments, R¹⁶ is selected from those depicted in Table A below. In some embodiments, R¹⁶ is selected from those depicted in Table A-2 below.

As defined generally above, m is 0, 1 or 2. In some embodiments, m is 0. In some embodiments, m is 1. In some embodiments, m is 2.

In some embodiments, Ring B is

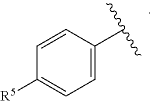

In some embodiments, Ring B is

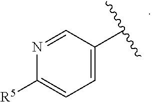

In some embodiments, Ring B is

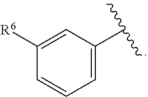

In some embodiments, Ring B is

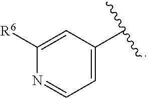

In some embodiments, Ring B is

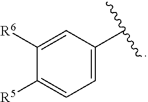

As defined generally above, R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments. R⁵ is an optionally substituted C₁₋₆ aliphatic group. In some embodiments. R⁵ is —OR. In some embodiments, R⁵ is —NR². In some embodiments, R⁵ is —C(═O)R. In some embodiments. R⁵ is —C(═O)OR. In some embodiments, R⁵ is —C(═O)NR₂. In some embodiments, R⁵ is-SO₂R. In some embodiments, R⁵ is-SO₂NR₂. In some embodiments, R⁵ is halogen. In some embodiments. R⁵ is C₁₋₆haloalkyl. In some embodiments. R⁵ is C₁₋₆haloalkoxy. In some embodiments, R⁵ is an optionally substituted 3-8 membered saturated or partially unsaturated monocyclic carbocyclic ring. In some embodiments. R⁵ is an optionally substituted 6-12 membered saturated or partially unsaturated bridged carbocyclic ring. In some embodiments, R⁵ is an optionally substituted 7-12 membered saturated or partially unsaturated bicyclic carbocyclic ring. In some embodiments, R⁵ is an optionally substituted phenyl. In some embodiments. R⁵ is an optionally substituted 8-10 membered bicyclic aromatic carbocyclic ring. In some embodiments. R⁵ is an optionally substituted 3-8 membered saturated or partially unsaturated monocyclic heterocyclic ring (having 1-2 heteroatoms independently selected from nitrogen, oxygen, and sulfur). In some embodiments. R⁵ is an optionally substituted 6-12 membered saturated or partially unsaturated bridged heterocyclic ring (having 1-4 heteroatoms independently selected from nitrogen, oxygen, and sulfur). In some embodiments, R⁵ is an optionally substituted 7-12 membered saturated or partially unsaturated bicyclic heterocyclic ring (having 1-4 heteroatoms independently selected from nitrogen, oxygen, and sulfur). In some embodiments, R⁵ is an optionally substituted 5-6 membered monocyclic heteroaromatic ring (having 1-4 heteroatoms independently selected from nitrogen, oxygen, and sulfur). In some embodiments. R⁵ is an optionally substituted 8-10 membered bicyclic heteroaromatic ring (having 1-5 heteroatoms independently selected from nitrogen, oxygen, and sulfur).

In some embodiments. R⁵ is F. In some embodiments, R⁵ is Cl. In some embodiments. R⁵ is-OCF₃. In some embodiments, R⁵ is cyclopropyl. In some embodiments, R⁵ is selected from those depicted in Table A below. In some embodiments. R⁵ is selected from those depicted in Table A-2 below.

In some embodiments, R⁶ is an optionally substituted C₁₋₆ aliphatic group. In some embodiments. R⁶ is —OR. In some embodiments, R⁶ is —NR². In some embodiments, R⁶ is —C(═O)R. In some embodiments. R⁶ is —C(═O)OR. In some embodiments, R⁶ is —C(═O)NR₂. In some embodiments, R⁶ is-SO₂R. In some embodiments, R⁶ is-SO₂NR₂. In some embodiments, R⁶ is halogen. In some embodiments. R⁶ is C₁₋₆haloalkyl. In some embodiments, R⁶ is C₁₋₆haloalkoxy. In some embodiments, R⁶ is an optionally substituted 3-8 membered saturated or partially unsaturated monocyclic carbocyclic ring. In some embodiments. R⁶ is an optionally substituted 6-12 membered saturated or partially unsaturated bridged carbocyclic ring. In some embodiments, R⁶ is an optionally substituted 7-12 membered saturated or partially unsaturated bicyclic carbocyclic ring. In some embodiments, R⁶ is an optionally substituted phenyl. In some embodiments. R⁶ is an optionally substituted 8-10 membered bicyclic aromatic carbocyclic ring. In some embodiments. R⁶ is an optionally substituted 3-8 membered saturated or partially unsaturated monocyclic heterocyclic ring (having 1-2 heteroatoms independently selected from nitrogen, oxygen, and sulfur). In some embodiments. R⁶ is an optionally substituted 6-12 membered saturated or partially unsaturated bridged heterocyclic ring (having 1-4 heteroatoms independently selected from nitrogen, oxygen, and sulfur). In some embodiments, R⁶ is an optionally substituted 7-12 membered saturated or partially unsaturated bicyclic heterocyclic ring (having 1-4 heteroatoms independently selected from nitrogen, oxygen, and sulfur). In some embodiments, R⁶ is an optionally substituted 5-6 membered monocyclic heteroaromatic ring (having 1-4 heteroatoms independently selected from nitrogen, oxygen, and sulfur). In some embodiments. R⁶ is an optionally substituted 8-10 membered bicyclic heteroaromatic ring (having 1-5 heteroatoms independently selected from nitrogen, oxygen, and sulfur).

In some embodiments, R⁶ is F. In some embodiments, R⁶ is Cl. In some embodiments, R⁶ is-OCF₃. In some embodiments, R⁶ is cyclopropyl. In some embodiments, R⁶ is cyclobutyl. In some embodiments. R⁶ is optionally substituted pyrazolyl. In some embodiments, R⁶ is optionally substituted pyridinyl. In some embodiments, R⁶ is optionally substituted pyrimidinyl. In some embodiments, R⁶ is optionally substituted pyridazinyl. In some embodiments, R⁶ is optionally substituted imidazolyl. In some embodiments. R⁶ is optionally substituted triazolyl. In some embodiments, R⁶ is optionally substituted oxazolyl. In some embodiments, R⁶ is optionally substituted thiazolyl. In some embodiments, R⁶ is optionally substituted oxadiazolyl. In some embodiments, R⁶ is optionally substituted thiadiazolyl. In some embodiments. R⁶ is optionally substituted oxetanyl. In some embodiments, R⁶ is optionally substituted azetidinyl. In some embodiments, R⁶ is optionally substituted piperidinyl. In some embodiments, R⁶ is optionally substituted piperazinyl. In some embodiments, R⁶ is selected from those depicted in Table A below. In some embodiments, R⁶ is selected from those depicted in Table A-2 below.

In some embodiments, R⁵ and R⁶ are independently a substituent selected from hydrogen and those shown below:

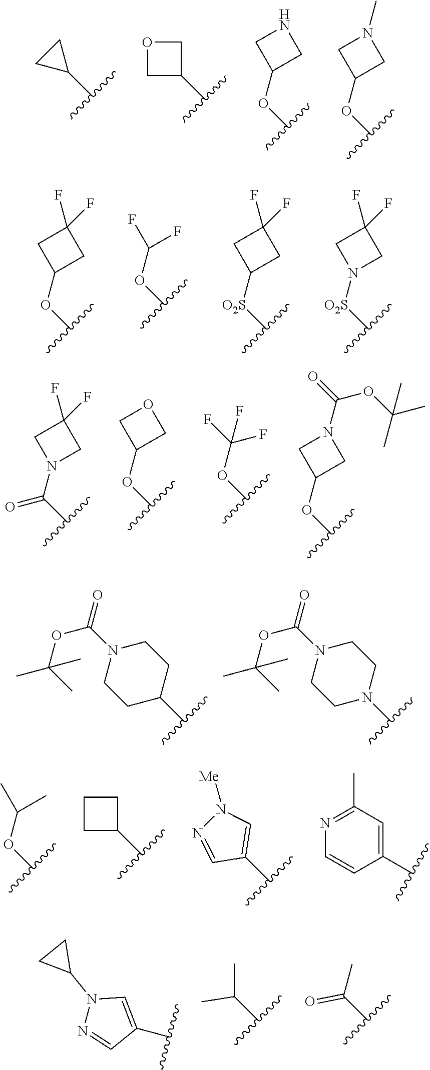

In some embodiments, R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁵ and R⁶ are taken together with their intervening atoms to form an optionally substituted 3-8 membered saturated or partially unsaturated monocyclic carbocyclic ring. In some embodiments, R⁵ and R⁶ are taken together with their intervening atoms to form an optionally substituted 6-12 membered saturated or partially unsaturated bridged carbocyclic ring. In some embodiments. R⁵ and R⁶ are taken together with their intervening atoms to form an optionally substituted 7-12 membered saturated or partially unsaturated bicyclic carbocyclic ring. In some embodiments, R⁵ and R⁶ are taken together with their intervening atoms to form an optionally substituted phenyl. In some embodiments, R⁵ and R⁶ are taken together with their intervening atoms to form an optionally substituted 8-10 membered bicyclic aromatic carbocyclic ring. In some embodiments, R⁵ and R⁶ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R⁵ and R⁶ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R⁵ and R⁶ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R⁵ and R⁶ are taken together with their intervening atoms to form an optionally substituted 5-6 membered monocyclic heteroaromatic ring (having 1-4 heteroatoms independently selected from nitrogen, oxygen, and sulfur). In some embodiments, R⁵ and R⁶ are taken together with their intervening atoms to form an optionally substituted 8-10 membered bicyclic heteroaromatic ring (having 1-5 heteroatoms independently selected from nitrogen, oxygen, and sulfur).

In some embodiments, R⁵ and R⁶ are taken together with their intervening atoms to form a dioxole ring.

As defined generally above, X⁷ is N, CH, or CR⁷. In some embodiments, X⁷ is N. In some embodiments, X⁷ is CH. In some embodiments, X⁷ is CR⁷. In some embodiments, X⁷ is CCH₃. In some embodiments, X⁷ is COH. In some embodiments, X⁷ is CF. In some embodiments, X⁷ is selected from those depicted in Table A below. In some embodiments, X⁷ is selected from those depicted in Table A-2 below.

As defined generally above, X⁸ is O, NR⁸, C(R⁸)₂, CHR⁸, SO₂, or C═O. In some embodiments, X⁸ is O. In some embodiments. X⁸ is NR⁸. In some embodiments, X⁸ is C(R⁸)₂. In some embodiments, X⁸ is CHR⁸. In some embodiments, X⁸ is SO₂. In some embodiments, X⁸ is CH₂. In some embodiments, X⁸ is C═O. In some embodiments, X⁸ is selected from those depicted in Table A below. In some embodiments, X⁸ is selected from those depicted in Table A-2 below.

As defined generally above, X⁹ is O, NR⁹, C(R⁹)₂, CHR⁹, SO₂, or C═O. In some embodiments, X⁹ is O. In some embodiments, X⁹ is NR⁹. In some embodiments, X⁹ is C(R⁹)₂. In some embodiments, X⁹ is CHR⁹. In some embodiments. X⁹ is SO₂. In some embodiments, X⁹ is CH₂. In some embodiments, X⁹ is C═O. In some embodiments, X⁹ is selected from those depicted in Table A below. In some embodiments, X⁹ is selected from those depicted in Table A-2 below.

As defined generally above, X¹⁰ is O, NR¹⁰, C(R¹⁰)₂, CHR¹⁰, SO₂, or C═O. In some embodiments, X¹⁰ is O. In some embodiments, X¹⁰ is NR¹⁰. In some embodiments, X¹⁰ is C(R¹⁰)₂. In some embodiments, X¹⁰ is CHR¹º. In some embodiments, X¹⁰ is SO₂. In some embodiments, X¹⁰ is C═O. In some embodiments, X¹⁰ is CH₂, CF₂, or O. In some embodiments, X¹⁰ is CH₂. In some embodiments, X¹⁰ is NR¹⁰, or O. In some embodiments, X¹⁰ is NMe, NH, or O. In some embodiments, X¹⁰ is selected from those depicted in Table A below. In some embodiments, X¹⁰ is selected from those depicted in Table A-2 below.

As defined generally above, X¹¹ is O, NR¹¹, C(R¹¹)₂, CHR¹¹, SO₂, or C═O. In some embodiments, X¹¹ is O. In some embodiments, X¹¹ is NR¹¹. In some embodiments, X¹¹ is C(R¹¹)₂. In some embodiments, X¹¹ is CHR¹¹. In some embodiments, X¹¹ is SO₂. In some embodiments, X¹¹ is CH₂. In some embodiments, X¹¹ is C═O. In some embodiments, X¹¹ is selected from those depicted in Table A below. In some embodiments, X¹¹ is selected from those depicted in Table A-2 below.

As defined generally above, X¹² is a direct bond, O, NR¹², C(R¹²)₂, CHR¹², —CH₂CH₂—, —OCH₂—, SO₂, or C═O. In some embodiments, X¹² is O. In some embodiments, X¹² is NR¹². In some embodiments, X¹² is C(R¹²)₂. In some embodiments, X¹² is CHR¹². In some embodiments, X¹² is CH₂. In some embodiments, X¹² is SO₂. In some embodiments, X¹² is C═O. In some embodiments, X¹² is —CH₂CH₂—. In some embodiments, X¹² is —OCH₂—. In some embodiments, X¹² is a direct bond. In some embodiments, X¹² is selected from those depicted in Table A below. In some embodiments, X¹² is selected from those depicted in Table A-2 below.

In some embodiments, when any of X⁷, X⁸, X⁹, X¹⁰, X¹¹, or X¹² is N, O or SO₂, then neither of the neighboring positions in Ring Bare N, O or SO₂.

In some embodiments, when any one of X⁸, X⁹, X¹⁰, X¹¹, or X¹² is C═O, then neither of the neighboring positions in Ring B are C═O or SO₂.

As defined generally above, R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R⁷ is an optionally substituted aliphatic group. In some embodiments, R⁷ is halogen. In some embodiments, R⁷ is —OR. In some embodiments, R⁷ is —NR². In some embodiments, R⁷ is —C(═O)R. In some embodiments, R⁷ is —C(═O)OR. In some embodiments, R⁷ is —C(═O)NR₂. In some embodiments, R⁷ is-SO₂R. In some embodiments, R⁷ is-SO₂NR₂. In some embodiments, R⁷ is C₁₋₆haloalkyl. In some embodiments, R⁷ is C₁₋₆haloalkoxy. In some embodiments, R⁷ is methyl. In some embodiments, R⁷ is OH. In some embodiments, R⁷ is F. In some embodiments, R⁷ is selected from those depicted in Table A below. In some embodiments, R⁷ is selected from those depicted in Table A-2 below.

As defined generally above, each of R⁸. R⁹. R¹⁰. R¹¹, and R¹² is independently selected from hydrogen, an optionally substituted C₁₋₆ aliphatic group, —OR, —CN, —NR², —C(═O)R, —C(═O)OR, —C(═O)NR₂, —SO₂R, —SO₂NR₂, C₁₋₆haloalkyl. C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁸ is hydrogen. In some embodiments, R⁸ is an optionally substituted C₁₋₆ aliphatic group. In some embodiments, R⁸—OR. In some embodiments, R⁸ is —NR². In some embodiments. R⁸ is —C(═O)R. In some embodiments, R⁸ is —C(═O)OR. In some embodiments, R⁸ is —C(═O)NR₂. In some embodiments, R⁸ is-SO₂R. In some embodiments, R⁸ is-SO₂NR₂. In some embodiments. R⁸ is C₁₋₆haloalkyl. In some embodiments, R⁸ is C₁₋₆haloalkoxy. In some embodiments, R⁸ is an optionally substituted 3-8 membered saturated or partially unsaturated monocyclic carbocyclic ring. In some embodiments, R⁸ is an optionally substituted 7-12 membered saturated or partially unsaturated bicyclic carbocyclic ring. In some embodiments, R⁸ is an optionally substituted phenyl. In some embodiments. R⁸ is an optionally substituted 8-10 membered bicyclic aromatic carbocyclic ring. In some embodiments. R⁸ is an optionally substituted 3-8 membered saturated or partially unsaturated monocyclic heterocyclic ring (having 1-2 heteroatoms independently selected from nitrogen, oxygen, and sulfur). In some embodiments. R⁸ is an optionally substituted 7-12 membered saturated or partially unsaturated bicyclic heterocyclic ring (having 1-4 heteroatoms independently selected from nitrogen, oxygen, and sulfur). In some embodiments, R⁸ is an optionally substituted 5-6 membered monocyclic heteroaromatic ring (having 1-4 heteroatoms independently selected from nitrogen, oxygen, and sulfur). In some embodiments. R⁸ is an optionally substituted 8-10 membered bicyclic heteroaromatic ring (having 1-5 heteroatoms independently selected from nitrogen, oxygen, and sulfur). In some embodiments, R⁸ is methyl. In some embodiments, R⁸ is —OH. In some embodiments, R⁸ is F. In some embodiments, R⁸ is methoxy. In some embodiments, R⁸ is —CH₂OH. In some embodiments, wherein X⁸ is C(R⁸)₂, each R⁸ is independently selected from any of the aforementioned substituents. In some embodiments, wherein X⁸ is C(R⁸)₂, both R⁸ are the same. In some embodiments, R⁸ is selected from those depicted in Table A below. In some embodiments, R⁸ is selected from those depicted in Table A-2 below.

In some embodiments, R¹ is hydrogen. In some embodiments, R⁹ is an optionally substituted C₁₋₆ aliphatic group. In some embodiments, R⁹—OR. In some embodiments, R⁹ is —NR². In some embodiments. R⁹ is —C(═O)R. In some embodiments, R⁹ is —C(═O)OR. In some embodiments, R⁹ is —C(═O)NR₂. In some embodiments, R⁹ is-SO₂R. In some embodiments, R⁹ is-SO₂NR₂. In some embodiments. R⁹ is C₁₋₆haloalkyl. In some embodiments, R⁹ is C₁₋₆haloalkoxy. In some embodiments, R⁹ is an optionally substituted 3-8 membered saturated or partially unsaturated monocyclic carbocyclic ring. In some embodiments, R⁹ is an optionally substituted 7-12 membered saturated or partially unsaturated bicyclic carbocyclic ring. In some embodiments, R⁹ is an optionally substituted phenyl. In some embodiments. R⁹ is an optionally substituted 8-10 membered bicyclic aromatic carbocyclic ring. In some embodiments. R⁹ is an optionally substituted 3-8 membered saturated or partially unsaturated monocyclic heterocyclic ring (having 1-2 heteroatoms independently selected from nitrogen, oxygen, and sulfur). In some embodiments. R⁹ is an optionally substituted 7-12 membered saturated or partially unsaturated bicyclic heterocyclic ring (having 1-4 heteroatoms independently selected from nitrogen, oxygen, and sulfur). In some embodiments, R⁹ is an optionally substituted 5-6 membered monocyclic heteroaromatic ring (having 1-4 heteroatoms independently selected from nitrogen, oxygen, and sulfur). In some embodiments. R⁹ is an optionally substituted 8-10 membered bicyclic heteroaromatic ring (having 1-5 heteroatoms independently selected from nitrogen, oxygen, and sulfur). In some embodiments, R⁹ is methyl. In some embodiments, R⁹ is —OH. In some embodiments, R¹ is F. In some embodiments, R⁹ is methoxy. In some embodiments, R⁹ is —CH₂OH. In some embodiments, wherein X⁹ is C(R⁹)₂, each R⁹ is independently selected from any of the aforementioned substituents. In some embodiments, wherein X⁹ is C(R⁹)₂, both R⁹ are the same. In some embodiments, R⁹ is selected from those depicted in Table A below. In some embodiments, R⁹ is selected from those depicted in Table A-2 below.

In some embodiments, R⁹ is optionally substituted pyrazolyl. In some embodiments, R⁹ is optionally substituted pyridinyl. In some embodiments, R⁹ is optionally substituted pyrimidinyl. In some embodiments. R⁹ is optionally substituted pyridazinyl. In some embodiments, R⁹ is optionally substituted imidazolyl. In some embodiments, R⁹ is optionally substituted triazolyl. In some embodiments, R⁹ is optionally substituted oxazolyl. In some embodiments, R⁹ is optionally substituted thiazolyl. In some embodiments, R¹ is optionally substituted oxadiazolyl. In some embodiments, R⁹ is optionally substituted thiadiazolyl. In some embodiments, R⁹ is optionally substituted oxetanyl. In some embodiments, R⁹ is optionally substituted azetidinyl. In some embodiments, R⁹ is optionally substituted piperidinyl. In some embodiments, R¹ is optionally substituted piperazinyl.

In some embodiments, R⁹ is substituted with an optionally substituted 3-6 membered saturated or partially unsaturated monocyclic carbocyclic ring. In some embodiments, R⁹ is substituted with an optionally substituted 5-8 membered saturated or partially unsaturated bicyclic carbocyclic ring. In some embodiments, R⁹ is substituted with an optionally substituted 3-6 membered saturated or partially unsaturated monocyclic heterocyclic ring. In some embodiments, R⁹ is substituted with an optionally substituted C₁₋₆ aliphatic group. In some embodiments, R⁹ is substituted with a methyl group. In some embodiments, R⁹ is substituted with a —CD₃ group. In some embodiments, R⁹ is substituted with a methoxy group. In some embodiments, R⁹ is substituted with a cyclopropyl group. In some embodiments, R⁹ is substituted with an optionally substituted

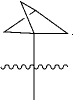 [SMILES: *C12CC(C1)C2]

In some embodiments, R⁹ is —OR, wherein R is an optionally substituted 5-6 membered heteroaryl ring (having 1-4 heteroatoms independently selected from nitrogen, oxygen, and sulfur). In some embodiments, R⁹ is —NHR, wherein R is an optionally substituted 5-6 membered heteroaryl ring (having 1-4 heteroatoms independently selected from nitrogen, oxygen, and sulfur). In some embodiments, R⁹ is-N(CH₃)R, wherein R is an optionally substituted 5-6 membered heteroaryl ring (having 1-4 heteroatoms independently selected from nitrogen, oxygen, and sulfur). In some embodiments, R¹ is —C(═O)N(CH₃)R, wherein R is an optionally substituted 5-6 membered heteroaryl ring (having 1-4 heteroatoms independently selected from nitrogen, oxygen, and sulfur). In some embodiments, R⁹ is —C(═O)NHR, wherein R is an optionally substituted 5-6 membered heteroaryl ring (having 1-4 heteroatoms independently selected from nitrogen, oxygen, and sulfur).

In some embodiments, R⁹ is a substituent selected from those shown below:

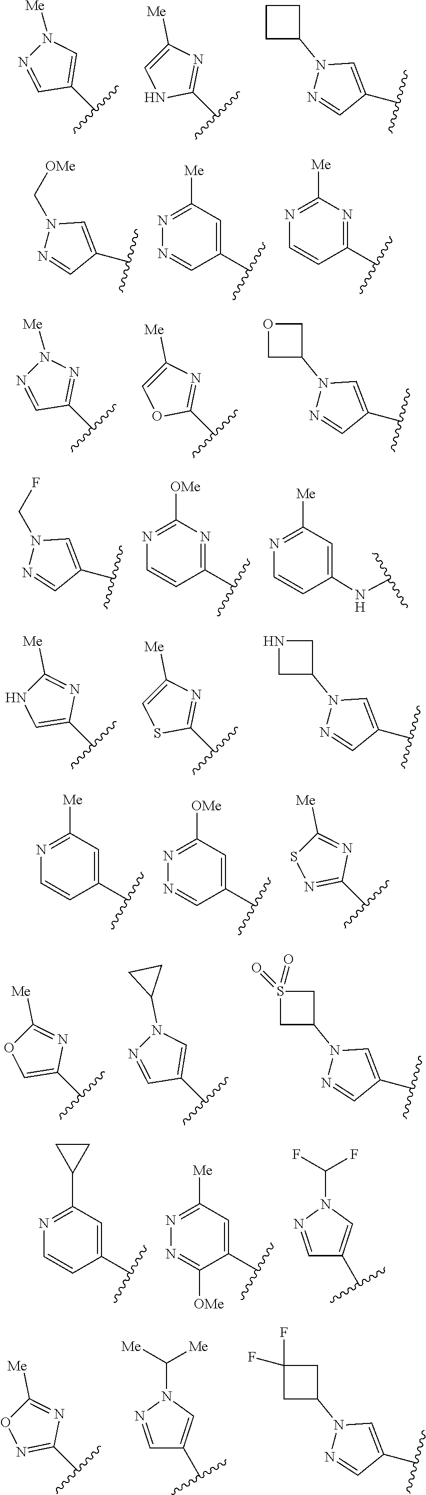

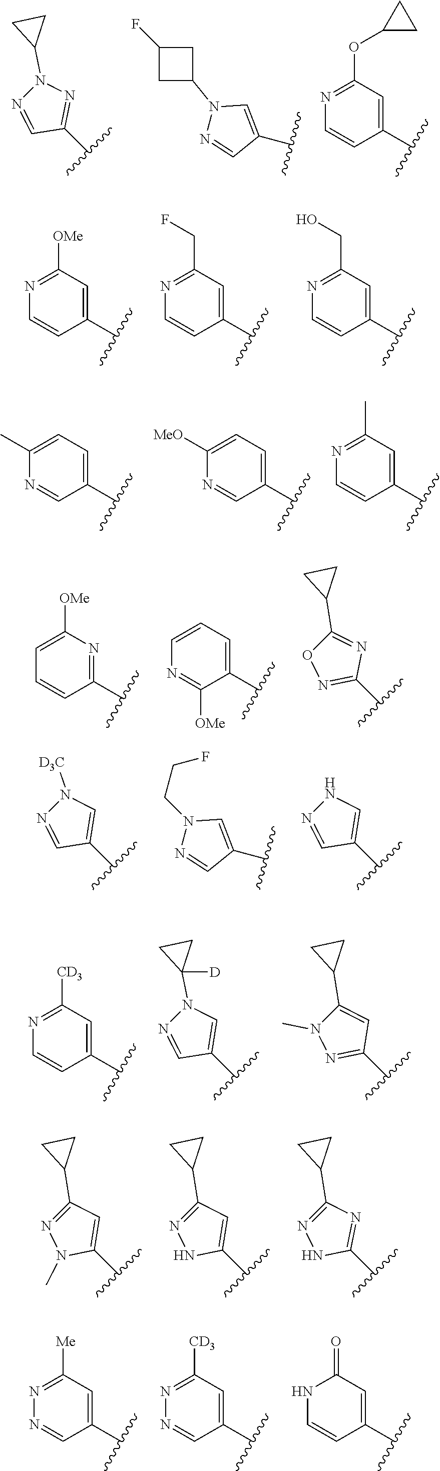

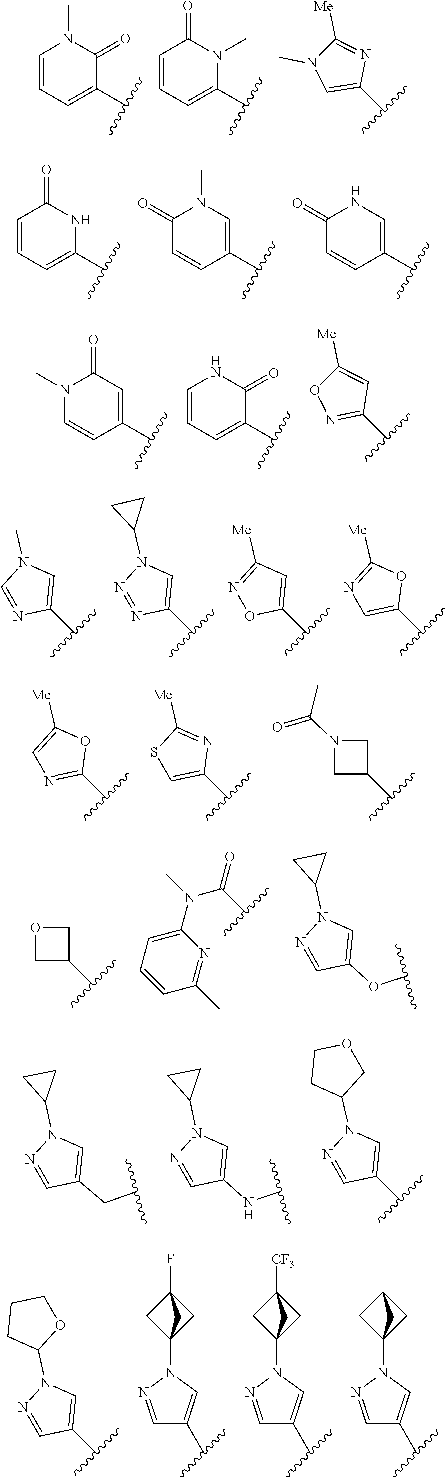

In some embodiments, R⁹ is methyl, tetrahydrofuran-3-yl.

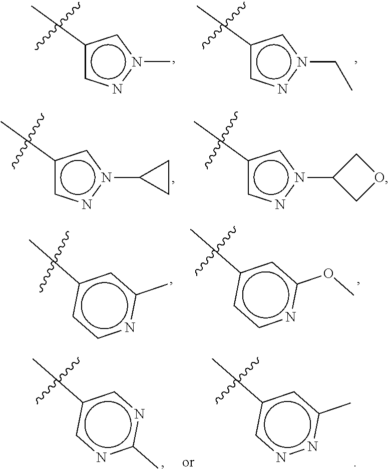

In some embodiments, R⁹ is methyl, tetrahydrofuran-3-yl.

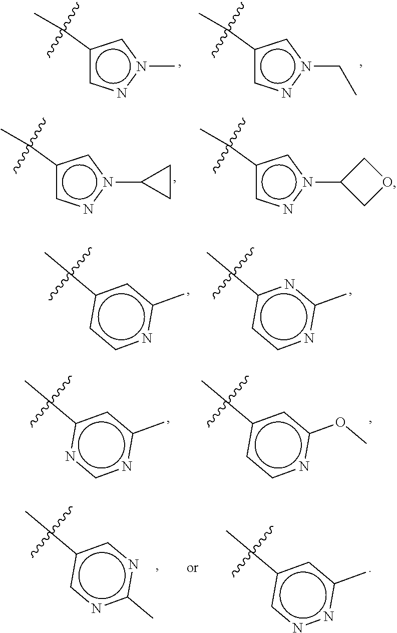

In some embodiments. R⁹ is

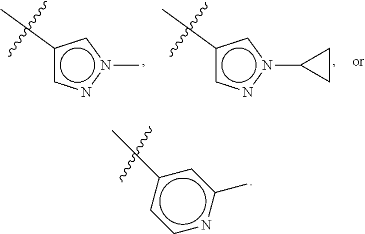

In some embodiments, R⁹ is

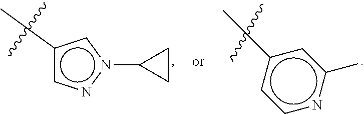

In some embodiments, R⁹ is

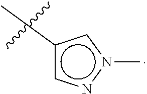

In some embodiments, R⁹ is

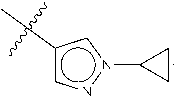

In some embodiments, R⁹ is

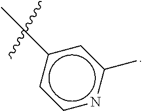

In some embodiments, R¹⁰ is hydrogen. In some embodiments, R¹⁰ is an optionally substituted C₁₋₆ aliphatic group. In some embodiments, R¹⁰—OR. In some embodiments, R¹⁰ is —NR². In some embodiments, R¹⁰ is —C(═O)R. In some embodiments, R¹⁰ is —C(═O)OR. In some embodiments, R¹⁰ is —C(═O)NR₂. In some embodiments, R¹⁰ is-SO₂R. In some embodiments, R¹⁰ is-SO₂NR₂. In some embodiments, R¹⁰ is C₁₋₆haloalkyl. In some embodiments, R¹⁰ is C₁₋₆haloalkoxy. In some embodiments, R¹⁰ is an optionally substituted 3-8 membered saturated or partially unsaturated monocyclic carbocyclic ring. In some embodiments, R¹⁰ is an optionally substituted 7-12 membered saturated or partially unsaturated bicyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 3-8 membered saturated or partially unsaturated monocyclic heterocyclic ring (having 1-2 heteroatoms independently selected from nitrogen, oxygen, and sulfur). In some embodiments, R¹⁰ is an optionally substituted 7-12 membered saturated or partially unsaturated bicyclic heterocyclic ring (having 1-4 heteroatoms independently selected from nitrogen, oxygen, and sulfur). In some embodiments, R¹⁰ is an optionally substituted 5-6 membered monocyclic heteroaromatic ring (having 1-4 heteroatoms independently selected from nitrogen, oxygen, and sulfur). In some embodiments, R¹⁰ is an optionally substituted 8-10 membered bicyclic heteroaromatic ring (having 1-5 heteroatoms independently selected from nitrogen, oxygen, and sulfur). In some embodiments, R¹⁰ is methyl. In some embodiments, R¹⁰ is —OH. In some embodiments, R¹⁰ is F. In some embodiments, R¹⁰ is methoxy. In some embodiments, R¹⁰ is —CH₂OH. In some embodiments, wherein X¹⁰ is C(R¹⁰)₂, each R¹⁰ is independently selected from any of the aforementioned substituents. In some embodiments, wherein X¹⁰ is C(R¹⁰)₂, both R¹⁰ are the same. In some embodiments, R¹⁰ is selected from those depicted in Table A below. In some embodiments, R¹⁰ is selected from those depicted in Table A-2 below.

In some embodiments, R¹¹ is hydrogen. In some embodiments, R¹¹ is an optionally substituted C₁₋₆ aliphatic group. In some embodiments, R¹¹—OR. In some embodiments, R¹¹ is —NR². In some embodiments. R¹¹ is —C(═O)R. In some embodiments, R¹¹ is —C(═O)OR. In some embodiments, R¹¹ is —C(═O)NR₂. In some embodiments, R¹¹ is-SO₂R. In some embodiments, R¹¹ is-SO₂NR₂. In some embodiments. R¹¹ is C₁₋₆haloalkyl. In some embodiments, R¹¹ is C₁₋₆haloalkoxy. In some embodiments, R¹¹ is an optionally substituted 3-8 membered saturated or partially unsaturated monocyclic carbocyclic ring. In some embodiments, R¹¹ is an optionally substituted 7-12 membered saturated or partially unsaturated bicyclic carbocyclic ring. In some embodiments, R¹¹ is an optionally substituted phenyl. In some embodiments. R¹¹ is an optionally substituted 8-10 membered bicyclic aromatic carbocyclic ring. In some embodiments. R¹¹ is an optionally substituted 3-8 membered saturated or partially unsaturated monocyclic heterocyclic ring (having 1-2 heteroatoms independently selected from nitrogen, oxygen, and sulfur). In some embodiments. R¹¹ is an optionally substituted 7-12 membered saturated or partially unsaturated bicyclic heterocyclic ring (having 1-4 heteroatoms independently selected from nitrogen, oxygen, and sulfur). In some embodiments, R¹¹ is an optionally substituted 5-6 membered monocyclic heteroaromatic ring (having 1-4 heteroatoms independently selected from nitrogen, oxygen, and sulfur). In some embodiments. R¹¹ is an optionally substituted 8-10 membered bicyclic heteroaromatic ring (having 1-5 heteroatoms independently selected from nitrogen, oxygen, and sulfur). In some embodiments, R¹¹ is methyl. In some embodiments, R¹¹ is —OH. In some embodiments, R¹¹ is F. In some embodiments, R¹¹ is methoxy. In some embodiments, R¹¹ is —CH₂OH. In some embodiments, wherein X¹¹ is C(R¹¹)₂, each R¹¹ is independently selected from any of the aforementioned substituents. In some embodiments, wherein X¹¹ is C(R¹¹)₂, both R¹¹ are the same. In some embodiments, R¹¹ is selected from those depicted in Table A below. In some embodiments, R¹¹ is selected from those depicted in Table A-2 below.

In some embodiments, R¹² is hydrogen. In some embodiments, R¹² is an optionally substituted C₁₋₆ aliphatic group. In some embodiments, R¹²—OR. In some embodiments, R¹² is —NR². In some embodiments. R¹² is —C(═O)R. In some embodiments, R¹² is —C(═O)OR. In some embodiments, R¹² is —C(═O)NR₂. In some embodiments, R¹² is-SO₂R. In some embodiments, R¹² is-SO₂NR₂. In some embodiments. R¹² is C₁₋₆haloalkyl. In some embodiments, R¹² is C₁₋₆haloalkoxy. In some embodiments, R¹² is an optionally substituted 3-8 membered saturated or partially unsaturated monocyclic carbocyclic ring. In some embodiments, R¹² is an optionally substituted 7-12 membered saturated or partially unsaturated bicyclic carbocyclic ring. In some embodiments, R¹² is an optionally substituted phenyl. In some embodiments. R¹² is an optionally substituted 8-10 membered bicyclic aromatic carbocyclic ring. In some embodiments. R¹² is an optionally substituted 3-8 membered saturated or partially unsaturated monocyclic heterocyclic ring (having 1-2 heteroatoms independently selected from nitrogen, oxygen, and sulfur). In some embodiments. R¹² is an optionally substituted 7-12 membered saturated or partially unsaturated bicyclic heterocyclic ring (having 1-4 heteroatoms independently selected from nitrogen, oxygen, and sulfur). In some embodiments, R¹² is an optionally substituted 5-6 membered monocyclic heteroaromatic ring (having 1-4 heteroatoms independently selected from nitrogen, oxygen, and sulfur). In some embodiments, R¹² is an optionally substituted 8-10 membered bicyclic heteroaromatic ring (having 1-5 heteroatoms independently selected from nitrogen, oxygen, and sulfur). In some embodiments, R¹² is methyl. In some embodiments, R¹² is —OH. In some embodiments, R¹² is F. In some embodiments, R¹² is methoxy. In some embodiments, R¹² is —CH₂OH. In some embodiments, wherein X¹² is C(R¹²)₂, each R¹² is independently selected from any of the aforementioned substituents. In some embodiments, wherein X¹² is C(R¹²)₂, both R¹² are the same. In some embodiments, R¹² is selected from those depicted in Table A below. In some embodiments, R¹² is selected from those depicted in Table A-2 below.

In some embodiments, Ring B is a substituent selected from those shown below:

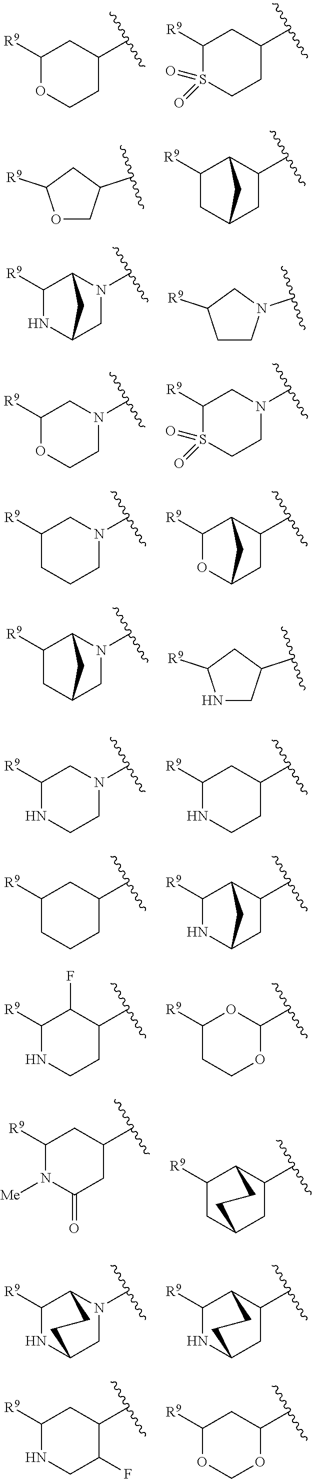

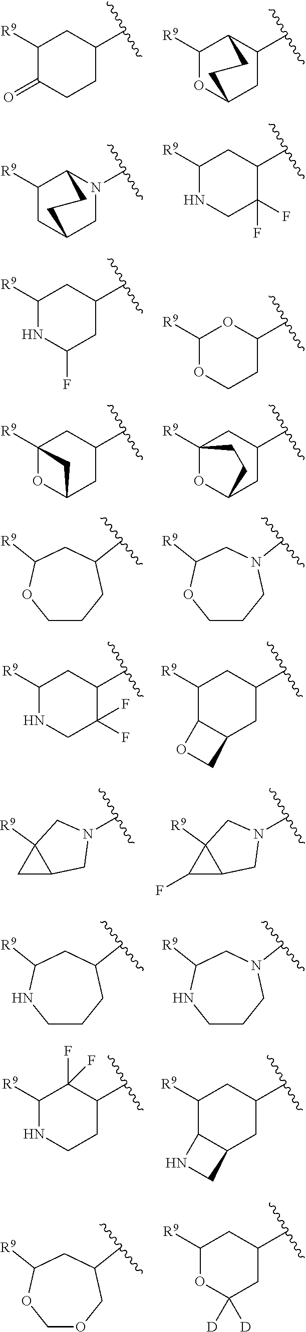

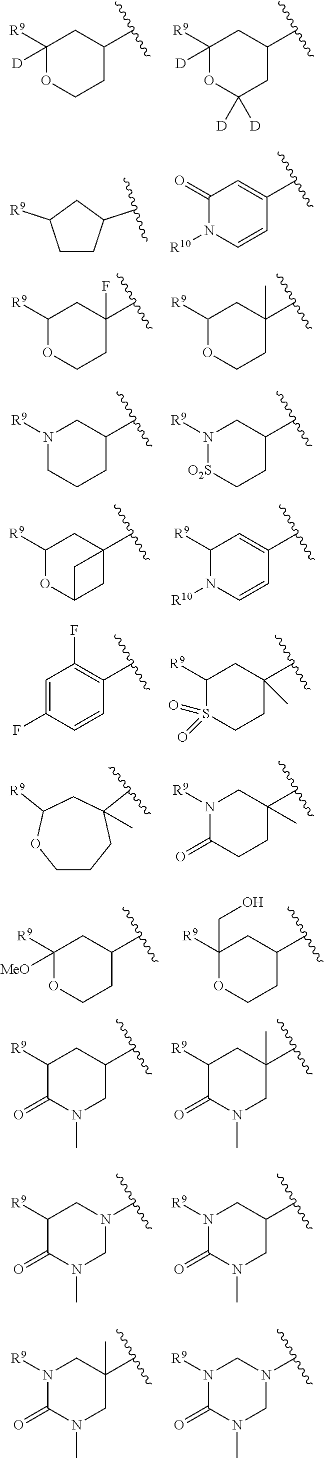

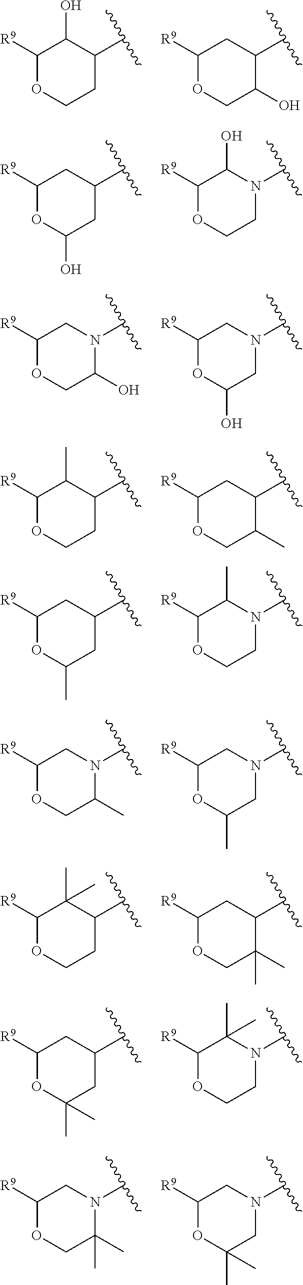

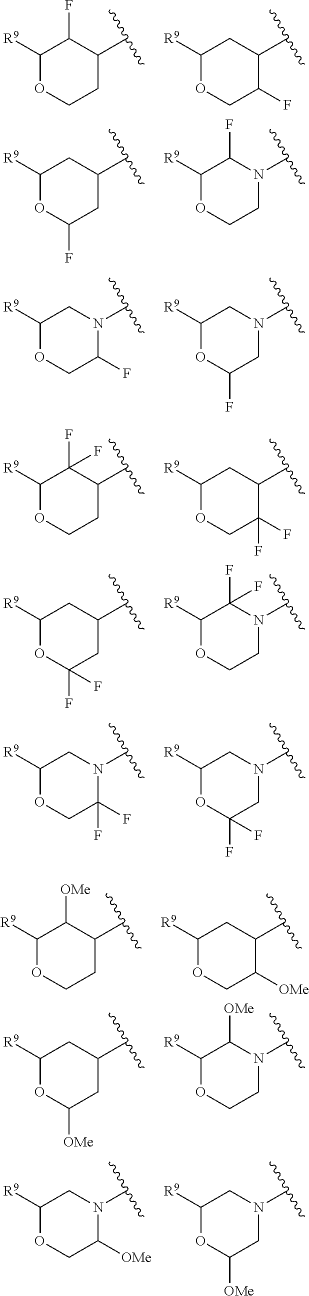

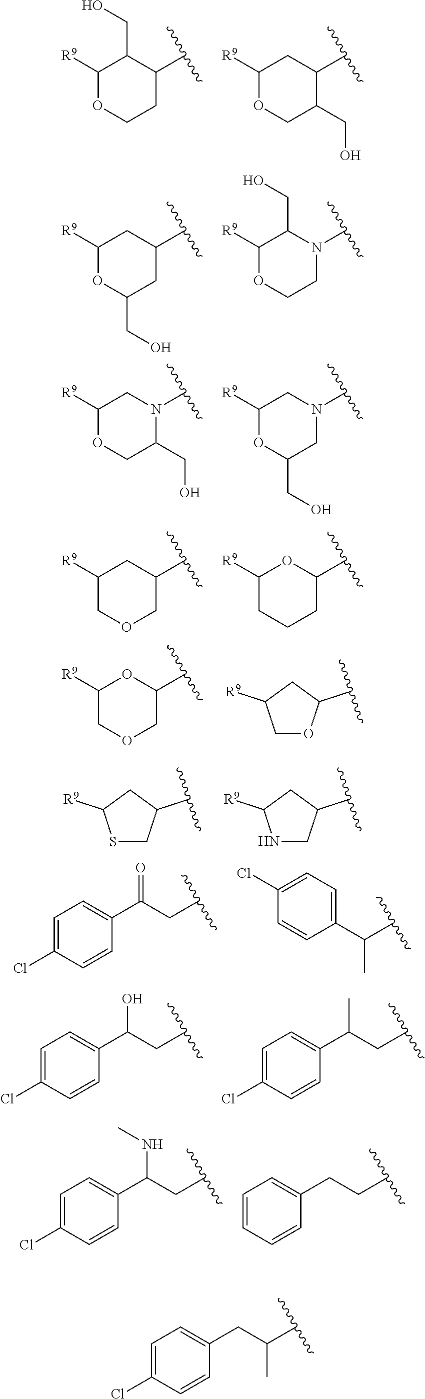

In some embodiments. Ring B is

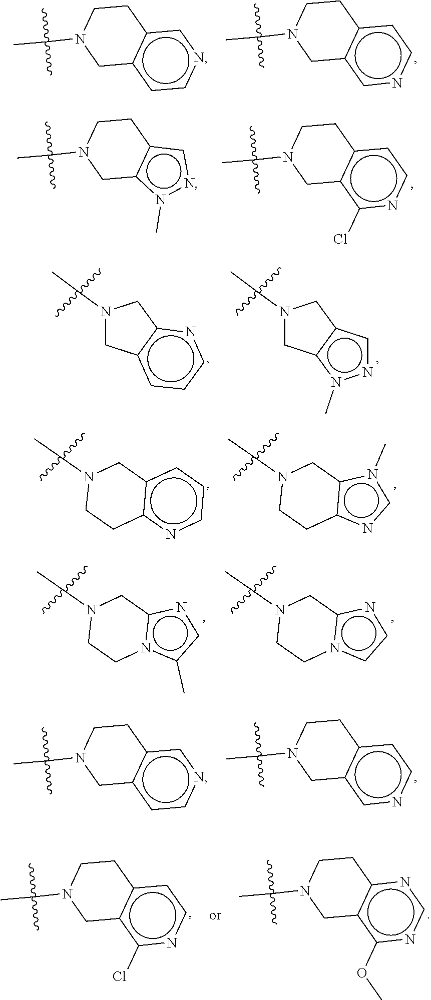

In some embodiments. Ring B is

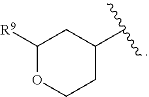

In some embodiments. Ring B is

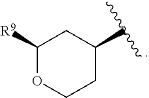

In some embodiments. Ring B is

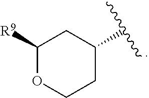

In some embodiments. Ring B is

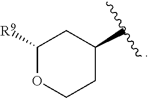

In some embodiments. Ring B is

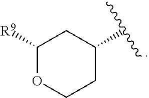

In some embodiments, Ring B is

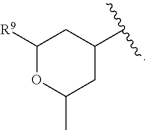

In some embodiments, Ring B is

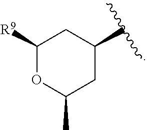

In some embodiments, Ring B is

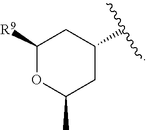

In some embodiments, Ring B is

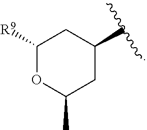

In some embodiments, Ring B is

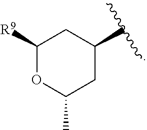

In some embodiments, Ring B is

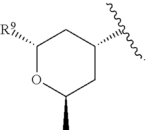

In some embodiments, Ring B is In some embodiments, Ring B is

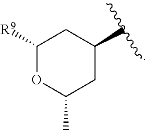

In some embodiments, Ring B is

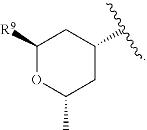

In some embodiments, Ring B is

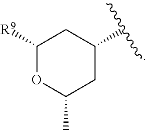

In some embodiments, Ring B is

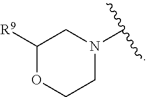

In some embodiments, Ring B is

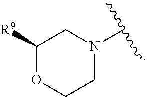

In some embodiments, Ring B is

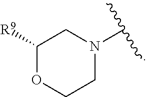

In some embodiments, Ring B is

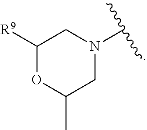

In some embodiments, Ring B is

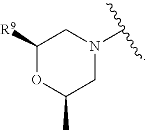

In some embodiments, Ring B is

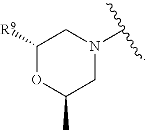

In some embodiments, Ring B is

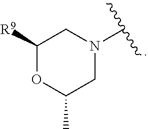

In some embodiments, Ring B is

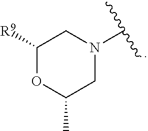

In some embodiments, Ring B is

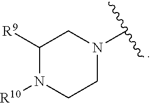

In some embodiments, Ring B is

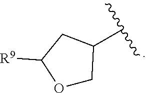

In some embodiments, Ring B is

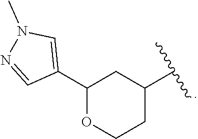 [SMILES: *C1CCOC(c2cnn(C)c2)C1]

In some embodiments, Ring B is

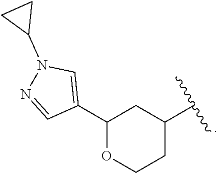 [SMILES: *C1CCOC(c2cnn(C3CC3)c2)C1]

In some embodiments, Ring B is

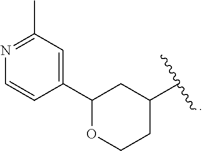 [SMILES: *C1CCOC(c2ccnc(C)c2)C1]

In some embodiments, Ring B is

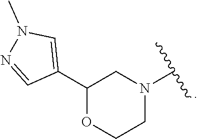 [SMILES: *N1CCOC(c2cnn(C)c2)C1]

In some embodiments, Ring B is.

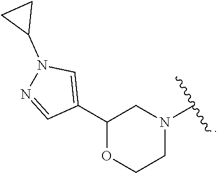 [SMILES: *N1CCOC(c2cnn(C3CC3)c2)C1]

In some embodiments, Ring B is

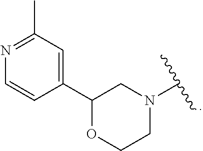 [SMILES: *N1CCOC(c2ccnc(C)c2)C1]

In some embodiments, Ring B is

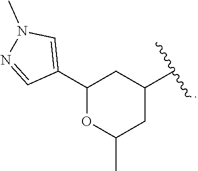 [SMILES: *C1CC(C)OC(c2cnn(C)c2)C1]

In some embodiments, Ring B is

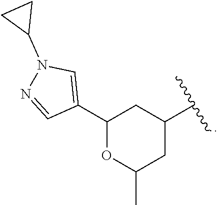 [SMILES: *C1CC(C)OC(c2cnn(C3CC3)c2)C1]

In some embodiments, Ring B is

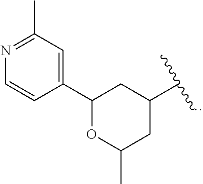 [SMILES: *C1CC(C)OC(c2ccnc(C)c2)C1]

In some embodiments, Ring B is

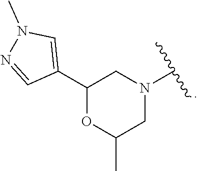 [SMILES: *N1CC(C)OC(c2cnn(C)c2)C1]

In some embodiments, Ring B is

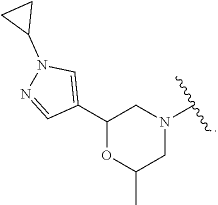 [SMILES: *N1CC(C)OC(c2cnn(C3CC3)c2)C1]

In some embodiments, Ring B is

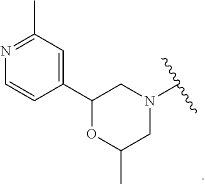 [SMILES: *N1CC(C)OC(c2ccnc(C)c2)C1]

In some embodiments, Ring B is

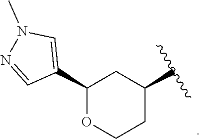 [SMILES: *[C@H]1CCO[C@@H](c2cnn(C)c2)C1]

In some embodiments, Ring B is

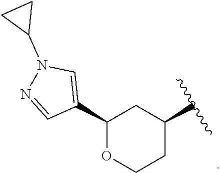 [SMILES: *[C@H]1CCO[C@@H](c2cnn(C3CC3)c2)C1]

In some embodiments, Ring B is

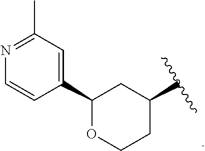 [SMILES: *[C@H]1CCO[C@@H](c2ccnc(C)c2)C1]

In some embodiments, Ring B is

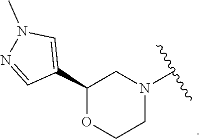 [SMILES: *N1CCO[C@@H](c2cnn(C)c2)C1]

In some embodiments, Ring B is

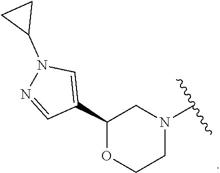 [SMILES: *N1CCO[C@@H](c2cnn(C3CC3)c2)C1]

In some embodiments, Ring B is

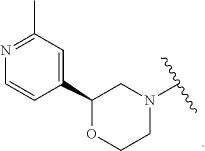 [SMILES: *N1CCO[C@@H](c2ccnc(C)c2)C1]

In some embodiments, Ring B is

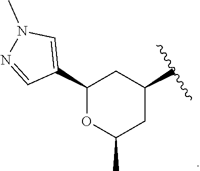 [SMILES: *[C@H]1C[C@@H](C)O[C@@H](c2cnn(C)c2)C1]

In some embodiments, Ring B is

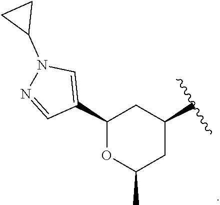 [SMILES: *[C@H]1C[C@@H](C)O[C@@H](c2cnn(C3CC3)c2)C1]

In some embodiments, Ring B is

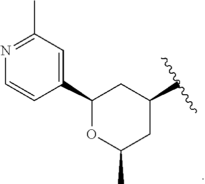 [SMILES: *[C@H]1C[C@@H](C)O[C@@H](c2ccnc(C)c2)C1]

In some embodiments, Ring B is

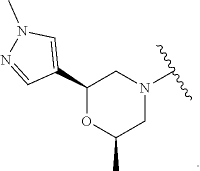 [SMILES: *N1C[C@@H](C)O[C@@H](c2cnn(C)c2)C1]

In some embodiments, Ring B is

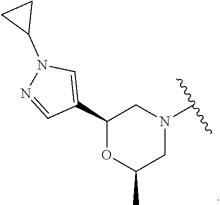 [SMILES: *N1C[C@@H](C)O[C@@H](c2cnn(C3CC3)c2)C1]

In some embodiments, Ring B is

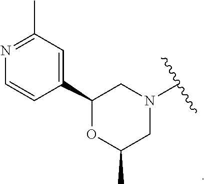 [SMILES: *N1C[C@@H](C)O[C@@H](c2ccnc(C)c2)C1]

In some embodiments, at least one hydrogen atom of the compound is a deuterium atom. In some embodiments, at least one C₁-C₆ aliphatic group of the compound is substituted with at least one deuterium atom. In some embodiments, at least one C₁-C₆alkyl group of the compound is substituted with at least one deuterium atom. In some embodiments, R² is —CD₃. In some embodiments, R³ is —CD₃. In some embodiments, R² and R³ are both-CD₃. In some embodiments, R⁴ is —CD₃.

Exemplary compounds of the invention are set forth in Table A, below. In some embodiments, the compound is a compound set forth in Table A, or a pharmaceutically acceptable salt thereof.

TABLE A
Exemplary Compounds

| I-# | Structure |
|---|---|
| I-1 | 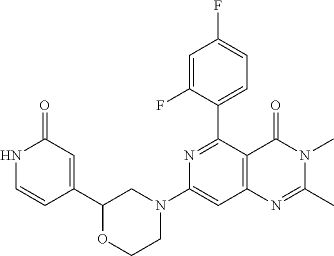 [SMILES: Cc1nc2cc(N3CCOC(c4cc[nH]c(=O)c4)C3)nc(-c3ccc(F)cc3F)c2c(=O)n1C] |
| I-2 | 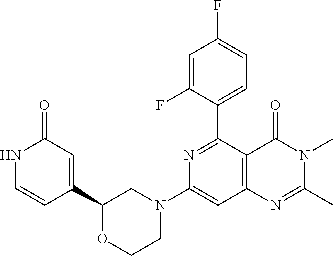 [SMILES: Cc1nc2cc(N3CCO[C@@H](c4cc[nH]c(=O)c4)C3)nc(-c3ccc(F)cc3F)c2c(=O)n1C] |
| I-3 | 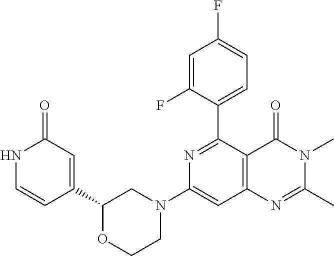 [SMILES: Cc1nc2cc(N3CCO[C@H](c4cc[nH]c(=O)c4)C3)nc(-c3ccc(F)cc3F)c2c(=O)n1C] |
| I-4 | 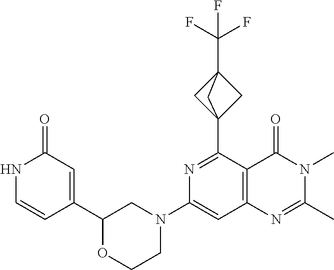 [SMILES: Cc1nc2cc(N3CCOC(c4cc[nH]c(=O)c4)C3)nc(C34CC(C(F)(F)F)(C3)C4)c2c(=O)n1C] |
| I-5 | 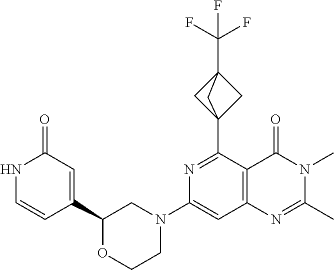 [SMILES: Cc1nc2cc(N3CCO[C@@H](c4cc[nH]c(=O)c4)C3)nc(C34CC(C(F)(F)F)(C3)C4)c2c(=O)n1C] |
| I-6 | 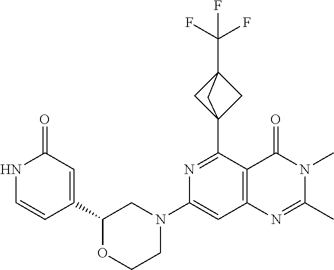 [SMILES: Cc1nc2cc(N3CCO[C@H](c4cc[nH]c(=O)c4)C3)nc(C34CC(C(F)(F)F)(C3)C4)c2c(=O)n1C] |
| I-7 | 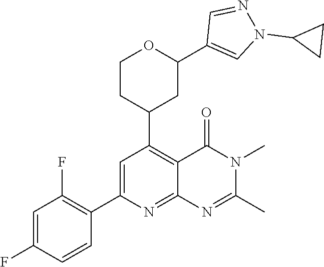 [SMILES: Cc1nc2nc(-c3ccc(F)cc3F)cc(C3CCOC(c4cnn(C5CC5)c4)C3)c2c(=O)n1C] |
| I-8 | 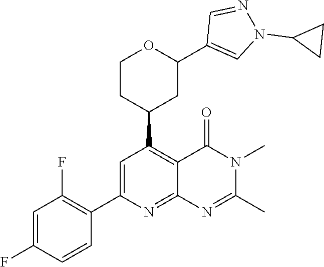 [SMILES: Cc1nc2nc(-c3ccc(F)cc3F)cc([C@H]3CCOC(c4cnn(C5CC5)c4)C3)c2c(=O)n1C] |
| I-9 | 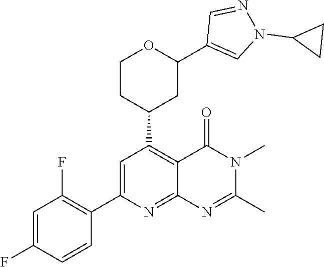 [SMILES: Cc1nc2nc(-c3ccc(F)cc3F)cc([C@@H]3CCOC(c4cnn(C5CC5)c4)C3)c2c(=O)n1C] |
| I-10 | 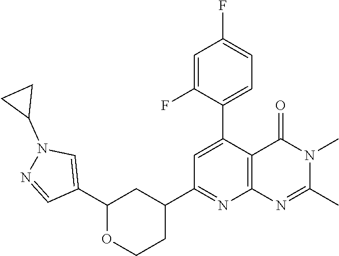 [SMILES: Cc1nc2nc(C3CCOC(c4cnn(C5CC5)c4)C3)cc(-c3ccc(F)cc3F)c2c(=O)n1C] |
| I-11 | 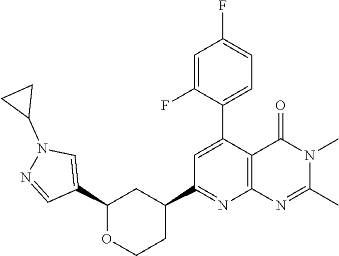 [SMILES: Cc1nc2nc([C@H]3CCO[C@@H](c4cnn(C5CC5)c4)C3)cc(-c3ccc(F)cc3F)c2c(=O)n1C] |
| I-12 | 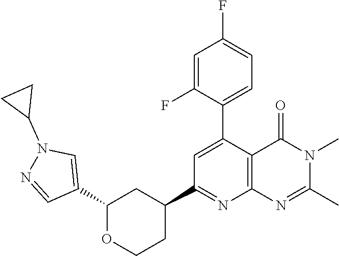 [SMILES: Cc1nc2nc([C@H]3CCO[C@H](c4cnn(C5CC5)c4)C3)cc(-c3ccc(F)cc3F)c2c(=O)n1C] |
| I-13 | 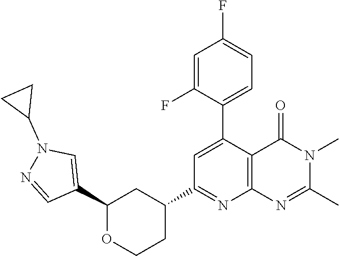 [SMILES: Cc1nc2nc([C@@H]3CCO[C@@H](c4cnn(C5CC5)c4)C3)cc(-c3ccc(F)cc3F)c2c(=O)n1C] |
| I-14 | 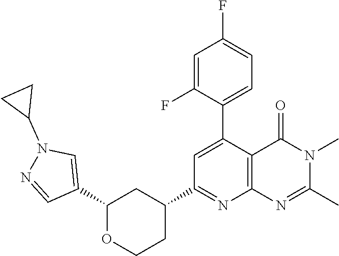 [SMILES: Cc1nc2nc([C@@H]3CCO[C@H](c4cnn(C5CC5)c4)C3)cc(-c3ccc(F)cc3F)c2c(=O)n1C] |
| I-15 | 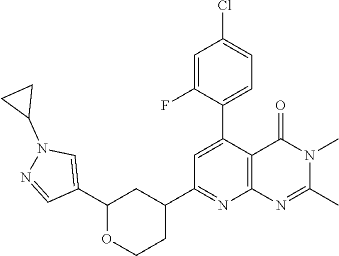 [SMILES: Cc1nc2nc(C3CCOC(c4cnn(C5CC5)c4)C3)cc(-c3ccc(Cl)cc3F)c2c(=O)n1C] |
| I-16 | 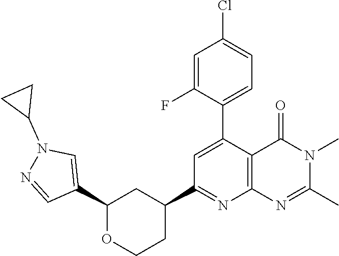 [SMILES: Cc1nc2nc([C@H]3CCO[C@@H](c4cnn(C5CC5)c4)C3)cc(-c3ccc(Cl)cc3F)c2c(=O)n1C] |
| I-17 | 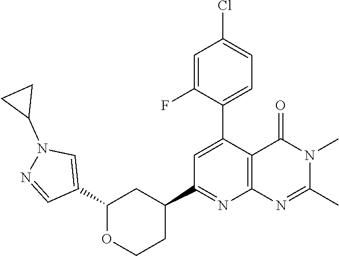 [SMILES: Cc1nc2nc([C@H]3CCO[C@H](c4cnn(C5CC5)c4)C3)cc(-c3ccc(Cl)cc3F)c2c(=O)n1C] |
| I-18 | 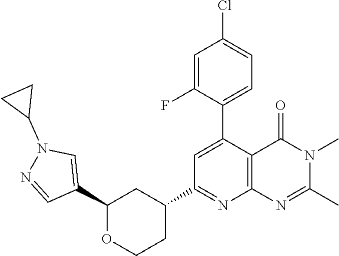 [SMILES: Cc1nc2nc([C@@H]3CCO[C@@H](c4cnn(C5CC5)c4)C3)cc(-c3ccc(Cl)cc3F)c2c(=O)n1C] |
| I-19 | 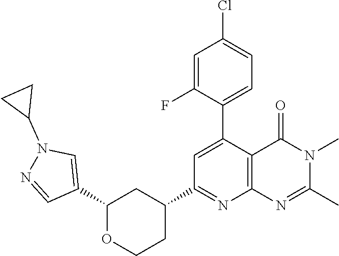 [SMILES: Cc1nc2nc([C@@H]3CCO[C@H](c4cnn(C5CC5)c4)C3)cc(-c3ccc(Cl)cc3F)c2c(=O)n1C] |
| I-20 | 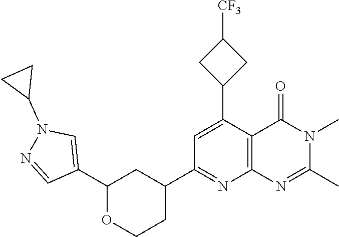 [SMILES: Cc1nc2nc(C3CCOC(c4cnn(C5CC5)c4)C3)cc(C3CC(C(F)(F)F)C3)c2c(=O)n1C] |
| I-21 | 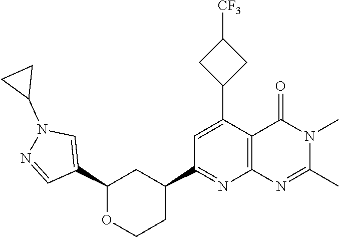 [SMILES: Cc1nc2nc([C@H]3CCO[C@@H](c4cnn(C5CC5)c4)C3)cc(C3CC(C(F)(F)F)C3)c2c(=O)n1C] |
| I-22 | 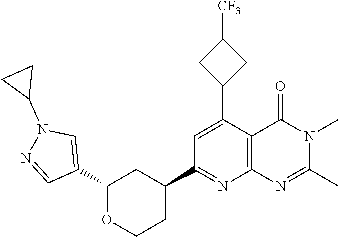 [SMILES: Cc1nc2nc([C@H]3CCO[C@H](c4cnn(C5CC5)c4)C3)cc(C3CC(C(F)(F)F)C3)c2c(=O)n1C] |
| I-23 | 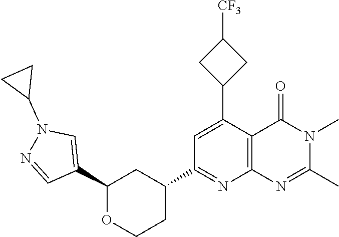 [SMILES: Cc1nc2nc([C@@H]3CCO[C@@H](c4cnn(C5CC5)c4)C3)cc(C3CC(C(F)(F)F)C3)c2c(=O)n1C] |
| I-24 | 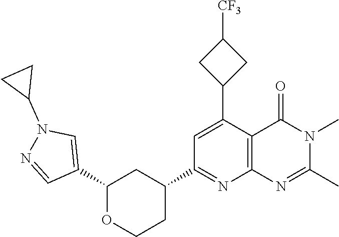 [SMILES: Cc1nc2nc([C@@H]3CCO[C@H](c4cnn(C5CC5)c4)C3)cc(C3CC(C(F)(F)F)C3)c2c(=O)n1C] |
| I-25 | 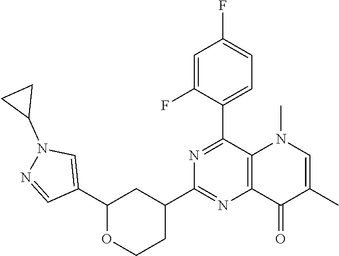 [SMILES: Cc1cn(C)c2c(-c3ccc(F)cc3F)nc(C3CCOC(c4cnn(C5CC5)c4)C3)nc2c1=O] |
| I-26 | 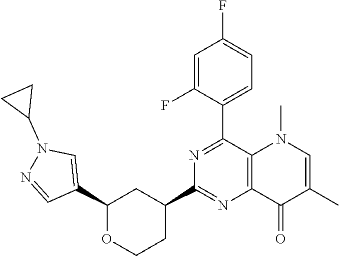 [SMILES: Cc1cn(C)c2c(-c3ccc(F)cc3F)nc([C@H]3CCO[C@@H](c4cnn(C5CC5)c4)C3)nc2c1=O] |
| I-27 | 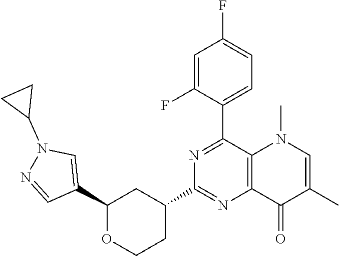 [SMILES: Cc1cn(C)c2c(-c3ccc(F)cc3F)nc([C@@H]3CCO[C@@H](c4cnn(C5CC5)c4)C3)nc2c1=O] |
| I-28 | 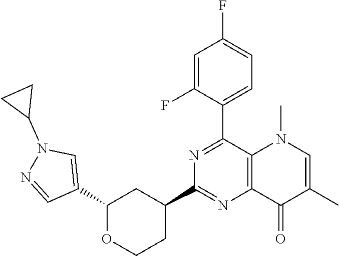 [SMILES: Cc1cn(C)c2c(-c3ccc(F)cc3F)nc([C@H]3CCO[C@H](c4cnn(C5CC5)c4)C3)nc2c1=O] |
| I-29 | 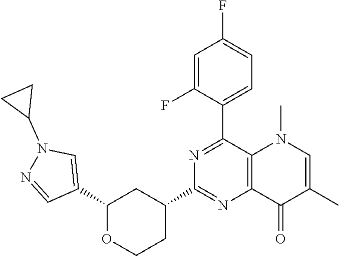 [SMILES: Cc1cn(C)c2c(-c3ccc(F)cc3F)nc([C@@H]3CCO[C@H](c4cnn(C5CC5)c4)C3)nc2c1=O] |
| I-30 | 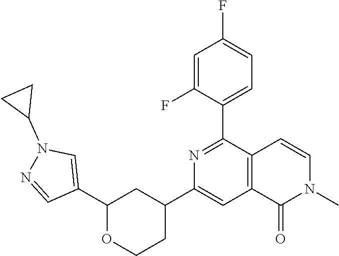 [SMILES: Cn1ccc2c(-c3ccc(F)cc3F)nc(C3CCOC(c4cnn(C5CC5)c4)C3)cc2c1=O] |
| I-31 | 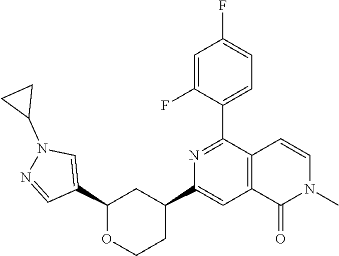 [SMILES: Cn1ccc2c(-c3ccc(F)cc3F)nc([C@H]3CCO[C@@H](c4cnn(C5CC5)c4)C3)cc2c1=O] |
| I-32 | 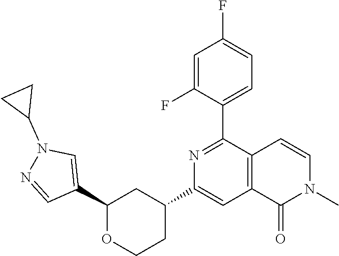 [SMILES: Cn1ccc2c(-c3ccc(F)cc3F)nc([C@@H]3CCO[C@@H](c4cnn(C5CC5)c4)C3)cc2c1=O] |
| I-33 | 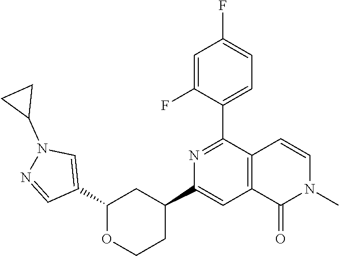 [SMILES: Cn1ccc2c(-c3ccc(F)cc3F)nc([C@H]3CCO[C@H](c4cnn(C5CC5)c4)C3)cc2c1=O] |
| I-34 | 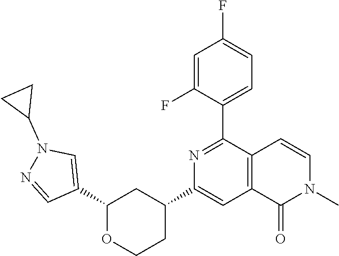 [SMILES: Cn1ccc2c(-c3ccc(F)cc3F)nc([C@@H]3CCO[C@H](c4cnn(C5CC5)c4)C3)cc2c1=O] |
| I-35 | 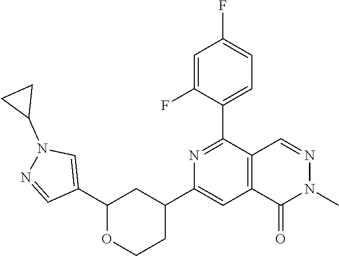 [SMILES: Cn1ncc2c(-c3ccc(F)cc3F)nc(C3CCOC(c4cnn(C5CC5)c4)C3)cc2c1=O] |
| I-36 | 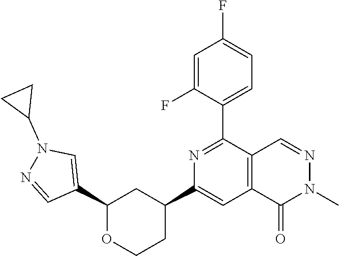 [SMILES: Cn1ncc2c(-c3ccc(F)cc3F)nc([C@H]3CCO[C@@H](c4cnn(C5CC5)c4)C3)cc2c1=O] |
| I-37 | 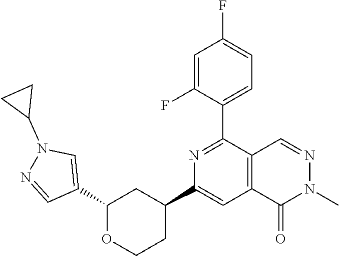 [SMILES: Cn1ncc2c(-c3ccc(F)cc3F)nc([C@H]3CCO[C@H](c4cnn(C5CC5)c4)C3)cc2c1=O] |
| I-38 | 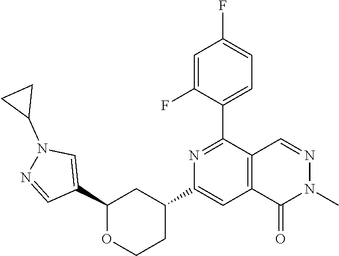 [SMILES: Cn1ncc2c(-c3ccc(F)cc3F)nc([C@@H]3CCO[C@@H](c4cnn(C5CC5)c4)C3)cc2c1=O] |
| I-39 | 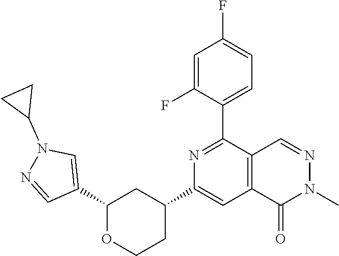 [SMILES: Cn1ncc2c(-c3ccc(F)cc3F)nc([C@@H]3CCO[C@H](c4cnn(C5CC5)c4)C3)cc2c1=O] |

Exemplary compounds of the invention are also set forth in Table A-2, below. In some embodiments, the compound is a compound set forth in Table A-2, or a pharmaceutically acceptable salt thereof.

TABLE A-2
Exemplary Compounds

| I-# | Structure |
|---|---|
| I-40 | 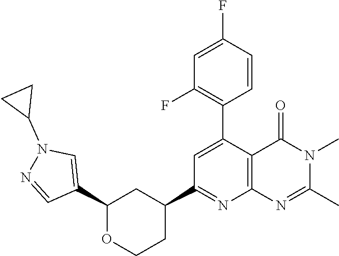 [SMILES: Cc1nc2nc([C@H]3CCO[C@@H](c4cnn(C5CC5)c4)C3)cc(-c3ccc(F)cc3F)c2c(=O)n1C] |
| I-41 | 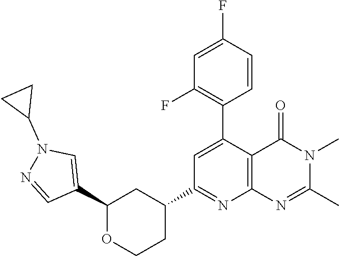 [SMILES: Cc1nc2nc([C@@H]3CCO[C@@H](c4cnn(C5CC5)c4)C3)cc(-c3ccc(F)cc3F)c2c(=O)n1C] |
| I-42 | 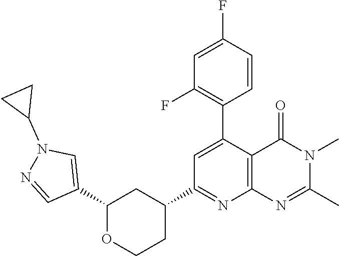 [SMILES: Cc1nc2nc([C@@H]3CCO[C@H](c4cnn(C5CC5)c4)C3)cc(-c3ccc(F)cc3F)c2c(=O)n1C] |
| I-43 | 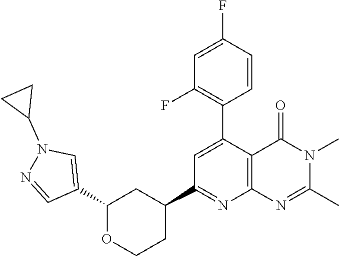 [SMILES: Cc1nc2nc([C@H]3CCO[C@H](c4cnn(C5CC5)c4)C3)cc(-c3ccc(F)cc3F)c2c(=O)n1C] |
| I-44 | 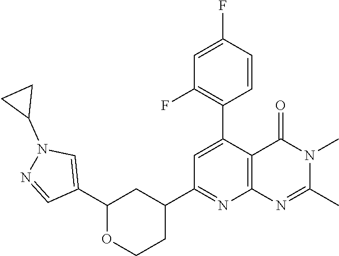 [SMILES: Cc1nc2nc(C3CCOC(c4cnn(C5CC5)c4)C3)cc(-c3ccc(F)cc3F)c2c(=O)n1C] |
| I-45 | 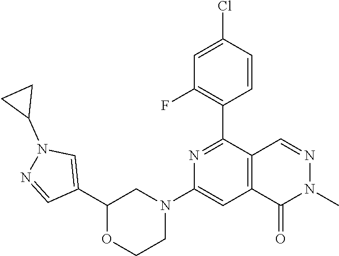 [SMILES: Cn1ncc2c(-c3ccc(Cl)cc3F)nc(N3CCOC(c4cnn(C5CC5)c4)C3)cc2c1=O] |
| I-46 | 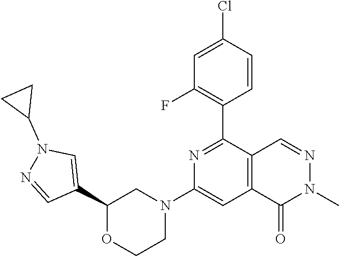 [SMILES: Cn1ncc2c(-c3ccc(Cl)cc3F)nc(N3CCO[C@@H](c4cnn(C5CC5)c4)C3)cc2c1=O] |
| I-47 | 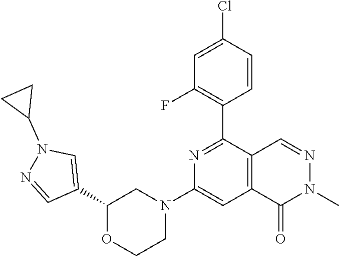 [SMILES: Cn1ncc2c(-c3ccc(Cl)cc3F)nc(N3CCO[C@H](c4cnn(C5CC5)c4)C3)cc2c1=O] |
| I-48 | 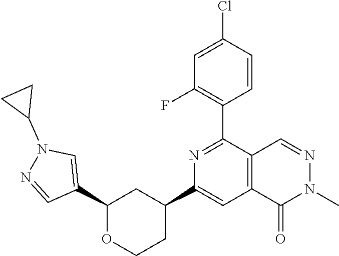 [SMILES: Cn1ncc2c(-c3ccc(Cl)cc3F)nc([C@H]3CCO[C@@H](c4cnn(C5CC5)c4)C3)cc2c1=O] |
| I-49 | 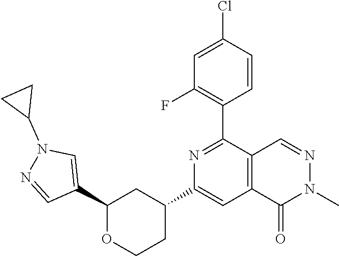 [SMILES: Cn1ncc2c(-c3ccc(Cl)cc3F)nc([C@@H]3CCO[C@@H](c4cnn(C5CC5)c4)C3)cc2c1=O] |
| I-50 | 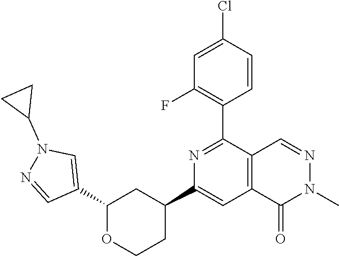 [SMILES: Cn1ncc2c(-c3ccc(Cl)cc3F)nc([C@H]3CCO[C@H](c4cnn(C5CC5)c4)C3)cc2c1=O] |
| I-51 | 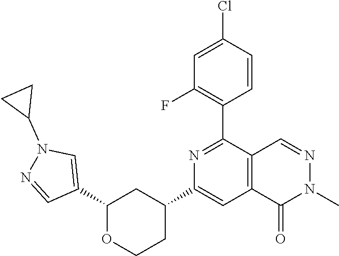 [SMILES: Cn1ncc2c(-c3ccc(Cl)cc3F)nc([C@@H]3CCO[C@H](c4cnn(C5CC5)c4)C3)cc2c1=O] |
| I-52 | 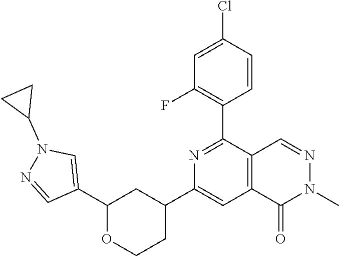 [SMILES: Cn1ncc2c(-c3ccc(Cl)cc3F)nc(C3CCOC(c4cnn(C5CC5)c4)C3)cc2c1=O] |
| I-53 | 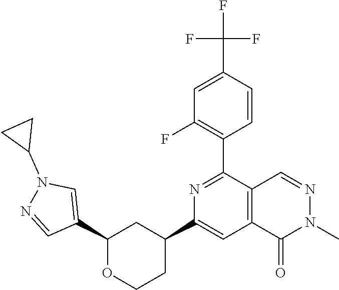 [SMILES: Cn1ncc2c(-c3ccc(C(F)(F)F)cc3F)nc([C@H]3CCO[C@@H](c4cnn(C5CC5)c4)C3)cc2c1=O] |
| I-54 | 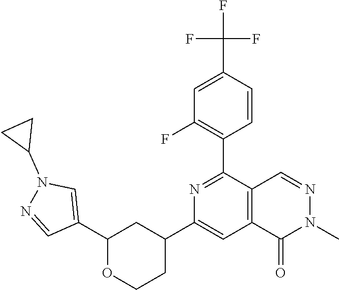 [SMILES: Cn1ncc2c(-c3ccc(C(F)(F)F)cc3F)nc(C3CCOC(c4cnn(C5CC5)c4)C3)cc2c1=O] |
| I-55 | 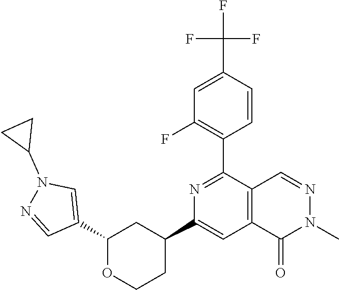 [SMILES: Cn1ncc2c(-c3ccc(C(F)(F)F)cc3F)nc([C@H]3CCO[C@H](c4cnn(C5CC5)c4)C3)cc2c1=O] |
| I-56 | 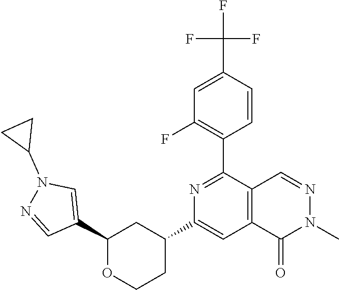 [SMILES: Cn1ncc2c(-c3ccc(C(F)(F)F)cc3F)nc([C@@H]3CCO[C@@H](c4cnn(C5CC5)c4)C3)cc2c1=O] |
| I-57 | 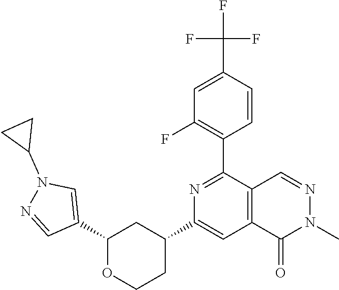 [SMILES: Cn1ncc2c(-c3ccc(C(F)(F)F)cc3F)nc([C@@H]3CCO[C@H](c4cnn(C5CC5)c4)C3)cc2c1=O] |
| I-58 | 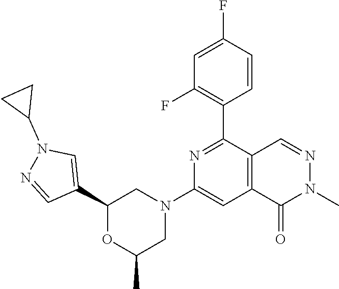 [SMILES: C[C@@H]1CN(c2cc3c(=O)n(C)ncc3c(-c3ccc(F)cc3F)n2)C[C@H](c2cnn(C3CC3)c2)O1] |
| I-59 | 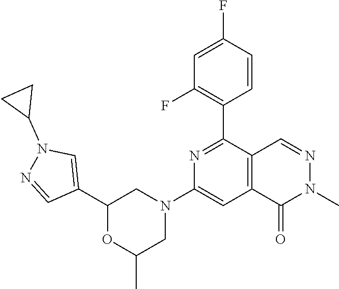 [SMILES: CC1CN(c2cc3c(=O)n(C)ncc3c(-c3ccc(F)cc3F)n2)CC(c2cnn(C3CC3)c2)O1] |
| I-60 | 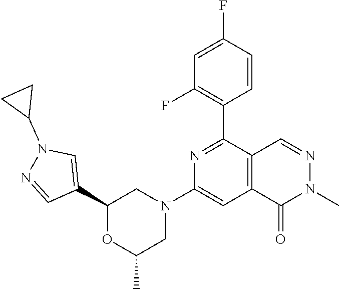 [SMILES: C[C@H]1CN(c2cc3c(=O)n(C)ncc3c(-c3ccc(F)cc3F)n2)C[C@H](c2cnn(C3CC3)c2)O1] |
| I-61 | 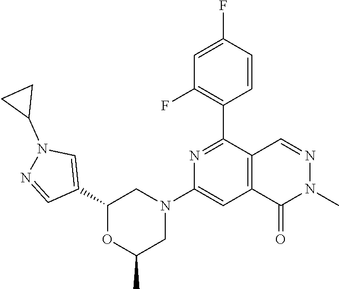 [SMILES: C[C@@H]1CN(c2cc3c(=O)n(C)ncc3c(-c3ccc(F)cc3F)n2)C[C@@H](c2cnn(C3CC3)c2)O1] |
| I-62 | 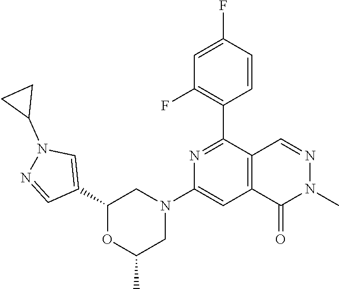 [SMILES: C[C@H]1CN(c2cc3c(=O)n(C)ncc3c(-c3ccc(F)cc3F)n2)C[C@@H](c2cnn(C3CC3)c2)O1] |
| I-63 | 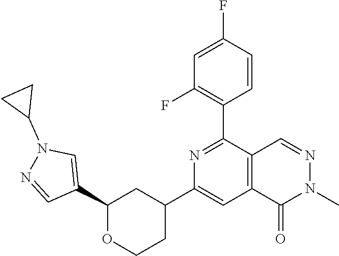 [SMILES: Cn1ncc2c(-c3ccc(F)cc3F)nc(C3CCO[C@@H](c4cnn(C5CC5)c4)C3)cc2c1=O] |
| I-64 | 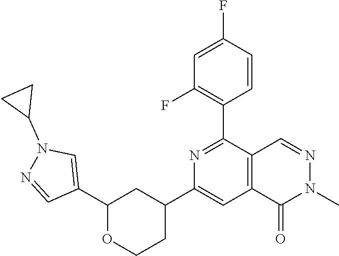 [SMILES: Cn1ncc2c(-c3ccc(F)cc3F)nc(C3CCOC(c4cnn(C5CC5)c4)C3)cc2c1=O] |
| I-65 | 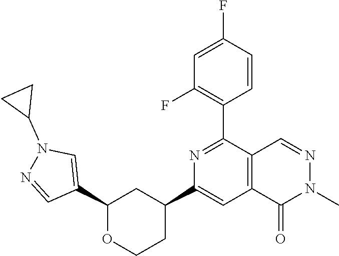 [SMILES: Cn1ncc2c(-c3ccc(F)cc3F)nc([C@H]3CCO[C@@H](c4cnn(C5CC5)c4)C3)cc2c1=O] |
| I-66 | 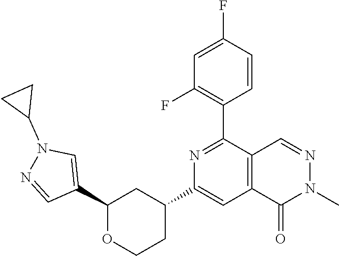 [SMILES: Cn1ncc2c(-c3ccc(F)cc3F)nc([C@@H]3CCO[C@@H](c4cnn(C5CC5)c4)C3)cc2c1=O] |
| I-67 | 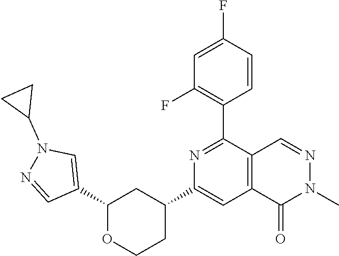 [SMILES: Cn1ncc2c(-c3ccc(F)cc3F)nc([C@@H]3CCO[C@H](c4cnn(C5CC5)c4)C3)cc2c1=O] |
| I-68 | 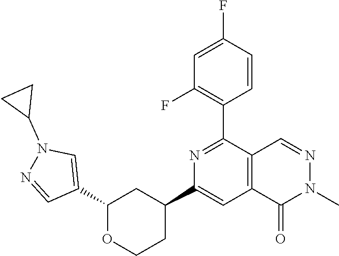 [SMILES: Cn1ncc2c(-c3ccc(F)cc3F)nc([C@H]3CCO[C@H](c4cnn(C5CC5)c4)C3)cc2c1=O] |
| I-69 | 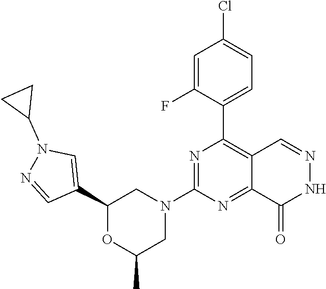 [SMILES: C[C@@H]1CN(c2nc(-c3ccc(Cl)cc3F)c3cn[nH]c(=O)c3n2)C[C@H](c2cnn(C3CC3)c2)O1] |
| I-70 | 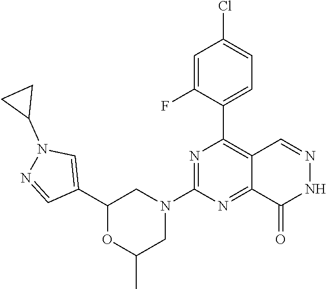 [SMILES: CC1CN(c2nc(-c3ccc(Cl)cc3F)c3cn[nH]c(=O)c3n2)CC(c2cnn(C3CC3)c2)O1] |
| I-71 | 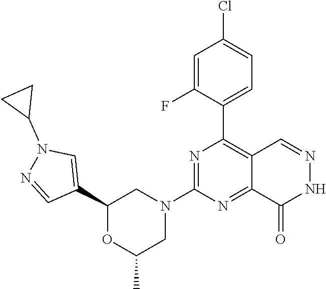 [SMILES: C[C@H]1CN(c2nc(-c3ccc(Cl)cc3F)c3cn[nH]c(=O)c3n2)C[C@H](c2cnn(C3CC3)c2)O1] |
| I-72 | 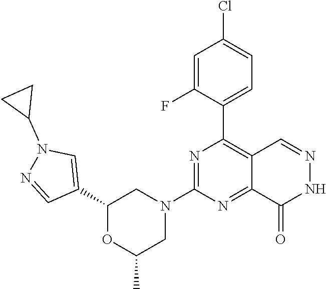 [SMILES: C[C@H]1CN(c2nc(-c3ccc(Cl)cc3F)c3cn[nH]c(=O)c3n2)C[C@@H](c2cnn(C3CC3)c2)O1] |
| I-73 | 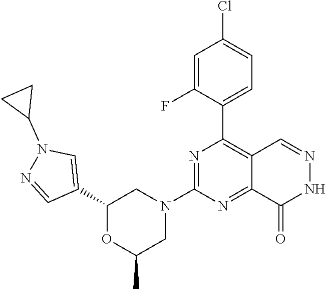 [SMILES: C[C@@H]1CN(c2nc(-c3ccc(Cl)cc3F)c3cn[nH]c(=O)c3n2)C[C@@H](c2cnn(C3CC3)c2)O1] |
| I-74 | 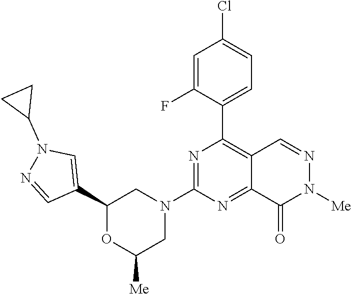 [SMILES: C[C@@H]1CN(c2nc(-c3ccc(Cl)cc3F)c3cnn(C)c(=O)c3n2)C[C@H](c2cnn(C3CC3)c2)O1] |
| I-75 | 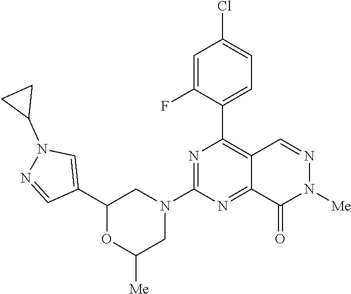 [SMILES: CC1CN(c2nc(-c3ccc(Cl)cc3F)c3cnn(C)c(=O)c3n2)CC(c2cnn(C3CC3)c2)O1] |
| I-76 | 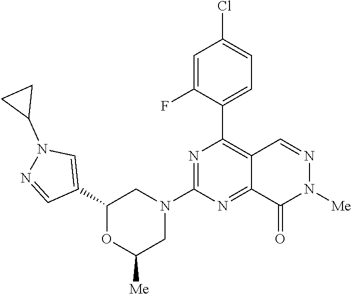 [SMILES: C[C@@H]1CN(c2nc(-c3ccc(Cl)cc3F)c3cnn(C)c(=O)c3n2)C[C@@H](c2cnn(C3CC3)c2)O1] |
| I-77 | 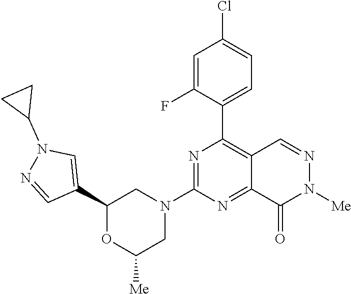 [SMILES: C[C@H]1CN(c2nc(-c3ccc(Cl)cc3F)c3cnn(C)c(=O)c3n2)C[C@H](c2cnn(C3CC3)c2)O1] |
| I-78 | 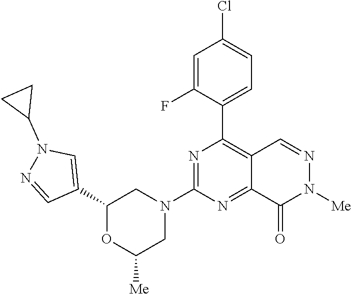 [SMILES: C[C@H]1CN(c2nc(-c3ccc(Cl)cc3F)c3cnn(C)c(=O)c3n2)C[C@@H](c2cnn(C3CC3)c2)O1] |
| I-79 | 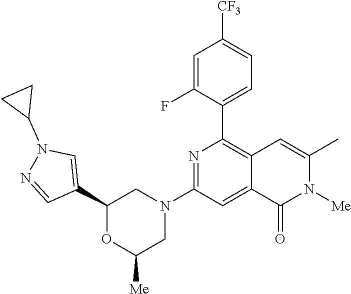 [SMILES: Cc1cc2c(-c3ccc(C(F)(F)F)cc3F)nc(N3C[C@@H](C)O[C@@H](c4cnn(C5CC5)c4)C3)cc2c(=O)n1C] |
| I-80 | 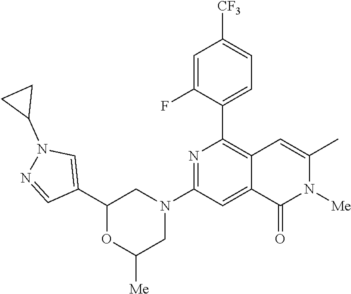 [SMILES: Cc1cc2c(-c3ccc(C(F)(F)F)cc3F)nc(N3CC(C)OC(c4cnn(C5CC5)c4)C3)cc2c(=O)n1C] |
| I-81 | 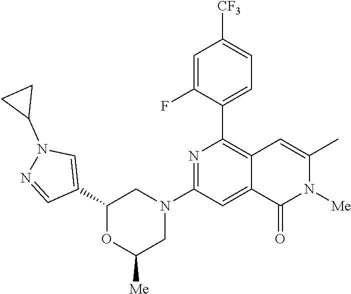 [SMILES: Cc1cc2c(-c3ccc(C(F)(F)F)cc3F)nc(N3C[C@@H](C)O[C@H](c4cnn(C5CC5)c4)C3)cc2c(=O)n1C] |
| I-82 | 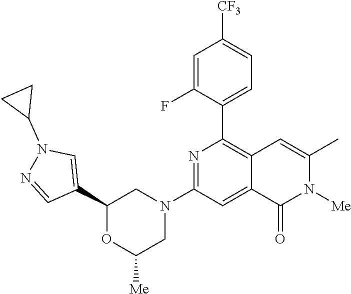 [SMILES: Cc1cc2c(-c3ccc(C(F)(F)F)cc3F)nc(N3C[C@H](C)O[C@@H](c4cnn(C5CC5)c4)C3)cc2c(=O)n1C] |
| I-83 | 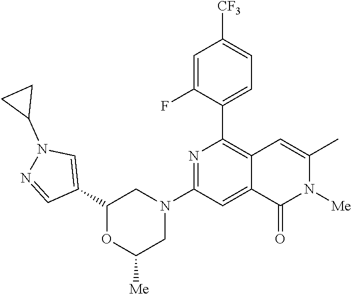 |
| I-84 | 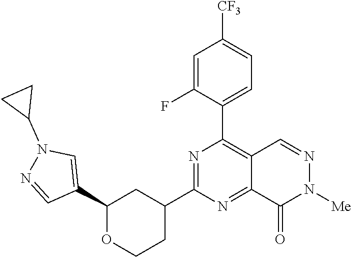 [SMILES: Cn1ncc2c(-c3ccc(C(F)(F)F)cc3F)nc(C3CCO[C@@H](c4cnn(C5CC5)c4)C3)nc2c1=O] |
| I-85 | 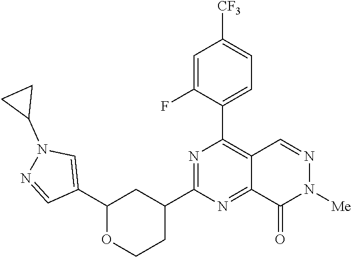 [SMILES: Cn1ncc2c(-c3ccc(C(F)(F)F)cc3F)nc(C3CCOC(c4cnn(C5CC5)c4)C3)nc2c1=O] |
| I-86 | 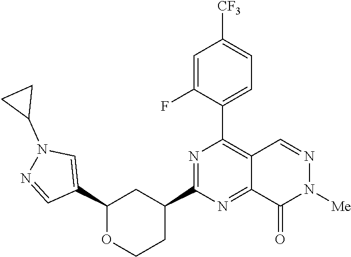 [SMILES: Cn1ncc2c(-c3ccc(C(F)(F)F)cc3F)nc([C@H]3CCO[C@@H](c4cnn(C5CC5)c4)C3)nc2c1=O] |
| I-87 | 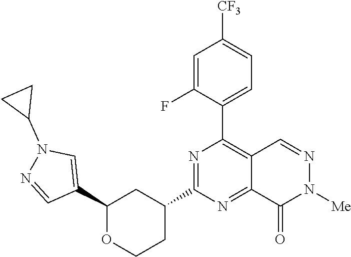 [SMILES: Cn1ncc2c(-c3ccc(C(F)(F)F)cc3F)nc([C@@H]3CCO[C@@H](c4cnn(C5CC5)c4)C3)nc2c1=O] |
| I-88 | 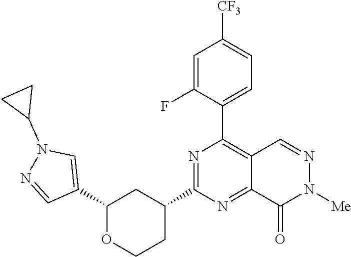 [SMILES: Cn1ncc2c(-c3ccc(C(F)(F)F)cc3F)nc([C@@H]3CCO[C@H](c4cnn(C5CC5)c4)C3)nc2c1=O] |
| I-89 | 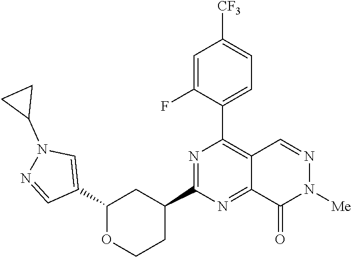 [SMILES: Cn1ncc2c(-c3ccc(C(F)(F)F)cc3F)nc([C@H]3CCO[C@H](c4cnn(C5CC5)c4)C3)nc2c1=O] |
| I-90 | 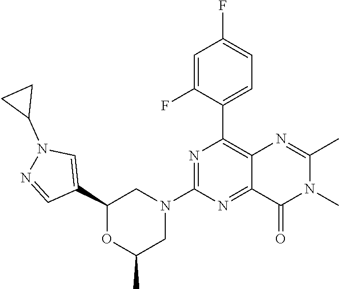 [SMILES: Cc1nc2c(-c3ccc(F)cc3F)nc(N3C[C@@H](C)O[C@@H](c4cnn(C5CC5)c4)C3)nc2c(=O)n1C] |
| I-91 | 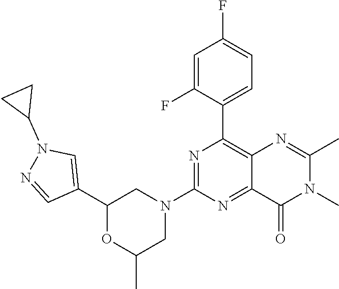 [SMILES: Cc1nc2c(-c3ccc(F)cc3F)nc(N3CC(C)OC(c4cnn(C5CC5)c4)C3)nc2c(=O)n1C] |
| I-92 | 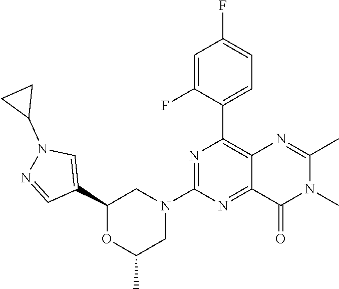 [SMILES: Cc1nc2c(-c3ccc(F)cc3F)nc(N3C[C@H](C)O[C@@H](c4cnn(C5CC5)c4)C3)nc2c(=O)n1C] |
| I-93 | 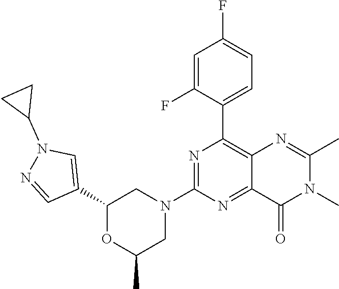 [SMILES: Cc1nc2c(-c3ccc(F)cc3F)nc(N3C[C@@H](C)O[C@H](c4cnn(C5CC5)c4)C3)nc2c(=O)n1C] |
| I-94 | 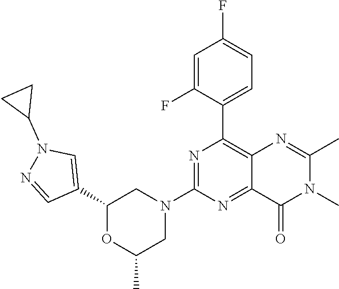 [SMILES: Cc1nc2c(-c3ccc(F)cc3F)nc(N3C[C@@H](c4cnn(C5CC5)c4)O[C@@H](C)C3)nc2c(=O)n1C] |
| I-95 | 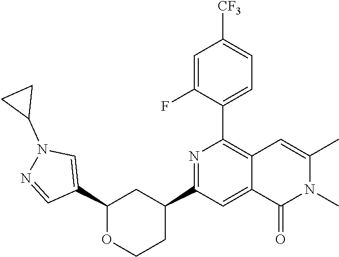 [SMILES: Cc1cc2c(-c3ccc(C(F)(F)F)cc3F)nc([C@H]3CCO[C@@H](c4cnn(C5CC5)c4)C3)cc2c(=O)n1C] |
| I-96 | 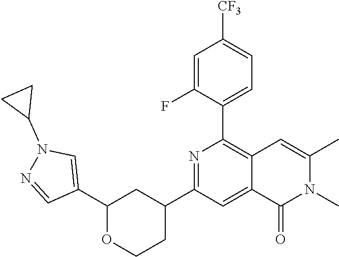 [SMILES: Cc1cc2c(-c3ccc(C(F)(F)F)cc3F)nc(C3CCOC(c4cnn(C5CC5)c4)C3)cc2c(=O)n1C] |
| I-97 | 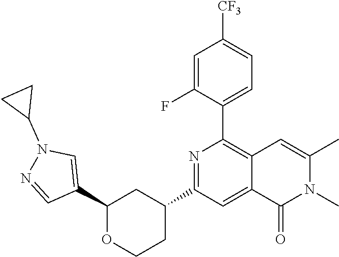 [SMILES: Cc1cc2c(-c3ccc(C(F)(F)F)cc3F)nc([C@@H]3CCO[C@@H](c4cnn(C5CC5)c4)C3)cc2c(=O)n1C] |
| I-98 | 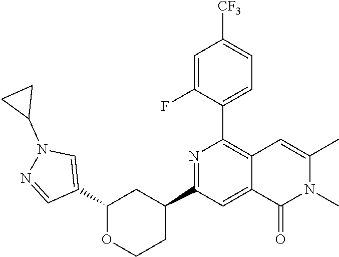 [SMILES: Cc1cc2c(-c3ccc(C(F)(F)F)cc3F)nc([C@H]3CCO[C@H](c4cnn(C5CC5)c4)C3)cc2c(=O)n1C] |
| I-99 | 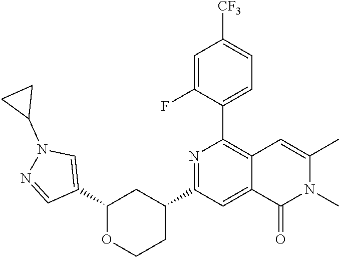 [SMILES: Cc1cc2c(-c3ccc(C(F)(F)F)cc3F)nc([C@@H]3CCO[C@H](c4cnn(C5CC5)c4)C3)cc2c(=O)n1C] |
| I-100 | 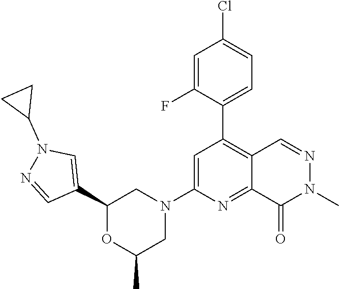 [SMILES: C[C@@H]1CN(c2cc(-c3ccc(Cl)cc3F)c3cnn(C)c(=O)c3n2)C[C@H](c2cnn(C3CC3)c2)O1] |
| I-101 | 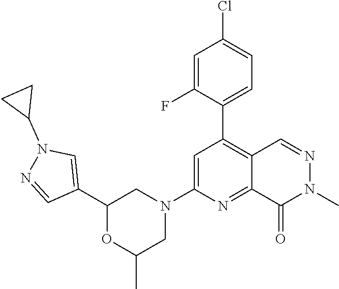 [SMILES: CC1CN(c2cc(-c3ccc(Cl)cc3F)c3cnn(C)c(=O)c3n2)CC(c2cnn(C3CC3)c2)O1] |
| I-102 | 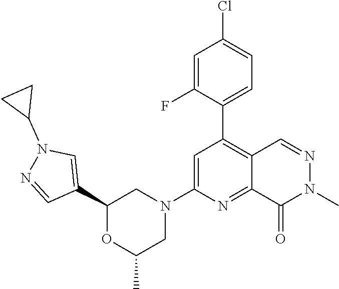 [SMILES: C[C@H]1CN(c2cc(-c3ccc(Cl)cc3F)c3cnn(C)c(=O)c3n2)C[C@H](c2cnn(C3CC3)c2)O1] |
| I-103 | 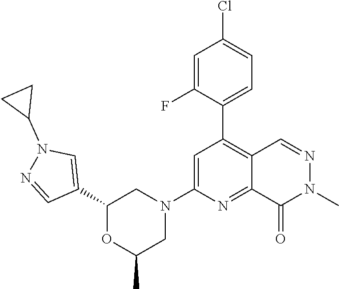 [SMILES: C[C@@H]1CN(c2cc(-c3ccc(Cl)cc3F)c3cnn(C)c(=O)c3n2)C[C@@H](c2cnn(C3CC3)c2)O1] |
| I-104 | 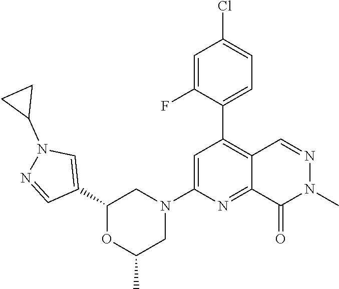 [SMILES: C[C@H]1CN(c2cc(-c3ccc(Cl)cc3F)c3cnn(C)c(=O)c3n2)C[C@@H](c2cnn(C3CC3)c2)O1] |
| I-105 | 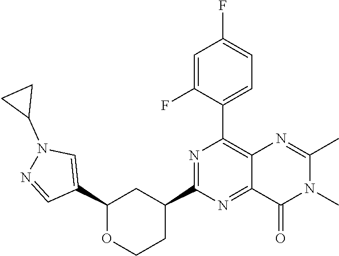 [SMILES: Cc1nc2c(-c3ccc(F)cc3F)nc([C@H]3CCO[C@@H](c4cnn(C5CC5)c4)C3)nc2c(=O)n1C] |
| I-106 | 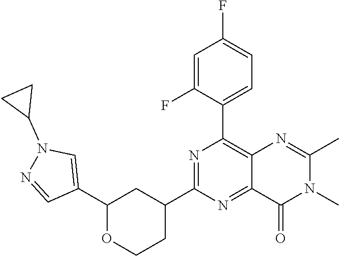 [SMILES: Cc1nc2c(-c3ccc(F)cc3F)nc(C3CCOC(c4cnn(C5CC5)c4)C3)nc2c(=O)n1C] |
| I-107 | 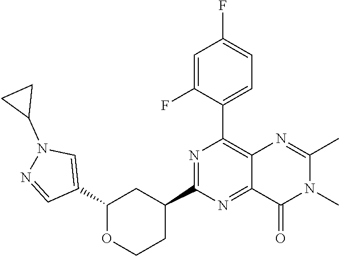 [SMILES: Cc1nc2c(-c3ccc(F)cc3F)nc([C@H]3CCO[C@H](c4cnn(C5CC5)c4)C3)nc2c(=O)n1C] |
| I-108 | 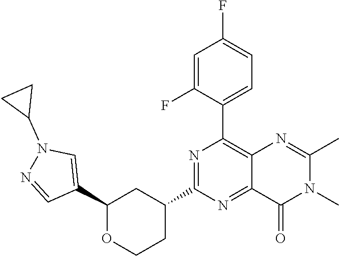 [SMILES: Cc1nc2c(-c3ccc(F)cc3F)nc([C@@H]3CCO[C@@H](c4cnn(C5CC5)c4)C3)nc2c(=O)n1C] |
| I-109 | 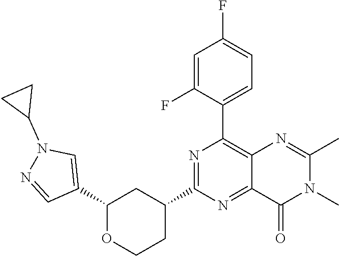 [SMILES: Cc1nc2c(-c3ccc(F)cc3F)nc([C@@H]3CCO[C@H](c4cnn(C5CC5)c4)C3)nc2c(=O)n1C] |
| I-110 | 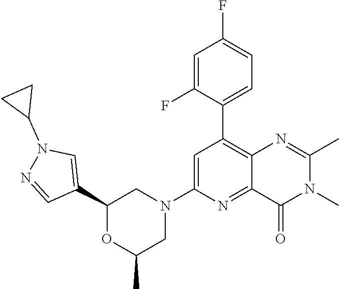 [SMILES: Cc1nc2c(-c3ccc(F)cc3F)cc(N3C[C@@H](C)O[C@@H](c4cnn(C5CC5)c4)C3)nc2c(=O)n1C] |
| I-111 | 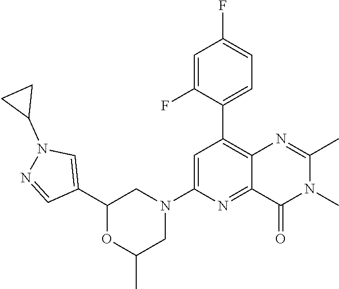 [SMILES: Cc1nc2c(-c3ccc(F)cc3F)cc(N3CC(C)OC(c4cnn(C5CC5)c4)C3)nc2c(=O)n1C] |
| I-112 | 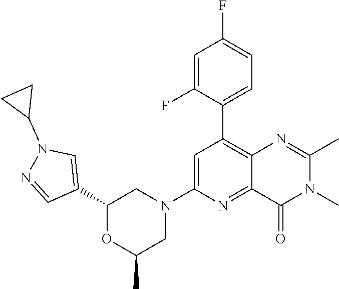 [SMILES: Cc1nc2c(-c3ccc(F)cc3F)cc(N3C[C@@H](C)O[C@H](c4cnn(C5CC5)c4)C3)nc2c(=O)n1C] |
| I-113 | 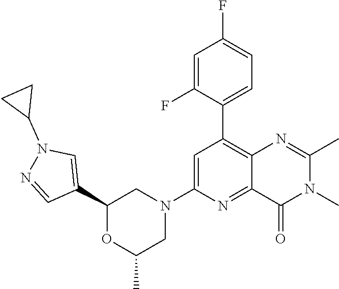 [SMILES: Cc1nc2c(-c3ccc(F)cc3F)cc(N3C[C@H](C)O[C@@H](c4cnn(C5CC5)c4)C3)nc2c(=O)n1C] |
| I-114 | 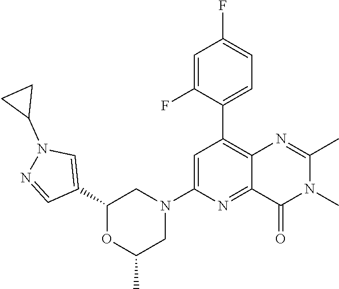 [SMILES: Cc1nc2c(-c3ccc(F)cc3F)cc(N3C[C@@H](c4cnn(C5CC5)c4)O[C@@H](C)C3)nc2c(=O)n1C] |
| I-115 | 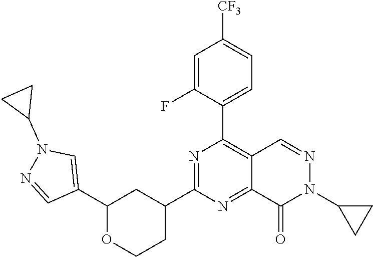 [SMILES: O=c1c2nc(C3CCOC(c4cnn(C5CC5)c4)C3)nc(-c3ccc(C(F)(F)F)cc3F)c2cnn1C1CC1] |
| I-116 | 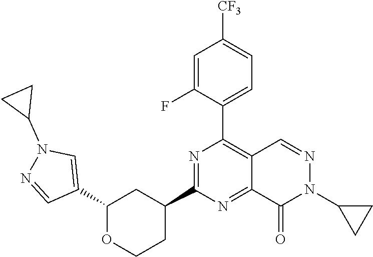 [SMILES: O=c1c2nc([C@H]3CCO[C@H](c4cnn(C5CC5)c4)C3)nc(-c3ccc(C(F)(F)F)cc3F)c2cnn1C1CC1] |
| I-117 | 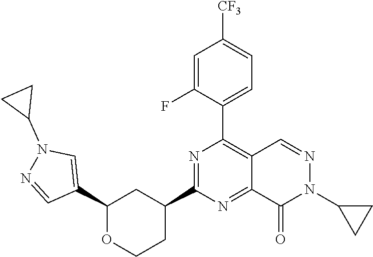 [SMILES: O=c1c2nc([C@H]3CCO[C@@H](c4cnn(C5CC5)c4)C3)nc(-c3ccc(C(F)(F)F)cc3F)c2cnn1C1CC1] |
| I-118 | 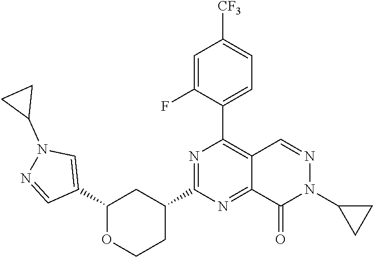 [SMILES: O=c1c2nc([C@@H]3CCO[C@H](c4cnn(C5CC5)c4)C3)nc(-c3ccc(C(F)(F)F)cc3F)c2cnn1C1CC1] |
| I-119 | 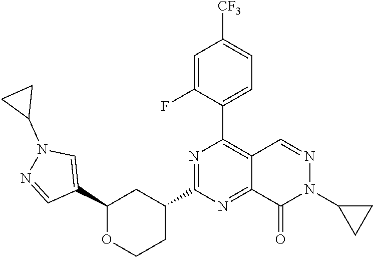 [SMILES: O=c1c2nc([C@@H]3CCO[C@@H](c4cnn(C5CC5)c4)C3)nc(-c3ccc(C(F)(F)F)cc3F)c2cnn1C1CC1] |
| I-120 | 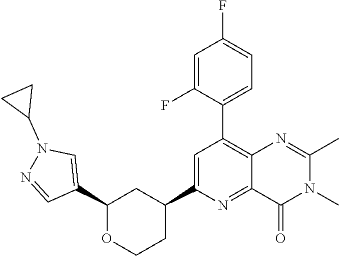 [SMILES: Cc1nc2c(-c3ccc(F)cc3F)cc([C@H]3CCO[C@@H](c4cnn(C5CC5)c4)C3)nc2c(=O)n1C] |
| I-121 | 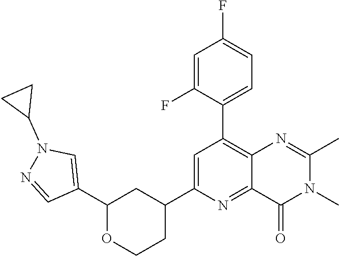 [SMILES: Cc1nc2c(-c3ccc(F)cc3F)cc(C3CCOC(c4cnn(C5CC5)c4)C3)nc2c(=O)n1C] |
| I-122 | 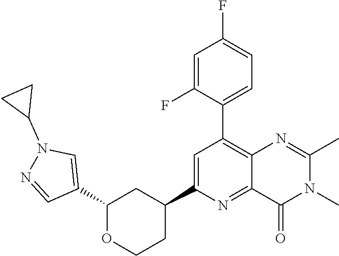 [SMILES: Cc1nc2c(-c3ccc(F)cc3F)cc([C@H]3CCO[C@H](c4cnn(C5CC5)c4)C3)nc2c(=O)n1C] |
| I-123 | 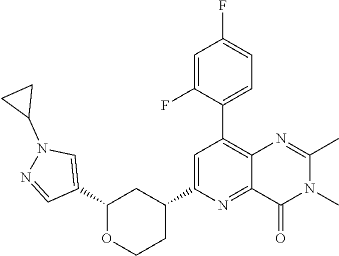 [SMILES: Cc1nc2c(-c3ccc(F)cc3F)cc([C@@H]3CCO[C@H](c4cnn(C5CC5)c4)C3)nc2c(=O)n1C] |
| I-124 | 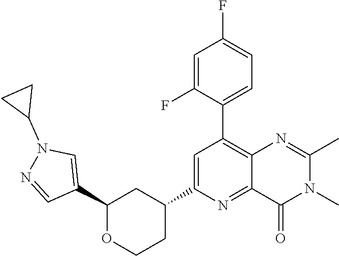 [SMILES: Cc1nc2c(-c3ccc(F)cc3F)cc([C@@H]3CCO[C@@H](c4cnn(C5CC5)c4)C3)nc2c(=O)n1C] |
| I-125 | 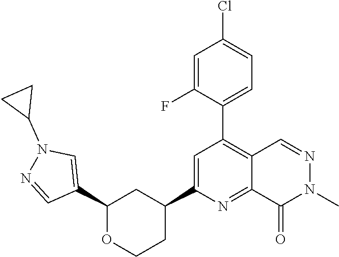 [SMILES: Cn1ncc2c(-c3ccc(Cl)cc3F)cc([C@H]3CCO[C@@H](c4cnn(C5CC5)c4)C3)nc2c1=O] |
| I-126 | 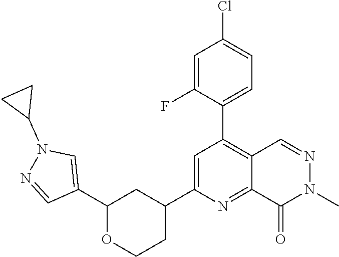 [SMILES: Cn1ncc2c(-c3ccc(Cl)cc3F)cc(C3CCOC(c4cnn(C5CC5)c4)C3)nc2c1=O] |
| I-127 | 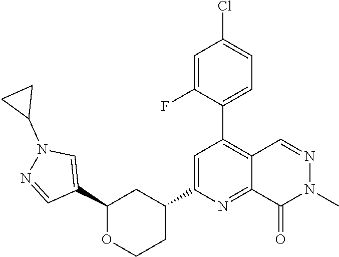 [SMILES: Cn1ncc2c(-c3ccc(Cl)cc3F)cc([C@@H]3CCO[C@@H](c4cnn(C5CC5)c4)C3)nc2c1=O] |
| I-128 | 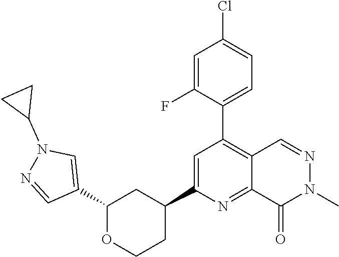 [SMILES: Cn1ncc2c(-c3ccc(Cl)cc3F)cc([C@H]3CCO[C@H](c4cnn(C5CC5)c4)C3)nc2c1=O] |
| I-129 | 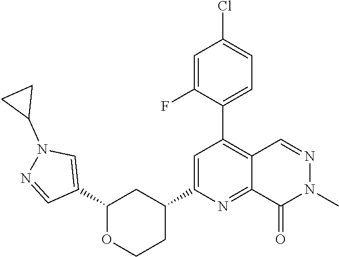 [SMILES: Cn1ncc2c(-c3ccc(Cl)cc3F)cc([C@@H]3CCO[C@H](c4cnn(C5CC5)c4)C3)nc2c1=O] |
| I-130 | 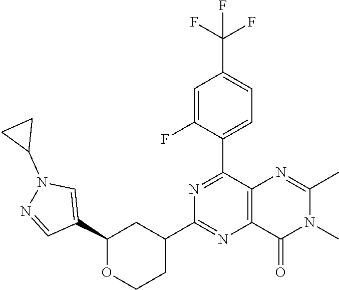 [SMILES: Cc1nc2c(-c3ccc(C(F)(F)F)cc3F)nc(C3CCO[C@@H](c4cnn(C5CC5)c4)C3)nc2c(=O)n1C] |
| I-131 | 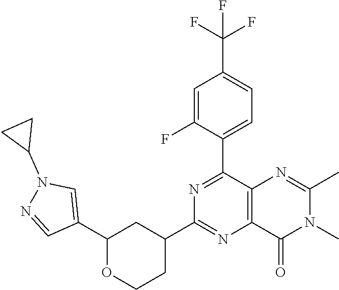 [SMILES: Cc1nc2c(-c3ccc(C(F)(F)F)cc3F)nc(C3CCOC(c4cnn(C5CC5)c4)C3)nc2c(=O)n1C] |
| I-132 | 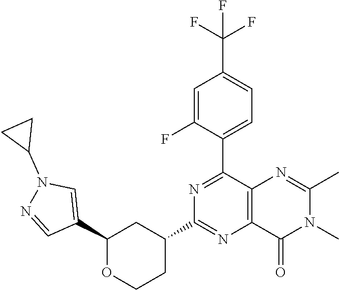 [SMILES: Cc1nc2c(-c3ccc(C(F)(F)F)cc3F)nc([C@@H]3CCO[C@@H](c4cnn(C5CC5)c4)C3)nc2c(=O)n1C] |
| I-133 | 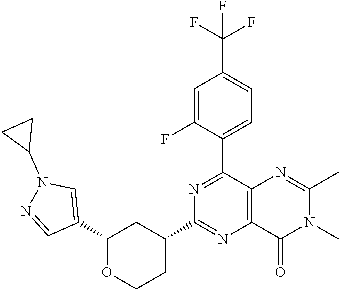 [SMILES: Cc1nc2c(-c3ccc(C(F)(F)F)cc3F)nc([C@@H]3CCO[C@H](c4cnn(C5CC5)c4)C3)nc2c(=O)n1C] |
| I-134 | 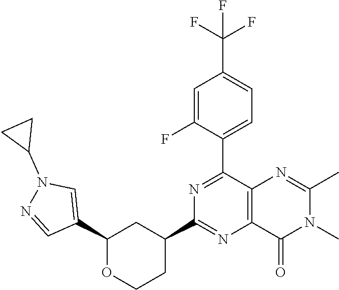 [SMILES: Cc1nc2c(-c3ccc(C(F)(F)F)cc3F)nc([C@H]3CCO[C@@H](c4cnn(C5CC5)c4)C3)nc2c(=O)n1C] |
| I-135 | 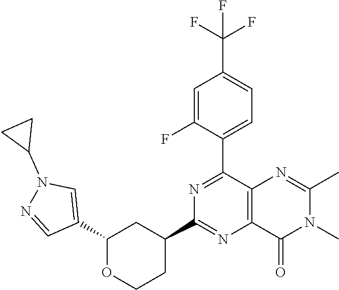 [SMILES: Cc1nc2c(-c3ccc(C(F)(F)F)cc3F)nc([C@H]3CCO[C@H](c4cnn(C5CC5)c4)C3)nc2c(=O)n1C] |
| I-136 | 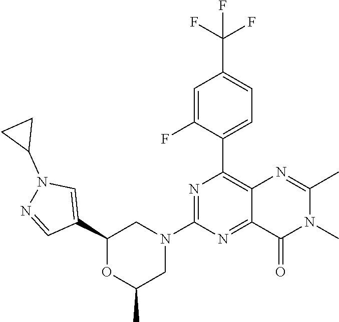 [SMILES: Cc1nc2c(-c3ccc(C(F)(F)F)cc3F)nc(N3C[C@@H](C)O[C@@H](c4cnn(C5CC5)c4)C3)nc2c(=O)n1C] |
| I-137 | 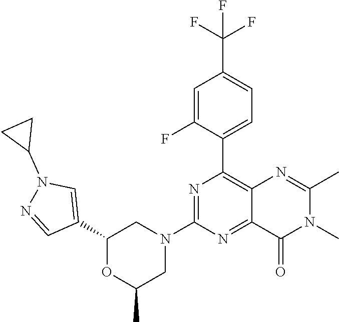 [SMILES: Cc1nc2c(-c3ccc(C(F)(F)F)cc3F)nc(N3C[C@@H](C)O[C@H](c4cnn(C5CC5)c4)C3)nc2c(=O)n1C] |
| I-138 | 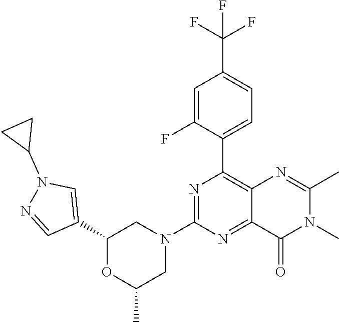 [SMILES: Cc1nc2c(-c3ccc(C(F)(F)F)cc3F)nc(N3C[C@@H](c4cnn(C5CC5)c4)O[C@@H](C)C3)nc2c(=O)n1C] |
| I-139 | 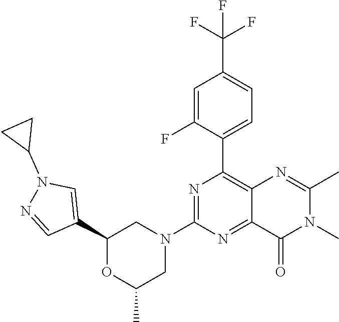 [SMILES: Cc1nc2c(-c3ccc(C(F)(F)F)cc3F)nc(N3C[C@H](C)O[C@@H](c4cnn(C5CC5)c4)C3)nc2c(=O)n1C] |
| I-140 | 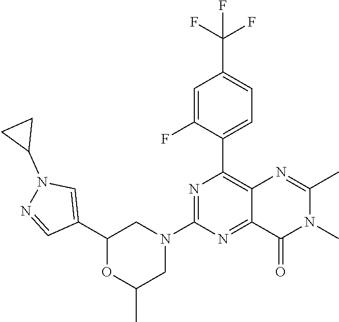 [SMILES: Cc1nc2c(-c3ccc(C(F)(F)F)cc3F)nc(N3CC(C)OC(c4cnn(C5CC5)c4)C3)nc2c(=O)n1C] |
| I-141 | 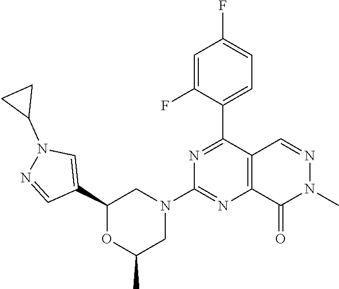 [SMILES: C[C@@H]1CN(c2nc(-c3ccc(F)cc3F)c3cnn(C)c(=O)c3n2)C[C@H](c2cnn(C3CC3)c2)O1] |
| I-142 | 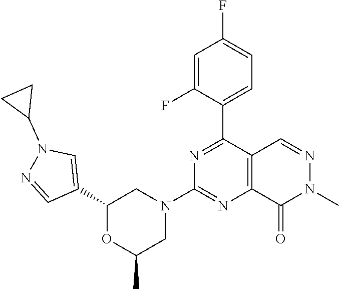 [SMILES: C[C@@H]1CN(c2nc(-c3ccc(F)cc3F)c3cnn(C)c(=O)c3n2)C[C@@H](c2cnn(C3CC3)c2)O1] |
| I-143 | 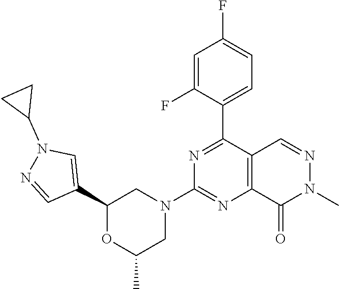 [SMILES: C[C@H]1CN(c2nc(-c3ccc(F)cc3F)c3cnn(C)c(=O)c3n2)C[C@H](c2cnn(C3CC3)c2)O1] |
| I-144 | 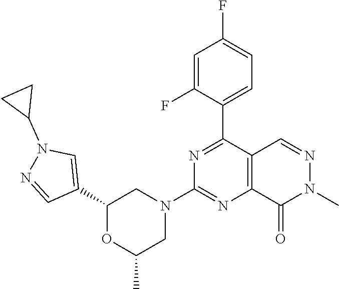 [SMILES: C[C@H]1CN(c2nc(-c3ccc(F)cc3F)c3cnn(C)c(=O)c3n2)C[C@@H](c2cnn(C3CC3)c2)O1] |
| I-145 | 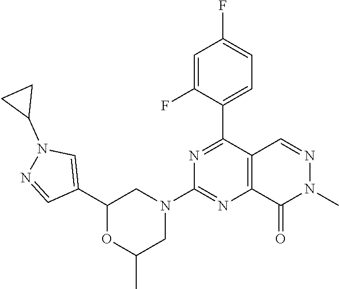 [SMILES: CC1CN(c2nc(-c3ccc(F)cc3F)c3cnn(C)c(=O)c3n2)CC(c2cnn(C3CC3)c2)O1] |
| I-146 | 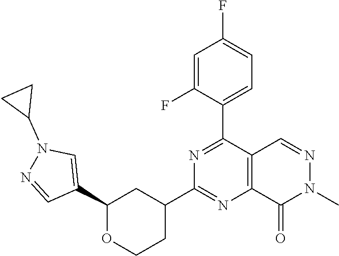 [SMILES: Cn1ncc2c(-c3ccc(F)cc3F)nc(C3CCO[C@@H](c4cnn(C5CC5)c4)C3)nc2c1=O] |
| I-147 | 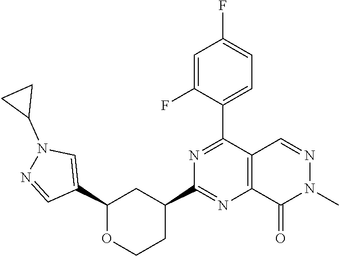 [SMILES: Cn1ncc2c(-c3ccc(F)cc3F)nc([C@H]3CCO[C@@H](c4cnn(C5CC5)c4)C3)nc2c1=O] |
| I-148 | 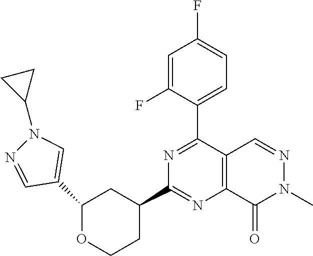 [SMILES: Cn1ncc2c(-c3ccc(F)cc3F)nc([C@H]3CCO[C@H](c4cnn(C5CC5)c4)C3)nc2c1=O] |
| I-149 | 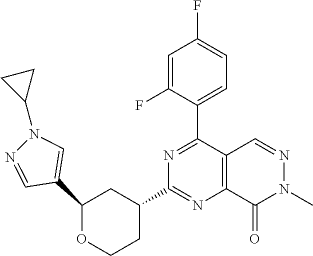 [SMILES: Cn1ncc2c(-c3ccc(F)cc3F)nc([C@@H]3CCO[C@@H](c4cnn(C5CC5)c4)C3)nc2c1=O] |
| I-150 | 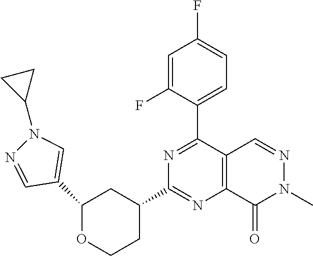 [SMILES: Cn1ncc2c(-c3ccc(F)cc3F)nc([C@@H]3CCO[C@H](c4cnn(C5CC5)c4)C3)nc2c1=O] |
| I-151 | 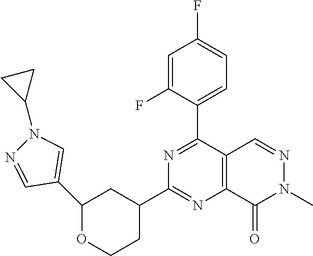 [SMILES: Cn1ncc2c(-c3ccc(F)cc3F)nc(C3CCOC(c4cnn(C5CC5)c4)C3)nc2c1=O] |
| I-152 | 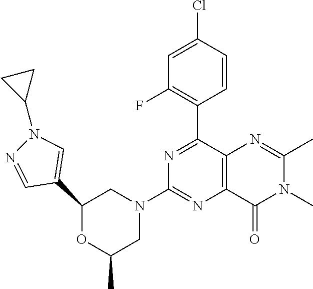 [SMILES: Cc1nc2c(-c3ccc(Cl)cc3F)nc(N3C[C@@H](C)O[C@@H](c4cnn(C5CC5)c4)C3)nc2c(=O)n1C] |
| I-153 | 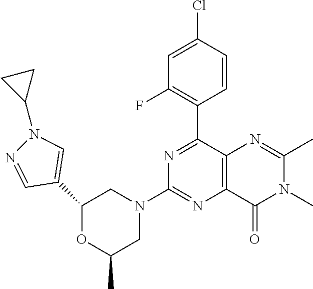 [SMILES: Cc1nc2c(-c3ccc(Cl)cc3F)nc(N3C[C@@H](C)O[C@H](c4cnn(C5CC5)c4)C3)nc2c(=O)n1C] |
| I-154 | 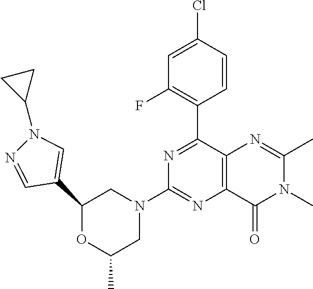 [SMILES: Cc1nc2c(-c3ccc(Cl)cc3F)nc(N3C[C@H](C)O[C@@H](c4cnn(C5CC5)c4)C3)nc2c(=O)n1C] |
| I-155 | 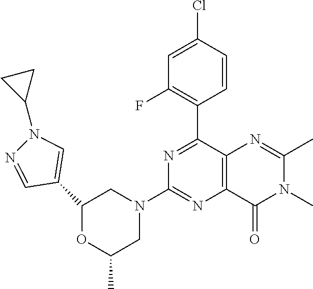 |
| I-156 | 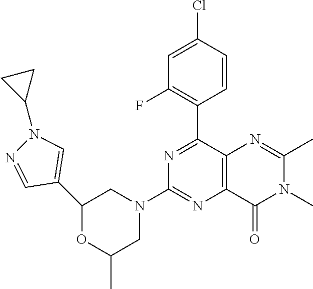 [SMILES: Cc1nc2c(-c3ccc(Cl)cc3F)nc(N3CC(C)OC(c4cnn(C5CC5)c4)C3)nc2c(=O)n1C] |
| I-157 | 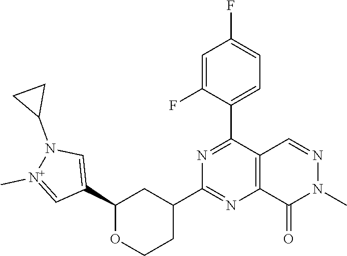 |
| I-158 | 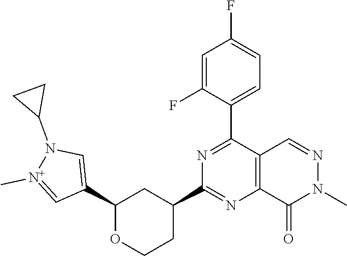 |
| I-159 | 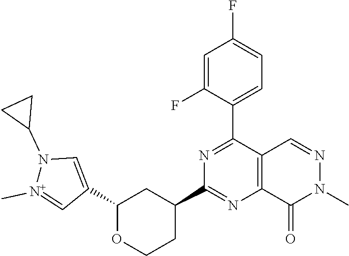 |
| I-160 | 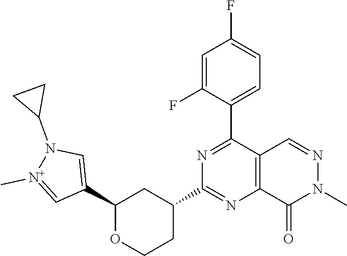 |
| I-161 | 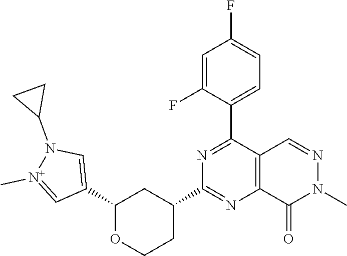 |
| I-162 | 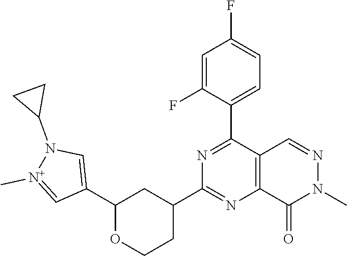 |
| I-163 | 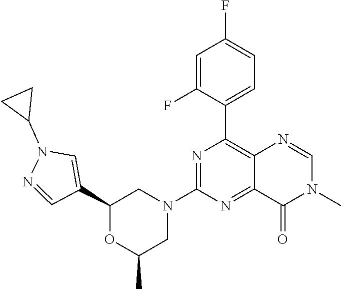 [SMILES: C[C@@H]1CN(c2nc(-c3ccc(F)cc3F)c3ncn(C)c(=O)c3n2)C[C@H](c2cnn(C3CC3)c2)O1] |
| I-164 | 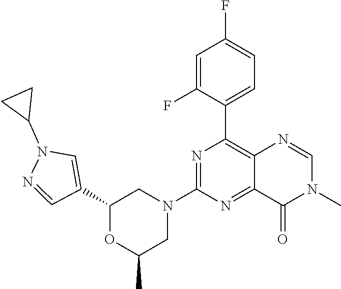 [SMILES: C[C@@H]1CN(c2nc(-c3ccc(F)cc3F)c3ncn(C)c(=O)c3n2)C[C@@H](c2cnn(C3CC3)c2)O1] |
| I-165 | 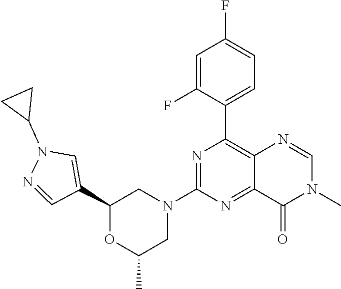 [SMILES: C[C@H]1CN(c2nc(-c3ccc(F)cc3F)c3ncn(C)c(=O)c3n2)C[C@H](c2cnn(C3CC3)c2)O1] |
| I-166 | 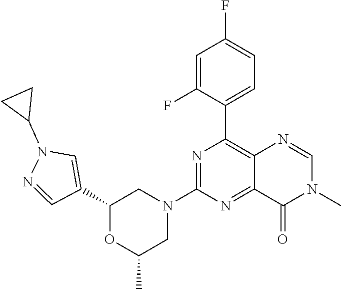 [SMILES: C[C@H]1CN(c2nc(-c3ccc(F)cc3F)c3ncn(C)c(=O)c3n2)C[C@@H](c2cnn(C3CC3)c2)O1] |
| I-167 | 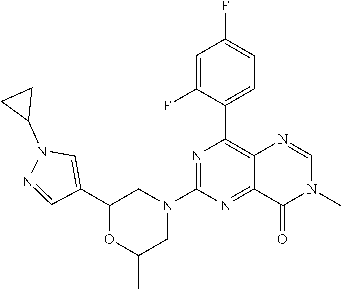 [SMILES: CC1CN(c2nc(-c3ccc(F)cc3F)c3ncn(C)c(=O)c3n2)CC(c2cnn(C3CC3)c2)O1] |
| I-168 | 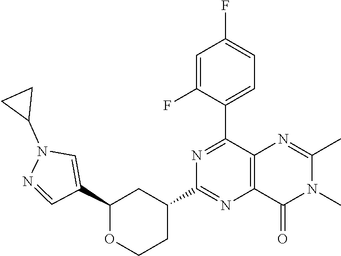 [SMILES: Cc1nc2c(-c3ccc(F)cc3F)nc([C@@H]3CCO[C@@H](c4cnn(C5CC5)c4)C3)nc2c(=O)n1C] |
| I-169 | 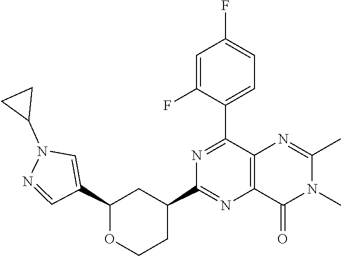 [SMILES: Cc1nc2c(-c3ccc(F)cc3F)nc([C@H]3CCO[C@@H](c4cnn(C5CC5)c4)C3)nc2c(=O)n1C] |
| I-170 | 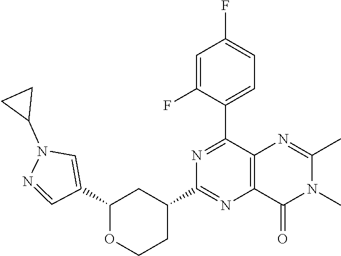 [SMILES: Cc1nc2c(-c3ccc(F)cc3F)nc([C@@H]3CCO[C@H](c4cnn(C5CC5)c4)C3)nc2c(=O)n1C] |
| I-171 | 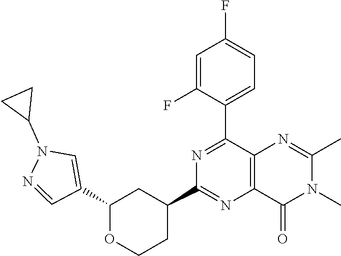 [SMILES: Cc1nc2c(-c3ccc(F)cc3F)nc([C@H]3CCO[C@H](c4cnn(C5CC5)c4)C3)nc2c(=O)n1C] |
| I-172 | 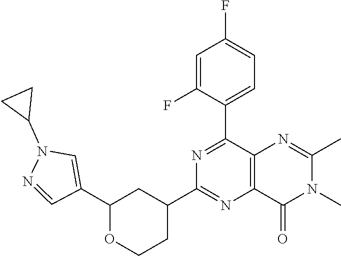 [SMILES: Cc1nc2c(-c3ccc(F)cc3F)nc(C3CCOC(c4cnn(C5CC5)c4)C3)nc2c(=O)n1C] |
| I-173 | 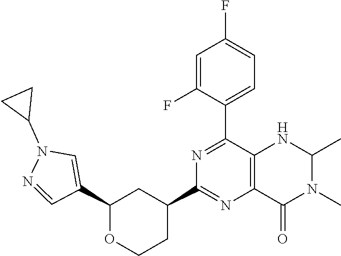 [SMILES: CC1Nc2c(nc([C@H]3CCO[C@@H](c4cnn(C5CC5)c4)C3)nc2-c2ccc(F)cc2F)C(=O)N1C] |
| I-174 | 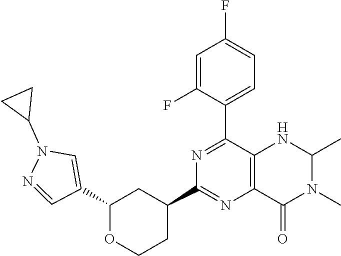 [SMILES: CC1Nc2c(nc([C@H]3CCO[C@H](c4cnn(C5CC5)c4)C3)nc2-c2ccc(F)cc2F)C(=O)N1C] |
| I-175 | 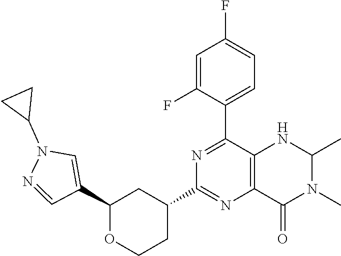 [SMILES: CC1Nc2c(nc([C@@H]3CCO[C@@H](c4cnn(C5CC5)c4)C3)nc2-c2ccc(F)cc2F)C(=O)N1C] |
| I-176 | 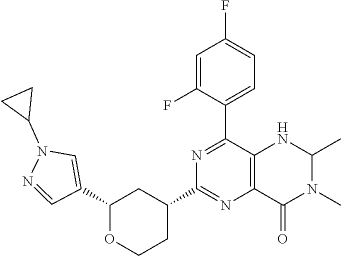 [SMILES: CC1Nc2c(nc([C@@H]3CCO[C@H](c4cnn(C5CC5)c4)C3)nc2-c2ccc(F)cc2F)C(=O)N1C] |
| I-177 | 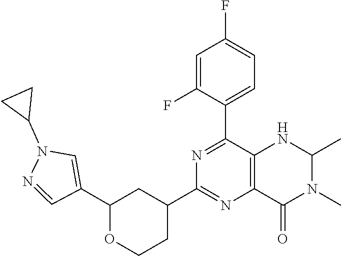 [SMILES: CC1Nc2c(nc(C3CCOC(c4cnn(C5CC5)c4)C3)nc2-c2ccc(F)cc2F)C(=O)N1C] |
| I-178 | 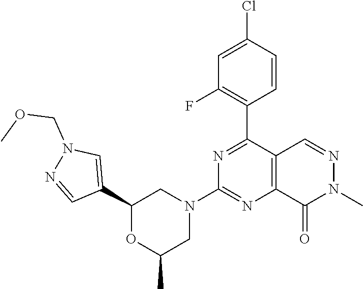 |
| I-179 | 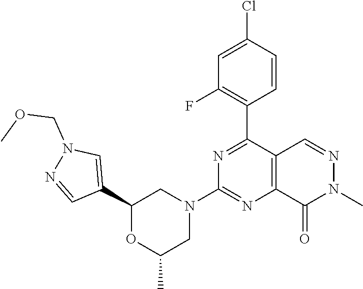 |
| I-180 | 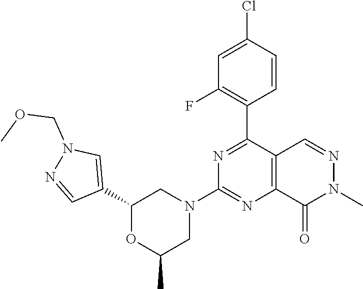 |
| I-181 | 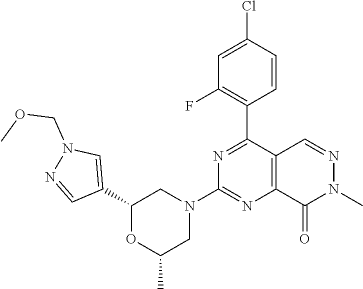 |
| I-182 | 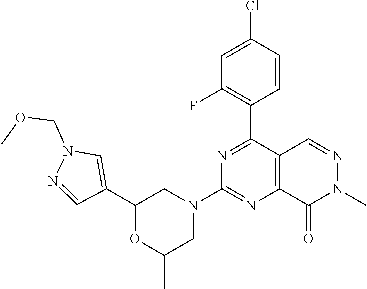 |
| I-183 | 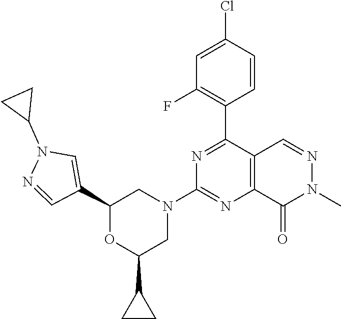 [SMILES: Cn1ncc2c(-c3ccc(Cl)cc3F)nc(N3C[C@@H](C4CC4)O[C@@H](c4cnn(C5CC5)c4)C3)nc2c1=O] |
| I-184 | 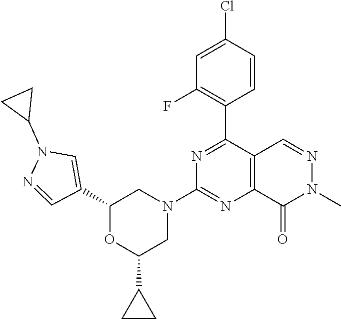 [SMILES: Cn1ncc2c(-c3ccc(Cl)cc3F)nc(N3C[C@@H](c4cnn(C5CC5)c4)O[C@@H](C4CC4)C3)nc2c1=O] |
| I-185 | 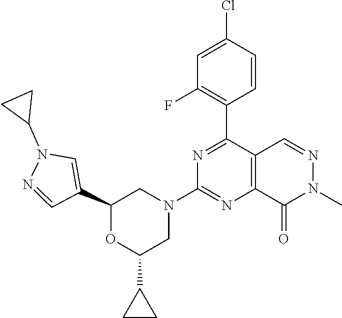 [SMILES: Cn1ncc2c(-c3ccc(Cl)cc3F)nc(N3C[C@H](c4cnn(C5CC5)c4)O[C@@H](C4CC4)C3)nc2c1=O] |
| I-186 | 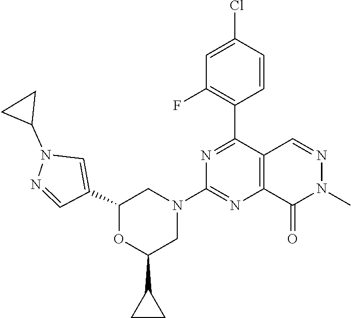 [SMILES: Cn1ncc2c(-c3ccc(Cl)cc3F)nc(N3C[C@@H](c4cnn(C5CC5)c4)O[C@H](C4CC4)C3)nc2c1=O] |
| I-187 | 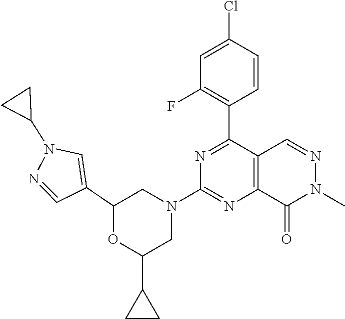 [SMILES: Cn1ncc2c(-c3ccc(Cl)cc3F)nc(N3CC(c4cnn(C5CC5)c4)OC(C4CC4)C3)nc2c1=O] |
| I-188 | 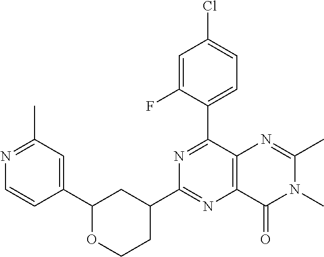 [SMILES: Cc1cc(C2CC(c3nc(-c4ccc(Cl)cc4F)c4nc(C)n(C)c(=O)c4n3)CCO2)ccn1] |
| I-189 | 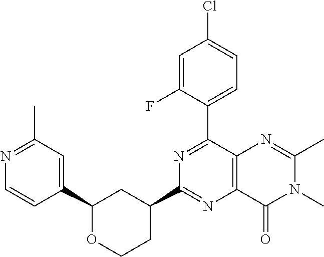 [SMILES: Cc1cc([C@H]2C[C@@H](c3nc(-c4ccc(Cl)cc4F)c4nc(C)n(C)c(=O)c4n3)CCO2)ccn1] |
| I-190 | 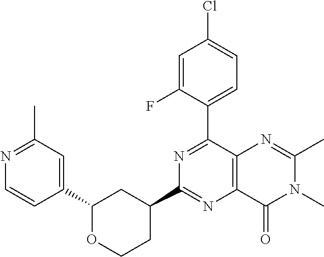 [SMILES: Cc1cc([C@@H]2C[C@@H](c3nc(-c4ccc(Cl)cc4F)c4nc(C)n(C)c(=O)c4n3)CCO2)ccn1] |
| I-191 | 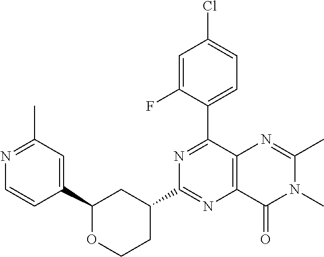 [SMILES: Cc1cc([C@H]2C[C@H](c3nc(-c4ccc(Cl)cc4F)c4nc(C)n(C)c(=O)c4n3)CCO2)ccn1] |
| I-192 | 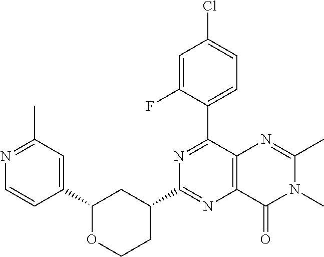 [SMILES: Cc1cc([C@@H]2C[C@H](c3nc(-c4ccc(Cl)cc4F)c4nc(C)n(C)c(=O)c4n3)CCO2)ccn1] |
| I-193 | 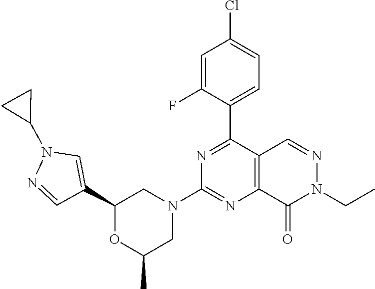 [SMILES: CCn1ncc2c(-c3ccc(Cl)cc3F)nc(N3C[C@@H](C)O[C@@H](c4cnn(C5CC5)c4)C3)nc2c1=O] |
| I-194 | 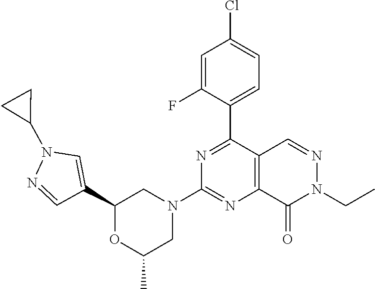 [SMILES: CCn1ncc2c(-c3ccc(Cl)cc3F)nc(N3C[C@H](C)O[C@@H](c4cnn(C5CC5)c4)C3)nc2c1=O] |
| I-195 | 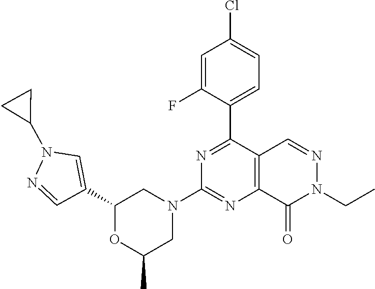 [SMILES: CCn1ncc2c(-c3ccc(Cl)cc3F)nc(N3C[C@@H](C)O[C@H](c4cnn(C5CC5)c4)C3)nc2c1=O] |
| I-196 | 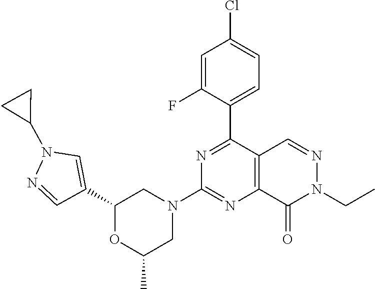 [SMILES: CCn1ncc2c(-c3ccc(Cl)cc3F)nc(N3C[C@@H](c4cnn(C5CC5)c4)O[C@@H](C)C3)nc2c1=O] |
| I-197 | 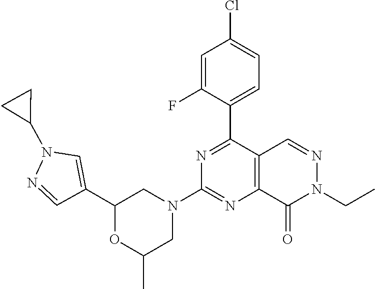 [SMILES: CCn1ncc2c(-c3ccc(Cl)cc3F)nc(N3CC(C)OC(c4cnn(C5CC5)c4)C3)nc2c1=O] |
| I-198 | 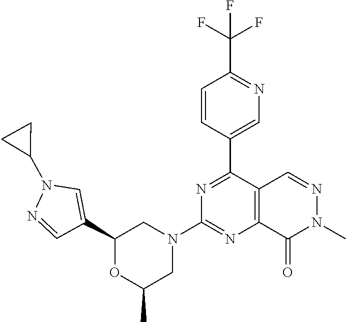 [SMILES: C[C@@H]1CN(c2nc(-c3ccc(C(F)(F)F)nc3)c3cnn(C)c(=O)c3n2)C[C@H](c2cnn(C3CC3)c2)O1] |
| I-199 | 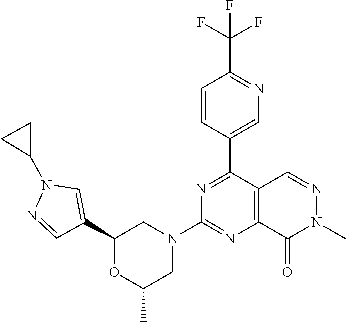 [SMILES: C[C@H]1CN(c2nc(-c3ccc(C(F)(F)F)nc3)c3cnn(C)c(=O)c3n2)C[C@H](c2cnn(C3CC3)c2)O1] |
| I-200 | 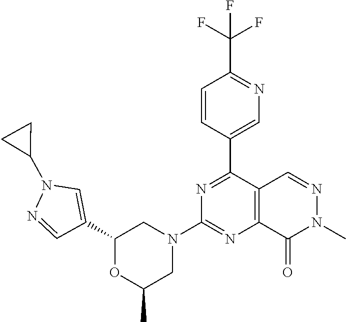 [SMILES: C[C@@H]1CN(c2nc(-c3ccc(C(F)(F)F)nc3)c3cnn(C)c(=O)c3n2)C[C@@H](c2cnn(C3CC3)c2)O1] |
| I-201 | 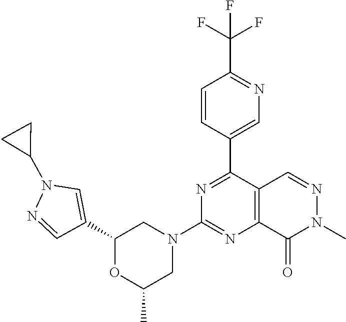 [SMILES: C[C@H]1CN(c2nc(-c3ccc(C(F)(F)F)nc3)c3cnn(C)c(=O)c3n2)C[C@@H](c2cnn(C3CC3)c2)O1] |
| I-202 | 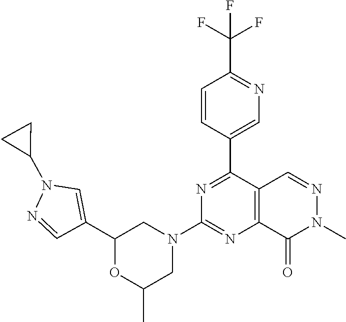 [SMILES: CC1CN(c2nc(-c3ccc(C(F)(F)F)nc3)c3cnn(C)c(=O)c3n2)CC(c2cnn(C3CC3)c2)O1] |
| I-203 | 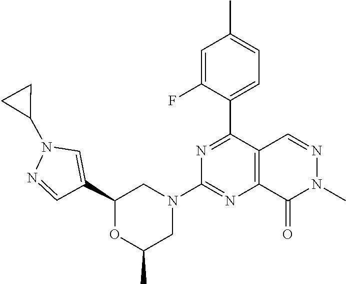 [SMILES: Cc1ccc(-c2nc(N3C[C@@H](C)O[C@@H](c4cnn(C5CC5)c4)C3)nc3c(=O)n(C)ncc23)c(F)c1] |
| I-204 | 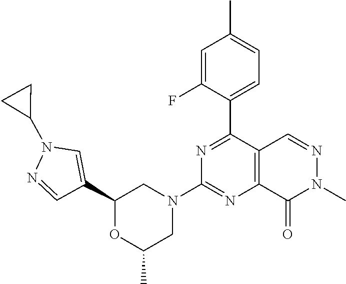 [SMILES: Cc1ccc(-c2nc(N3C[C@H](C)O[C@@H](c4cnn(C5CC5)c4)C3)nc3c(=O)n(C)ncc23)c(F)c1] |
| I-205 | 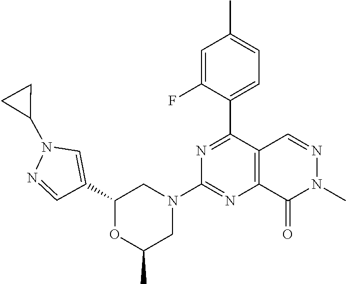 [SMILES: Cc1ccc(-c2nc(N3C[C@@H](C)O[C@H](c4cnn(C5CC5)c4)C3)nc3c(=O)n(C)ncc23)c(F)c1] |
| I-206 | 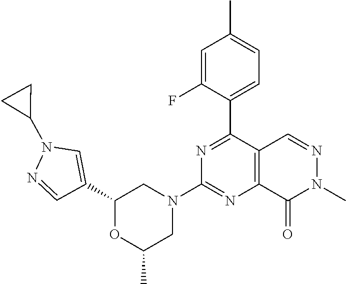 [SMILES: Cc1ccc(-c2nc(N3C[C@@H](c4cnn(C5CC5)c4)O[C@@H](C)C3)nc3c(=O)n(C)ncc23)c(F)c1] |
| I-207 | 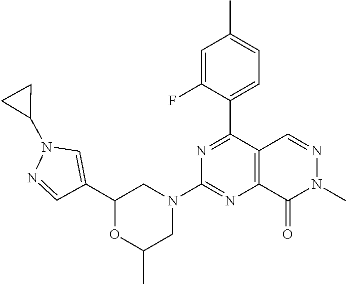 [SMILES: Cc1ccc(-c2nc(N3CC(C)OC(c4cnn(C5CC5)c4)C3)nc3c(=O)n(C)ncc23)c(F)c1] |
| I-208 | 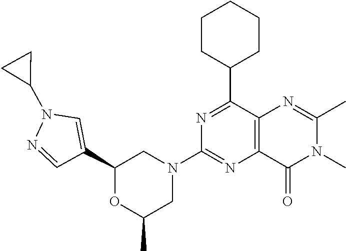 [SMILES: Cc1nc2c(C3CCCCC3)nc(N3C[C@@H](C)O[C@@H](c4cnn(C5CC5)c4)C3)nc2c(=O)n1C] |
| I-209 | 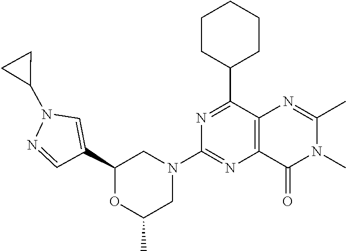 [SMILES: Cc1nc2c(C3CCCCC3)nc(N3C[C@H](C)O[C@@H](c4cnn(C5CC5)c4)C3)nc2c(=O)n1C] |
| I-210 | 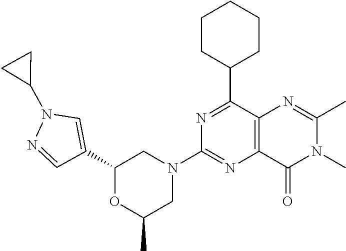 [SMILES: Cc1nc2c(C3CCCCC3)nc(N3C[C@@H](C)O[C@H](c4cnn(C5CC5)c4)C3)nc2c(=O)n1C] |
| I-211 | 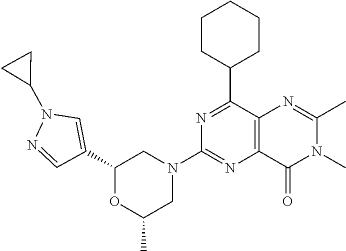 [SMILES: Cc1nc2c(C3CCCCC3)nc(N3C[C@@H](c4cnn(C5CC5)c4)O[C@@H](C)C3)nc2c(=O)n1C] |
| I-212 | 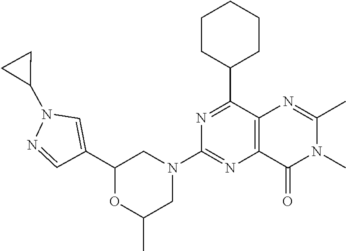 [SMILES: Cc1nc2c(C3CCCCC3)nc(N3CC(C)OC(c4cnn(C5CC5)c4)C3)nc2c(=O)n1C] |
| I-213 | 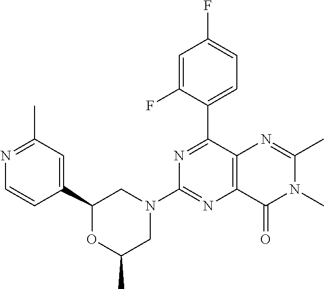 [SMILES: Cc1cc([C@H]2CN(c3nc(-c4ccc(F)cc4F)c4nc(C)n(C)c(=O)c4n3)C[C@@H](C)O2)ccn1] |
| I-214 | 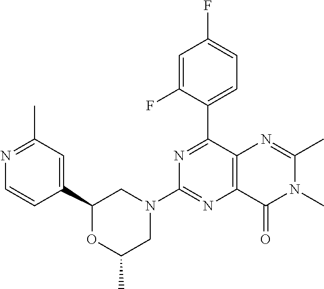 [SMILES: Cc1cc([C@H]2CN(c3nc(-c4ccc(F)cc4F)c4nc(C)n(C)c(=O)c4n3)C[C@H](C)O2)ccn1] |
| I-215 | 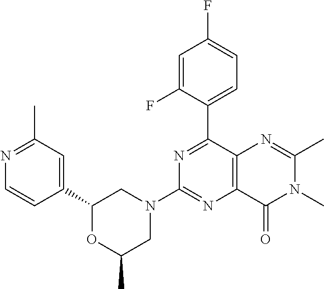 [SMILES: Cc1cc([C@@H]2CN(c3nc(-c4ccc(F)cc4F)c4nc(C)n(C)c(=O)c4n3)C[C@@H](C)O2)ccn1] |
| I-216 | 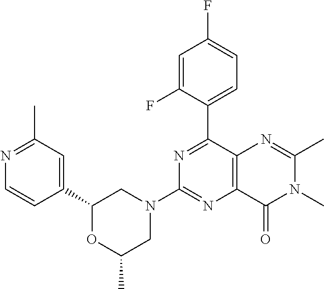 [SMILES: Cc1cc([C@@H]2CN(c3nc(-c4ccc(F)cc4F)c4nc(C)n(C)c(=O)c4n3)C[C@H](C)O2)ccn1] |
| I-217 | 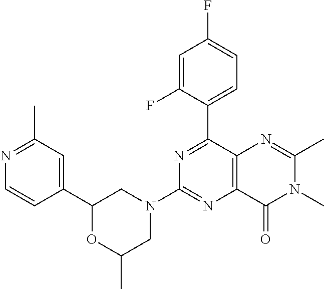 [SMILES: Cc1cc(C2CN(c3nc(-c4ccc(F)cc4F)c4nc(C)n(C)c(=O)c4n3)CC(C)O2)ccn1] |
| I-218 | 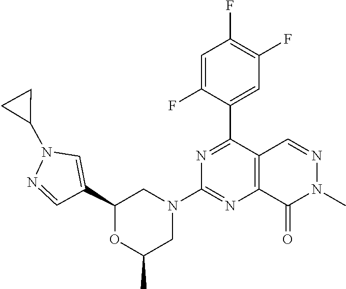 [SMILES: C[C@@H]1CN(c2nc(-c3cc(F)c(F)cc3F)c3cnn(C)c(=O)c3n2)C[C@H](c2cnn(C3CC3)c2)O1] |
| I-219 | 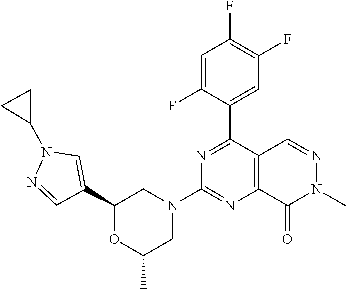 [SMILES: C[C@H]1CN(c2nc(-c3cc(F)c(F)cc3F)c3cnn(C)c(=O)c3n2)C[C@H](c2cnn(C3CC3)c2)O1] |
| I-220 | 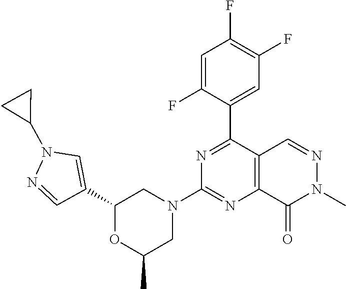 [SMILES: C[C@@H]1CN(c2nc(-c3cc(F)c(F)cc3F)c3cnn(C)c(=O)c3n2)C[C@@H](c2cnn(C3CC3)c2)O1] |
| I-221 | 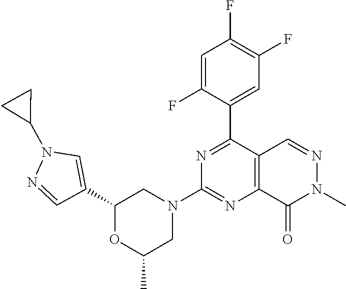 [SMILES: C[C@H]1CN(c2nc(-c3cc(F)c(F)cc3F)c3cnn(C)c(=O)c3n2)C[C@@H](c2cnn(C3CC3)c2)O1] |
| I-222 | 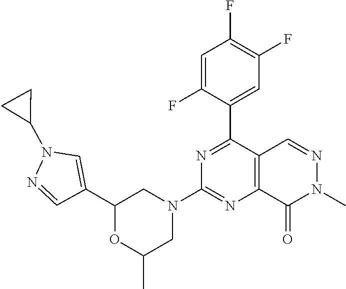 [SMILES: CC1CN(c2nc(-c3cc(F)c(F)cc3F)c3cnn(C)c(=O)c3n2)CC(c2cnn(C3CC3)c2)O1] |

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the invention provides a pharmaceutical composition comprising a compound of the present disclosure, or a tautomer thereof, or a pharmaceutically acceptable salt of said compound or said tautomer, and a pharmaceutically acceptable excipient.

In another aspect, the invention provides a compound of the present disclosure, or a tautomer thereof, or a pharmaceutically acceptable salt of said compound or said tautomer, or a pharmaceutical composition comprising said compound, or said tautomer, or said salt, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted; TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R⁴⁷H variant has been identified in genome-wide studies as being associated with increased risk for late-onset AD with an overall adjusted odds ratio (for populations of all ages) of 2.3, second only to the strong genetic association of ApoE to Alzheimer's. The R⁴⁷H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R⁴⁷H mutation are supportive of a novel loss-of-microglial barrier function for the carriers of the mutation, with the R⁴⁷H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et, al. 2019. A variety of animal models also suggest that Toll-Like Receptor (TLR) signaling is important in the pathogenesis of Rheumatoid Arthritis (RA) via persistent expression of pro-inflammatory cytokines by macrophages. Signaling through TREM2/DAP12 inhibits TLR responses by reducing MAPK (Erk 1/2) activation, suggesting that TREM2 activation may act as a negative regulator of TLR driven RA pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the invention provides a compound of the present disclosure, or a tautomer thereof, or a pharmaceutically acceptable salt of said compound or said tautomer, or a pharmaceutical composition thereof for use in treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the invention provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the invention provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSFIR

CSFIR is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSFIR is composed of a highly glycosylated extracellular ligand-binding domain, a trans-membrane domain and an intracellular tyrosine-kinase domain. Binding of CSF-1 to CSFIR results in the formation of receptor homodimers and subsequent auto-phosphorylation of several tyrosine residues in the cytoplasmic domain, notably Syk. In the brain. CSFIR is predominantly expressed in microglial cells. It has been found that microglia in CSFIR +/−patients are depleted and show increased apoptosis (Oosterhof et al . . . 2018).

The present invention relates to the unexpected discovery that administration of a TREM2 agonist can rescue the loss of microglia in cells having mutations in CSFIR. It has been previously shown that TREM2 agonist antibody 4D9 increases ATP luminescence (a measure of cell number and activity) in a dose dependent manner when the levels of M-CSF in media are reduced to 5 ng/ml (Schlepckow et al. EMBO Mol Med . . . 2020) and that TREM2 agonist AL002c increases ATP luminescence when M-CSF is completely removed from the media (Wang et al. J. Exp. Med.; 2020, 217 (9): e20200785). This finding suggests that TREM2 agonism can compensate for deficiency in CSFIR signaling caused by a decrease in the concentration of its ligand. In a 5×FAD murine Alzheimer's disease model of amyloid pathology, doses of a CSFIR inhibitor that almost completely eliminate microglia in the brains of wild-type animals show surviving microglia clustered around the amyloid plaques (Spangenberg et al. Nature Communications 2019). Plaque amyloid has been demonstrated in the past to be a ligand for TREM2, and it has been shown that microglial engagement with amyloid is dependent on TREM2 (Condello et al. Nat Comm., 2015). The present invention relates to the unexpected discovery that it is activation of TREM2 that rescued the microglia in the presence of the CSFIR inhibitor, and that this effect is also observed in patients suffering from loss of microglia due to CSFIR mutation. This discovery has not been previously taught or suggested in the available art.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSFIR, suggesting a reduced level of signaling on the macrophage colony-stimulating factor (M-CSF)/CSFIR axis (Rademakers et al, Nat Genet 2012: Konno et al, Neurology 2018). In one aspect, the present invention relates to the surprising discovery that activation of the TREM2 pathway can rescue the loss of microglia in CSFIR +/−ALSP patients, preventing microglia apoptosis, thereby treating the ALSP condition.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of Colony stimulating factor 1 receptor (CSFIR, also known as macrophage colony-stimulating factor receptor/M-CSFR, or cluster of differentiation 115/CD115).

In one aspect, the invention provides a compound of the present disclosure, or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of CSFIR.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the invention provides a method of treating or preventing a disease or disorder associated with dysfunction of CSFIR in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSFIR gene affecting the function of CSFIR. In some embodiments, the mutation in the CSFIR gene is a mutation that causes a decrease in CSFIR activity or a cessation of CSFIR activity. In some embodiments, the disease or disorder is caused by a heterozygous CSFIR mutation. In some embodiments, the disease or disorder is caused by a homozygous CSFIR mutation. In some embodiments, the disease or disorder is caused by a splice mutation in the esflr gene. In some embodiments, the disease or disorder is caused by a missense mutation in the csflr gene. In some embodiments, the disease or disorder is caused by a mutation in the catalytic kinase domain of CSFIR. In some embodiments, the disease or disorder is caused by a mutation in an immunoglobulin domain of CSFIR. In some embodiments, the disease or disorder is caused by a mutation in the ectodomain of CSF1R. In some embodiments, the disease or disorder is a disease or disorder resulting from a change (e.g. increase, decrease or cessation) in the activity of CSFIR. In some embodiments, the disease or disorder is a disease or disorder resulting from a decrease or cessation in the activity of CSFIR. CSFIR related activities that are changed in the disease or disorder include, but are not limited to: decrease or loss of microglia function; increased microglia apoptosis: decrease in Src signaling: decrease in Syk signaling: decreased microglial proliferation: decreased microglial response to cellular debris: decreased phagocytosis; and decreased release of cytokines in response to stimuli. In some embodiments, the disease or disorder is caused by a loss-of-function mutation in CSFIR. In some embodiments, the loss-of-function mutation results in a complete cessation of CSFIR function. In some embodiments, the loss-of-function mutation results in a partial loss of CSFIR function, or a decrease in CSFIR activity.

In another aspect, the invention provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSFIR. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSFIR dysfunction, or having a mutation in a gene affecting the function of CSFIR, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra—and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82 (5): 813-827). It has been shown that severe microglia loss and damage is an early feature in patients with cerebral form of x-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018:141; 2329-2342). These findings emphasize microglia/monocytes/macrophages as crucial therapeutic targets for preventing or stopping myelin destruction in patients with X-linked adrenoleukodystrophy.

The present invention relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/ml (Schlepckow et al, EMBO Mol Med., 2020) and that TREM2 agonist AL002c increases ATP luminescence when M-CSF is completely removed from the media (Wang et al, J. Exp. Med.: 2020, 217 (9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present invention relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of ATP-binding cassette transporter 1 (ABCD1).

In one aspect, the invention provides a compound of the present disclosure, or a tautomer thereof, or a pharmaceutically acceptable salt of said compound or said tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of ABCD1.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the invention provides a method of treating or preventing a disease or disorder associated with dysfunction of ABCD1 in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the invention provides a method of treating or preventing X-linked adrenoleukodystrophy (x-ALD). Globoid cell leukodystrophy (also known as Krabbe disease). Metachromatic leukodystrophy (MLD). Cerebral autosomal dominant arteriopathy with subcortical infarcts and leukoencephalopathy (CADASIL). Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (CALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, or a tautomer thereof, or a pharmaceutically acceptable salt of said compound or said tautomer, or a pharmaceutical composition thereof.

### Autism Spectrum Disorders

It has been found that TREM2 deficient mice exhibit symptoms reminiscent of autism spectrum disorders (ASDs) (Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present invention relates to the unexpected discovery that activation of TREM2, using a compound of the present invention, can rescue microglia in subjects suffering from an ASD. This discovery has not been previously taught or suggested in the available art.

In another aspect, the present invention provides a compound of the present disclosure, or a tautomer thereof, or a pharmaceutically acceptable salt of said compound or said tautomer, or a pharmaceutical composition thereof for use in treating autism or autism spectrum disorders.

In yet another aspect, the present invention provides a compound of the present disclosure, or a tautomer thereof, or a pharmaceutically acceptable salt of said compound or said tautomer, or a pharmaceutical composition thereof for use in the preparation of a medicament for treating autism or autism spectrum disorders.

In yet another aspect, the present invention provides a method of treating autism or autism spectrum disorders in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, or a pharmaceutically acceptable salt thereof with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastercomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ, of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl. (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

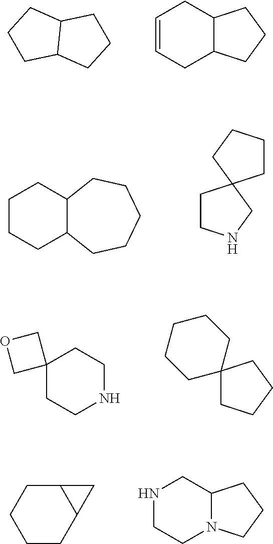

Exemplary bridged bicyclics include:

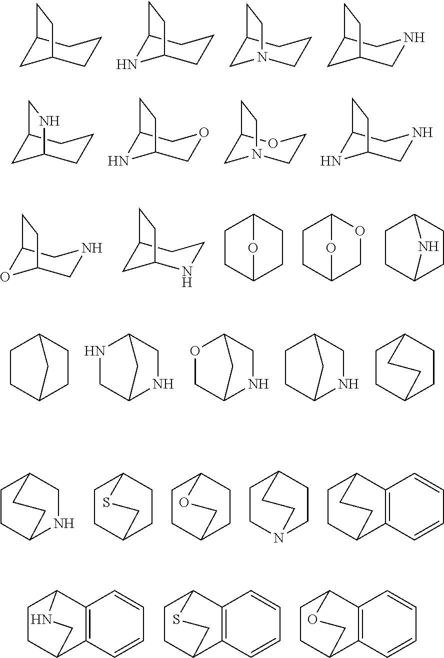

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon: the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms: having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring.” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group.” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆Rº; —(CH₂)₀₋₆ORº; —O(CH₂)₀₋₆Rº, —O—(CH₂)₀₋₆C(O)ORº; —(CH₂)₀₋₆CH(ORº) 2; —(CH₂)₀₋₆SRº; —(CH₂)₀₋₆Ph, which Ph may be substituted with Rº; —(CH₂)₀₋₄₆O(CH₂)₀₋₁Ph which Ph may be substituted with Rº; —CH═CHPh, which Ph may be substituted with Rº; —(CH₂)₀₋₆O(CH₂)₀₋₁-pyridyl which pyridyl may be substituted with Rº; —NO₂; —CN; —N₃; —(CH₂)₀₋₆ N(Rº)₂; —(CH₂)₀₋₆N(Rº)C(O) Rº; —N(Rº)C(S)Rº; —(CH₂)₀₋₆N(Rº)C(O)NRº₂; —N(Rº)C(S)NRº₂; —(CH₂)₀₋₆N(Rº)C(O)ORº; —N(Rº))N(Rº)C(O)Rº; —N(Rº))N(Rº)C(O)NRº₂; —) N(Rº))N(Rº)C(O)ORº); —(CH₂)₀₋₆C(O)Rº; —C(S)Rº; —(CH₂)₀₋₆C(O)ORº; —(CH₂)₀₋₆C(O)SRº; —(CH₂)₀₋₆C(O)OSiRº₃; —(CH₂)₀₋₆OC(O)Rº; —OC(O) (CH₂)₀₋₆SRº, —(CH₂)₀₋₆SC(O)Rº; —(CH₂)₀₋₆C(O)NRº₂; —C(S)NRº₂; —C(S)SRº; —SC(S)SRº, —(CH₂)₀₋₆—(OC(O)NRº₂; —C(O)N(ORº) Rº; —C(O) C(O)Rº; —C(O) CH₂C(O)Rº; —C(NORº) Rº; —(CH₂)₀₋₆SSRº; —(CH₂)₀₋₆S(O) 2Rº; —(CH₂)₀₋₆S(O)₂ORº; —(CH₂)₀₋₆OS(O)₂Rº; —S(O)₂NRº₂; —(CH₂)₀₋₆—S(O) Rº; —N(Rº)S(O)₂NRº₂; —N(Rº)S(O)₂Rº; —N(OR) Rº; —C(NH) NRº₂; —P(O)₂Rº; —P(O) Rº)₂; —P(O)(OR); —OP(O) (RºORº; —OP(O) Rº)₂; —OP(O)(ORº) 2; SiRº₃; —(C₁₋₄ straight or branched alkylene)O—N(Rº)₂; or —(C₁₋₄ straight or branched alkylene) C(O)O—N(Rº)₂, wherein each Rº may be substituted as defined below and is independently hydrogen. C₁₋₆ aliphatic, —CH₂Ph, —O(CH₂)₀₋₁Ph, —CH₂—(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of Rº, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on Rº (or the ring formed by taking two independent occurrences of Rº together with their intervening atoms), are independently halogen, —(CH₂)₀₋₂R^{●}, —) (haloR^{●}, —(CH₂)₀₋₂OH, —(CH₂)₀₋₂OR^{●}, —(CH₂)₀₋₂CH(OR^{●})₂; —O(haloR^{●}, —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene) C(O)OR, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of Rº include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen. C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include; —O(CR*₂)₂₋₃O—, wherein each independent occurrence of R* is selected from hydrogen. C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}), -(haloR^{●}, —OH, —OR^{●}, —O(haloR*), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}2, —C(O) R^{†}, —C(O)OR^{†}, —C(O) C(O)R^{†}, —C(O) CH₂C(O)R^{†}, —S(O)₂R^{†}, —S(O)₂NR^{†}2, —C(S)NR^{†}₂, —C(NH) NR^{†}₂, or —N(R^{†}) S(O); R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{●}, —) (haloR^{●}, —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

The terms “patient” and “subject” as used herein refer to humans and mammals, including, but not limited to, primates, cows, sheep, goats, horses, dogs, cats, rabbits, rats, and mice. In one embodiment the subject is a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

The terms “C₁₋₃alkyl,” “C₁₋₅alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₅alky, or C₁₋₆alkyl include, but are not limited to, methyl, ethyl, n-propyl, isopropyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR^{#}, wherein R^{#}represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.01.3]pentanyl,

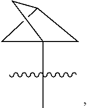 [SMILES: *C12CC(C1)C2]

tricyclo[2.1.1.0^{1,3}]hexanyl, tricyclo[3.1.1.0^{1,5}]hexanyl, and tricyclo[3.2.1.0^{1,5}]octanyl.

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following scheme are the variables as defined for Formula I, unless otherwise noted. All starting materials are either commercially available, for example, from Merck Sigma-Aldrich Inc, and Enamine Ltd, or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein. As used below, Z is a leaving group, which can include but is not limited to, halogens (e.g, fluoride, chloride, bromide, iodide), sulfonates (e.g. mesylate, tosylate, benzenesulfonate, brosylate, nosylate, triflate), diazonium, and the like. As used below, in certain embodiments Y is an organometal coupling reagent group, which can include but are not limited to, boronic acids and esters, organotin and organozinc reagents.

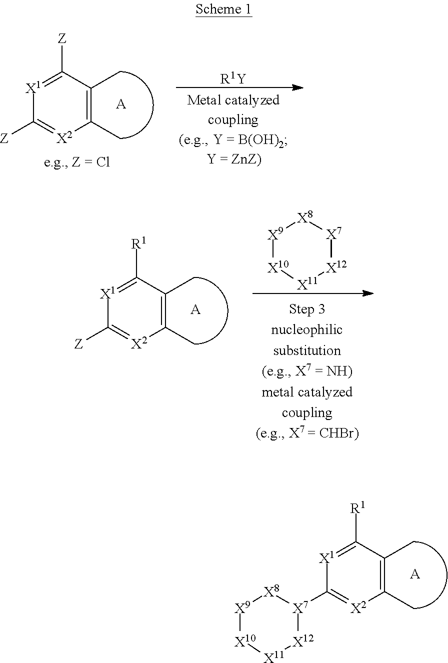

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### EXAMPLES

This section provides specific examples of compounds of Formula I and methods of making the same.

### List of Abbreviations

| | |
|---|---|
| aq or aq. | aqueous |
| DCM | dichloromethane |
| DMAP | 4-dimethylaminopyridine |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | equivalent |
| ESI or ES | electrospray ionization |
| Et | ethyl |
| EtOAc or EA | ethyl acetate |
| g | gram(s) |
| h or hr | hour(s) |
| HPLC | high pressure liquid chromatography |
| iPr | isopropyl |
| iPr₂NEt or DIPEA | N-ethyl diisopropylamine (Hunig's base) |
| LC MS, LCMS, | liquid chromatography mass spectroscopy |
| LC-MS or LC/MS | |
| m/z | mass divided by charge |
| Me | methyl |
| CH₃CN | acetonitrile |
| MeOH | methanol |
| mg | milligrams |
| min | minutes |
| mL | milliliters |
| MS | mass spectra |
| n-BuLi | n-butyllithium |
| NMR | nuclear magnetic resonance |
| PE | Petroleum ether |
| Ph | phenyl |
| RT or rt or r.t. | room temperature |
| RuPhos Pd G3 | (2-Dicyclohexylphosphino-2′,6′-diisopropoxy- |
| | 1,1′-biphenyl)[2-(2′-amino-1,1′- |
| | biphenyl)]palladium(II) methanesulfonate |
| sat. | saturated |
| SFC | supercritical fluid chromatography |
| TEA or Et₃N | triethylamine |
| THF | tetrahydrofuran |
| Xantphos Pd G3 | [(4,5-Bis(diphenylphosphino)-9,9- |
| | dimethylxanthene)-2-(2′-amino-1,1′- |
| | biphenyl)]palladium(II) methanesulfonate |
| PE | Petroleum ether |

### General Analytical and Purification Methods

Provided in this section are descriptions of the general analytical and purification methods used to prepare the specific compounds provided herein.

### Chromatography:

Unless otherwise indicated, crude product-containing residues were purified by passing the crude material or concentrate through either a Biotage brand silica gel column pre-packed with flash silica (SiO₂) or reverse phase flash silica (C18) and eluting the product off the column with a solvent gradient as indicated. For example, a description of silica gel (0-40% EtOAc/hexane) means the product was obtained by elution from the column packed with silica using a solvent gradient of 0% to 40% EtOAc in hexanes.

### Preparative HPLC Method:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150×30 mm) or (b) Waters X-select CSH column (5 micron, C18, 100×30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

### Analytical HPLC Method:

Where so indicated, the compounds described herein were analyzed using an Aglilent 1100 series instrument with DAD detector.

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2×25 cm, 5 μm or (b) Chiralpak AD-H 2×15 cm, 5 μm.

Some CP Analytical-SFC experiments were run on SFC Method Station (Thar, Waters) with the following conditions: Column temperature: 40° C., Mobile phase: CO₂/Methanol (0.2% Methanol Ammonia)=Flow: 4.0 ml/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18 (3) (150×25 mm, 10μ); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

### Mass Spectra (MS)

Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### Compound Names

The compounds disclosed and described herein have been named using the IUPAC naming function of ChemDraw Professional 17.0.

### SPECIFIC EXAMPLES

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example 1-Synthesis of Compounds I-40 and I-42: 7-[(2R,4S)-2-(1-cyclopropylpyrazol-4-yl)tetrahydropyran-4-yl]-5-(2,4-difluorophenyl)-2,3-dimethyl-pyrido[2,3-d]pyrimidin-4-one and 7-[(2S,4R)-2-(1-cyclopropylpyrazol-4-yl)tetrahydropyran-4-yl]-5-(2,4-difluorophenyl)-2,3-dimethyl-pyrido[2,3-d]pyrimidin-4-one

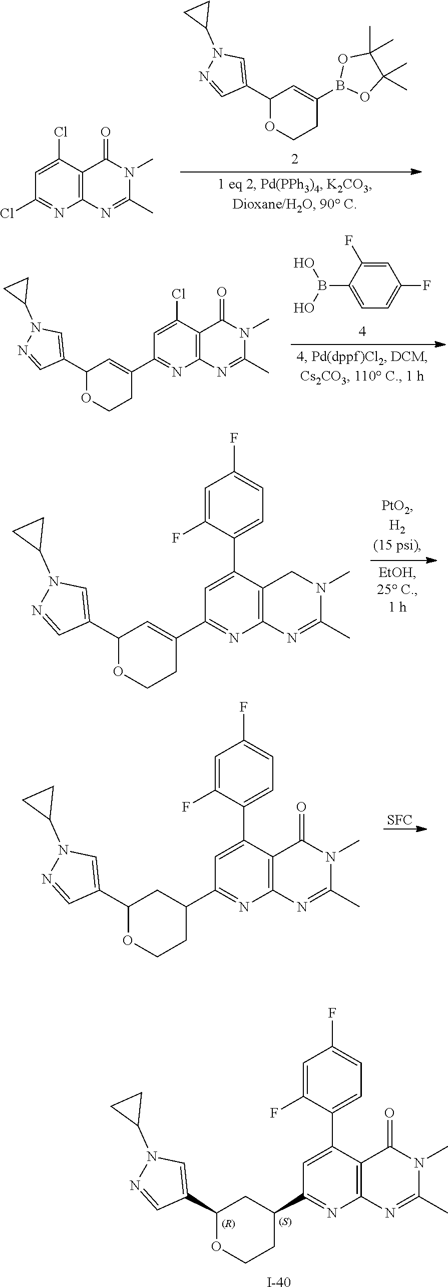

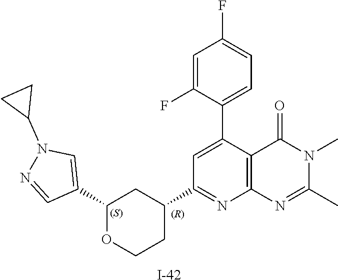

Step 1: To a mixture of 1-cyclopropyl-4-[4-(4,4,5,5-tetramethyl-1,3,2-dioxaborolan-2-yl)-3,6-dihydro-2H-pyran-6-yl]pyrazole (1.00 eq, 681 mg, 2.16 mmol), 5,7-dichloro-2,3-dimethyl-pyrido[2,3-d]pyrimidin-4-one (1.00 eq, 526 mg, 2.16 mmol) and K₂CO₃ (3.00 eq, 893 mg, 6.47 mmol) in 1,4-Dioxane (25 mL) and Water (5 mL) was added Pd(PPh₃)₄ (0.1000 eq, 249 mg, 0.216 mmol). The reaction mixture was stirred at 90° C. under N₂ for 10 h. The reaction mixture was cooled to 25° C., poured into water (100 mL), extracted with EtOAc (100 mL×2). The organic phase was separated dried over Na₂SO₄, filtered and concentrated in vacuum. The residue was purified by silica gel chromatography (PE:EA=2:1˜0:1) to give product 5-chloro-7-[6-(1-cyclopropylpyrazol-4-yl)-3, 6-dihydro-2H-pyran-4-yl]-2, 3-dimethyl-pyrido[2, 3-d]pyrimidin-4-one (460 mg, 1.16 mmol, 53.65% yield) as a yellow oil which was checked by LCMS. MS (ESI): m/z=398.0 [M+H]+.

Step 2: To a mixture of 2-(2,4-difluorophenyl)-4,4,5,5-tetramethyl-1,3,2-dioxaborolane (1.50 eq, 416 mg, 1.73 mmol), 5-chloro-7-[6-(1-cyclopropylpyrazol-4-yl)-3,6-dihydro-2H-pyran-4-yl]-2,3-dimethyl-pyrido[2,3-d]pyrimidin-4-one (1.00 eq, 460 mg, 1.16 mmol) and Cs₂CO₃ (3.00 eq, 1127 mg, 3.47 mmol) in 1,4-Dioxane (12 mL)/Water (2.4 mL) was added Pd(dppf)Cl₂ (0.100 eq, 85 mg, 0.116 mmol). The reaction mixture was stirred at 100° C., for 1 h. LCMS showed the reaction was completed and desired MS (476.2 [M+1]+, ESI pos) was found. The reaction mixture was concentrated in vacuum. The residue was purified by silica gel chromatography (PE:EA=1:1˜0:1) to give 7-[6-(1-cyclopropylpyrazol-4-yl)-3,6-dihydro-2H-pyran-4-yl]-5-(2,4-difluorophenyl)-2,3-dimethyl-pyrido[2,3-d]pyrimidin-4-one (450 mg, 0.946 mmol, 81.85% % yield) as a yellow solid which was checked by LCMS. LCMS: (M+H)+=476.2

Step 3: To a solution of 7-[6-(1-cyclopropylpyrazol-4-yl)-3,6-dihydro-2H-pyran-4-yl]-5-(2,4-difluorophenyl)-2,3-dimethyl-1,2-dihydropyrido[2,3-d]pyrimidin-4-one (1.00 eq, 450 mg, 0.942 mmol) in Ethanol (20 mL) was added PtO₂ (0.931 eq, 199 mg, 0.877 mmol), the mixture stirred at 25° C., for 1 h under H₂. LCMS showed the reaction was complected. The crude mixture was filtered through a pad of celite. The filtrate was concentrated in vacuo. The crude product was pre-purified by column chromatography on silica gel eluted with petroleum ether/ethyl acetate (EA/MeOH)=1:0 to 100:3 to give product 7-[2-(1-cyclopropylpyrazol-4-yl)tetrahydropyran-4-yl]-5-(2,4-difluorophenyl)-2,3-dimethyl-pyrido[2,3-d]pyrimidin-4-one (300 mg, 0.603 mmol, 64.00% yield) obtained as yellow oil. Confirmed by LCMS MS (ESI): m/z=478.3 [M+H]+

Step 4: The racemate 7-[2-(1-cyclopropylpyrazol-4-yl)tetrahydropyran-4-yl]-5-(2,4 difluorophenyl)-2,3-dimethyl-pyrido[2,3-d]pyrimidin-4-one CO₂/MeOH [0.1% NH₃H₂O MeOH], B %: 35%-35%, DAICEL CHIRALPAK AD (250 mm*₃₀ mm, 10 um) to give I-40 (enantiopure, 63.5 mg, retention time=2.251 min) and I-42 (enantiopure, 58.4 mg, retention time=0.757 min). Absolute stereochemistry arbitrarily assigned.

I-40 (P2, enantiopure): LCMS: (M+H)⁺=478.1; purity=96.3% (UV 220 nm); Retention time=0.676 min. LCMS CP Method A. 1H NMR (400 MHz, CHLOROFORM-d) δ=7.48 (d, J=3.3 Hz, 2H), 7.26-7.20 (m, 1H), 7.10 (s, 1H), 7.02-6.88 (m, 2H), 4.51 (dd, J=1.9, 11.2 Hz, 1H), 4.30-4.18 (m, 1H), 3.81-3.70 (m, 1H), 3.58-3.54 (m, 1H), 3.53 (s, 3H), 3.36-3.16 (m, 1H), 2.71 (s, 3H), 2.26 (br d, J=12.3 Hz, 1H), 2.19-1.92 (m, 3H), 1.15-1.04 (m, 2H), 1.03-0.91 (m, 2H)

I-42 (P1, enantiopure): LCMS: (M+H)+=478.2; purity=98.4% (UV 220 nm); Retention time=0.862 min. ¹H NMR (400 MHz, CHLOROFORM-d) δ=7.47 (d, J=3.4 Hz, 2H), 7.26-7.19 (m, 1H), 7.10 (s, 1H), 7.03-6.85 (m, 2H), 4.56-4.45 (m, 1H), 4.24 (br dd, J=3.3, 11.4 Hz, 1H), 3.82-3.69 (m, 1H), 3.58-3.54 (m, 1H), 3.52 (s, 3H), 3.26 (ddd, J=3.5, 8.5, 11.7 Hz, 1H), 2.71 (s, 3H), 2.35-2.20 (m, 1H), 2.18-1.92 (m, 3H), 1.13-1.04 (m, 2H), 1.02-0.92 (m, 2H)

### Example 2-Synthesis of Compound I-45:5-(4-chloro-2-fluorophenyl)-7-(2-(1-cyclopropyl-1H-pyrazol-4-yl) morpholino)-2-methylpyrido[3,4-d]pyridazin-1 (2H)-one

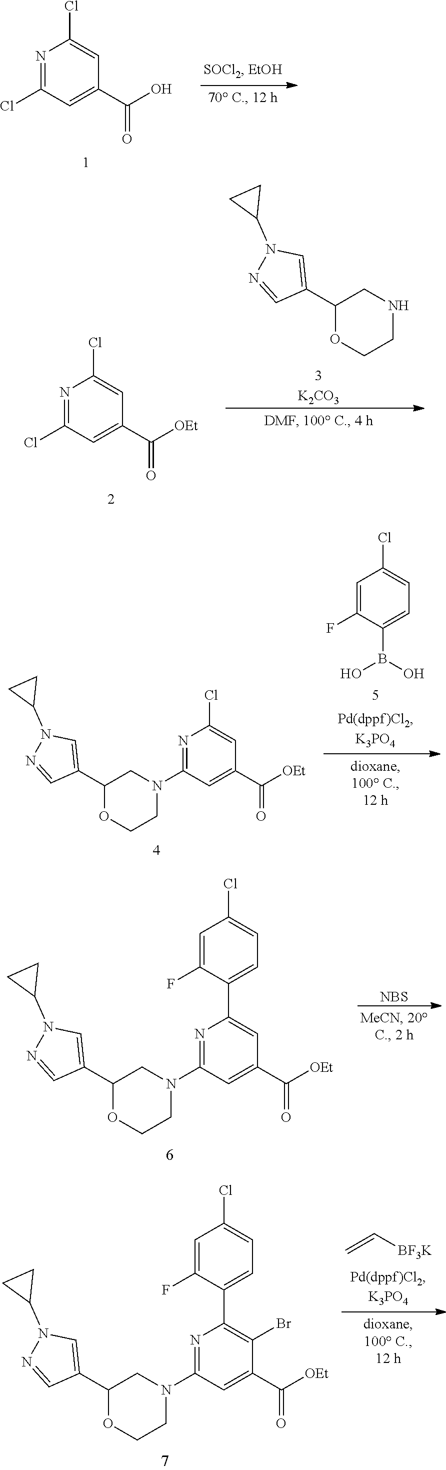

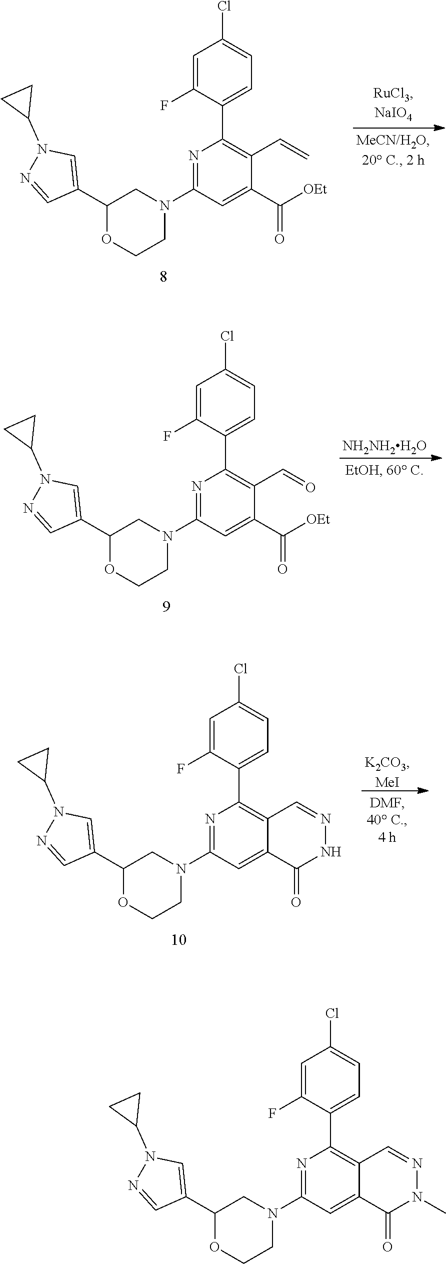

Step 1: ethyl 2,6-dichloroisonicotinate. To a solution of 2,6-dichloropyridine-4-carboxylic acid (5.00 g, 26.0 mmol, 1.00 eq) in ethanol (60 mL) was added thionyl chloride (9.5 mL, 130 mmol, 5.00 eq). The reaction was stirred at 70° C., for 12 hours. LCMS showed starting material was consumed and desired m/z detected. The mixture was concentrated under vacuum and purified by silica gel column chromatography (eluting with Ethyl acetate/Petroleum ether, 10% to 20%) to give ethyl 2,6-dichloropyridine-4-carboxylate (4.80 g, 21.8 mmol, 83.8% yield).

Step 2: ethyl 2-chloro-6-(2-(1-cyclopropyl-1H-pyrazol-4-yl) morpholino) isonicotinate. To a solution of ethyl 2,6-dichloropyridine-4-carboxylate (500 mg, 2.27 mmol, 1.0 eq) and 2-(1-cyclopropylpyrazol-4-yl) morpholine (395 mg, 2.04 mmol, 0.9 eq) in DMF (10 mL) was added K₂CO₃ (942 mg, 6.82 mmol, 3.0 eq). The reaction was stirred at 100° C., for 12 hours. LCMS showed starting material consumed and desired product m/z detected. After cooling to ambient temperature, the mixture was filtered through celite, and the filtrate was concentrated under vacuum. The residue was concentrated under vacuum, and purified by silica gel column chromatography (eluting with Ethyl acetate/Petroleum ether, 10% to 30%) to give ethyl 2-chloro-6-[2-(1-cyclopropylpyrazol-4-yl) morpholin-4-yl]pyridine-4-carboxylate (600 mg, 1.59 mmol, 70.1% yield).

Step 3: ethyl 2-(4-chloro-2-fluorophenyl)-6-(2-(1-cyclopropyl-1H-pyrazol-4-yl) morpholino) isonicotinate. To a solution of ethyl 2-chloro-6-[2-(1-cyclopropylpyrazol-4-yl) morpholin-4-yl]pyridine-4-carboxylate (600 mg, 1.59 mmol, 1.0 eq) and (4-chloro-2-fluoro-phenyl) boronic acid (833 mg, 4.78 mmol, 3.0 eq) in 1,4-dioxane (20 mL) was added Pd(dppf)Cl₂ (130 mg, 0.16 mmol, 0.10 eq) and K₃PO₄ (1.01 g, 4.78 mmol, 3.0 eq). The reaction was stirred at 100° C., for 12 hours under N₂. After cooling to ambient temperature, the mixture was filtered through celite, and the filtrate was concentrated under vacuum. The residue was concentrated under vacuum, and purified by silica gel column chromatography (eluting with Ethyl acetate/Petroleum ether, 10% to 30%) to give ethyl 2-(4-chloro-2-fluoro-phenyl)-6-[2-(1-cyclopropylpyrazol-4-yl) morpholin-4-yl]pyridine-4-carboxylate (600 mg, 0.96 mmol, 60.0% yield).

Step 4: ethyl 3-bromo-2-(4-chloro-2-fluorophenyl)-6-(2-(1-cyclopropyl-1H-pyrazol-4-yl) morpholino) isonicotinate. To a solution of ethyl 2-(4-chloro-2-fluoro-phenyl)-6-[2-(1-cyclopropylpyrazol-4-yl) morpholin-4-yl]pyridine-4-carboxylate (550 mg, 1.17 mmol, 1.0 eq) in MeCN (20 mL) was added NBS (208 mg, 1.17 mmol, 1.0 eq). The reaction was stirred at 20° C., for 2 hours. The reaction was concentrated and then purified by flash column chromatography eluting 30% ethyl acetate in petroleum ether. The desired fractions were concentrated to dryness in vacuo to give ethyl 3-bromo-2-(4-chloro-2-fluoro-phenyl)-6-[2-(1-cyclopropylpyrazol-4-yl) morpholin-4-yl]pyridine-4-carboxylate (500 mg, 0.64 mmol, 66% purity, 54.5% yield).

Step 5: ethyl 2-(4-chloro-2-fluorophenyl)-6-(2-(1-cyclopropyl-1H-pyrazol-4-yl) morpholino)-3-vinylisonicotinate. To a solution of ethyl 3-bromo-2-(4-chloro-2-fluoro-phenyl)-6-[2-(1-cyclopropylpyrazol-4-yl) morpholin-4-yl]pyridine-4-carboxylate (500 mg, 0.91 mmol, 1.0 eq) and potassium vinyltrifluoroborate (609 mg. 4.55 mmol, 5.0 eq) in 1,4-dioxane (30 mL) was added Pd(dppf)Cl₂ (74 mg, 0.091 mmol, 0.10 eq) and K₂PO₄ (964 mg. 4.55 mmol, 5.0 eq). The reaction was stirred at 100° C. for 12 hours under N₂. After cooling to ambient temperature, the mixture was filtered through celite, and the filtrate was concentrated under vacuum. The residue was purified by silica gel column chromatography (eluting with Ethyl acetate/Petroleum ether, 10% to 30%) and prep-HPLC (FA) to give ethyl 2-(4-chloro-2-fluoro-phenyl)-6-[2-(1-cyclopropylpyrazol-4-yl) morpholin-4-yl]-3-vinyl-pyridine-4-carboxylate (90 mg, 0.18 mmol, 19.9% yield).

Step 6: ethyl 2-(4-chloro-2-fluorophenyl)-6-(2-(1-cyclopropyl-1H-pyrazol-4-yl) morpholino)-3-formylisonicotinate. To a solution of ethyl 2-(4-chloro-2-fluoro-phenyl)-6-[2-(1-cyclopropylpyrazol-4-yl) morpholin-4-yl]-3-vinyl-pyridine-4-carboxylate (40 mg, 0.081 mmol, 1.0 eq) in MeCN (6 mL) and water (1 mL) was added sodium metaperiodate (34 mg. 0.16 mmol, 2.0 eq) and RuCl₃ (0.83 mg, 0.004 mmol, 0.05 eq). The reaction was stirred at 20° C., for 2 hours. LCMS showed starting material consumed and desired product m/z detected. The reaction mixture was taken up in EtOAc (20 mL) and the organics washed with saturated Na₂S₂O; solution (2×10 mL). The organics were then separated and dried (Na₂SO₄) before concentration to dryness. The crude was then purified by flash column chromatography eluting with 40% ethyl acetate in petroleum ether. The desired fractions were concentrated to dryness in vacuo to give ethyl 2-(4-chloro-2-fluoro-phenyl)-6-[2-(1-cyclopropylpyrazol-4-yl) morpholin-4-yl]-3-formyl-pyridine-4-carboxylate (15 mg, 0.03 mmol, 37.4% yield).

Step 7: To a solution of ethyl 2-(4-chloro-2-fluoro-phenyl)-6-[2-(1-cyclopropylpyrazol-4-yl) morpholin-4-yl]-3-formyl-pyridine-4-carboxylate (15 mg, 0.03 mmol, 1.0 eq) in ethanol (1 mL) was added NH₂NH₂H₂O (5.6 mg, 0.09 mmol, 3.0 eq). The reaction was stirred at 80° C., for 4 hours. LCMS showed starting material consumed and desired product m/z detected. The mixture was concentrated to give crude 5-(4-chloro-2-fluoro-phenyl)-7-[2-(1-cyclopropylpyrazol-4-yl) morpholin-4-yl]-2H-pyrido[3,4-d]pyridazin-1-one (15 mg, 0.032 mmol, 106.9% yield). The crude product was used for the next step without purification.

Step 8: 5-(4-chloro-2-fluorophenyl)-7-(2-(1-cyclopropyl-1H-pyrazol-4-yl) morpholino)-2-methylpyrido[3,4-d]pyridazin-1 (2H)-one. To a solution of 5-(4-chloro-2-fluoro-phenyl)-7-[2-(1-cyclopropylpyrazol-4-yl) morpholin-4-yl]-2H-pyrido[3,4-d]pyridazin-1-one (10 mg, 0.021 mmol, 1.0 eq) in DMF (1 mL) was added K₂CO₃ (5.9 mg, 0.043 mmol, 2.0 eq) and Mel (4.0 μL, 0.064 mmol, 3.0 eq). The reaction was stirred at 20° C. LCMS showed starting material consumed and desired product detected. The mixture was filtered through celite and the filtrate was purified by prep-HPLC (FA) to give 5-(4-chloro-2-fluoro-phenyl)-7-[2-(1-cyclopropylpyrazol-4-yl) morpholin-4-yl]-2-methyl-pyrido[3,4-d]pyridazin-1-one (3.5 mg, 0.0073 mmol, 34.2% yield).

¹H NMR (400 MHz, DMSO) δ 7.83 (d, J=3.4 Hz, 1H), 7.71 (s, 1H), 7.62 (s, 1H), 7.48 (t, J=8.9 Hz, 1H), 7.44 (s, 1H), 7.35-7.32 (m, 1H), 7.30-7.27 (m, 1H), 4.62 (d, J=9.0 Hz, 1H), 4.55 (d, J=13.7 Hz, 1H), 4.27 (d, J=12.9 Hz, 1H), 4.12 (d, J=10.6 Hz, 1H), 3.86-3.82 (m, 1H), 3.80 (s, 3H), 3.76-3.71 (m, 1H), 3.33-3.25 (m, 1H), 3.18-3.12 (m, 1H), 1.25-1.21 (m, 2H), 1.19-1.14 (m, 2H).

### Example 3-Synthesis of Compound I-48:5-(4-chloro-2-fluoro-phenyl)-7-[(2R,4S)-2-(1-cyclopropylpyrazol-4-yl)tetrahydropyran-4-yl]-2-methyl-pyrido[3,4-d]pyridazin-1-one

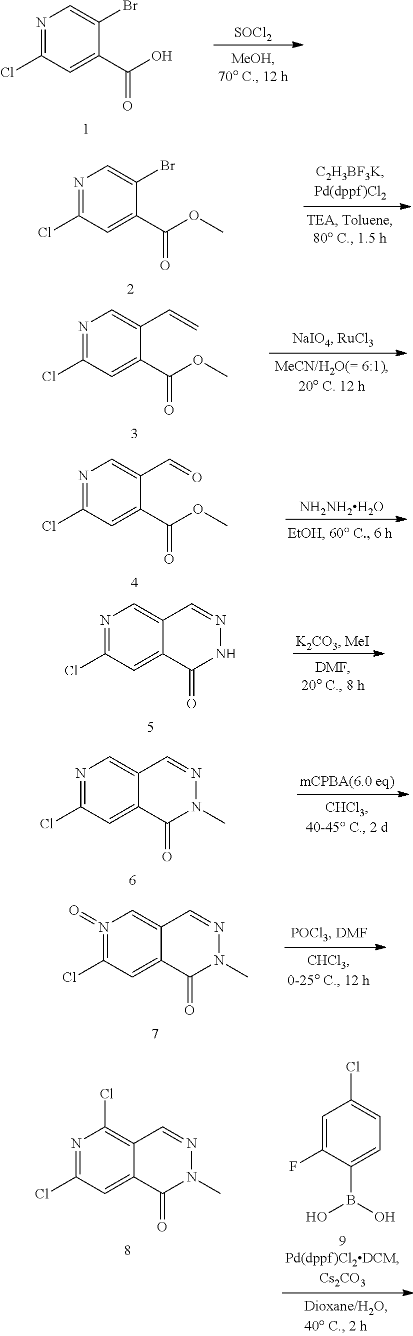

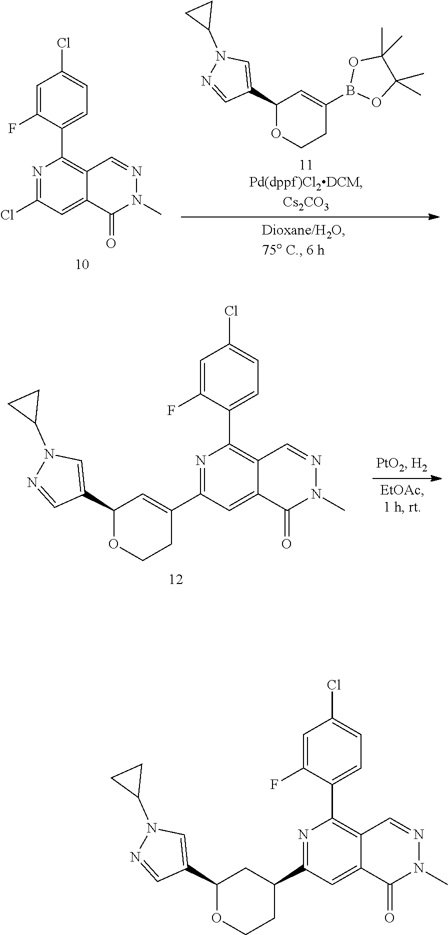 [SMILES: Cn1ncc2c(-c3ccc(Cl)cc3F)nc(Cl)cc2c1=O ; CC1(C)OB(C2=C[C@H](c3cnn(C4CC4)c3)OCC2)OC1(C)C ; Cn1ncc2c(-c3ccc(Cl)cc3F)nc(C3=C[C@H](c4cnn(C5CC5)c4)OCC3)cc2c1=O ; Cn1ncc2c(-c3ccc(Cl)cc3F)nc([C@H]3CCO[C@@H](c4cnn(C5CC5)c4)C3)cc2c1=O]

Step 1: To a solution of 5-bromo-2-chloro-pyridine-4-carboxylic acid (15.0 g, 63.4 mmol, 1.0 eq) in methanol (100 mL) was added thionyl chloride (22.6 g, 190 mmol, 3.0 eq) dropwise at 0° C. Then the mixture was heated to 70° C., and stirred at 70° C., for 12 hours. LCMS indicated that the starting material was consumed, and desired compound was detected. The mixture was concentrated under reduced pressure. The crude was purified by column chromatography (SiO₂, Petroleum ether:ethyl acetate=0-10%) to obtain methyl 5-bromo-2-chloro-pyridine-4-carboxylate (14.5 g, 57.9 mmol, 91.3% yield). ¹H NMR (400 MHz, DMSO) δ 8.77 (s, 1H), 7.87 (d, J=9.1 Hz, 1H), 3.91 (s, 3H).

Step 2: To a solution of methyl 5-bromo-2-chloro-pyridine-4-carboxylate (14.7 g, 58.7 mmol, 1.0 eq) in toluene (100 mL) were added potassium vinyltrifluoroborate (23.6 g, 176 mmol, 3.0 eq), TEA (10 mL, 117 mmol, 2.0 eq) and Pd(dppf)Cl₂ (3.84 g, 4.69 mmol, 0.08 eq). The mixture was heated at 80° C. for 1.5 hours. LCMS indicated that the starting material was consumed, and desired compound was detected. The mixture was filtered through diatomite and the filtrate was concentrated under reduced pressure. The crude product was purified by column chromatography (SiO₂, Petroleum ether:ethyl acetate=0-10%) to give methyl 2-chloro-5-vinyl-pyridine-4-carboc xylate (5.45 g. 27.6 mmol, 46.9% yield) as a white solid. ¹H NMR (400 MHz, DMSO) δ 8.84-8.75 (m, 1H), 7.81-7.73 (m, 1H), 7.18-7.06 (m, 1H), 5.96 (d, J=17.6 Hz, 1H), 5.52 (d, J=11.2 Hz, 1H), 3.87 (d, J=10.1 Hz, 3H)

Step 3: To a solution of methyl 2-chloro-5-vinyl-pyridine-4-carboxylate (4.0 g, 20.2 mmol, 1.0 eq) in MeCN (36 mL)/water (6 mL) were added sodium metaperiodate (8.66 g, 40.5 mmol, 2.0 eq) and RuCl₃ (210 mg, 1.01 mmol, 0.05 eq). The mixture was stirred at 25° C., for 12 hours. LCMS indicated that the starting material was consumed, and desired compound was detected. The mixture was filtered with diatomite. The mixture was quenched with Na₂SO₃ (a.q.) and extracted with EtOAc (30 mL*₃). The organic was dried over Na₂SO₄ and concentrated under reduced pressure. The crude product was purified by column chromatography (SiO₂, Petroleum ether:ethyl acetate=0-100%) to give the product methyl 2-chloro-5-formyl-pyridine-4-carboxylate (2.26 g, 11.3 mmol, 55.9% yield)

¹H NMR (400 MHz, DMSO) δ 10.36-10.27 (m, 1H), 8.90 (s, 1H), 7.93 (d, J=12.1 Hz, 1H), 3.93 (s, 3H).

Step 4: a solution of methyl 2-chloro-5-formyl-pyridine-4-carboxylate (5.00 g, 25.1 mmol. 1.0 eq) in ethanol (50 mL) was added NH₂NH₂H₂O (2.5 mL. 50.1 mmol, 2.0 eq). The mixture was stirred at 60° C., for 3 hours. LCMS indicated that the starting material was consumed, and desired compound was detected. The mixture was concentrated under reduced pressure to give the product 7-chloro-2H-pyrido[3,4-d]pyridazin-1-one (4.75 g, 26.2 mmol). The crude was used directly for next step without further purification. ¹H NMR (400 MHz, DMSO) δ 9.20 (s, 1H), 8.54 (s, 1H), 8.12 (d, J=15.3 Hz, 1H).

Step 5: To a solution of 7-chloro-2H-pyrido[3,4-d]pyridazin-1-one (4.75 g. 26.2 mmol, 1.0 eq) in DMF (35 mL) were added K₂CO₃ (10.9 g, 78.5 mmol, 3.0 eq) and Mel (11.1 g. 78.5 mmol, 3.0 eq). The mixture was stirred at 20° C., for 12 hours. LCMS indicated that the starting material was consumed, and desired compound was detected. The solution was filtered with diatomite and the filtrate was concentrated under reduced vacuum. The crude product was purified by column chromatography (SiO₂, MeOH: DCM=0-10%) to give 7-chloro-2-methyl-pyrido[3,4-d]pyridazin-1-one (3.20 g. 16.4 mmol, 62.5% yield) as a yellow solid. ¹H NMR (400 MHz, DMSO) δ 9.21 (s, 1H), 8.58 (s, 1H), 8.11 (s, 1H), 3.73 (s, 3H).

Step 6: To a solution of 7-chloro-2-methyl-pyrido[3,4-d]pyridazin-1-one (3.2 g, 16.4 mmol, 1.0 eq) in chloroform (30 mL) was added mCPBA (8.47 g, 49.1 mmol, 3.0 eq). The mixture was stirred at 40° C., for 48 hours. LCMS indicated that the starting material was consumed, and desired compound was detected. The mixture was quenched with Na₂SO₃ and adjusted pH to 7-8 by NaHCO₃. The mixture was extracted with ethyl acetate (100 mL×3). The organic phase was dried over Na₂SO₄ and concentrated under reduced pressure. The crude product was purified by column chromatography (SiO₂, Petroleum ether:ethyl acetate=0-100%) to give the desired product 7-chloro-2-methyl-6-oxo-pyrido[3,4-d]pyridazin-1-one (1.45 g. 6.85 mmol, 41.9% yield) ¹H NMR (400 MHz, DMSO) δ 9.14 (s, 1H), 8.43 (s, 1H), 8.32 (s, 1H), 3.70 (s, 3H).

Step 7: To a solution of 7-chloro-2-methyl-6-oxo-pyrido[3,4-d]pyridazin-1-one (500 mg. 2.36 mmol, 1.0 eq) in chloroform (5 mL) were added DMF (86 mg, 1.18 mmol, 0.5 eq) and POCl₃ (0.33 mL, 3.54 mmol, 1.5 eq) at 0° C., and the mixture was stirred at 25° C., for 12 hours. LCMS indicated that the starting material was consumed, and desired compound was detected. The mixture was concentrated under reduced pressure then washed with water (20 mL) and extracted with dichloromethane (30 mL×3). The organic phase was dried over Na₂SO₄ and concentrated under reduced pressure. The crude was purified by column chromatography (SiO₂, Petroleum ether:ethyl acetate=0-100%) to give 5,7-dichloro-2-methyl-pyrido[3,4-d]pyridazin-1-one (93 mg. 0.40 mmol, 17.1% yield) as a green solid. ¹H NMR (400 MHz, DMSO) δ 8.57 (s, 1H), 8.17 (s, 1H), 3.75 (s, 3H).

Step 8: To a solution of (4-chloro-2-fluoro-phenyl) boronic acid (76 mg, 0.44 mmol, 1.0 eq) in 1,4-dioxane (6 mL) were added Cs₂CO₃ (283 mg, 0.87 mmol, 2.0 eq), 5,7-dichloro-2-methyl-pyrido[3,4-d]pyridazin-1-one (100 mg, 0.44 mmol, 1.0 eq) and Pd(dppf)Cl₂·DCM (32 mg, 0.044 mmol, 0.10 eq) under N₂ at 25° C. The mixture was stirred at 40° C., for 1 hour. LCMS indicated that the starting material was consumed and desired compound was detected. The mixture was diluted with water (10 mL) and extracted with ethyl acetate (20 mL×3). The organic phase was dried over Na₂SO₄ and concentrated under reduced pressure. The crude was purified by column chromatography (SiO₂, Petroleum ether:ethyl acetate=0-50%) to give the product 7-chloro-5-(4-chloro-2-fluoro-phenyl)-2-methyl-pyrido[3,4-d]pyridazin-1-one (106 mg, 0.33 mmol, 75.2% yield)

¹H NMR (400 MHz, DMSO) δ 8.24 (s, 1H), 8.16 (d, J=2.9 Hz, 1H), 7.71 (dd, J=16.8, 8.9 Hz, 2H), 7.55 (dd, J=8.3, 1.9 Hz, 1H), 3.75 (s, 3H).

Step 9: To a solution of 1-cyclopropyl-4-[(6R)-4-(4,4,5,5-tetramethyl-1,3,2-dioxaborolan-2-yl)-3,6-dihydro-2H-pyran-6-yl]pyrazole (59 mg, 0.185 mmol, 1.2 eq) and 7-chloro-5-(4-chloro-2-fluoro-phenyl)-2-methyl-pyrido[3,4-d]pyridazin-1-one (50 mg, 0.154 mmol, 1.0 eq) in 1,4-dioxane (6 mL) and water (2 mL) were added Cs₂CO₃ (100 mg, 0.31 mmol, 2.0 eq) and Pd(dppf)Cl₂·DCM (11 mg, 0.0154 mmol, 0.1 eq) under N₂. The mixture was stirred at 75° C., for 8 hours. LCMS indicated that the starting material was consumed, and desired compound was detected. The mixture was diluted with water (10 mL) and extracted with ethyl acetate (20 mL×3). The organic phase was dried over Na₂SO₄ and concentrated under reduced pressure. The crude product was purified by column chromatography (SiO₂, Petroleum ether:ethyl acetate=0-100%) to give the product 5-(4-chloro-2-fluoro-phenyl)-7-[(6R)-6-(1-cyclopropylpyrazol-4-yl)-3,6-dihydro-2H-pyran-4-yl]-2-methyl-pyrido[3,4-d]pyridazin-1-one (62 mg, 0.13 mmol, 84.1% yield) ¹H NMR (400 MHz, DMSO) δ 8.23 (s, 1H), 8.11 (d, J=3.2 Hz, 1H), 7.77 (d, J=7.8 Hz, 1H), 7.73-7.68 (m, 2H), 7.53 (dd, J=8.3, 1.9 Hz, 1H), 7.45-7.37 (m, 1H), 7.17-7.11 (m, 1H), 5.38 (d, J=2.5 Hz, 1H), 4.04-3.95 (m, 1H), 3.94 (s, 1H), 3.81 (dd, J=12.5, 7.6 Hz, 1H), 3.76 (d, J=4.4 Hz, 3H), 3.68 (ddd, J=11.2, 7.4, 3.9 Hz, 1H), 1.02-0.98 (m, 2H), 0.94-0.89 (m, 2H).

Step 10: To a solution of 5-(4-chloro-2-fluoro-phenyl)-7-[(6R)-6-(1-cyclopropylpyrazol-4-yl)-3,6-dihydro-2H-pyran-4-yl]-2-methyl-pyrido[3,4-d]pyridazin-1-one (110 mg, 0.23 mmol, 1.0 eq) in ethyl acetate (5 mL) was added PtO₂ (157 mg, 0.069 mmol, 0.3 eq) under H₂ at 20° C. The mixture was stirred at 20° C., for 3 hours. LCMS indicated that the starting material was consumed, and desired compound was detected. The mixture was filtered with diatomite and concentrated under reduced pressure. The crude product was purified by column chromatography (SiO₂, Petroleum ether:ethyl acetate=0-100%) and prep-HPLC (Instrument: SHIMADZU LC-20AP-4, column: Gemini, mobile phase: ACN—H₂O (0.1% TFA), gradient: 70%-80%) to afford 5-(4-chloro-2-fluoro-phenyl)-7-[(2R,4S)-2-(1-cyclopropylpyrazol-4-yl)tetrahydropyran-4-yl]-2-methyl-pyrido[3,4-d]pyridazin-1-one (29 mg, 0.061 mmol, 26.3% yield) LC-MS: Rt: 1.309 min, m/z: 480.05 [M+H]⁺. 100% purity at 214 nm. ¹H NMR (400 MHz, DMSO) δ 8.10-8.08 (m, 2H), 7.72-7.67 (m, 3H), 7.53 (dd, J=8.3, 2.0 Hz, 1H), 7.37 (s, 1H), 4.51-4.44 (m, 1H), 4.07 (dd, J=10.9, 3.3 Hz, 1H), 3.74 (s, 3H), 3.68 (dd, J=9.4, 6.6 Hz, 1H), 3.65-3.60 (m, 1H), 3.43-3.36 (m, 1H), 2.15 (d, J=13.0 Hz, 1H), 1.93-1.79 (m, 3H), 0.97-0.96 (m, 2H), 0.93-0.87 (m, 2H). HPLC: Rt: 3.59 min, 99.1% purit at 214 nm

### Example 3-Synthesis of Compound I-53: 7-[(2R, 4S)-2-(1-cyclopropylpyrazol-4-yl)tetrahydropyran-4-yl]-5-[2-fluoro-4-(trifluoro omethyl)phenyl]-2-methyl-pyrido[3,4-d]pyridazin-1-one

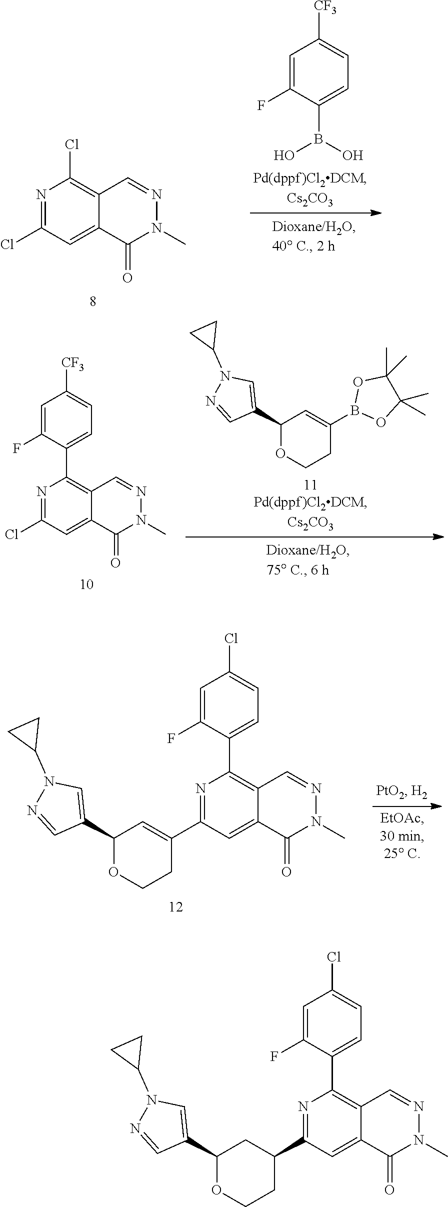 [SMILES: Cn1ncc2c(Cl)nc(Cl)cc2c1=O ; OB(O)c1ccc(C(F)(F)F)cc1F ; Cn1ncc2c(-c3ccc(C(F)(F)F)cc3F)nc(Cl)cc2c1=O ; CC1(C)OB(C2=C[C@H](c3cnn(C4CC4)c3)OCC2)OC1(C)C ; Cn1ncc2c(-c3ccc(Cl)cc3F)nc(C3=C[C@H](c4cnn(C5CC5)c4)OCC3)cc2c1=O ; Cn1ncc2c(-c3ccc(Cl)cc3F)nc([C@H]3CCO[C@@H](c4cnn(C5CC5)c4)C3)cc2c1=O]

Step 1: To a solution of [2-fluoro-4-(trifluoromethyl)phenyl]boronic acid (84 mg, 0.40 mmol, 1.0 eq) in 1,4-dioxane (6 mL) were added Cs₂CO₃ (263 mg, 0.81 mmol, 2.0 eq), 5,7-dichloro-2-methyl-pyrido[3,4-d]pyridazin-1-one (93 mg, 0.40 mmol, 1.0 eq) (Compound 8 from Example 2) and Pd(dppf)Cl₂·DCM (30 mg, 0.040 mmol, 0.1 eq) under N₂ at 25° C. The mixture was stirred at 40° C., for 2 hours. LCMS indicated that the starting material was consumed, and desired compound was detected. The mixture was diluted with water (10 mL) and extracted with ethyl acetate (20 mL×3). The organic phase was dried over Na₂SO₄ and concentrated under reduced pressure. The crude was purified by column chromatography (SiO₂, Petroleum ether:ethyl acetate=0-50%) to give the product 7-chloro-5-[2-fluoro-4-(trifluoromethyl)phenyl]-2-methyl-pyrido[3,4-d]pyridazin-1-one (51 mg, 0.14 mmol, 35.3% yield) as a white solid.

Step 2: To a solution of 1-cyclopropyl-4-[(6R)-4-(4,4,5,5-tetramethyl-1,3,2-dioxaborolan-2-yl)-3,6-dihydro-2H-pyran-6-yl]pyrazole (50 mg, 0.16 mmol, 1.1 eq) and 7-chloro-5-[2-fluoro-4-(trifluoromethyl)phenyl]-2-methyl-pyrido[3,4-d]pyridazin-1-one (51 mg, 0.14 mmol, 1.0 eq) in 1,4-dioxane (6 mL) and water (2 mL) were added Pd(dppf)Cl₂·DCM (10 mg, 0.014 mmol, 0.1 eq) and Cs₂CO₃ (93 mg, 0.29 mmol, 2.0 eq) under N₂. The mixture was stirred at 75° C., for 8 hours. LCMS indicated that the starting material was consumed, and desired compound was detected. The mixture was diluted with water (10 mL) and extracted with ethyl acetate (20 mL×3). The organic phase was dried over Na₂SO₄ and concentrated under reduced pressure. The crude product was purified by column chromatography (SiO₂, Petroleum ether:ethyl acetate=0-100%) to give the product 7-[(6R)-6-(1-cyclopropylpyrazol-4-yl)-3,6-dihydro-2H-pyran-4-yl]-5-[2-fluoro-4-(trifluoromethyl)phenyl]-2-methyl-pyrido[3,4-d]pyridazin-1-one (68 mg, 0.13 mmol, 93.2% yield)

Step 3: To a solution of 7-[(6R)-6-(1-cyclopropylpyrazol-4-yl)-3,6-dihydro-2H-pyran-4-yl]-5-[2-fluoro-4-(trifluoromethyl)phenyl]-2-methyl-pyrido[3,4-d]pyridazin-1-one (62 mg, 0.12 mmol, 1.0 eq) in ethyl acetate (5 mL) was added PtO₂ (28 mg, 0.12 mmol, 1.0 eq) under H₂ at 20° C. The mixture was stirred at 20° C., for 40 minutes. LCMS indicated that the starting material was consumed, and desired compound was detected. The mixture was filtered with diatomite and concentrated under reduced pressure. The crude product was purified by column chromatography (SiO₂, Petroleum ether:ethyl acetate=0-100%) and prep-HPLC (Instrument: SHIMADZU LC-20AP-4, column: Gemini, mobile phase: ACN—H₂O (0.1% TFA), gradient: 75%-85%) to afford 7-[(2R)-2-(1-cyclopropylpyrazol-4-yl)tetrahydropyran-4-yl]-5-[2-fluoro-4-(trifluoromethyl)phenyl]-2-methyl-pyrido[3,4-d]pyridazin-1-one (16 mg, 0.032 mmol, 26.0% yield) LC-MS: Rt: 1.47 min, m/z: 514.2 [M+H]⁺. 100% purity at 214 nm. ¹H NMR (400 MHz, DMSO) δ 8.13-8.11 (m, 2H), 7.95-7.88 (m, 2H), 7.84-7.77 (m, 1H), 7.71 (s, 1H), 7.37 (s, 1H), 4.48 (d, J=9.5 Hz, 1H), 4.07 (dd, J=11.2, 2.6 Hz, 1H), 3.74 (s, 3H), 3.70-3.60 (m, 2H), 2.16 (d, J=12.7 Hz, 1H), 1.92-1.79 (m, 3H), 1.00-0.94 (m, 2H), 0.93-0.87 (m, 2H). HPLC: Rt: 4.09 min, 97.9% purit at 214 nm.

### Example 4-Synthesis of Compound I-58: 7-[(2S,6R)-2-(1-cyclopropylpyrazol-4-yl)-6-methyl-morpholin-4-yl]-5-(2,4-difluorophenyl)-2-methyl-pyrido[3,4-d]pyridazin-1-one

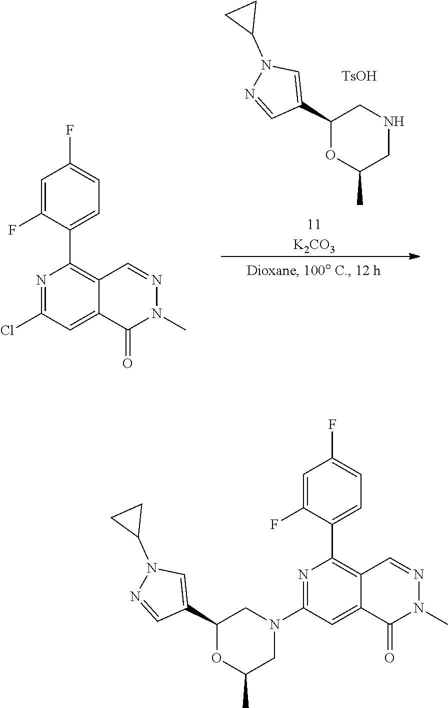 [SMILES: Cn1ncc2c(-c3ccc(F)cc3F)nc(Cl)cc2c1=O ; C[C@@H]1CNC[C@H](c2cnn(C3CC3)c2)O1 ; "K2CO3" ; "TsOH" ; C[C@@H]1CN(c2cc3c(=O)n(C)ncc3c(-c3ccc(F)cc3F)n2)C[C@H](c2cnn(C3CC3)c2)O1]

Step 1: To a solution of 7-chloro-5-(2,4-difluorophenyl)-2-methyl-pyrido[3,4-d]pyridazin-1-one (50 mg, 0.16 mmol, 1.0 eq) in 1,4-dioxane (5 mL) were added (2S,6R)-2-(1-cyclopropylpyrazol-4-yl)-6-methyl-morpholine 4-methylbenzenesulfonic acid (68 mg, 0.18 mmol, 1.1 eq) and K₂CO₃ (45 mg, 0.33 mmol, 2.0 eq) at 25° C. The mixture was stirred at 100° C., for 12 hours. LCMS indicated that the starting material was consumed, and desired compound was detected. The mixture was filtered with diatomite and concentrated under reduced pressure. The crude product was purified by column chromatography (SiO₂, Petroleum ether:ethyl acetate=0-50%) and prep-HPLC (Instrument: SHIMADZU LC-20AP-4, column: Gemini, mobile phase: ACN—H₂O (0.1% TFA), gradient: 75%-85%) to afford 7-[(2S,6R)-2-(1-cyclopropylpyrazol-4-yl)-6-methyl-morpholin-4-yl]-5-(2,4-difluorophenyl)-2-methyl-pyrido[3,4-d]pyridazin-1-one (33 mg, 0.069 mmol, 42.4% yield) LC-MS: Rt: 1.32 min, m/z: 479.1 [M+H]⁺. 100% purity at 214 nm. ¹H NMR (400 MHz, DMSO) δ 7.84 (s, 1H), 7.81 (d, J=3.2 Hz, 1H), 7.67 (td, J=8.5, 6.7 Hz, 1H), 7.48-7.39 (m, 3H), 7.28 (td, J=8.3, 2.2 Hz, 1H), 4.55 (dd, J=10.9, 2.4 Hz, 1H), 4.44 (dd, J=27.2, 12.4 Hz, 2H), 3.80-3.72 (m, 1H), 3.69 (dd, J=7.1, 3.6 Hz, 1H), 3.66 (s, 3H), 3.02-2.95 (m, 1H), 2.70 (dd, J=20.5, 9.5 Hz, 1H), 1.21 (d, J=6.2 Hz, 3H), 1.03-0.97 (m, 2H), 0.97-0.90 (m, 2H). HPLC: Rt: 3.64 min, 97.8% purit at 214 nm

### Example 5-Synthesis of Compound I-63: 7-[(2R)-2-(1-cyclopropylpyrazol-4-yl)tetrahydropyran-4-yl]-5-(2,4-difluorophenyl)-2-methyl-pyrido[3,4-d]pyridazin-1-one

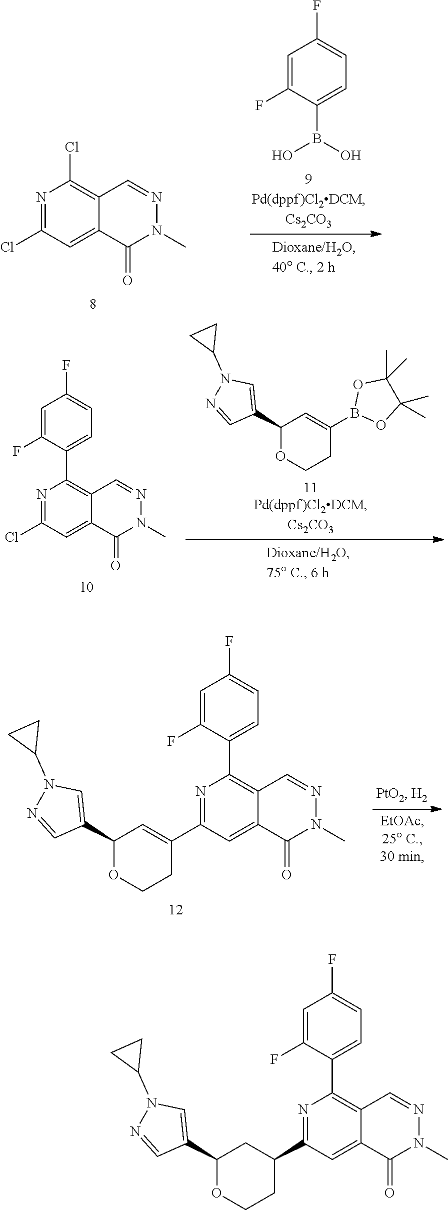 [SMILES: Cn1ncc2c(Cl)nc(Cl)cc2c1=O ; OB(O)c1ccc(F)cc1F ; Cn1ncc2c(-c3ccc(F)cc3F)nc(Cl)cc2c1=O ; CC1(C)OB(C2=C[C@H](c3cnn(C4CC4)c3)OCC2)OC1(C)C ; Cn1ncc2c(-c3ccc(F)cc3F)nc(C3=C[C@H](c4cnn(C5CC5)c4)OCC3)cc2c1=O ; Cn1ncc2c(-c3ccc(F)cc3F)nc([C@H]3CCO[C@@H](c4cnn(C5CC5)c4)C3)cc2c1=O]

Step 1: To a solution of (2,4-difluorophenyl) boronic acid (84 mg, 0.54 mmol, 1.0 eq) in 1,4-dioxane (6 mL) were added Cs₂CO₃ (348 mg, 1.07 mmol, 2.0 eq), 5,7-dichloro-2-methyl-pyrido[3,4-d]pyridazin-1-one (123 mg, 0.54 mmol, 1.0 eq) and Pd(dppf)Cl₂·DCM (39 mg, 0.054 mmol, 0.1 eq) under N₂ at 25° C. The mixture was stirred at 40° C., for 6 hours. LCMS indicated that the starting material was consumed, and desired compound was detected. The mixture was diluted with water (10 mL) and extracted with ethyl acetate (20 mL×3). The organic phase was dried over Na₂SO₄ and concentrated under reduced pressure. The crude was purified by column chromatography (SiO₂, Petroleum ether:ethyl acetate=0-50%) to give the product 7-chloro-5-(2,4-difluorophenyl)-2-methyl-pyrido[3,4-d]pyridazin-1-one (82 mg, 0.27 mmol, 49.9% yield) as a white solid. ¹H NMR (400 MHz, DMSO) δ 8.23 (d, J=0.7 Hz, 1H), 8.16-8.13 (m, 1H), 7.74 (td, J=8.6, 6.5 Hz, 1H), 7.55 (ddd, J=10.6, 9.5, 2.5 Hz, 1H), 7.36 (td, J=8.6, 2.4 Hz, 1H), 3.75 (s, 3H).

Step 2: To a solution of 1-cycl opropyl-4-[(6R)-4-(4,4,5,5-tetramethyl-1,3,2-dioxaborolan-2-yl)-3,6-dihydro-2H-pyran-6-yl]pyrazole (45 mg. 0.14 mmol, 1.1 eq) and 7-chloro-5-(2,4-difluorophenyl)-2-methyl-pyrido[3,4-d]pyridazin-1-one (40 mg. 0.13 mmol, 1.0 eq) in 1,4-dioxane (6 mL) and water (2 mL) were added Cs₂CO₃ (85 mg, 0.26 mmol, 2.0 eq) and Pd(dppf)Cl₂·DCM (9.5 mg, 0.013 mmol, 0.1 eq) under N₂. The mixture was stirred at 75° C., for 8 hours. LCMS indicated that the starting material was consumed, and desired compound was detected. The mixture was diluted with water (10 mL) and extracted with ethyl acetate (20 mL×3). The organic phase was dried over Na₂SO₄ and concentrated under reduced pressure. The crude product was purified by column chromatography (SiO₂, Petroleum ether:ethyl acetate=0-100%) to give the product 7-[(6R)-6-(1-cyclopropylpyrazol-4-yl)-3,6-dihydro-2H-pyran-4-yl]-5-(2,4-difluorophenyl)-2-methyl-pyrido[3,4-d]pyridazin-1-one (48 mg, 0.10 mmol, 80.0% yield) ¹H NMR (400 MHz, DMSO) δ 8.23 (s, 1H), 8.09 (d, J=3.3 Hz, 1H), 7.78 (s, 1H), 7.77-7.71 (m, 1H), 7.50 (dd, J=13.8, 5.8 Hz, 1H), 7.41 (s, 1H), 7.36-7.31 (m, 1H), 7.14 (s, 1H), 5.38 (d, J=2.5 Hz, 1H), 4.06-3.92 (m, 2H), 3.81 (dt, J=11.6, 5.9 Hz, 1H), 3.75 (s, 3H), 3.67 (td, J=7.4, 3.8 Hz, 1H), 1.17 (t, J=7.1 Hz, 1H), 1.02-1.00 (m, 2H), 0.94-0.89 (m, 2H).

Step 3: To a solution of 7-[(6R)-6-(1-cyclopropylpyrazol-4-yl)-3,6-dihydro-2H-pyran-4-yl]-5-(2,4-difluorophenyl)-2-methyl-pyrido[3,4-d]pyridazin-1-one (56 mg. 0.12 mmol, 1.0 eq) in ethyl acetate (5 mL) was added PtO₂ (28 mg, 0.12 mmol, 1.0 eq) under H₂ at 25° C. The mixture was stirred at 25° C., for 40 minutes. LCMS indicated that the starting material was consumed and desired compound was detected. The mixture was filtered with diatomite and concentrated under pressure. The crude product was purified by column chromatography (SiO₂, Petroleum ether:ethyl acetate=0-50%) and prep-HPLC (Instrument: SHIMADZU LC-20AP-4, column: Gemini, mobile phase: ACN—H₂O (0.1% TFA), gradient: 75%-85%) to 7-[(2R)-2-(1-cyclopropylpyrazol-4-yl)tetrahydropyran-4-×1]-5-(2,4-difluorophenyl)-2-methyl-pyrido]3,4-d]pyridazin-1-one (17 mg, 0.038 mmol, 31.0% yield) LC-MS: Rt: 1.22 min, m/z: 464.1 [M+H]⁺. 100% purity at 214 nm. ¹H NMR (400 MHz, DMSO) δ 8.08-8.07 (m, 2H), 7.74-7.70 (m, 2H), 7.51-7.49 (m, 1H), 7.39-7.30 (m, 2H), 4.47 (d, J=9.6 Hz, 1H), 4.07 (dd, J=11.4, 2.7 Hz, 1H), 3.74 (s, 3H), 3.72-3.58 (m, 2H), 3.38 (d, J=11.5 Hz, 1H), 2.16 (d, J=12.9 Hz, 1H), 2.01-1.80 (m, 3H), 1.02-0.94 (m, 2H), 0.93-0.87 (m, 2H). HPLC: Rt: 3.37 min, 97.6% purit at 214 nm.

### Example 6-Synthesis of Compound I-69 and I-74: 4-(4-chloro-2-fluorophenyl)-2-((2S,6R)-2-(1-cyclopropyl-1H-pyrazol-4-yl)-6-methylmorpholino)-7-methylpyrimido[4,5-d]pyridazin-8 (7H)-one and 4-(4-chloro-2-fluorophenyl)-2-((2S,6R)-2-(1-cyclopropyl-1H-pyrazol-4-yl)-6-methylmorpholino)pyrimido[4,5-d]pyridazin-8 (7H)-one, respectively

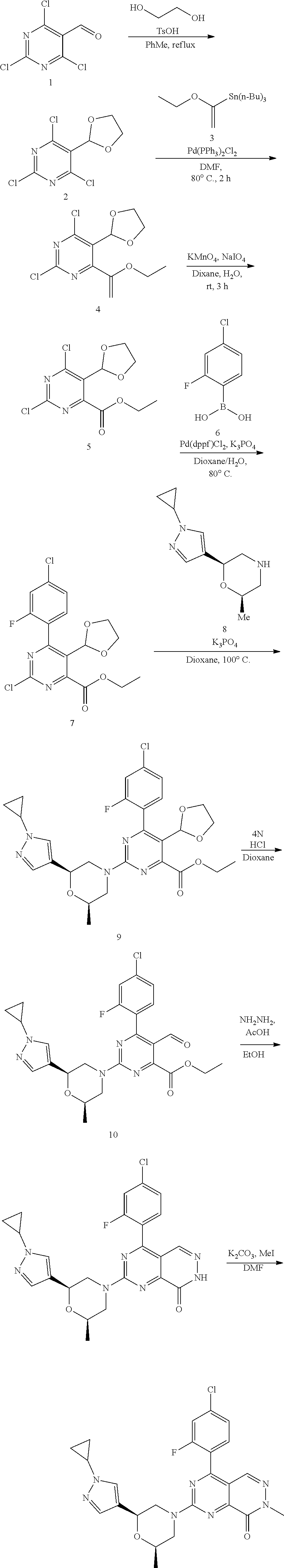

Step 1: To a solution of 2,4,6-trichloropyrimidine-5-carbaldehyde (20 g, 94.6 mmol, 1.00 eq) and ethylene glycol (5.87 g, 94.6 mmol, 3 eq) in toluene (200 mL) was added TsOH (3.25 g, 18.9 mmol, 0.2 eq). The mixture was refluxed at 120° C., for 8 hours. The mixture was evaporated and purified by silica gel column chromatography (eluting with ethyl acetate/petroleum ether, 10% to 20%) to give desired product (22 g, 86.1 mmol, 90% yield) as a white solid. ¹H NMR (400 MHz, DMSO) δ 6.21 (d, J=7.0 Hz, 1H), 4.12 (dd, J=68.4, 6.1 Hz, 4H).

Step 2: To a solution of 2,4,6-trichloro-5-methyl-pyrimidine (20 g, 78.28 mmol, 1.0 eq) and Pd(PPh₃)₂Cl₂ (11.0 g, 15.66 mmol, 0.2 eq) in dimethylformamide (200 mL) under nitrogen was added 1-ethoxyvinyl-tri-N-butyltin (28.27 g, 78.28 mmol, 1.0 eq) dropwise. The mixture was heated at 80° C., for 12 hours, then cooled and poured into a solution of potassium fluoride (2 g) in water (100 mL). The mixture was extracted with ethyl acetate (2×500 mL), and the organic phase was washed with water (100 mL). The residue was concentrated under vacuum and purified by silica gel column chromatography (Petroleum ether:ethyl acetate=10:1) to give the desired product (15 g, 42.95 mmol, 74.6%) as a yellow oil. ¹H NMR (400 MHz, CDCl₃) δ 6.12 (s, 1H), 4.68 (s, 1H), 4.60 (s, 1H), 4.27 (d, J=1.1 Hz, 2H), 4.05 (d, J=1.0 Hz, 3H), 3.94 (d, J=7.0 Hz, 2H), 1.39 (t, J=7.0 Hz, 3H).

Step 3: The suspension of sodium metaperiodate (20 g, 113.4 mmol, 2.2 eq) in water (100 mL) was sonicated until a clear solution was obtained. Then to the mixture was added a solution of 2,4-dichloro-5-(1,3-dioxolan-2-yl)-6-(1-ethoxyvinyl)pyrimidine (15 g, 51.3 mmol, 1.0 eq) in 1,4-dioxane (150 mL), followed by potassium permanganate (1.63 mg, 10.2 mmol, 0.2 eq). The mixture was stirred at room temperature for 2 hours. The mixture was filtered. The filtrate was diluted with saturated solutions of sodium bicarbonate and sodium chloride and extracted twice with ethyl acetate. The organic phase was dried over sodium sulfate, filtered through a pad of silica gel and concentrated in vacuum. Column chromatography on silica gel using a 0-30% gradient of ethyl acetate in petroleum ether yielded the title product (8.5 g. 29.0 mmol, 53%).

Step 4: To a solution of ethyl 2,6-dichloro-5-(1,3-dioxolan-2-yl)pyrimidine-4-carboxylate (1.5 g. 5.12 mmol, 1.0 eq) and (4-chloro-2-fluoro-phenyl) boronic acid (2.17 g. 10.24 mmol, 2 eq) in 1,4-dioxane: H₂O (=10:1, 20 mL) were added Pd(dppf)Cl₂ (749 mg, 1.02 mmol, 0.2 eq) and K₂PO₄ (116 mg, 0.55 mmol, 2 eq). The mixture was refluxed at 60° C., for 3 hours. LC-MS showed desired product. The mixture was evaporated and purified by flash column (petoleum ether:ethyl acetate=95:5) to afford desired product (1.5 g. 3.87 mmol, 75.7%) as a white solid. ¹H NMR (400 MHz, DMSO) δ 7.72-7.60 (m, 2H), 7.49 (dd, J=8.3, 1.9 Hz, 1H), 5.88 (s, 1H), 4.45-4.32 (m, 2H), 4.03-3.72 (m, 5H), 1.33 (t, J=7.1 Hz, 3H).

Step 5: To a solution of ethyl 2-chloro-6-(4-chloro-2-fluoro-phenyl)-5-(1,3-dioxolan-2-yl)pyrimidine-4-carboxylate (1.5 g. 3.87 mmol, 1.0 eq) and (2S,6R)-2-(1-cyclopropylpyrazol-4-yl)-6-methyl-morpholine (803 mg. 3.87 mmol, 1 eq) in 1,4-dioxane (20 mL) was added K₃PO₄ (1.64 g. 7.75 mmol, 2 eq). The mixture was refluxed at 100° C., for 2 hours. LC-MS indicated that the starting material was consumed and desired product detected. The mixture was evaporated and purified by flash column (petroleum ether:ethyl acetate=1:1) to afford desired product (1.2 g. 2.15 mmol, 55.6%) as a yellow solid. ¹H NMR (400 MHz, CDCl3) δ 7.51 (s, 1H), 7.39 (t, J=7.9 Hz, 1H), 7.26-7.13 (m, 2H), 5.66 (d, J=0.5 Hz, 1H), 5.30 (s, 1H), 4.78 (d, J=13.2 Hz, 1H), 4.67 (d, J=13.0 Hz, 1H), 4.51 (dd, J=10.8, 2.3 Hz, 1H), 4.47-4.33 (m, 2H), 3.91-3.75 (m, 3H), 3.78-3.64 (m, 1H), 3.56 (dt, J=10.9, 3.7 Hz, 1H), 2.95 (dd, J=13.0, 11.3 Hz, 1H), 2.71 (dd, J=13.1, 10.9 Hz, 1H), 1.41 (t, J=7.2 Hz, 3H), 1.27 (d, J=6.2 Hz, 3H), 1.10 (d, J=3.2 Hz, 1H), 1.06-0.94 (m, 2H).

Step 6: A mixture of ethyl 2,6-dichloro-5-(1,3-dioxolan-2-yl)pyrimidine-4-carboxylate (300 mg, 0.54 mmol, 1.0 eq) and HCl in 1,4-dioxane (4 N, 5 mL) was refluxed at 25° C., for 2 hours. LC-MS indicated that the starting material was consumed and about 90% desired product was detected. The mixture was evaporated and purified by flash column (petroleum ether:ethyl acetate=1:1) to afford desired product (250 mg, 0.47 mmol, 97%) as a yellow oil.

Step 7: To a solution of ethyl 2-chloro-6-(4-chloro-2-fluoro-phenyl)-5-formyl-pyrimidine-4-carboxylate (300 mg, 0.58 mmol, 1.0 eq) in ethanol (5 mL) were added NH₂NH₂H₂O (29 mg, 0.06 mmol, 0.1 eq) and CH₃COOH (27 mg, 0.58 mmol, 1 eq). The mixture was refluxed at 60° C., for 4 hours. LC-MS showed desired product. The mixture was evaporated and purified by flash column (CH₂Cl₂:MeOH=95:5) to afford desired product (200 mg, 0.42 mmol, 71.1%) as a yellow solid. ¹H NMR (400 MHz, DMSO) δ 12.85 (s, 1H), 7.85-7.81 (m, 2H), 7.73-7.70 (m, 2H), 7.59-7.42 (m, 2H), 4.83-4.81 (m, 1H), 4.71-4.69 (m, 1H), 4.55-4.53 (m, 1H), 3.15-3.13 (m, 1H), 2.85-2.81 (m, 1H), 1.23-1.19 (m, 3H), 1.04-1.02 (m, 2H), 0.95-0.93 (m, 2H).

Step 8: To a solution of 4-(4-chloro-2-fluoro-phenyl)-2-[(2S,6R)-2-(1-cyclopropylpyrazol-4-yl)-6-methyl-morpholin-4-yl]-7H-pyrimido[4,5-d]pyridazin-8-one (100 mg, 0.21 mmol, 1.0 eq) and K₂CO₃ (57 mg, 0.42 mmol, 2 eq) in DMF (5 mL) and MeCN (5 mL) was added CH₃I (59 mg, 0.42 mmol, 2 eq). The mixture was refluxed at 25° C., for 12 hours. LC-MS indicated that the starting material was consumed, and desired product detected. The mixture was evaporated and purified by prep-HPLC to give 4-(4-chloro-2-fluorophenyl)-2-((2S,6R)-2-(1-cyclopropyl-1H-pyrazol-4-yl)-6-methylmorpholino)-7-methylpyrimido[4,5-d]pyridazin-8 (7H)-one (40 mg, 0.08 mmol, 38.5% yield). ¹H NMR (400 MHz, DMSO) δ 7.93-7.80 (m, 2H), 7.73-7.71 (m, 2H), 7.60-7.40 (m, 2H), 4.83 (t, J=12.5 Hz, 1H), 4.73 (d, J=11.1 Hz, 1H), 4.53 (s, 1H), 3.68 (s, 3H), 3.16-3.14 (m, 1H), 2.85-2.83 (m, 1H), 1.23-1.19 (m, 3H), 1.05-1.01 (m, 2H), 0.96-0.93 (m, 2H).

### Example 7: Synthesis of Compound I-79: 7-((2S,6R)-2-(1-cyclopropyl-1H-pyrazol-4-yl)-6-methylmorpholino)-5-(2-fluoro-4-(trifluoromethyl)phenyl)-2,3-dimethyl-2,6-naphthyridin-1 (2H)-one

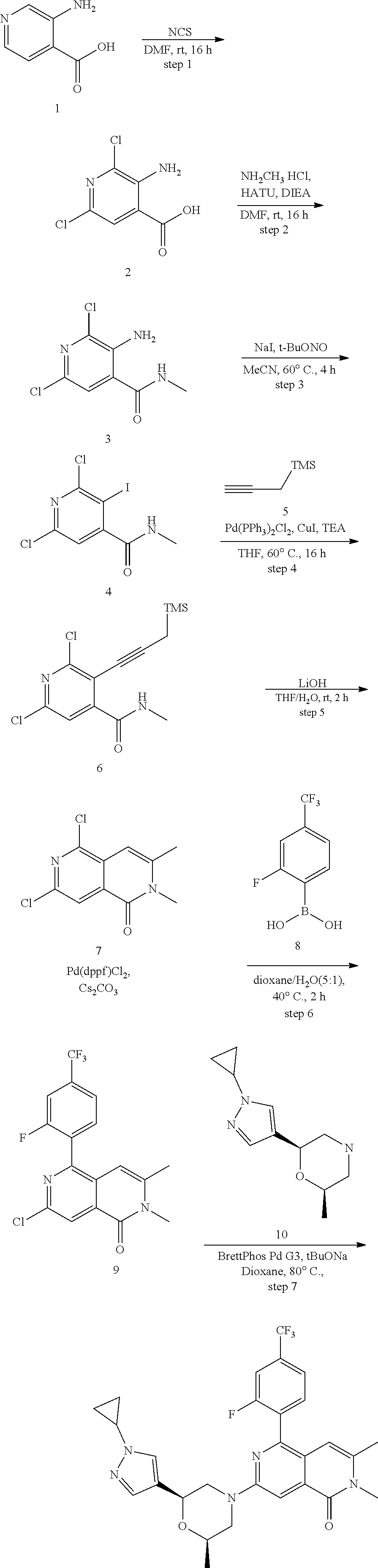

Step 1: A mixture of 3-aminopyridine-4-carboxylic acid (1 g, 7.24 mmol, 1 eq) and NCS (2.58 g, 14.48 mmol, 2 eq) in DMF (5 mL) was stirred at 35° C., for 16 hours. LCMS showed the starting material was consumed and one major peak with desired mass was detected. The solution was diluted with water (100 mL) and extracted with EtOAc (30 mL×3). The combined organic layers were washed with brine (5×100 mL), dried over sodium sulfate and filtered. The filter was concentrated under reduced pressure to afford crude 3-amino-2,6-dichloroisonicotinic acid (1.3 g, yield=78.1%) which was used for next step directly. LC-MS [M+H]⁺=206.9 R.T=0.938 min

Step 2: To a solution of 3-amino-2,6-dichloroisonicotinic acid (1 g, 4.83 mmol, 1 eq) in DMF (10 mL) was added HATU (2.76 g, 7.25 mmol, 1.5 eq). The resulting mixture was stirred for 10 min. Then DIEA (1.87 g, 14.5 mmol, 3 eq) and methylamine hydrochloride (0.39 g, 5.80 mmol, 1.2 eq) were added. The mixture was further stirred at 25° C., for 16 hours. LCMS showed the starting material was consumed and one major peak with desired mass was detected. The solution was diluted with water (100 mL) and extracted with EtOAc (20 mL×3). The combined organic layers were washed with brine (5×50 mL), dried over sodium sulfate and filtered. The filter was concentrated under reduced pressure and purified by flash column chromatography (Petroleum ether:Ethyl acetate=80:20) to afford 3-amino-2,6-dichloro-N-methylisonicotinamide as an off-white solid (500 mg, yield=42.3%). LC-MS [M+H]⁺=219.9 R.T=0.847 min

Step 3: To a solution of 3-amino-2,6-dichloro-N-methylisonicotinamide (4 g, 18.18 mmol, 1 eq) in MeCN (50 mL) were added CuI (17.31 g, 90.88 mmol, 5 eq) and 1-BuONO. The mixture was stirred at 60° C., for 4 hours. LCMS showed the starting material was consumed and one major peak with desired mass was detected. The mixture was filtered. The filter was diluted with water (200 mL) and extracted with EtOAc (100 mL×3). The combined organic layers were washed with brine (3×200 mL), dried over sodium sulfate and filtered. The filter was concentrated under reduced pressure. The residue was purified by flash column chromatography (Petroleum ether/Ethyl acetate=80:20) to afford 2,6-dichloro-3-iodo-N-methylisonicotinamide (500 mg, yield=7.5%) as an off-white solid. LC-MS [M+H]⁺=330.8 R.T=0.923 min

Step 4: To a solution of 2,6-dichloro-3-iodo-N-methylisonicotinamide (1.5 g. 4.53 mmol, 1 eq) in THF (50 mL) were added trimethyl(prop-2-yn-1-yl) silane (1.02 g. 9.07 mmol, 2 eq), Pd(PPh₃)₂Cl₂ (0.32 g. 0.45 mmol, 0.1 eq), CuI (0.26 g. 1.36 mmol, 0.3 eq), and triethylamine (1.38 g, 13.60 mmol, 3 eq). The resulting mixture was stirred at 60° C., for 16 hours under N₂. LCMS showed the starting material was consumed and one major peak with desired mass was detected. The mixture was filtered and the filter was concentrated under reduced pressure. The residue was purified by flash column chromatography (Petroleum ether:Ethyl acetate=90:10) to afford 2,6-dichloro-N-methyl-3-(3-(trimethylsilyl) prop-1-yn-1-yl) isonicotinamide (400 mg, yield=25.2%) as a black solid. LC-MS [M+H]⁺=315.0 R.T=1.361 min

Step 5: To a solution of 2,6-dichloro-N-methyl-3-(3-trimethylsilylprop-1-ynyl)pyridine-4-carboxamide (200 mg, 0.63 mmol, 1 eq) in THF/H₂O (10:1, 5.5 mL) was added LiOH (30.45 mg, 1.27 mmol, 2 eq), stirred at 25° C., for 16 h. LCMS showed the starting material was consumed and one major peak with desired mass was detected. The solution was diluted with water (20 mL) and extracted with EtOAc (5 mL×3). The combined organic layers were washed with brine (3×10 mL), dried over sodium sulfate and filtered. The filter was concentrated under reduced pressure. The residue was purified by flash column chromatography (Petroleum ether:Ethyl acetate=90:10) to afford 5,7-dichloro-2,3-dimethyl-2,6-naphthyridin-1 (2H)-one (20 mg, yield=11.7%) as a light yellow solid. LC-MS [M+H]⁺=242.9 R.T=1.127 min ¹H NMR (400 MHz, DMSO) δ 8.01 (s, 1H), 6.72 (s, 1H), 3.54 (s, 3H), 2.52 (s, 3H).

Step 6: A mixture of 5,7-dichloro-2,3-dimethyl-2,6-naphthyridin-1 (2H)-one (80 mg, 0.33 mmol, 1 eq), (2-fluoro-4-(trifluoromethyl)phenyl) boronic acid (82 mg. 0.39 mmol, 1.2 eq), Cs₂CO₃ (321 mg. 0.99 mmol, 3 eq) and Pd(dppf)Cl₂ (48.2 mg. 0.066 mmol, 0.2 eq) in dioxane/H₂O (5:1, 6 mL) was stirred at 40° C., for 2 hours under N₂. LCMS showed the starting material was consumed and one major peak with desired mass was detected. The mixture was filtered and the filter was concentrated under reduced pressure. The residue was purified by TLC (Petroleum ether/Ethyl acetate=50:50) to afford 7-chloro-5-(2-fluoro-4-(trifluoromethyl)phenyl)-2,3-dimethyl-2,6-naphthyridin-1 (2H)-one (40 mg, yield=26.2%) as a light yellow solid. LC-MS [M+H]⁺=371.0 R.T=1.382 min

Step 7: A mixture of 7-chloro-5-(2-fluoro-4-(trifluoromethyl)phenyl)-2,3-dimethyl-2,6-naphthyridin-1 (2H)-one (100 mg, 0.27 mmol, 1 eq), (2S,6R)-2-(1-cyclopropylpyrazol-4-yl)-6-methyl-morpholine (83.86 mg, 0.40 mmol, 1.5 eq), Brettphos-Pd-G3 (48.90 mg, 0.054 mmol, 0.2 eq) and t-BuONa (77.77 mg, 0.81 mmol, 3 eq) in dioxane (5 mL) was stirred at 80° C., for 2 hours. LCMS showed the starting material was consumed and one major peak with desired mass was detected. The mixture was filtered and the filter was concentrated under reduced pressure. The residue was purified by flash column chromatography (Petroleum ether:Ethyl acetate=70:30) and prep-HPLC (Gemini 5 um C18 150*21.2 mm, mobile phase: ACN—H₂O (0.1% TFA); gradient: 5-95) and lyophilized to afford 7-((2S,6R)-2-(1-cyclopropyl-1H-pyrazol-4-yl)-6-methylmorpholino)-5-(2-fluoro-4-(trifluoromethyl)phenyl)-2,3-dimethyl-2,6-naphthyridin-1 (2H)-one (23.66 mg, yield=14.7%) as a yellow solid. LC-MS [M+H]⁺=542.0 R.T=1.478 min. ¹H NMR (400 MHz, DMSO) δ 7.69 (d, J=10.0 Hz, 1H), 7.84 (s, 1H), 7.75 (s, 1H), 7.75 (s, 1H), 7.48 (s, 1H), 7.46 (s, 1H), 6.05 (d, J=2.4 Hz, 1H), 4.57 (dd, J=10.8, 2.4 Hz, 1H), 4.24 (dd, J=22.8, 11.6 Hz, 2H), 3.83-3.74 (m, 1H), 3.71-3.65 (m, 1H), 3.51 (s, 3H), 2.84 (dd, J=12.0, 11.2 Hz, 1H), 2.58 (dd, J=12.4, 10.8 Hz, 1H), 2.32 (s, 3H), 1.21 (d, J=6.0 Hz, 3H), 1.04-0.99 (m, 2H), 0.95-0.90 (m, 2H).

### Example 8-Synthesis of Compound I-84: 2-((2R)-2-(1-cyclopropyl-1H-pyrazol-4-yl)tetrahydro-2H-pyran-4-yl)-4-(2-fluoro-4-(trifluoromethyl)phenyl)-7-methylpyrimido[4,5-d]pyridazin-8 (7H)-one

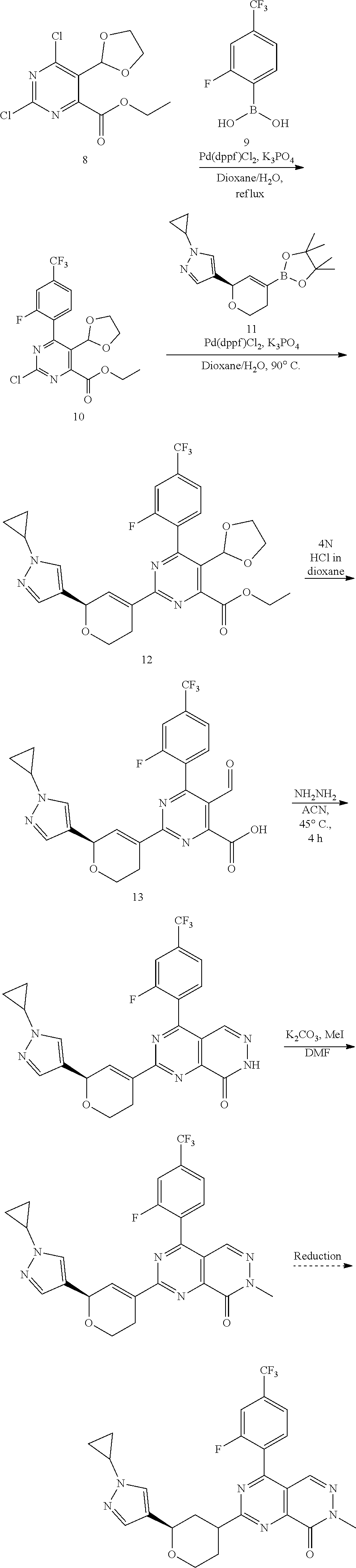

Step 1: To a solution of ethyl 2,6-dichloro-5-(1,3-dioxolan-2-yl)pyrimidine-4-carboxylate (1.0 g, 3.41 mmol, 1.0 eq) and [2-fluoro-4-(trifluoro methyl)phenyl]boronic acid (709 mg, 3.41 mmol, 1.0 eq) in 1,4-dioxane:H₂O=10:1 (15 mL) were added Pd(dppf)Cl₂ (499 mg, 0.68 mmol, 0.2 eq) and K₃PO₄ (1.45 g, 6.82 mmol, 2 eq). The mixture was refluxed at 60° C., for 6 hours. LC-MS showed desired product. The mixture was evaporated and purified by flash column (Petroleum ether:Ethyl acetate=95:5) to afford desired product (1.1 g, 2.61 mmol, 77%) as a white oil.

Step 2: To a solution of ethyl 2-chloro-5-(1,3-dioxolan-2-yl)-6-[2-fluoro-4-(trifluoromethyl)phenyl]pyrimidine-4-carboxylate (1.1 g, 2.61 mmol, 1.0 eq) and 1-cyclopropyl-4-[(6R)-4-(4,4,5,5-tetramethyl-1,3,2-dioxaborolan-2-yl)-3,6-dihydro-2H-pyran-6-yl]pyrazole (992 mg, 3.14 mmol, 1.2 eq) in 1,4-dioxane (15 mL) were added K₃PO₄ (1.1 g, 5.23 mmol, 2 eq) and Pd(dppf)Cl₂ (383 mg, 0.52 mmol, 0.2 eq). The mixture was refluxed at 60° C., for 24 hours. LC-MS showed desired product. The mixture was evaporated and purified by flash column (Petroleum ether:Ethyl acetate=1:1) to afford desired product (1.4 g, 2.44 mmol, 93%) as a yellow solid.

¹H NMR (400 MHz, CDCl₃) δ 7.64 (s, 1H), 7.56-7.48 (m, 2H), 7.49-7.39 (m, 3H), 5.94 (s, 1H), 5.39 (d, J=2.4 Hz, 1H), 4.46 (d, J=7.2 Hz, 2H), 4.08-4.00 (m, 1H), 3.84 (td, J=5.7, 2.6 Hz, 4H), 3.59-3.52 (m, 1H), 2.73 (dd, J=28.6, 9.1 Hz, 2H), 1.44 (d, J=7.2 Hz, 3H), 1.17-1.06 (m, 2H), 1.00 (d, J=7.0 Hz, 2H).

Step 3: To a solution of ethyl 2-[(6R)-6-(1-cyclopropylpyrazol-4-yl)-3,6-dihydro-2H-pyran-4-yl]-5-(1,3-dioxolan-2-yl)-6-[2-fluoro-4-(trifluoromethyl)phenyl]pyrimidine-4-carboxylate (250 mg, 0.44 mmol, 1.0 eq) was added HCl in dioxane (2 mL, 4 mmol/L). The mixture was refluxed at 25° C., for 12 hours. LC-MS showed that the starting material was consumed and about 90% acid formed (M+H=503). After evaporation, crude product (1 g) was obtained as a yellow oil, which was used directly for next step.

Step 4: To a solution of ethyl 2-chloro-6-(4-chloro-2-fluoro-phenyl)-5-formyl-pyrimidine-4-carboxylate (250 mg. 0.50 mmol, 1.0 eq) in ethanol were added NH₂NH₂H₂O (24.9 mg, 0.50 mmol, 1 eq) and CH& COOH (29.9 mg, 0.50 mmol, 1 eq). The mixture was refluxed at 40° C., for 6 hours. LC-MS showed desired product. The mixture was evaporated and purified by flash column (CH₂Cl₂:MeOH=95:5) to afford desired product (70 mg. 0.14 mmol, 28%) as a yellow oil. ¹H NMR (400 MHz, CDCl3) δ 8.06 (d, J=4.1 Hz, 1H), 7.82 (d, J=7.2 Hz, 1H), 7.74-7.67 (m, 2H), 7.60 (d, J=9.6 Hz, 1H), 7.50 (d, J=9.2 Hz, 2H), 5.46 (d, J=2.6 Hz, 1H), 4.20-4.04 (m, 2H), 3.92 (m, J=11.6, 7.1, 4.7 Hz, 1H), 3.57 (m, J=7.3, 3.8 Hz. 1H), 3.04-2.82 (m, 2H), 1.17-1.05 (m, 2H), 1.00 (d, J=7.2 Hz, 2H).

Step 5: To a solution of 2-[(6R)-6-(1-cyclopropylpyrazol-4-yl)-3,6-dihydro-2H-pyran-4-4-yl]-4-[2-fluoro-4-(trifluoromethyl)phenyl]-7H-pyrimido[4,5-d]pyridazin-8-one (140 mg, 0.28 mmol, 1.00 eq) and K₂CO₃ (77.64 mg, 0.56 mmol, 2 eq) in DMF (2 mL) was added CH₃I (79.7 mg, 0.56 mmol, 2 eq). The mixture was refluxed at 25° C., for 12 hours. LC-MS showed desired product. The mixture was evaporated and purified by flash column (dichloromethane:methanol=95:5) to afford desired product (70 mg, 0.14 mmol, 49%) as a yellow solid.

Step 6: To a solution of 2-[(6R)-6-(1-cyclopropylpyrazol-4-yl)-3,6-dihydro-2H-pyran-4-yl]-4-[2-fluoro-4-(trifluoromethyl)phenyl]-7-methyl-pyrimido[4,5-d]pyridazin-8-one (70 mg, 0.28 mmol, 1.00 eq) in EtOAc (2 mL) was added PtO₂ (9.30 mg, 0.04 mmol, 0.3 eq). The mixture was refluxed at 25° C. for 0.5 hour under H₂. LC-MS showed desired product. The mixture was evaporated and purified by prep-HPLC to afford desired product (22 mg, 0.04 mmol, 31%) as a white solid. LC-MS [M+H]⁺=515.4 R.T=1.234 min. ¹H NMR (400 MHz, DMSO) δ 8.23-8.20 (m, 1H), 7.99-7.96 (m, 1H), 7.83-7.77 (m, 3H), 7.56 (s, 1H), 4.62-4.39 (m, 1H), 4.21-4.19 (m, 1H), 3.77-3.71 (m, 4H), 3.69-3.61 (m, 1H), 3.53-3.50 (m, 2H), 2.25-2.20 (m, 1H), 2.07-1.84 (m, 2H), 1.03-0.96 (m, 2H), 0.95-0.87 (m, 2H).

### Example 9-Synthesis of Compound 1-90: 6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one

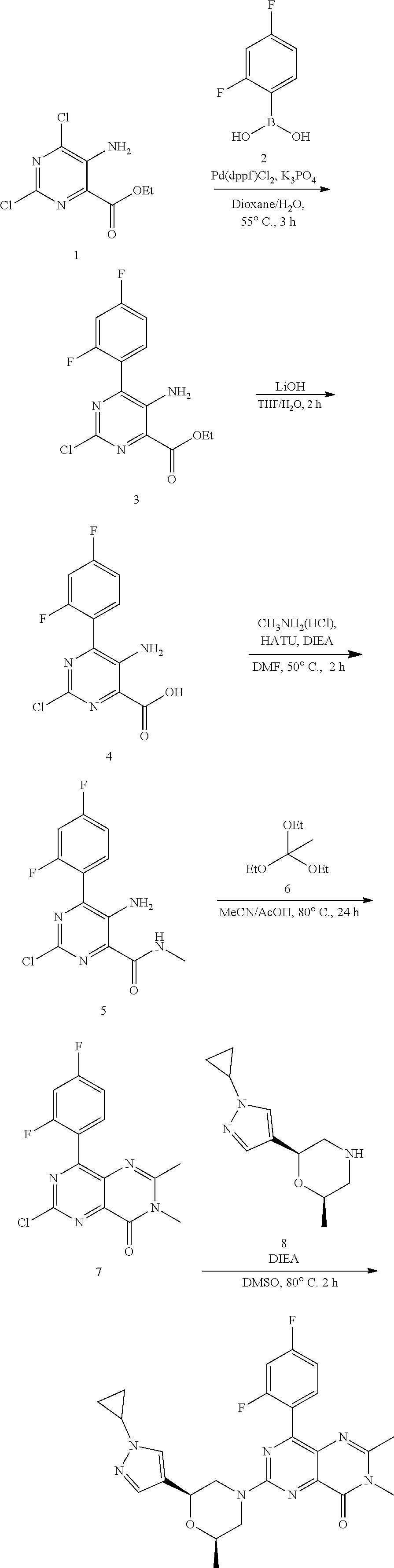

Step 1: To a solution of ethyl 5-amino-2,6-dichloro-pyrimidine-4-carboxylate (2.5 g, 10.6 mmol, 1.0 eq) and (2,4-difluorophenyl) boronic acid (1.67 g, 10.6 mmol, 1.0 eq) in 1,4-dioxane:H₂O=10:1 (66 mL) were added Pd(dppf)Cl₂ (1.55 g, 2.11 mmol, 0.2 eq) and K₃PO₄ (4.5 g, 21.2 mmol, 2.0 eq), then the mixture was stirred at 55° C., for 2 hours. LC-MS showed that the starting material was consumed, and 80% desired product was detected. The mixture was evaporated and purified by flash column (Petroleum ether:ethyl acetate=85:15) to afford product as a yellow solid (2.0 g, 60% yield). LC-MS [M+H]⁺=314, R.T=1.323 min. ¹H NMR (400 MHz, DMSO) δ 7.66-7.64 (m, 1H), 7.48 (td, J=10.3, 2.4 Hz, 1H), 7.30 (td, J=8.4, 2.3 Hz, 1H), 6.70 (s, 2H), 4.38 (q, J=7.1 Hz, 2H), 1.35 (t, J=7.1 Hz, 3H).

Step 2: To a solution of ethyl 5-amino-2-chloro-6-(2,4-difluorophenyl)pyrimidine-4-carboxylate (2 g, 6.38 mmol, 1.0 eq) in THF (30 mL) was added LiOH (0.77 g, 31.88 mmol, 5.0 eq) in H₂O (30 mL). After stirring for 1 hour, LCMS showed the starting material was consumed. The solution was diluted with water (30 mL) and 2M HCl aqueous solution (30 mL) and extracted with DCM (3×100 mL). The combined organic layer was dried over Na₂SO₄ and concentrated in vacuo to afford the crude product (2 g. 109% yield). ¹H NMR (400 MHz, DMSO) δ 13.83 (s, 1H), 7.63-7.61 (m, 1H), 7.47 (td, J=10.2, 2.4 Hz, 1H), 7.29 (td, J=8.5, 2.5 Hz, 1H), 6.70 (s, 2H).

Step 3: To a mixture of 5-amino-2-chloro-6-(2,4-difluorophenyl)pyrimidine-4-carboxylic acid [2 g. 7.0 mmol, 1.0 eq], methylamine hydrochloride[709 mg, 10.5 mmol, 1.5 eq] and HATU [4.0 g. 10.5 mmol, 1.5 eq] in DMF (20 mL) stirred under nitrogen at 20° C. was added diisopropylethylamine[2.7 g, 21 mmol, 3.0 eq]. The reaction mixture was stirred at 50° C., for 1 hour. LCMS (SY-2021-01-078-A) indicated that the starting material was consumed and ˜70% desired product was detected. The reaction mixture was diluted with H₂O (100 mL) and extracted with EtOAc (3×200 mL). The combined organic was dried over MgSO₄, filtered and concentrated in vacuo to afford a residue. The residue was purified by flash column (Petroleum ether:ethyl acetate=3:1) to afford the desired product as a yellow solid (2.0 g, 95.6% yield). LC-MS [M+H]⁺=299, R.T=1.058 min

Step 4: The mixture of 5-amino-2-chloro-6-(2,4-difluorophenyl)-N-methyl-pyrimidine-4-carboxamide (500 mg, 1.67 mmol, 1.0 eq) in 1,1,1-triethoxyethane/AcOH (5 mL/5 mL) was stirred at 80° C. for 24 hours. The reaction was allowed to cool to room temperature and diluted with H₂O (50 mL) and extracted with EtOAc (50 mL×3). The combined organic phase was washed with brine (20 mL×2), dried over Na₂SO₄, filtrated and concentrated under reduced pressure and purified by flash column (DCM:MeOH=95:5) to afford the desired product as a white solid (350 mg, 65% yield). ¹H NMR (400 MHz, CDCl3) δ 7.74-7.68 (m, 1H), 7.07 (td, J=8.3, 2.3 Hz, 1H), 7.01-6.92 (m, 1H), 3.68 (s, 3H), 2.60 (s, 3H).

Step 5: To a mixture of 6-chloro-8-(2,4-difluorophenyl)-2,3-dimethyl-pyrimido[5,4-d]pyrimidin-4-one (40 mg, 0.12 mmol, 1.0 eq) and (2S,6R)-2-(1-cyclopropylpyrazol-4-yl)-6-methyl-morpholine (38 mg, 0.19 mmol, 1.5 eq) in DMSO (2 mL) was added DIPEA (48 mg, 0.37 mmol, 3.0 eq) at 25° C. The mixture was stirred at 80° C., for 2 hours. LCMS (SY-2022-02-080-1A) indicated that the starting material was consumed and ˜80% desired product was detected. The reaction was allowed to room temperature and diluted with H₂O (20 mL) and extracted with EtOAc (20 mL×3). The combined organic phase was washed with brine (20 mL×2), dried over Na₂SO₄, filtrated and concentrated under reduced pressure and purified by pre-HPLC (ACN—H₂O (0.1% NH₃); gradient: 5-95) and lyophilized to afford the desired product as a yellow solid (38 mg, 62% yield). LC-MS [M+H]⁺=494.2, R.T=1.135 min. 1H NMR (400 MHz, DMSO) δ 7.85 (s, 1H), 7.73-7.67 (m, 7.9 Hz, 1H), 7.47 (s, 1H), 7.44-7.38 (m, 1H), 7.26-7.22 (m, 1H), 4.65 (t, J=11.5 Hz, 2H), 4.52 (dd, J=10.9, 2.4 Hz, 1H), 3.78-3.64 (m, 2H), 3.51 (s, 3H), 3.05-3.00 (m, 1H), 2.79-2.67 (m, 1H), 2.44 (s, 3H), 1.21 (d, J=6.1 Hz, 3H), 1.05-1.00 (m, 2H), 0.96-0.91 (m, 2H).

Step 6: (To a solution of 6-chloro-8-(2,4-difluorophenyl)-2,3-dimethyl-pyrimido[5,4-d]pyrimidin-4-one (200 mg, 0.62 mmol, 1.0 eq) and 1-cyclopropyl-4-[(6R)-4-(4,4,5,5-tetramethyl-1,3,2-dioxaborolan-2-yl)-3,6-dihydro-2H-pyran-6-yl]pyrazole (294 mg, 0.93 mmol, 1.5 eq) in 1,4-Dioxane/H₂O (10/1 mL) were added K₂PO₄ (263 mg, 1.24 mmol, 2.0 eq) and Pd(dppf)Cl₂ (91 mg, 0.124 mmol, 0.2 eq), then the mixture was refluxed at 80° C., for 3 hours. LC-MS showed desired product. After cooling, the solution was diluted with H₂O (100 mL) and extracted with ethyl acetate (3×100 mL). The combined organic layer was dried over Na₂SO₄ and concentrated in vacuo to give crude product. The crude product was purified by flash column (DCM:MeOH=50:1) to obtain the desired product as a brown solid (200 mg, 67% yield). LC-MS [M+H]⁺=477.3, R.T=1.257 min.

Step 7: To a solution of 6-[(6R)-6-(1-cyclopropylpyrazol-4-yl)-3,6-dihydro-2H-pyran-4-yl]-8-(2,4-difluorophenyl)-2,3-dimethyl-pyrimido[5,4-d]pyrimidin-4-one (200 mg, 0.42 mmol, 1.0 eq) in EtOAc (5 mL) was added PtO₂ (40 mg), then the mixture was refluxed at 20° C., for 2 hours under H₂. LC-MS showed desired product. The reaction solution was filtrated and concentrated under reduced pressure and purified by pre-HPLC (ACN—H₂O (0.1% NH₃); gradient: 5-95) and lyophilized to afford the desired product as a white solid (60 mg, 30% yield). LC-MS [M+H]⁺=479.2, R.T=1.219 min. ¹H NMR (400 MHz, DMSO) δ 7.77-7.72 (m, 1H), 7.71 (s, 1H), 7.45 (td, J=10.1, 2.5 Hz, 1H), 7.37 (s, 1H), 7.29 (td, J=8.5, 2.4 Hz, 1H), 4.50-4.46 (m, 1H), 4.10-4.06 (m, 1H), 3.73-3.68 (m, 1H), 3.67-3.62 (m, 2H), 3.56 (s, 3H), 3.46-3.37 (m, 1H), 2.53 (s, 3H), 2.24-2.18 (m, 1H), 2.01-1.79 (m, 3H), 1.02-0.96 (m, 2H), 0.93-0.87 (m, 2H).

### Example 10-Synthesis of Compound I-95: 7-((2R,4S)-2-(1-cyclopropyl-1H-pyrazol-4-yl)tetrahydro-2H-pyran-4-yl)-5-(2-fluoro-4-(trifluoromethyl)phenyl)-2,3-dimethyl-2,6-naphthyridin-1 (2H)-one

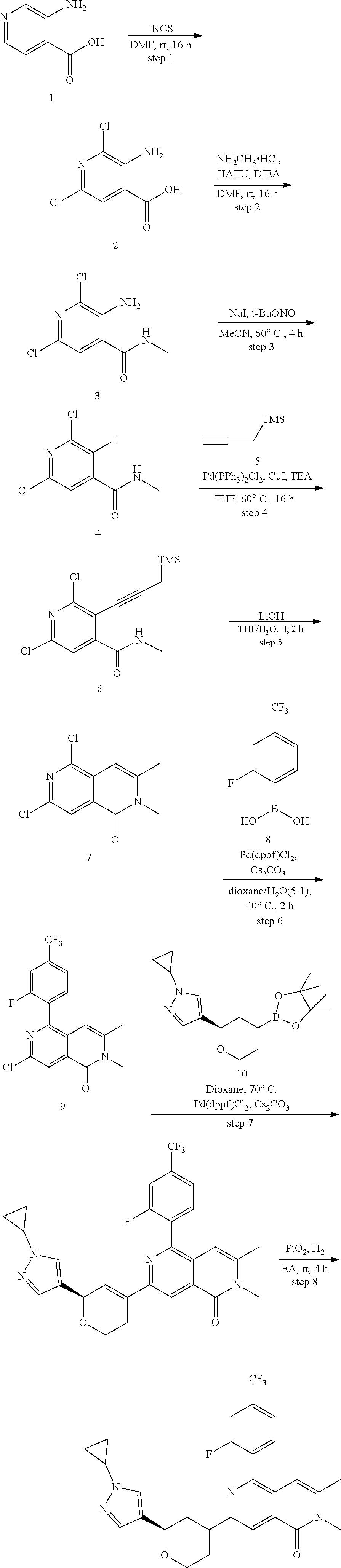

Step 1: A mixture of 3-aminopyridine-4-carboxylic acid (1 g, 7.24 mmol, 1 eq) and NCS (2.58 g, 14.48 mmol, 2 eq) in DMF (5 mL) was stirred at 35° C., for 16 hours. LCMS showed the starting material was consumed and one major peak with desired mass was detected. The solution was diluted with water (100 mL) and extracted with EtOAc (30 mL×3). The combined organic layers were washed with brine (5×100 mL), dried over sodium sulfate and filtered. The filter was concentrated under reduced pressure to afford crude 3-amino-2,6-dichloroisonicotinic acid (1.3 g, yield=78.1%) which was used for next step directly. LC-MS [M+H]⁺=206.9 R.T=0.938 min

Step 2: To a solution of 3-amino-2,6-dichloroisonicotinic acid (1 g, 4.83 mmol, 1 eq) in DMF (10 mL) was added HATU (2.76 g, 7.25 mmol, 1.5 eq). The resulting mixture was stirred for 10 min. Then DIEA (1.87 g, 14.5 mmol, 3 eq) and methylamine hydrochloride (0.39 g, 5.80 mmol, 1.2 eq) were added. The mixture was further stirred at 25° C., for 16 hours. LCMS showed the starting material was consumed and one major peak with desired mass was detected. The solution was diluted with water (100 mL) and extracted with EtOAc (20 mL×3). The combined organic layers were washed with brine (5×50 mL), dried over sodium sulfate and filtered. The filter was concentrated under reduced pressure and purified by flash column chromatography (Petroleum ether:Ethyl acetate=80:20) to afford 3-amino-2,6-dichloro-N-methylisonicotinamide as an off-white solid (500 mg, yield=42.3%). LC-MS [M+H]⁺=219.9. R.T=0.847 min

Step 3: To a solution of 3-amino-2,6-dichloro-N-methylisonicotinamide (4 g, 18.18 mmol, 1 eq) in MeCN (50 mL) were added CuI (17.31 g, 90.88 mmol, 5 eq) and 1-BuONO. The mixture was stirred at 60° C., for 4 hours. LCMS showed the starting material was consumed and one major peak with desired mass was detected. The mixture was filtered. The filter was diluted with water (200 mL) and extracted with EtOAc (100 mL×3). The combined organic layers were washed with brine (3×200 mL), dried over sodium sulfate and filtered. The filter was concentrated under reduced pressure. The residue was purified by flash column chromatography (Petroleum ether/Ethyl acetate=80:20) to afford 2,6-dichloro-3-iodo-N-methylisonicotinamide (500 mg, yield=7.5%) as an off-white solid. LC-MS [M+H]⁺=330. R.T=0.923 min

Step 4: To a solution of 2,6-dichloro-3-iodo-N-methylisonicotinamide (1.5 g, 4.53 mmol, 1 eq) in THF (50 mL) were added trimethyl(prop-2-yn-1-yl) silane (1.02 g, 9.07 mmol, 2 eq), Pd(PPh₃)₂Cl₂ (0.32 g. 0.45 mmol, 0.1 eq), CuI (0.26 g. 1.36 mmol, 0.3 eq), and triethylamine (1.38 g, 13.60 mmol, 3 eq). The resulting mixture was stirred at 60° C., for 16 hours under N₂. LCMS showed the starting material was consumed and one major peak with desired mass was detected. The mixture was filtered and the filter was concentrated under reduced pressure. The residue was purified by flash column chromatography (Petroleum ether:Ethyl acetate=90:10) to afford 2,6-dichloro-N-methyl-3-(3-(trimethylsilyl) prop-1-yn-1-yl) isonicotinamide (400 mg, yield=25.2%) as a black solid. LC-MS [M+H]⁺=315.0 R.T=1.361 min

Step 5: To a solution of 2,6-dichloro-N-methyl-3-(3-trimethylsilylprop-1-ynyl)pyridine-4-carboxamide (200 mg, 0.63 mmol, 1 eq) in THF/H₂O (10:1, 5.5 mL) was added LiOH (30.45 mg, 1.27 mmol, 2 eq), stirred at 25° C., for 16 h. LCMS showed the starting material was consumed and one major peak with desired mass was detected. The solution was diluted with water (20 mL) and extracted with EtOAc (5 mL×3). The combined organic layers were washed with brine (3×10 mL), dried over sodium sulfate and filtered. The filter was concentrated under reduced pressure. The residue was purified by flash column chromatography (Petroleum ether:Ethyl acetate=90:10) to afford 5,7-dichloro-2,3-dimethyl-2,6-naphthyridin-1 (2H)-one (20 mg, yield=11.7%) as a light yellow solid. LC-MS [M+H]⁺=242.9 R.T=1.127 min ¹H NMR (400 MHz, DMSO) δ 8.01 (s, 1H), 6.72 (s, 1H), 3.54 (s, 3H), 2.52 (s, 3H).

Step 6: A mixture of 5,7-dichloro-2,3-dimethyl-2,6-naphthyridin-1 (2H)-one (80 mg. 0.33 mmol, 1 eq), (2-fluoro-4-(trifluoromethyl)phenyl) boronic acid (82 mg, 0.39 mmol, 1.2 eq), Cs₂CO₃ (321 mg. 0.99 mmol, 3 eq) and Pd(dppf)Cl₂ (48.2 mg. 0.066 mmol, 0.2 eq) in dioxane/H₂O (5:1, 6 mL) was stirred at 40° C., for 2 hours under N₂. LCMS showed the starting material was consumed and one major peak with desired mass was detected. The mixture was filtered and the filter was concentrated under reduced pressure. The residue was purified by TLC (Petroleum ether/Ethyl acetate=50:50) to afford 7-chloro-5-(2-fluoro-4-(trifluoromethyl)phenyl)-2,3-dimethyl-2,6-naphthyridin-1 (2H)-one (40 mg, yield=26.2%) as a light yellow solid. LC-MS [M+H]⁺=371.0 R.T=1.382 min

Step 7: A mixture of 7-chloro-5-(2-fluoro-4-(trifluoromethyl)phenyl)-2,3-dimethyl-2,6-naphthyridin-1 (2H)-one (100 mg, 0.27 mmol, 1 eq), 1-cyclopropyl-4-[(6R)-4-(4,4,5,5-tetramethyl-1,3,2-dioxaborolan-2-yl)-3,6-dihydro-2H-pyran-6-yl]pyrazole (93.82 mg. 0.30 mmol, 1.1 eq), Pd(dppf)Cl₂ (39.47 mg, 0.054 mmol, 0.2 eq) and Cs₂CO₃ (262.99 mg, 0.81 mmol, 3 eq) in dioxane/H₂O (5:1, 6 mL) was stirred at 40° C., for 2 hours. LCMS showed the starting material was consumed and one major peak with desired mass was detected. The mixture was filtered and the filter was concentrated under reduced pressure. The residue was purified by flash column chromatography (Petroleum ether:Ethyl acetate=20:80) to afford (R)-7-(6-(1-cyclopropyl-1H-pyrazol-4-yl)-3,6-dihydro-2H-pyran-4-yl)-5-(2-fluoro-4-(trifluoromethyl)phenyl)-2,3-dimethyl-2,6-naphthyridin-1 (2H)-one (80 mg, yield=45.2%) as a light yellow solid. LC-MS [M+H]⁺=525.1 R.T=1.392 min

Step 8: To a solution of (R)-7-(6-(1-cyclopropyl-1H-pyrazol-4-yl)-3,6-dihydro-2H-pyran-4-yl)-5-(2-fluoro-4-(trifluoromethyl)phenyl)-2,3-dimethyl-2,6-naphthyridin-1 (2H)-one (80 mg, 0.15 mmol, 1 eq) in EtOAc (5 mL) was added PtO₂ (3.46 mg. 0.015 mmol, 0.1 eq). The mixture was stirred at 25° C. under H₂ for 6 hours. LCMS showed the starting material was consumed and one major peak with desired mass was detected. The mixture was filtered and the filter was concentrated under reduced pressure. The residue was purified by prep-HPLC (Gemini 5 um C18 150*21.2 mm, mobile phase:ACN—H₂O (0.1% TFA); gradient: 5-95) to afford 7-((2R,4S)-2-(1-cyclopropyl-1H-pyrazol-4-yl)tetrahydro-2H-pyran-4-yl)-5-(2-fluoro-4-(trifluoromethyl)phenyl)-2,3-dimethyl-2,6-naphthyridin-1 (2H)-one (9.87 mg, yield=12.1%) as a yellow solid. LC-MS [M+H]⁺=527.1 R.T=1.349 min ¹H NMR (400 MHz, DMSO) & 8.01 (s, 1H), 7.90 (d, J=10.0 Hz, 1H), 7.79 (d, J=4.4 Hz, 2H), 7.72 (s, 1H), 7.37 (s, 1H), 6.22 (d, J=2.8 Hz, 1H), 4.46 (dd, J=11.2, 1.6 Hz, 1H), 4.07 (dd, J=11.2, 3.2 Hz, 1H), 3.70-3.60 (m, 2H), 3.55 (s, 3H), 3.33-3.22 (m, 1H), 2.39 (s, 3H), 2.15 (d, J=12.8 Hz, 1H), 1.90 (d, J=12.8 Hz, 1H), 1.86-1.75 (m, 2H), 1.01-0.96 (m, 2H), 0.93-0.87 (m, 2H).

### Example 11-Synthesis of Compound I-100: 4-(4-chloro-2-fluorophenyl)-2-((2S,6R)-2-(1-cyclopropyl-1H-pyrazol-4-yl)-6-methylmorpholino)-7-methylpyrido[2,3-d]pyridazin-8 (7H)-one

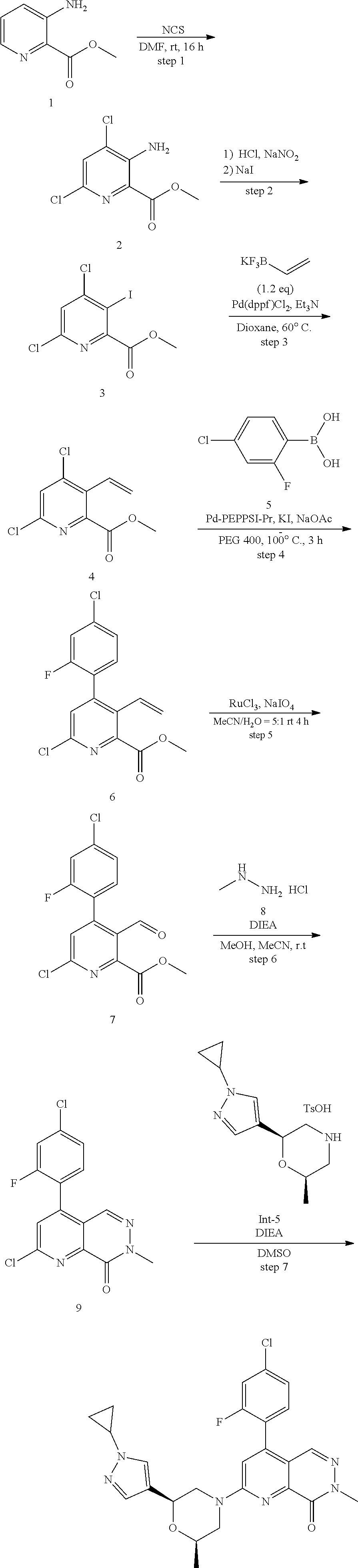

Step 1: To a stirred solution of methyl 3-aminopyridine-2-carboxylate (32.50 g, 214 mmol, 1.0 eq) in DMF (500 mL) was added NCS (57.05 g, 427 mmol, 2.0 eq). The mixture was stirred at 35° C., overnight. The mixture was poured into 4 L of water, filtrated to obtain methyl 3-amino-4,6-dichloro-pyridine-2-carboxylate (40.6 g, 184 mmol, 86.0% yield) LC-MS [M+H]⁺=220.9 R.T=0.61 min

Step 2: To a stirred solution of methyl 3-amino-4,6-dichloro-pyridine-2-carboxylate (221 mg, 1.00 mmol, 1.0 eq) in 1.5 mL of 12 M HCl was added dropwise NaNO₂ (103 mg, 1.50 mmol, 1.5 eq) in 0.5 mL of water at 0° C. The mixture was stirred at 0° C., for 30 min. To a stirred solution of NaI (498 mg, 3.00 mmol, 3.0 eq) in 4 mL of water and 4 mL of DCM was added above solution dropwise at 0° C. The reaction mixture was poured into water (50 mL) and extracted with DCM (100 mL×2). The organic layer was washed with Na₂S₂O₃ and brine, dried by Na₂SO₄. The solution was concentrated to give methyl 4,6-dichloro-3-iodo-pyridine-2-carboxylate (300 mg, 0.90 mmol, 90.4% yield). LC-MS [M+H]⁺=331.8 R.T=1.37 min

Step 3: To a stirred solution of methyl 4,6-dichloro-3-iodo-pyridine-2-carboxylate (8.00 g, 24.1 mmol, 1.0 eq) in 1,4-dioxane (160 mL) were added potassium vinyltrifluoroborate (6.51 g, 48.2 mmol, 2.0 eq), TEA (17 mL, 121 mmol, 5.0 eq), Pd(dppf)Cl₂ (3.17 g, 4.34 mmol, 0.18 eq). The mixture was stirred at 100° C., for 6 hours. The reaction mixture was filtered and extracted with ethyl acetate (500 mL×3). The combined organic layers were washed with brine (300 mL), dried over Na₂SO₄, filtered and concentrated under reduced pressure to give a residue. The residue was purified by column chromatography to afford methyl 4,6-dichloro-3-vinyl-pyridine-2-carboxylate (3.50 g, 15.1 mmol, 62.6% yield) LC-MS [M+H]⁺=231.8 R.T=1.21 min

Step 4: To a stirred solution of methyl 4,6-dichloro-3-vinyl-pyridine-2-carboxylate (160 mg, 0.69 mmol, 1.0 eq) in PEG 400 (2 mL) were added (4-chloro-2-fluoro-phenyl) boronic acid (81 mg, 0.46 mmol, 0.67 eq), KI (114 mg, 0.69 mmol, 1.0 eq), NaOAc (113 mg, 1.38 mmol, 2.0 eq), and Pd PEPPSI-IPr (470 mg, 0.69 mmol, 1.0 eq). The mixture was stirred at 100° C., for 4 hours. The mixture was diluted with water (20 mL) and extracted with ethyl acetate (50 mL×2). The combined organic layers were washed with brine (50 mL) and dried over Na₂SO₄. The solution was filtered and the filtrate was concentratecxxd under reduced pressure. The crude product was purified by column chromatography on silica gel to obtain methyl 6-chloro-4-(4-chloro-2-fluoro-phenyl)-3-vinyl-pyridine-2-carboxylate (40 mg, 0.12 mmol, 17.8% yield). LC-MS [M+H]⁺=326.0 R.T=1.56 min

Step 5: To a stirred solution of methyl 6-chloro-4-(4-chloro-2-fluoro-phenyl)-3-vinyl-pyridine-2-carboxylate (690 mg, 2.12 mmol, 1.0 eq) in MeCN (17 mL) and water (3 mL) were added RuCl₃ (88 mg, 0.42 mmol, 0.2 eq), and NaIO₄ (1.37 g, 6.35 mmol, 3.0 eq). The mixture was stirred at room temperature for 4 hours. The reaction mixture was diluted by addition of H₂O (50 mL) and extracted with ethyl acetate (100 mL×2). The residue was purified by column chromatography to afford methyl 6-chloro-4-(4-chloro-2-fluoro-phenyl)-3-formyl-pyridine-2-carboxylate (55 mg, 0.17 mmol, 7.92% yield). LC-MS [M+H]=328.0 R.T=1.426 min

Step 6: To a stirred solution of methyl 6-chloro-4-(4-chloro-2-fluoro-phenyl)-3-formyl-pyridine-2-carboxylate (110 mg, 0.34 mmol, 1.0 eq) in MeCN (1 mL) and methanol (1 mL) were added methylhydrazine; hydrochloride (55 mg, 0.67 mmol, 2.0 eq), DIEA (0.18 mL, 1.01 mmol, 3.0 eq). The mixture was stirred at 20° C., for 2 hours. The solid was filtered and dried to obtain 2-chloro-4-(4-chloro-2-fluoro-phenyl)-7-methyl-pyrido[2,3-d]pyridazin-8-one (60 mg, 0.19 mmol, 55.2% yield). LC-MS [M+H]⁺=323.9 R.T=1.29 min

Step 7: To a stirred solution of 2-chloro-4-(4-chloro-2-fluoro-phenyl)-7-methyl-pyrido[2,3-d]pyridazin-8-one (60 mg, 0.19 mmol, 1.0 eq) in DMSO (4 mL) and DIEA (8.1 mL) was added (2S,6R)-2-(1-cyclopropylpyrazol-4-yl)-6-methyl-morpholine-4-methylbenzenesulfonic acid (140 mg, 0.37 mmol, 2.0 eq). The mixture was stirred at 100° C., for 1 hour. The reaction was poured into water and filtrated to obtain 4-(4-chloro-2-fluoro-phenyl)-2-[(2S,6R)-2-(1-cyclopropylpyrazol-4-yl)-6-methyl-morpholin-4-yl]-7-methyl-pyrido[2,3-d]pyridazin-8-one (75 mg, 0.15 mmol, 81.9% yield). LC-MS [M+H]⁺=495.1 R.T=1.37 min ¹H NMR (400 MHz, DMSO) δ 7.84 (s, 1H), 7.70-7.67 (m, 2H), 7.59 (t, 1H), 7.52 (m, 1H), 7.48 (s, 2H), 4.55-4.52 (m, 1H), 3.76-3.70 (m, 2H), 3.68 (s, 3H), 3.01 (t, 1H), 2.72-2.50 (m, 1H), 1.21 (d, 3H), 1.01-0.99 (m, 2H), 0.95-0.94 (m, 2H).

### Example 12-Synthesis of Compound I-105: 6-((2R,4S)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one

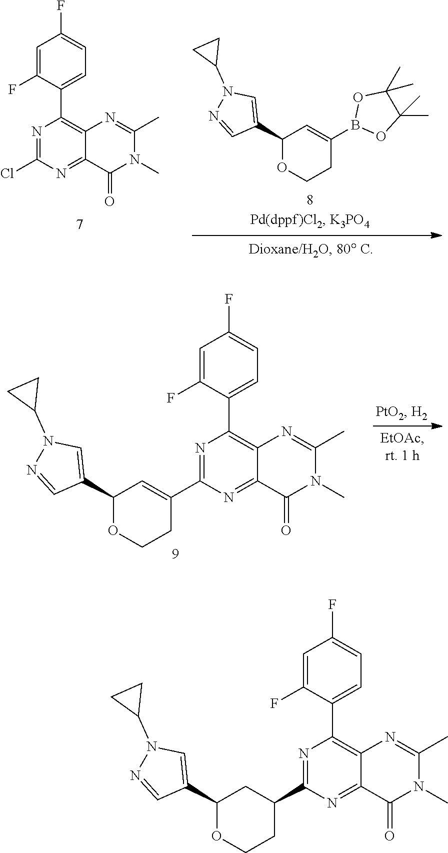 [SMILES: Cc1nc2c(-c3ccc(F)cc3F)nc(Cl)nc2c(=O)n1C ; CC1(C)OB(C2=C[C@H](c3cnn(C4CC4)c3)OCC2)OC1(C)C ; Cc1nc2c(-c3ccc(F)cc3F)nc(C3=C[C@H](c4cnn(C5CC5)c4)OCC3)nc2c(=O)n1C ; Cc1nc2c(-c3ccc(F)cc3F)nc([C@H]3CCO[C@@H](c4cnn(C5CC5)c4)C3)nc2c(=O)n1C]

Step 6: To a solution of 6-chloro-8-(2,4-difluorophenyl)-2,3-dimethyl-pyrimido[5,4-d]pyrimidin-4-one (200 mg, 0.62 mmol, 1.0 eq) (Compound 7 in Example 6) and 1-cyclopropyl-4-[(6R)-4-(4,4,5,5-tetramethyl-1,3,2-dioxaborolan-2-yl)-3,6-dihydro-2H-pyran-6-yl]pyrazole (294 mg, 0.93 mmol, 1.5 eq) in 1,4-Dioxane/H₂O (10/1 mL) were added K₃PO₄ (263 mg, 1.24 mmol, 2.0 eq) and Pd(dppf)Cl₂ (91 mg, 0.124 mmol, 0.2 eq), then the mixture was refluxed at 80° C., for 3 hours. LC-MS showed desired product. After cooling, the solution was diluted with H₂O (100 mL) and extracted with ethyl acetate (3×100 mL). The combined organic layer was dried over Na₂SO₄ and concentrated in vacuo to give crude product. The crude product was purified by flash column (DCM:MeOH=50:1) to obtain the desired product as a brown solid (200 mg, 67% yield). LC-MS [M+H]⁺=477.3, R.T=1.257 min.

Step 7: To a solution of 6-[(6R)-6-(1-cyclopropylpyrazol-4-yl)-3,6-dihydro-2H-pyran-4-yl]-8-(2,4-difluorophenyl)-2,3-dimethyl-pyrimido[5,4-d]pyrimidin-4-one (200 mg, 0.42 mmol, 1.0 eq) in EtOAc (5 mL) was added PtO₂ (40 mg), then the mixture was refluxed at 20° C., for 2 hours under H₂. LC-MS showed desired product. The reaction solution was filtrated and concentrated under reduced pressure and purified by pre-HPLC (ACN—H₂O (0.1% NH₃); gradient: 5-95) and lyophilized to afford the desired product as a white solid (60 mg, 30% yield). LC-MS [M+H]⁺=479.2, R.T=1.219 min. ¹H NMR (400 MHz, DMSO) δ 7.77-7.72 (m, 1H), 7.71 (s, 1H), 7.45 (td, J=10.1, 2.5 Hz, 1H), 7.37 (s, 1H), 7.29 (td, J=8.5, 2.4 Hz, 1H), 4.50-4.46 (m, 1H), 4.10-4.06 (m, 1H), 3.73-3.68 (m, 1H), 3.67-3.62 (m, 2H), 3.56 (s, 3H), 3.46-3.37 (m, 1H), 2.53 (s, 3H), 2.24-2.18 (m, 1H), 2.01-1.79 (m, 3H), 1.02-0.96 (m, 2H), 0.93-0.87 (m, 2H).

### Example 13-Synthesis of Compound I-120: 6-((2R,4S)-2-(1-cyclopropyl-1H-pyrazol-4-yl)tetrahydro-2H-pyran-4-yl)-8-(2,4-difluorophenyl)-2,3-dimethylpyrido[3,2-d]pyrimidin-4 (3H)-one

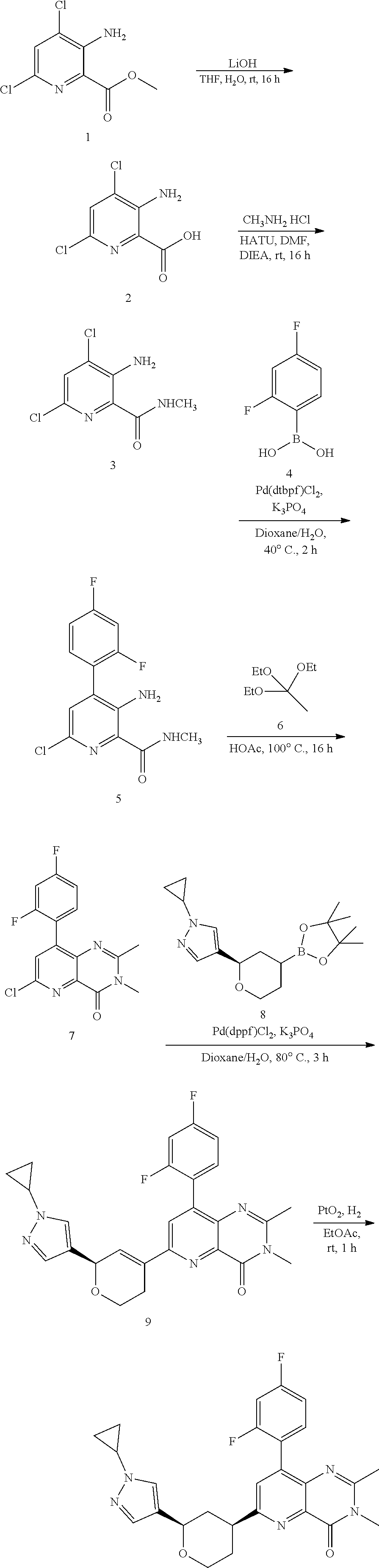

Step 1: To a solution of methyl 3-amino-4,6-dichloro-pyridine-2-carboxylate (20 g, 90.5 mmol, 1.0 eq) in THF (200 mL) was added a solution of LiOH (10.86 g, 452 mmol, 5.0 eq) in H₂O (100 mL). After stirring for 2 hours, LCMS showed the starting material was consumed. The mixture was added 2M HCl (200 mL) and extracted with DCM (3×500 mL). The combined organic layer was dried over Na₂SO₄ and concentrated in vacuo to afford crude product (20 g, 106% yield). LC-MS [M+H]⁺=205, R.T=0.977 min.

Step 2: To a mixture of 3-amino-4,6-dichloro-pyridine-2-carboxylic acid [20 g, 96.6 mmol, 1.0 eq], methylamine hydrochloride[9.8 g, 145 mmol, 1.5 eq], and HATU [55 g, 145 mmol, 1.5 eq] in DMF (200 mL) was added diisopropylethylamine[37.5 g, 290 mmol, 3.0 eq]. The reaction mixture was stirred at 25° C., for 3 hours. LCMS (SY-2022-02-087-1A) indicated that the starting material was consumed and ˜70% desired product was detected. The reaction mixture was diluted with H₂O (300 mL) and extracted with EtOAc (3×500 mL). The combined organic was dried over MgSO₄, filtered and concentrated in vacuo to give a crude product. The product was purified by flash column (Petroleum ether/Ethyl acetate=3:1) to give the desired product as a yellow solid (20 g, 94% yield). ¹H NMR (400 MHz, DMSO) δ 8.54 (d, J=4.4 Hz, 1H), 7.73 (s, 1H), 7.22 (s, 2H), 2.77 (d, J=4.8 Hz, 3H).

Step 3: To a solution of 3-amino-4,6-dichloro-N-methyl-pyridine-2-carboxamide (1 g, 4.54 mmol, 1.0 eq) and (2,4-difluorophenyl) boronic acid (717 mg, 4.54 mmol, 1.0 eq) in 1,4-dioxane:H₂O=10:1 (11 mL) were added Pd (dtbpf) Cl₂ (591 mg, 0.91 mmol, 0.2 eq) and K₃PO₄ (1.93 g, 9.09 mmol, 2.0 eq). The mixture was stirred at 40° C., for 3 hours. LC-MS showed that the starting material was consumed, and 60% desired product was detected. The mixture was concentrated under reduced pressure and purified by pre-HPLC (ACN—H₂O (0.1% NH₃); gradient: 5-95) and lyophilized to afford the desired product as a white solid (500 mg, 37% yield). LC-MS [M+H]⁺=298, R.T=1.325 min. ¹H NMR (400 MHz, DMSO) δ 8.51 (d, J=4.4 Hz, 1H), 7.54-7.39 (m, 2H), 7.31 (s, 1H), 7.24 (t, J=8.0 Hz, 1H), 6.80 (s, 2H), 2.79 (d, J=4.6 Hz, 3H).

Step 4: To a mixture of 3-amino-6-chloro-4-(2,4-difluorophenyl)-N-methyl-pyridine-2-carboxamide (500 mg, 1.68 mmol, 1.0 eq) in a solution of 1,1,1-triethoxyethane/AcOH (4 mL/2 mL). The mixture was stirred at 80° C., for 16 hours. The reaction was allowed to cool to room temperature and diluted with H₂O (20 mL) and extracted with EtOAc (50 mL×3). The combined organic phase was washed with brine (20 mL×2), dried over anhydrous sodium sulfate, filtrated and concentrated under reduced pressure and purified by flash column (DCM:MeOH=95:5) to afford the desired product as a white solid (450 mg, 83% yield). ¹H NMR (400 MHz, CDCl3) § 7.62 (d, J=1.0 Hz, 1H), 7.48-7.44 (m, 1H), 7.06-6.93 (m, 2H), 3.67 (s, 3H), 2.56 (s, 3H).

Step 5: To a solution of 6-chloro-8-(2,4-difluorophenyl)-2,3-dimethyl-pyrido[3,2-d]pyrimidin-4-one (200 mg. 0.62 mmol, 1.0 eq) and 1-cyclopropyl-4-[(6R)-4-(4,4,5,5-tetramethyl-1,3,2-dioxaborolan-2-yl)-3,6-dihydro-2H-pyran-6-yl]pyrazole (236 mg, 0.75 mmol, 1.2 eq) in 1,4-dioxane/H₂O (10/1 mL) were added K₃PO₄ (264 mg, 1.24 mmol, 2.0 eq) and Pd(dppf)Cl₂ (91 mg. 0.124 mmol, 0.2 eq). Then the mixture was refluxed at 80° C., for 3 hours. LC-MS showed desired product. After cooling, the solution was diluted with H₂O (100 mL) and extracted with ethyl acetate (3×100 mL). The combined organic layer was dried over Na₂SO₄ and concentrated in vacuo to afford the crude product. The crude product was purified by flash column (DCM:MeOH=50:1) to obtain the desired product as a brown solid (200 mg, 66% yield). LC-MS [M+H]⁺=476, R.T=1.251 min.

Step 6: To a solution of (R)-6-(6-(1-cyclopropyl-1H-pyrazol-4-yl)-3,6-dihydro-2H-pyran-4-yl)-8-(2,4-difluorophenyl)-2,3-dimethylpyrido[3,2-d]pyrimidin-4 (3H)-one (100 mg, 0.21 mmol, 1.0 eq) in EtOAc (5 mL) was added PtO₂ (25 mg). Then the mixture was refluxed at 20° C., for 2 hours under H₂. LC-MS showed desired product. The reaction solution was filtrated and concentrated under reduced pressure and purified by pre-HPLC (ACN—H₂O (0.1% NH₃); gradient: 5-95) and lyophilized to afford the desired product as a white solid (10 mg, 10% yield). LC-MS [M+H]⁺=478.2, R.T=1.229 min. ¹H NMR (400 MHz, DMSO) δ 7.76 (s, 1H), 7.72 (s, 1H), 7.60-7.56 (m, 1H), 7.43-7.39 (m, 1H), 7.38 (s, 1H), 7.28-7.21 (m, 1H), 4.48-4.42 (m, 1H), 4.08-4.02 (m, 1H), 3.71-3.61 (m, 2H), 3.55 (s, 3H), 2.48 (s, 3H), 2.36-2.30 (m, 1H), 2.15-2.10 (m, 1H), 1.89-1.82 (m, 3H), 1.02-0.96 (m, 2H), 0.94-0.88 (m, 2H).

### Example 14-Synthesis of Compound I-115: 7-cyclopropyl-2-(1-cyclopropyl-1H-pyrazol-4-yl)tetrahydro-2H-pyran-4-yl)-4-(2-fluoro-4-(trifluoromethyl)phenyl)pyrimido[4,5-d]pyridazin-8 (7H)-one

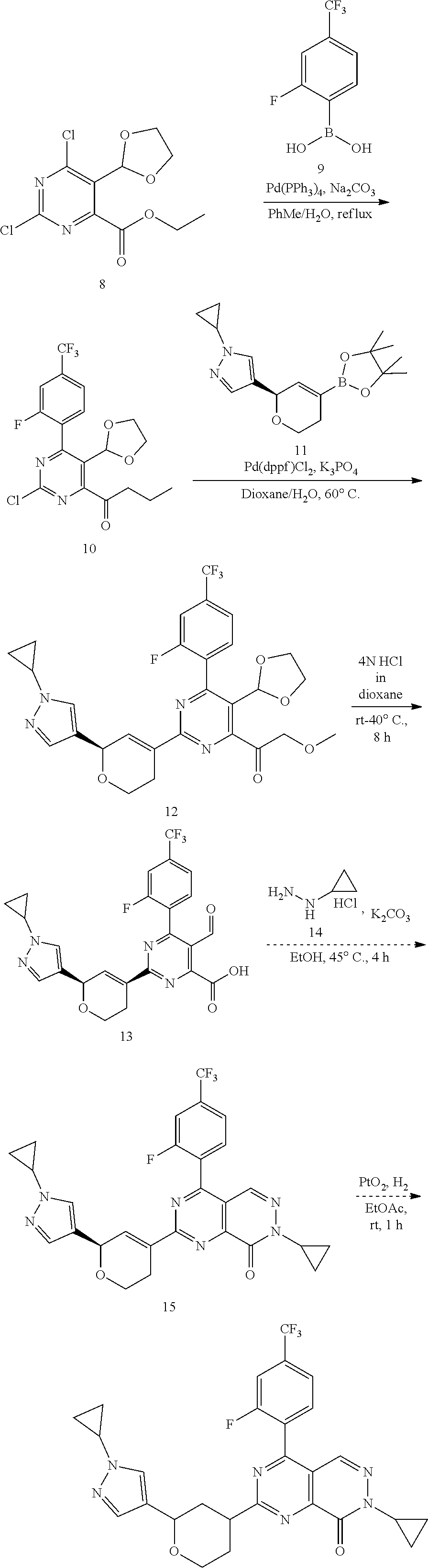

Step 1: To a solution of ethyl 2,6-dichloro-5-(1,3-dioxolan-2-yl)pyrimidine-4-carboxylate (2 g. 6.82 mmol. 1.0 eq) and [2-fluoro-4-(trifluoromethyl)phenyl]boronic acid (1.42 g. 6.82 mmol, 1 eq) in 1,4-dioxane:H₂O=10:1 (50 mL) were added Pd(dppf)Cl₂ (1 g. 1.36 mmol, 0.2 eq) and K₂PO₄ (2.17 g. 10.24 mmol, 1.5 eq). Then the mixture was refluxed at 60° C., for 6 hours. LC-MS showed desired product. The LCMS indicated that the starting material was consumed, and desired product detected. The mixture was evaporated and purified by flash column (Petroleum ether:EtOAc=95:5) to afford desired product (2.2 g. 5.23 mmol, 77%) as a white oil.

Step 2: To a solution of ethyl 2-chloro-5-(1,3-dioxolan-2-yl)-6-[2-fluoro-4-(trifluoromethyl)phenyl]pyrimidine-4-carboxylate (2.2 g. 5.23 mmol, 1.0 eq) and 1-cyclopropyl-4-[(6R)-4-(4,4,5,5-tetramethyl-1,3,2-dioxaborolan-2-yl)-3,6-dihydro-2H-pyran-6-yl]pyrazole (1.65 g. 5.23 mmol, 1 eq) in 1,4-dioxane (150 mL) were added K₃PO₄ (2.22 g, 10.46 mmol. 2 eq) and Pd(dppf)Cl₂ (765 mg, 1.05 mmol. 0.2 eq). The mixture was refluxed at 60° C., for 24 hours. LC-MS showed desired product. The LCMS indicated that the starting meterial was consumed and desired product detected. The mixture was evaporated and purified by flash column (Petroleum ether:EtOAc=1:1) to afford desired product (1.1 g. 1.91 mmol, 36.6%) as a yellow solid.

Step 3: To a solution of ethyl 2-[(6R)-6-(1-cyclopropylpyrazol-4-yl)-3,6-dihydro-2H-pyran-4-yl]-5-(1,3-dioxolan-2-yl)-6-[2-fluoro-4-(trifluoromethyl)phenyl]pyrimidine-4-carboxylate (250 mg. 0.44 mmol. 1.0 eq) was added HCl in dioxane (2 mL, 4 mmol/L). The mixture was refluxed at 25° C. for 12 hours. LC-MS showed desired product. The reaction was purified by flash column (CH₂Cl₂:MeOH=95:5) to afford desired product (400 mg. 0.80 mmol, 41.5%) as a yellow solid.

Step 4: To a mixture of 2-[(6R)-6-(1-cyclopropylpyrazol-4-yl)-3,6-dihydro-2H-pyran-4-yl]-6-[2-fluoro-4-(trifluoromethyl)phenyl]-5-formyl-pyrimidine-4-carboxylic acid (200 mg. 0.40 mmol, 1.0 eq) and cyclopropylhydrazine hydrochloride (65 mg. 0.60 mmol, 1.5 eq) in ethanol (2.5 mL) was added K₂CO₃ (165 mg, 1.19 mmol, 3 eq). The mixture was stirred at 25° C., for 4 hours. LC-MS showed that the starting material was consumed and about 60% desired product. The reaction was purified by flash column (CH₂Cl₂:MeOH=95:5) to afford desired product (70 mg. 0.13 mmol, 32.7%) as a yellow product.

Step 5: To a solution of 7-cyclopropyl-2-[(6R)-6-(1-cyclopropylpyrazol-4-yl)-3,6-dihydro-2H-pyran-4-yl]-4-[2-fluoro-4-(trifluoromethyl)phenyl]pyrimido[4,5-d]pyridazin-8-one (70 mg. 0.13 mmol, 1.0 eq) in EtOAc (2 mL) was added PtO₂ (8.85 mg. 0.04 mmol, 0.3 eq). The mixture was refluxed at 25° C., for 0.5 hour under H₂. LC-MS showed desired product. The mixture was evaporated and purified by prep-HPLC to afford desired product (10 mg. 0.04 mmol, 14.2%) as a white solid. LCMS: 541.2 [M+H⁺], R.T, ═1.427. ¹H NMR (400 MHz, MeOD) δ 8.20 (dd, J=9.3, 3.6 Hz, 1H), 7.96 (d, J=7.3 Hz, 1H), 7.85-7.79 (m, 2H), 7.75 (d, J=5.0 Hz, 1H), 7.53 (d, J=4.8 Hz, 1H), 4.63 (dd, J=11.4, 1.9 Hz, 1H), 4.27-4.16 (m, 2H), 3.95-3.81 (m, 1H), 3.63 (m, J=11.1, 7.2, 3.8 Hz, 2H), 2.38 (d, J=13.2 Hz, 1H), 2.18-2.07 (m, 2H), 1.25-1.18 (m, 2H), 1.14-0.99 (m, 6H).

### Example 15-Synthesis of Compound I-110: 6-((2S,6R)-2-(1-cyclopropyl-1H-pyrazol-4-yl)-6-methylmorpholino)-8-(2,4-difluorophenyl)-2,3-dimethylpyrido[3,2-d]pyrimidin-4 (3H)-one

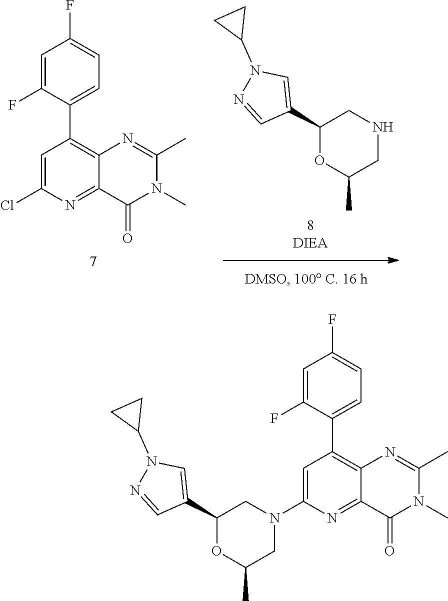 [SMILES: Cc1nc2c(-c3ccc(F)cc3F)cc(Cl)nc2c(=O)n1C ; C[C@@H]1CNC[C@H](c2cnn(C3CC3)c2)O1 ; Cc1nc2c(-c3ccc(F)cc3F)cc(N3C[C@@H](C)O[C@@H](c4cnn(C5CC5)c4)C3)nc2c(=O)n1C]

To a mixture of 6-chloro-8-(2,4-difluorophenyl)-2,3-dimethyl-pyrimido[5,4-d]pyrimidin-4-one (50 mg, 0.155 mmol, 1.0 eq) and (2S,6R)-2-(1-cyclopropylpyrazol-4-yl)-6-methyl-morpholine (48 mg, 0.233 mmol, 1.5 eq) in DMSO (2 mL) was added DIPEA (60 mg, 0.466 mmol, 3.00 eq) at 25° C. The mixture was stirred at 100° C., for 16 hours. LCMS indicated that the starting material was consumed and ˜50% desired product was detected. The reaction was allowed to cool to room temperature and diluted with H₂O (10 mL) and extracted with EtOAc (20 mL×3). The combined organic phase was washed with brine (20 mL×2), dried over anhydrous sodium sulfate, filtrated and concentrated under reduced pressure and purified by pre-HPLC (ACN—H₂O (0.1% NH₃); gradient: 5-95) and lyophilized to afford the desired product as a yellow solid (22 mg, 29% yield). LC-MS [M+H]⁺=493.3, R.T=1.283 min. ¹H NMR (400 MHz, DMSO) δ 7.83 (s, 1H), 7.58-7.52 (m, 1H), 7.47 (s, 1H), 7.45 (s, 1H), 7.37 (td, J=9.7, 2.4 Hz, 1H), 7.21 (td, J=8.7, 2.6 Hz, 1H), 4.57-4.40 (m, 3H), 3.80-3.65 (m, 2H), 3.50 (s, 3H), 2.93-2.82 (m, 1H), 2.65-2.60 (m, 1H), 2.41 (s, 3H), 1.21 (d, J=6.2 Hz, 3H), 1.06-1.00 (m, 2H), 0.97-0.91 (m, 2H).

### Example 16-Synthesis of Compound I-125: 4-(4-chloro-2-fluorophenyl)-2-((2S,6R)-2-(1-cyclopropyl-1H-pyrazol-4-yl)-6-methylmorpholino)-7-methylpyrido[2,3-d]pyridazin-8 (7H)-one

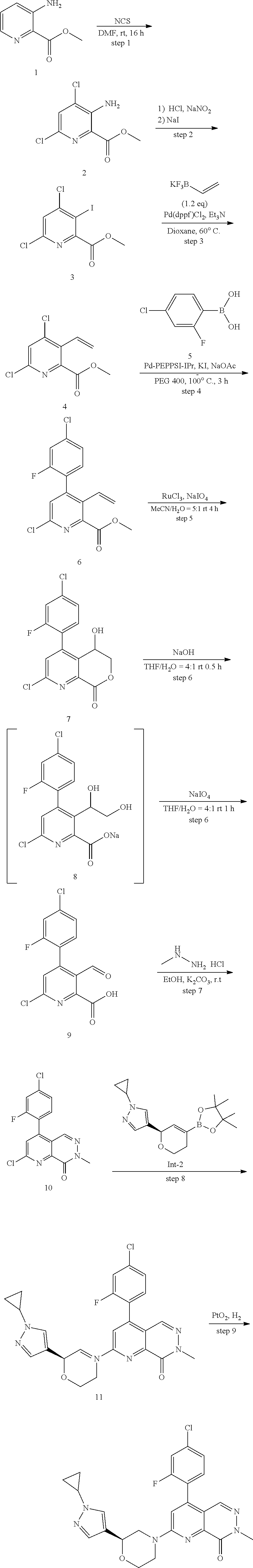

Step 1: To a stirred solution of methyl 3-aminopyridine-2-carboxylate (32.5 g, 214 mmol, 1.0 eq) in DMF (500 mL) was added NCS (57.05 g, 427 mmol, 2.0 eq). The mixture was stirred at 35° C., overnight. The mixture was poured into 4 L of water, filtrated to obtain methyl 3-amino-4,6-dichloro-pyridine-2-carboxylate (40.6 g, 184 mmol, 86.0% yield) LC-MS [M+H]⁺=220.9 R.T=0.61 min

Step 2: To a stirred solution of methyl 3-amino-4,6-dichloro-pyridine-2-carboxylate (221 mg, 1.00 mmol, 1.0 eq) in 1.5 mL of 12 M HCl was added dropwise NaNO₂ (103 mg, 1.50 mmol, 1.5 eq) in 0.5 mL of water at 0° C. The mixture was stirred at 0° C., for 30 min. To a stirred solution of NaI (498 mg, 3.00 mmol, 3.0 eq) in 4 mL of water and 4 mL of DCM was added above solution dropwise at 0° C. The reaction mixture was poured into water (50 mL) and extracted with DCM (100 mL×2). The organic layer was washed with Na₂S₂O₃ and brine, dried by Na₂SO₄. The solution was concentrated to give methyl 4,6-dichloro-3-iodo-pyridine-2-carboxylate (300 mg, 0.90 mmol, 90.4% yield). LC-MS [M+H]⁺=331.8 R.T=1.37 min

Step 3: To a stirred solution of methyl 4,6-dichloro-3-iodo-pyridine-2-carboxylate (8.00 g, 24.1 mmol, 1.0 eq) in 1,4-dioxane (160 mL) were added potassium vinyltrifluoroborate (6.51 g, 48.2 mmol, 2.0 eq), TEA (17 mL, 121 mmol, 5.0 eq), Pd(dppf)Cl₂ (3.17 g. 4.34 mmol, 0.18 eq). The mixture was stirred at 100° C., for 6 hours. The reaction mixture was filtered and extracted with ethyl acetate (500 mL×3). The combined organic layers were washed with brine (300 mL), dried over Na₂SO₄, filtered and concentrated under reduced pressure to give a residue. The residue was purified by column chromatography to afford methyl 4,6-dichloro-3-vinyl-pyridine-2-carboxylate (3.50 g, 15.1 mmol, 62.6% yield) LC-MS [M+H]⁺=231.8 R.T=1.21 min

Step 4: To a stirred solution of methyl 4,6-dichloro-3-vinyl-pyridine-2-carboxylate (160 mg, 0.69 mmol, 1.0 eq) in PEG 400 (2 mL) were added (4-chloro-2-fluoro-phenyl) boronic acid (81 mg. 0.46 mmol, 0.67 eq), KI (114 mg, 0.69 mmol, 1.0 eq), NaOAc (113 mg, 1.38 mmol, 2.0 eq), and Pd PEPPSI-IPr (470 mg, 0.69 mmol, 1.0 eq). The mixture was stirred at 100° C., for 4 hours. The mixture was diluted with water (20 mL) and extracted with ethyl acetate (50 mL×2). The combined organic layers were washed with brine (50 mL) and dried over Na₂SO₄. The solution was filtered and the filtrate was concentrated under reduced pressure. The crude product was purified by column chromatography on silica gel to obtain methyl 6-chloro-4-(4-chloro-2-fluoro-phenyl)-3-vinyl-pyridine-2-carboxylate (40 mg, 0.12 mmol, 17.8% yield). LC-MS [M+H]⁺=326.0 R.T=1.56 min

Step 5: To a stirred solution of methyl 6-chloro-4-(4-chloro-2-fluoro-phenyl)-3-vinyl-pyridine-2-carboxylate (690 mg, 2.12 mmol, 1.0 eq) in MeCN (17 mL) and water (3 mL) were added RuCl₃ (88 mg, 0.42 mmol, 0.2 eq), NaIO₄ (1.37 g, 6.35 mmol, 3.0 eq). The mixture was stirred at room temperature for 4 hour. The reaction mixture was diluted by addition of H₂O (50 mL) and extracted with ethyl acetate (100 mL×2). The residue was purified by column chromatography to afford 2-chloro-4-(4-chloro-2-fluorophenyl)-5-hydroxy-5,6-dihydro-8H-pyrano[3,4-b]pyridin-8-one (470 mg, 1.43 mmol, 67.6% yield). LC-MS [M+H]⁺=327.9 R.T=1.09 min

Step 6: To a solution of 2-chloro-4-(4-chloro-2-fluorophenyl)-5-hydroxy-5,6-dihydro-8H-pyrano[3,4-b]pyridin-8-one (270 mg, 0.82 mmol, 1.0 eq) in THF (9 mL) was added 1M NaOH (2.25 mL, 2.25 mmol, 2.7 eq). The mixture was stirred at room temperature for 0.5 h. TLC showed that the starting material was consumed completely and then NaIO₄ (527 mg. 2.46 mmol, 3 eq) was added. The mixture was stirred at room temperature for 1.5 h. The reaction mixture was diluted by addition of H₂O (50 mL) and adjusted to pH ˜3. The aqueous layers were extracted with ethyl acetate (50 mL×2). The combined organic layers were washed with brine (100 mL) and dried over Na₂SO₄. The solvent was filtered and the filtrate was concentrated under reduced pressure to obtain 6-chloro-4-(4-chloro-2-fluorophenyl)-3-formylpicolinic acid (260 mg. 0.83 mmol, 100% yield). LC-MS [M+H]⁺=313.9 [M−H]⁺=312.0 R.T=1.23 min.

Step 7: To a stirred solution of 6-chloro-4-(4-chloro-2-fluoro-phenyl)-3-formyl-pyridine-2-carboxylic acid (45 mg. 0.14 mmol, 1.0 eq) in ethanol (2 mL) was added methylhydrazine hydrochloride (9.9 mg, 0.22 mmol, 1.5 eq), K₂CO₃ (59 mg, 0.43 mmol, 3.0 eq). The mixture was stirred at room temperature for 2 hours. The reaction was poured into water and filtrated to obtain 2-chloro-4-(4-chloro-2-fluoro-phenyl)-7-methyl-pyrido[2,3-d]pyridazin-8-one (16 mg, 0.049 mmol, 34.5% yield). LC-MS [M+H]⁺=323.9 R.T=1.29 min

Step 8: To a stirred solution of 2-chloro-4-(4-chloro-2-fluoro-phenyl)-7-methyl-pyrido[2,3-d]pyridazin-8-one (16 mg. 0.049 mmol, 1.0 eq) in 1,4-dioxane (2 mL) and water (0.25 mL) was added 1-cyclopropylpyrazole-4,4,5,5-tetramethyl-2-[(6S)-6-methyl-3,6-dihydro-2H-pyran-4-yl]-1,3,2-dioxaborolane (18 mg, 0.054 mmol, 1.1 eq), Pd(dppf)Cl₂; DCM (4.0 mg, 0.0049 mmol, 0.1 eq), Cs₂CO₃ (32 mg, 0.099 mmol, 2.0 eq). The mixture was heated to 40° C. under N₂ for 2 hours. The reaction was poured into water and filtrated to obtain 4-(4-chloro-2-fluoro-phenyl)-2-[(6R)-6-(1-cyclopropylpyrazol-4-yl)-3,6-dihydro-2H-pyran-4-yl]-7-methyl-pyrido[2,3-d]pyridazin-8-one (18 mg, 0.038 mmol, 76.3% yield). LC-MS [M+H]⁺=478.1 R.T=1.339 min

Step 9: To a stirred solution of 4-(4-chloro-2-fluoro-phenyl)-2-[(6R)-6-(1-cyclopropylpyrazol-4-yl)-3,6-dihydro-2H-pyran-4-yl]-7-methyl-pyrido[2,3-d]pyridazin-8-one (64 mg. 0.13 mmol, 1.0 eq) in methanol (6 mL) was added PtO₂ (12 mg, 0.054 mmol, 0.4 eq). The mixture was stirred at H₂ for 4 hours. The reaction was filtrated and the filtrate was purified by prep-HPLC to obtain 4-(4-chloro-2-fluoro-phenyl)-2-[(2R,4S)-2-(1-cyclopropylpyrazol-4-yl)tetrahydropyran-4-yl]-7-methyl-pyrido[2,3-d]pyridazin-8-one (12 mg, 0.024 mmol, 18.2% yield) LC-MS [M+H]⁺=480.1 R.T=1.202 min ¹H NMR (400 MHz, DMSO) δ 8.00 (d, 1H), 7.90 (s, 1H), 7.76-7.70 (m, 2H), 7.65 (t, 1H), 7.55 (dd, 1H), 7.38 (s, 1H), 4.49-4.47 (m, 1H), 4.11-4.08 (m, 1H), 3.75 (s, 3H), 3.72-3.63 (m, 2H), 3.40-3.39 (m, 1H), 2.17-2.14 (m, 1H), 1.92-1.89 (m, 3H), 0.99-0.97 (m, 2H), 0.93-0.90 (m, 2H).

### Example 17-Synthesis of Compounds I-130, I-132 and I-134: 6-[(6R)-6-(1-cyclopropylpyrazol-4-yl)-3,6-dihydro-2H-pyran-4-yl]-8-[2-fluoro-4-(trifluoromethyl)phenyl]-2,3-dimethyl-pyrimido[5,4-d]pyrimidin-4-one (1-130), 6-[(2R,4S)-2-(1-cyclopropylpyrazol-4-yl)tetrahydropyran-4-yl]-8-[2-fluoro-4-(trifluoromethyl)phenyl]-2,3-dimethyl-pyrimido[5,4-d]pyrimidin-4-one (1-134), and 6-[(2R,4R)-2-(1-cyclopropylpyrazol-4-yl)tetrahydropyran-4-yl]-8-[2-fluoro-4-(trifluoromethyl)phenyl]-2,3-dimethyl-pyrimido[5,4-d]pyrimidin-4-one (I-132)

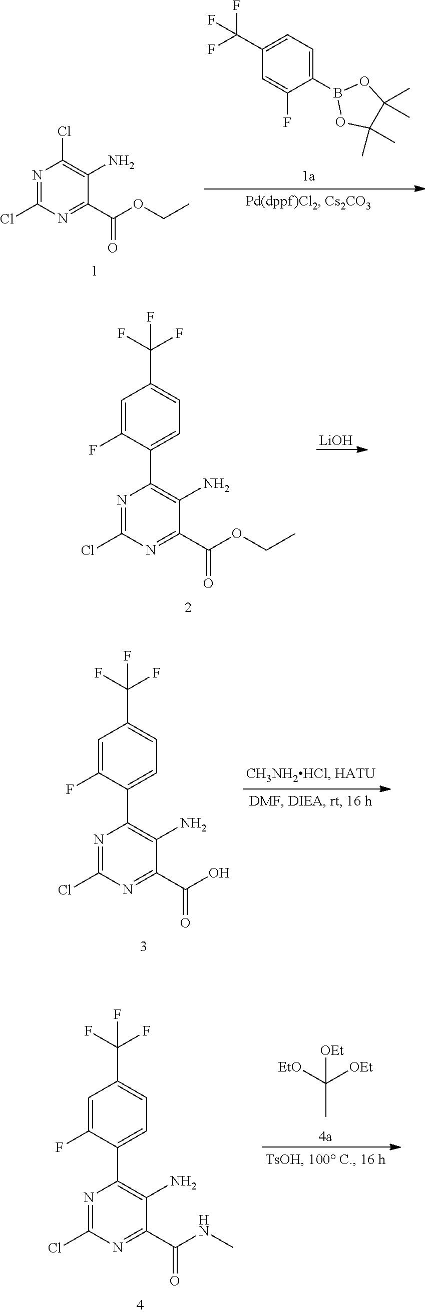

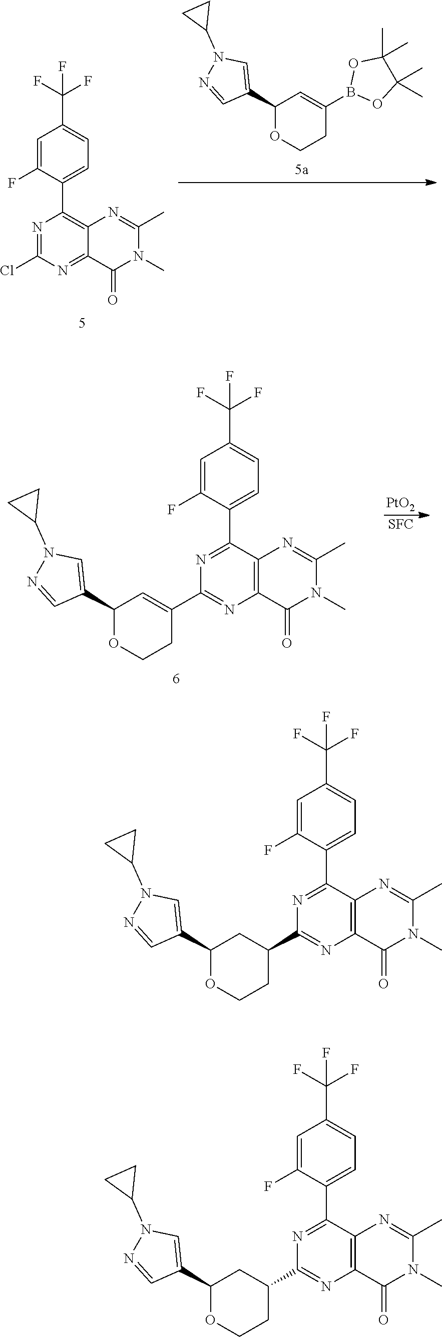

Step 1: A solution of ethyl 5-amino-2,6-dichloro-pyrimidine-4-carboxylate (1.00 eq, 2.00 g, 8.47 mmol) and 2-[2-fluoro-4-(trifluoromethyl)phenyl]-4,4,5,5-tetramethyl-1,3,2-dioxaborolane (1.10 eq, 2.70 g, 9.32 mmol) in 1,4-Dioxane (80 mL) and Water (8 mL) was added Cs₂CO₃ (1.20 eq, 3.30 g, 10.2 mmol) and Pd(dppf)Cl₂·CH₂Cl₂ (0.100 eq, 687 mg, 0.847 mmol) at N₂ atmosphere, then stirred at 40° C., for 2 hours. LCMS showed the starting material was consumed completely and a major peak with desired MS (LCMS: (M+H)+=364; purity=65.2% (UV 220 nm); Retention time=0.614 min) was detected. The mixture was concentrated under vacuum to give a crude. The crude was purified by flash column (PE:EtOAc=3:1; UV, Rf=0.3) and concentrated under vacuum to give ethyl 5-amino-2-chloro-6-[2-fluoro-4-(trifluoromethyl)phenyl]pyrimidine-4-carboxylate (2.80 g. 7.28 mmol, 85.87% yield) as orange solid. LCMS: (M+H)+=364; purity=94.5% (UV 220 nm), Retention time=0.606 min. ¹H NMR (400 MHz, CHLOROFORM-d) δ=7.75-7.66 (m, 1H), 7.65-7.59 (m, 1H), 7.57-7.48 (m, 1H), 6.01-5.71 (m, 2H), 4.60-4.40 (m, 2H), 1.51-1.41 (m, 3H)

Step 2: A mixture of ethyl 5-amino-2-chloro-6-[2-fluoro-4-(trifluoromethyl)phenyl]pyrimidine-4-carboxylate (1.00 eq, 2750 mg. 7.56 mmol) in Methanol (55 mL), THF (55 mL), and Water (55 mL) was added LiOH. H₂O (1.20 eq, 380 mg, 9.07 mmol). The mixture was stirred at 20° C., for 1 h. LCMS (SS-2022-04-037-24-PIA) showed a major peak with desired MS (LCMS: (M+H)+=336; purity=95.4% (UV 220 nm); Retention time=0.521 min) was detected. The mixture was added 1 N HCl (aq.) to pH=3˜4. The mixture was extracted with EtOAc (200 mL×3). The organic phases were dried over anhydrous Na₂SO₄ to give 5-amino-2-chloro-6-[2-fluoro-4-(trifluoromethyl)phenyl]pyrimidine-4-carboxylic acid (2500 mg, 7.31 mmol, 96.73% yield) as orange solid (crude). The crude was used for next step directly. LCMS: (M+H)+=335.9; purity=98.2% (UV 220 nm); Retention time=0.676 min. ¹H NMR (400 MHz, DMSO-d6) δ=7.96-7.87 (m, 1H), 7.85-7.75 (m, 2H), 7.12-6.44 (m, 2H).

Step 3: A mixture of 5-amino-2-chloro-6-[2-fluoro-4-(trifluoromethyl)phenyl]pyrimidine-4-carboxylic acid (1.00 eq, 2500 mg, 7.45 mmol) in DMF (125 mL) was added HATU (2.00 eq. 5664 mg, 14.9 mmol). The mixture was stirred for 30 mins. The mixture was added MeNH₂·HCl (5.00 eq. 2515 mg, 37.2 mmol) and DIPEA (5.00 eq, 6.5 mL, 37.2 mmol). The mixture was stirred at 20° C., for 12 h. LCMS showed a major peak with desired MS (LCMS: (M+H)+=349; purity=90.2% (UV 220 nm); Retention time=0.575 min) was detected. The mixture was concentrated under vacuum to give a crude. The crude was purified by flash column (PE:EtOAc=10:1 to 0:1, Rf=0.3, UV) to give 5-amino-2-chloro-6-[2-fluoro-4-(trifluoromethyl)phenyl]-N-methyl-pyrimidine-4-carboxamide (2.40 g. 6.83 mmol, 91.67% yield) as yellow solid. LCMS: (M+H)+=349.1; purity=99.2% (UV 220 nm); Retention time=0.864 min. ¹H NMR (400 MHz, CHLOROFORM-d) δ=8.11-7.89 (m, 1H), 7.75-7.64 (m, 1H), 7.64-7.57 (m, 1H), 7.55-7.46 (m, 1H), 6.28-6.01 (m, 2H), 3.08-2.97 (m, 3H).

Step 4: A mixture of 5-amino-2-chloro-6-[2-fluoro-4-(trifluoromethyl)phenyl]-N-methyl-pyrimidine-4-carboxamide (1.00 eq, 1000 mg, 2.87 mmol) in TRIETHYL ORTHOACETATE (18.9 eq, 10 mL, 54.2 mmol) was added TsOH (3.00 eq, 1480 mg, 8.60 mmol). The mixture was stirred at 100° C., for 12 h. LCMS (SS-2022-04-037-36-PIA) showed the starting material was consumed completely and a peak with desired MS (LCMS: (M+H)+=373; purity=44.2% (UV 220 nm); Retention time=0.719 min) was detected. The final mixture was concentrated under vacuum to give a crude. The crude was purified by flash column (PE:EtOAc=1:1, Rf=0.2, UV) and concentrated under vacuum to give 6-chloro-8-[2-fluoro-4-(trifluoromethyl)phenyl]-2,3-dimethyl-pyrimido[5,4-d]pyrimidin-4-one (1.30 g. 3.49 mmol, 121.62% yield) as yellow solid. LCMS: (M+H)+=373.1; purity=98.3% (UV 220 nm); Retention time=0.562 min. ¹H NMR (400 MHz, CHLOROFORM-d) δ=7.88-7.75 (m, 1H), 7.66-7.56 (m, 1H), 7.54-7.44 (m, 1H), 3.74-3.65 (m, 3H), 2.66-2.55 (m, 3H).

Step 5: To a solution of 6-chloro-8-[2-fluoro-4-(trifluoromethyl)phenyl]-2,3-dimethyl-pyrimido[5,4-d]pyrimidin-4-one (1.00 eq. 500 mg, 1.34 mmol), 1-cyclopropyl-4-[(6R)-4-(4,4,5,5-tetramethyl-1,3,2-dioxaborolan-2-yl)-3,6-dihydro-2H-pyran-6-yl]pyrazole (1.30 eq. 551 mg, 1.74 mmol) and K₂CO₃ (2.00 eq, 225 mg, 2.68 mmol) in 1,4-Dioxane (10 mL) and Water (2 mL), [1,1′-Bis(diphenylphosphino) ferrocene]dichloropalladium (II) (0.1000 eq. 98 mg. 0.134 mmol) was added and purged with N₂ for 3 times, the reaction solution was stirred at 100° C., for 2 h. LCMS (SS-2022-04-037-39-PIA) showed the reactant was consumed completely and a major peak with desired mass was detected (LCMS: (M+H)+=527.2; purity=76.9% (UV 220 nm); Retention time=0.622 min). The reaction solution was concentrated under reduced pressure to give a crude, which was then purified by flash column (PE:EtOAc=1:1, Rf=0.3) to give 6-[(6R)-6-(1-cyclopropylpyrazol-4-yl)-3,6-dihydro-2H-pyran-4-yl]-8-[2-fluoro-4-(trifluoromethyl)phenyl]-2,3-dimethyl-pyrimido[5,4-d]pyrimidin-4-one (600 mg, 1.11 mmol, 82.65% yield) as yellow. LCMS (M+H)+=527.2; purity=97.3% (220 nm); Retention time=0.762 min. ¹H NMR (400 MHz, CHLOROFORM-d) δ=7.89-7.78 (m, 1H), 7.67-7.48 (m, 5H), 4.72 (dd, J=3.3, 10.0 Hz, 1H), 4.66-4.55 (m, 2H), 3.77-3.66 (m, 3H), 3.65-3.52 (m, 1H), 3.37-3.21 (m, 1H), 3.01-2.84 (m, 1H), 2.69-2.56 (m, 3H), 1.18-1.11 (m, 2H), 1.07-0.98 (m, 2H)

Step 6: A mixture of 6-[(6R)-6-(1-cyclopropylpyrazol-4-yl)-3,6-dihydro-2H-pyran-4-yl]-8-[2-fluoro-4-(trifluoromethyl)phenyl]-2,3-dimethyl-pyrimido[5,4-d]pyrimidin-4-one (1.00 eq. 600 mg. 1.14 mmol) in Ethanol (20 mL) was added PtO₂ (0.831 eq. 215 mg, 0.947 mmol). The mixture was degassed with N₂ for 3 times and degassed with H₂ for 3 times. The mixture was stirred at 20° C., for 0.5 h under H₂ balloon (15 psi). LCMS showed the reaction was completed and a major peak with desired MS (LCMS: (M+H)+=529.2; purity=73.3% (UV 220 nm); Retention time=0.743 min). The mixture was degassed with N₂ for 3 times. The mixture was filtered celite and the filtrate was concentrated under vacuum to give a crude product. The crude was purified by flash column (PE:EtOAc=1:1 to 0:1, Rf=0.3, UV) and concentrated under vacuum to give 6-[(2R)-2-(1-cyclopropylpyrazol-4-yl)tetrahydropyran-4-yl]-8-[2-fluoro-4-(trifluoromethyl)phenyl]-2,3-dimethyl-pyrimido[5,4-d]pyrimidin-4-one (350 mg. 0.651 mmol, 57.12% yield) as white solid. 350 mg product was sent for SFC separation (Sample preparation: Add CH3OH 20 mL into sample; Instrument: Waters 80Q; Mobile Phase: 40% ETOH (0.1% NH₃·H₂O) in Supercritical CO₂; Flow Rate: 70 g/min; Cycle Time: 3.8 min, total time: 45 min; Single injection volume: 3.5 mL; Back Pressure: 100 bar to keep the CO₂ in Supercritical flow) and then lyophilized to give 6-[(2R,4R)-2-(1-cyclopropylpyrazol-4-yl)tetrahydropyran-4-yl]-8-[2-fluoro-4-(trifluoromethyl)phenyl]-2,3-dimethyl-pyrimido[5,4-d]pyrimidin-4-one (53 mg, 0.0930 mmol, 8.16% yield) as white solid and 6-[(2R,4S)-2-(1-cyclopropylpyrazol-4-yl)tetrahydropyran-4-yl]-8-[2-fluoro-4-(trifluoromethyl)phenyl]-2,3-dimethyl-pyrimido[5,4-d]pyrimidin-4-one (201 mg, 0.357 mmol, 31.35% yield) as white solid.

I-134: LCMS: (M+H)+=529.2; purity=98.7% (UV 220 nm); Retention time=0.596 min. ¹H NMR (400 MHz, CHLOROFORM-d) δ=7.83-7.76 (m, 1H), 7.63-7.56 (m, 1H), 7.52-7.44 (m, 3H), 4.56-4.46 (m, 1H), 4.31-4.16 (m, 1H), 3.84-3.72 (m, 1H), 3.71-3.65 (m, 3H), 3.63-3.47 (m, 2H), 2.64-2.56 (m, 3H), 2.37-2.27 (m, 1H), 2.23-2.02 (m, 3H), 1.12-1.05 (m, 2H), 1.01-0.93 (m, 2H)

I-132; LCMS: (M+H)+=529.2; purity=93.4% (UV 220 nm); Retention time=0.598 min. ¹H NMR (400 MHz, CHLOROFORM-d) δ=7.81 (t, J=7.3 Hz, 1H), 7.59 (d, J=7.9 Hz, 1H), 7.50 (d, J=9.9 Hz, 1H), 7.45 (d, J=4.3 Hz, 2H), 4.89 (dd, J=3.5, 7.8 Hz, 1H), 3.94-3.84 (m, 2H), 3.77-3.70 (m, 1H), 3.70-3.66 (m, 3H), 3.56 (td, J=3.6, 7.3 Hz, 1H), 2.73-2.63 (m, 1H), 2.60 (s, 3H), 2.37-2.22 (m, 2H), 2.19-2.08 (m, 1H), 1.15-1.06 (m, 2H), 1.03-0.94 (m, 2H)

### Example 18-Synthesis of Compound I-136: 6-[(2S,6R)-2-(1-cyclopropylpyrazol-4-yl)-6-methyl-morpholin-4-yl]-8-[2-fluoro-4-(trifluoromethyl)phenyl]-2,3-dimethyl-pyrimido[5,4-d]pyrimidin-4-one

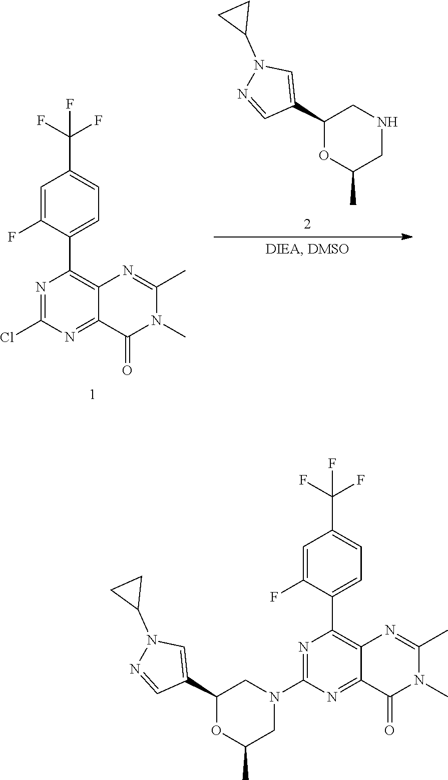 [SMILES: Cc1nc2c(-c3ccc(C(F)(F)F)cc3F)nc(Cl)nc2c(=O)n1C ; C[C@@H]1CNC[C@H](c2cnn(C3CC3)c2)O1 ; Cc1nc2c(-c3ccc(C(F)(F)F)cc3F)nc(N3C[C@@H](C)O[C@@H](c4cnn(C5CC5)c4)C3)nc2c(=O)n1C]

Step 1: To a solution of 6-chloro-8-[2-fluoro-4-(trifluoromethyl)phenyl]-2,3-dimethyl-pyrimido[5,4-d]pyrimidin-4-one (1.00 eq, 100 mg, 0.268 mmol) and (2S,6R)-2-(1-cyclopropylpyrazol-4-yl)-6-methyl-morpholine (1.00 eq, 56 mg, 0.268 mmol) in DMSO (2.5 mL) was added DIEA (5.00 eq, 0.22 mL, 1.34 mmol). The mixture was stirred at 100° C., for 20 min. The reaction mixture was clear brown solution. LCMS showed 14.7% starting material remained and a major peak with desired MS (LCMS: (M+H)+=544.2; purity=77.83% (UV 220 nm); Retention time=0.644 min) was detected. The final mixture was purified by prep-HPLC (FA, Column: phenomenex luna C18 150*25 mm*₁₀ μm, the condition was water (FA)-ACN; Gradient Time (min): 10; Flow Rate (mL/min): 25) and lyophilized to give 6-[(2S,6R)-2-(1-cyclopropylpyrazol-4-yl)-6-methyl-morpholin-4-yl]-8-[2-fluoro-4-(trifluoromethyl)phenyl]-2,3-dimethyl-pyrimido[5,4-d]pyrimidin-4-one (100 mg, 0.183 mmol, 68.30% yield) as yellow solid. LCMS: (M+H)+=544.2; purity=100% (UV 220 nm); Retention time=0.639 min. ¹H NMR (400 MHz, CHLOROFORM-d) δ=7.82-7.68 (m, 1H), 7.59-7.50 (m, 3H), 7.49-7.42 (m, 1H), 5.07-4.71 (m, 2H), 4.64-4.52 (m, 1H), 3.89-3.72 (m, 1H), 3.62 (s, 3H), 3.60-3.51 (m, 1H), 3.10-2.98 (m, 1H), 2.88-2.72 (m, 1H), 2.58-2.43 (m, 3H), 1.38-1.26 (m, 3H), 1.17-1.06 (m, 2H), 1.05-0.95 (m, 2H) SFC showed 100% purity.

### Example 19-Synthesis of Compound I-141: 2-[(2S, 6R)-2-(1-cyclopropylpyrazol-4-yl)-6-methyl-morpholin-4-yl]-4-(2,4-difluorophenyl)-7-methyl-pyrimido[4,5-d]pyridazin-8-one

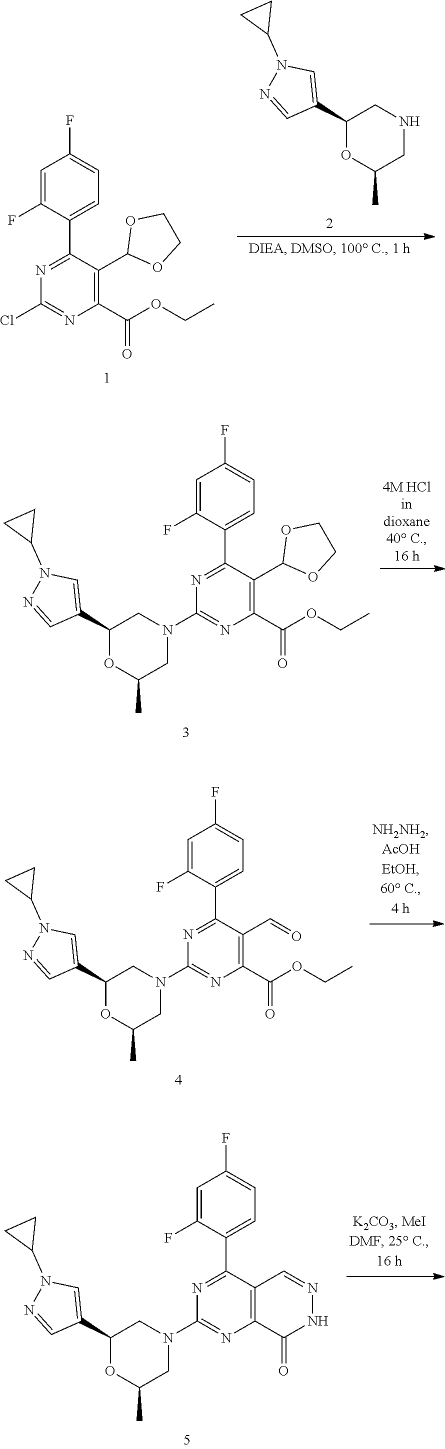 [SMILES: CCOC(=O)c1nc(Cl)nc(-c2ccc(F)cc2F)c1C1OCCO1 ; C[C@@H]1CNC[C@H](c2cnn(C3CC3)c2)O1 ; CCOC(=O)c1nc(N2C[C@@H](C)O[C@@H](c3cnn(C4CC4)c3)C2)nc(-c2ccc(F)cc2F)c1C1OCCO1 ; CCOC(=O)c1nc(N2C[C@@H](C)O[C@@H](c3cnn(C4CC4)c3)C2)nc(-c2ccc(F)cc2F)c1C=O ; C[C@@H]1CN(c2nc(-c3ccc(F)cc3F)c3cn[nH]c(=O)c3n2)C[C@H](c2cnn(C3CC3)c2)O1]

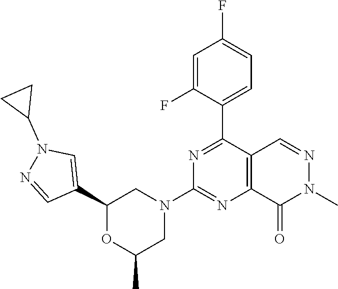 [SMILES: C[C@@H]1CN(c2nc(-c3ccc(F)cc3F)c3cnn(C)c(=O)c3n2)C[C@H](c2cnn(C3CC3)c2)O1]

Step 1: A solution of ethyl 2-chloro-6-(2,4-difluorophenyl)-5-(1,3-dioxolan-2-yl)pyrimidine-4-carboxylate (1.00 eq. 1000 mg. 2.70 mmol) in DMSO (10 mL) was added (2S,6R)-2-(1-cyclopropylpyrazol-4-yl)-6-methyl-morpholine (1.10 eq. 615 mg. 2.97 mmol) and DIEA (5.00 eq. 2.2 mL. 13.5 mmol), then stirred at 100° C., for 1 hour. LCMS showed 64% of desired MS. The reaction mixture was extracted with ethyl acetate (100 mL*3). The combined organic layers were dried over Na₂SO₄, filtered and concentrated under reduced pressure to give a residue. The residue was purified by column chromatography on silica gel eluting with PE/EtOAc (1:0 to 50:1) (TLC, PE:EtOAc=0:1, Rf=0.40) to afford ethyl 2-[(2S,6R)-2-(1-cyclopropylpyrazol-4-yl)-6-methyl-morpholin-4-×1]-6-(2,4-difluorophenyl)-5-(1,3-dioxolan-2-yl)pyrimidine-4-carboxylate (900 mg, 1.50 mmol, 55.45% yield) as off-white solid, which confirmed by LCMS. [M+H]⁺=542.3; purity=95% (220 nm); Retention time=0.668 min.

Step 2: A solution of ethyl 2-[(2S, 6R)-2-(1-cyclopropylpyrazol-4-yl)-6-methyl-morpholin-4-yl]-6-(2, 4-difluorophenyl)-5-(1,3-dioxolan-2-yl)pyrimidine-4-carboxylate (1.00 eq. 540 mg. 0.997 mmol) in HCl dioxane (5.0 mL) was stirred at 40° C., for 16 hours. LCMS showed 79% of desired product. The reaction mixture was concentrated under reduced pressure to give ethyl 2-[(2S, 6R)-2-(1-cyclopropylpyrazol-4-yl)-6-methyl-morpholin-4-yl]-6-(2,4-difluorophenyl)-5-formyl-pyrimidine-4-carboxylate (500 mg. 0.794 mmol, 79.63% yield) as yellow oil. [M+H]+=498.2; purity=79% (220 nm); Retention time=0.794 min.

Step 3: To a solution of ethyl 2-[(2S,6R)-2-(1-cyclopropylpyrazol-4-yl)-6-methyl-morpholin-4-yl]-6-(2,4-difluorophenyl)-5-formyl-pyrimidine-4-carboxylate (1.00 eq. 500 mg, 1.01 mmol) in Ethanol (4 mL) were added NH₂NH₂H₂O (1.50 eq. 75 mg, 1.51 mmol) and AcOH (1.00 eq, 139 mg, 1.01 mmol). The mixture was refluxed at 60° C., for 4 hours. LCMS showed 47.9% of desired mass. The reaction mixture was extracted with ethyl acetate (20 mL*3). The combined organic layers were dried over Na₂SO₄, filtered and concentrated under reduced pressure to give a residue. The residue was purified by column chromatography on silica gel eluting with DCM/MeOH (0:0 to 10:1) (TLC, PE:EtOAc=0:1, Rf=0.55) to afford 2-[(2S,6R)-2-(1-cyclopropylpyrazol-4-yl)-6-methyl-morpholin-4-yl]-4-(2,4-difluorophenyl)-7H-pyrimido[4,5-d]pyridazin-8-one (300 mg. 0.322 mmol, 32.06% yield) as yellow solid, which confirmed by LCMS. [M+H]⁺=466.3; purity=50% (220 nm); Retention time=0.578 min.

Step 4: A solution of 2-[(2S,6R)-2-(1-cyclopropylpyrazol-4-yl)-6-methyl-morpholin-4-yl]-4-(2,4-difluorophenyl)-7H-pyrimido[4,5-d]pyridazin-8-one (1.00 eq. 300 mg. 0.645 mmol) in DMF (4 mL) was added K₂CO₃ (2.00 eq. 178 mg, 1.29 mmol) and CH₃I (2.00 eq. 0.080 mL, 1.29 mmol) and stirred at 25° C., for 16 hours. LCMS showed 24% of desired mass and 49% di-methylation by-product (1-cyclopropyl-4-((2S,6R)-4-(4-(2,4-difluorophenyl)-7-methyl-8-oxo-7,8-dihydropyrimido[4,5-d]pyridazin-2-yl)-6-methylmorpholin-2-yl)-2-methyl-1H-pyrazol-2-ium). The reaction mixture was extracted with ethyl acetate (50 mL*3). The combined organic layers were dried over Na₂SO₄, filtered and concentrated under reduced pressure to give a residue. The residue was purified by prep-HPLC (Column, [Phenomenex luna C18 250*50 mm*₁₀ um]; mobile phase: [ACN] and [H2O](conditions: [water (0.225% FA)-ACN], B %: 45%-60%; Detector, UV 254 nm. RT: [22 min]) to afford 2-[(2S,6R)-2-(1-cyclopropylpyrazol-4-yl)-6-methyl-morpholin-4-yl]-4-(2,4-difluorophenyl)-7-methyl-pyrimido[4,5-d]pyridazin-8-one (47 mg, 0.0980 mmol, 15.21% yield) as light yellow solid, which confirmed by LCMS and H NMR, F NMR, HPLC, SFC. [M+H]⁺=480.2; purity=100% (220 nm); Retention time=0.913 min. ¹H NMR (400 MHz, CHLOROFORM-d) δ=7.78 (br d, J=3.9 Hz, 1H), 7.74-7.45 (m, 3H), 7.23-6.93 (m, 2H), 5.38-4.77 (m, 2H), 4.58 (br s, 1H), 3.84 (br s, 4H), 3.58 (br d, J=1.5 Hz, 1H), 3.20-3.00 (m, 1H), 2.96-2.74 (m, 1H), 1.35 (br s, 3H), 1.19-0.93 (m, 4H).

### Example 20-Synthesis of Compound I-146: 2-[(2R)-2-(1-cyclopropylpyrazol-4-yl)tetrahydropyran-4-yl]-4-(2,4-difluorophenyl)-7-methyl-pyrimido[4,5-d]pyridazin-8-one

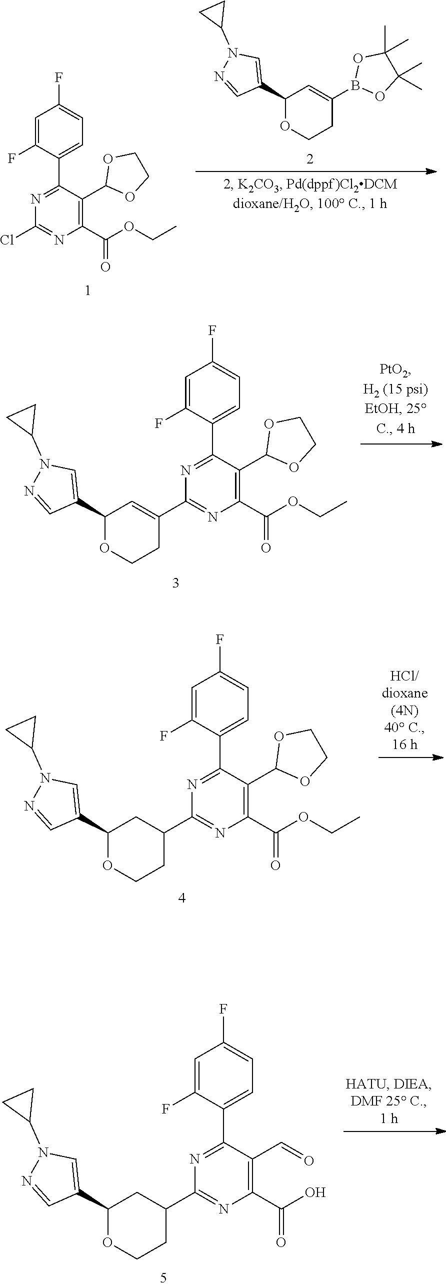 [SMILES: CCOC(=O)c1nc(Cl)nc(-c2ccc(F)cc2F)c1C1OCCO1 ; CC1(C)OB(C2=C[C@H](c3cnn(C4CC4)c3)OCC2)OC1(C)C ; CCOC(=O)c1nc(C2=C[C@H](c3cnn(C4CC4)c3)OCC2)nc(-c2ccc(F)cc2F)c1C1OCCO1 ; CCOC(=O)c1nc(C2CCO[C@@H](c3cnn(C4CC4)c3)C2)nc(-c2ccc(F)cc2F)c1C1OCCO1 ; O=Cc1c(C(=O)O)nc(C2CCO[C@@H](c3cnn(C4CC4)c3)C2)nc1-c1ccc(F)cc1F]

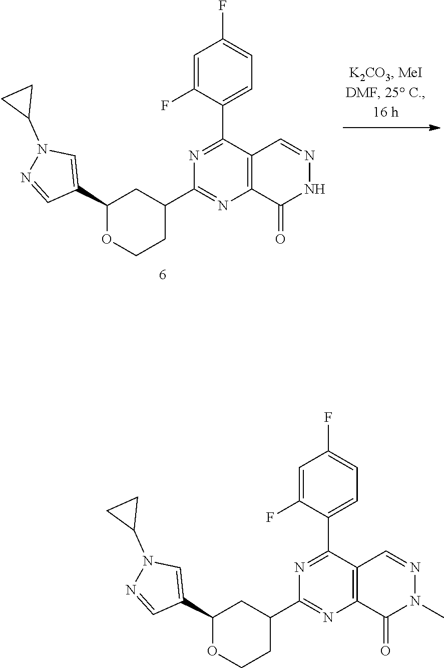 [SMILES: O=c1[nH]ncc2c(-c3ccc(F)cc3F)nc(C3CCO[C@@H](c4cnn(C5CC5)c4)C3)nc12 ; Cn1ncc2c(-c3ccc(F)cc3F)nc(C3CCO[C@@H](c4cnn(C5CC5)c4)C3)nc2c1=O]

Step 1: A solution of ethyl 2-chloro-6-(2,4-difluorophenyl)-5-(1,3-dioxolan-2-yl)pyrimidine-4-carboxylate (1.00 eq, 2000 mg, 5.39 mmol) and 1-cyclopropyl-4-[(6R)-4-(4,4,5,5-tetramethyl-1,3,2-dioxaborolan-2-yl)-3,6-dihydro-2H-pyran-6-yl]pyrazole (1.10 eq, 1876 mg, 5.93 mmol) in 1,4-Dioxane (15 mL) and Water (1.5 mL) was added K₂CO₃ (3.00 eq. 2237 mg, 16.2 mmol) and Pd(dppf)Cl₂; CH₂Cl₂ (0.100 eq. 437 mg. 0.539 mmol), purged with N₂ three times, then stirred at 100° C. for 1 hour. LCMS showed 52.6% of desired mass. The reaction mixture was extracted with ethyl acetate (100 mL*3). The combined organic layers were dried over Na₂SO₄, filtered and concentrated under reduced pressure to give a residue. The residue was purified by column chromatography on silica gel eluting with PE/EtOAc (1:0 to 50:1) (TLC, PE:EtOAc-0:1, Rf=0.40) to afford ethyl 2-[(6R)-6-(1-cyclopropylpyrazol-4-yl)-3,6-dihydro-2H-pyran-4-yl]-6-(2,4-difluorophenyl)-5-(1,3-dioxolan-2-yl)pyrimidine-4-carboxylate (1900 mg, 3.26 mmol, 60.43% yield) as off-white solid, which confirmed by LCMS. [M+H]+=525.3; purity=91.4% (220 nm); Retention time=0.655 min

Step 2: A solution of ethyl 2-[(6R)-6-(1-cyclopropylpyrazol-4-yl)-3,6-dihydro-2H-pyran-4-yl]-6-(2,4-difluorophenyl)-5-(1,3-dioxolan-2-yl)pyrimidine-4-carboxylate (1.00 eq. 1400 mg. 2.67 mmol) in Ethanol (20 mL) was added PtO₂ (1.16 eq. 700 mg. 3.08 mmol) at N₂ atmosphere, then stirred at 25° C., for 4 hours under H₂ (15 PSI). LCMS showed 77% of desired mass. The reaction mixture was filtered through celite, the filtrate was evaporated under reduced pressure to give the crude product ethyl 2-[(2R)-2-(1-cyclopropylpyrazol-4-yl)tetrahydropyran-4-yl]-6-(2,4-difluorophenyl)-5-(1,3-dioxolan-2-yl)pyrimidine-4-carboxylate (1100 mg, 1.71 mmol, 64.18% yield) as white solid, which confirmed by LCMS. [M+H]+=527.2; purity=82% (220 nm); Retention time=0.935 min.

Step 3: A solution of ethyl ethyl 2-[(2R)-2-(1-cyclopropylpyrazol-4-yl)tetrahydropyran-4-yl]-6-(2,4-difluorophenyl)-5-(1,3-dioxolan-2-yl)pyrimidine-4-carboxylate (1.00 eq. 1100 mg. 2.09 mmol) in HCl⋅dioxane (10 mL) was stirred at 40° C., for 16 hours. LCMS showed 41% desired mass and 38% ethyl 2-[(2R)-2-(1-cyclopropylpyrazol-4-yl)tetrahydropyran-4-yl]-6-(2,4-difluorophenyl)-5-formyl-pyrimidine-4-carboxylate. The reaction mixture was concentrated under reduced pressure to give 2-[(2R)-2-(1-cyclopropylpyrazol-4-yl)tetrahydropyran-4-yl]-6-(2,4-difluorophenyl)-5-formyl-pyrimidine-4-carboxylic acid (1300 mg, 1.17 mmol, 80% yield) as yellow oil, without further workup. [M+H]+=454.2; purity=41% (220 nm); Retention time=0.865 min.

Step 4: A solution of 2-[(2R)-2-(1-cyclopropylpyrazol-4-yl)tetrahydropyran-4-yl]-6-(2,4-difluorophenyl)-5-formyl-pyrimidine-4-carboxylic acid (1.00 eq. 1300 mg. 2.86 mmol) HATU (1.50 eq. 1632 mg. 4.29 mmol) and DIEA (5.00 eq. 2.4 mL, 14.3 mmol) in DMF (10 mL), stirred at 25° C., for 20 minutes, then was added hydrazine (1.50 eq. 0.14 mL, 4.29 mmol) at 0° C., and stirred at 25° C., for 1 hour. LCMS showed 25% 2-[(2R)-2-(1-cyclopropylpyrazol-4-yl)tetrahydropyran-4-yl]-4-(2,4-difluorophenyl)-7H-pyrimido[4,5-d]pyridazin-8-one and 46% 2-((2R)-2-(1-cyclopropyl-1H-pyrazol-4-yl)tetrahydro-2H-pyran-4-yl)-6-(2,4-difluorophenyl)-5-((E)-hydrazineylidenemethyl)pyrimidine-4-carboxylic acid. The reaction mixture was extracted with ethyl acetate (200 mL*3). The combined organic layers were dried over Na₂SO₄, filtered and concentrated under reduced pressure to give a residue. The residue was purified by prep-HPLC (Column, [Phenomenex luna C18 250*50 mm*10 um]; mobile phase: [ACN] and [H2O](conditions: [water (0.225% FA)-ACN], B %: 30%-50%; Detector, UV 254 nm. RT: [22 min]) to afford 2-[(2R)-2-(1-cyclopropylpyrazol-4-yl)tetrahydropyran-4-yl]-4-(2,4-difluorophenyl)-7H-pyrimido[4,5-d]pyridazin-8-one (200 mg, 0.431 mmol, 15.06% yield) as yellow solid, which confirmed by LCMS. [M+H]+=451.2; purity=97% (220 nm); Retention time=0.530 min.

Step 5: A solution of 2-[(2R)-2-(1-cyclopropylpyrazol-4-yl)tetrahydropyran-4-yl]-4-(2,4-difluorophenyl)-7H-pyrimido[4,5-d]pyridazin-8-one (1.00 eq, 145 mg, 0.322 mmol) in DMF (2 mL) was added K₂CO₃ (2.00 eq, 89 mg, 0.644 mmol) and CH₃I (2.00 eq, 0.040 mL, 0.644 mmol), then stirred at 25° C., for 16 hours. LCMS showed 22.1% of desired mass and 39% of 1-cyclopropyl-4-((2R)-4-(4-(2,4-difluorophenyl)-7-methyl-8-oxo-7,8-dihydropyrimido[4,5-d]pyridazin-2-yl)tetrahydro-2H-pyran-2-yl)-2-methyl-1H-pyrazol-2-ium (2D NMR confirmed di-methlation BP). The reaction mixture was extracted with ethyl acetate (50 mL*3). The combined organic layers were dried over Na₂SO₄, filtered and concentrated under reduced pressure to give a residue. The residue was purified by prep-HPLC (Column, [Phenomenex luna C18 250*50 mm*10 um]; mobile phase: [ACN] and [H2O](conditions: [water (0.225% FA)-ACN], B %: 45%-60%; Detector, UV 254 nm. RT: [22 min]) to afford 2-[(2R)-2-(1-cyclopropylpyrazol-4-yl)tetrahydropyran-4-yl]-4-(2,4-difluorophenyl)-7-methyl-pyrimido[4,5-d]pyridazin-8-one (22 mg 0.00476 mmol, 14.8% yield) as yellow solid, which confirmed by LCMS and H NMR, F NMR, HPLC SFC. [M+H]+=465.1; purity=100% (220 nm); Retention time=0.887 min. ¹H NMR (400 MHz, CDCl₃) δ=8.12-8.02 (m, 1H), 7.73 (q, J=7.3 Hz, 1H), 7.49 (br d, J=2.3 Hz, 2H), 7.19 (br t, J=8.1 Hz, 1H), 7.09 (br t, J=9.3 Hz, 1H), 4.55 (br d, J=11.2 Hz, 1H), 4.26 (br dd, J=3.2, 11.0 Hz, 1H), 3.94 (s, 3H), 3.80 (br t, J=11.8 Hz, 1H), 3.71-3.61 (m, 1H), 3.60-3.52 (m, 1H), 2.35 (br d, J=13.1 Hz, 1H), 2.22-2.04 (m, 3H), 1.10 (br d, J=1.0 Hz, 2H), 1.00 (br d, J=6.8 Hz, 2H)

### Example 21: Synthesis of Compound I-152: 8-(4-chloro-2-fluoro-phenyl)-6-[(2S,6R)-2-(1-cyclopropylpyrazol-4-yl)-6-methyl-morpholin-4-yl]-2,3-dimethyl-pyrimido[5,4-d]pyrimidin-4-one

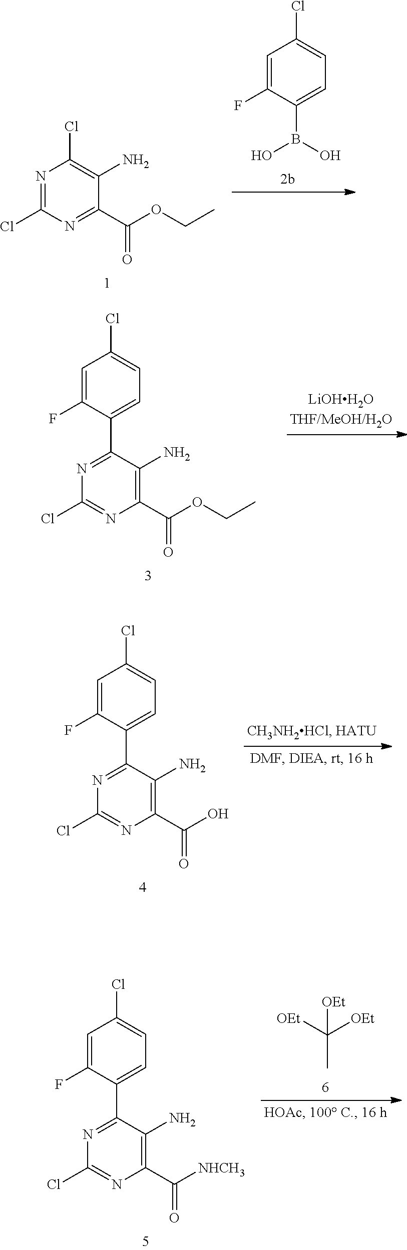 [SMILES: CCOC(=O)c1nc(Cl)nc(Cl)c1N ; OB(O)c1ccc(Cl)cc1F ; CCOC(=O)c1nc(Cl)nc(-c2ccc(Cl)cc2F)c1N ; Nc1c(C(=O)O)nc(Cl)nc1-c1ccc(Cl)cc1F ; CNC(=O)c1nc(Cl)nc(-c2ccc(Cl)cc2F)c1N ; CCOC(C)(OCC)OCC]

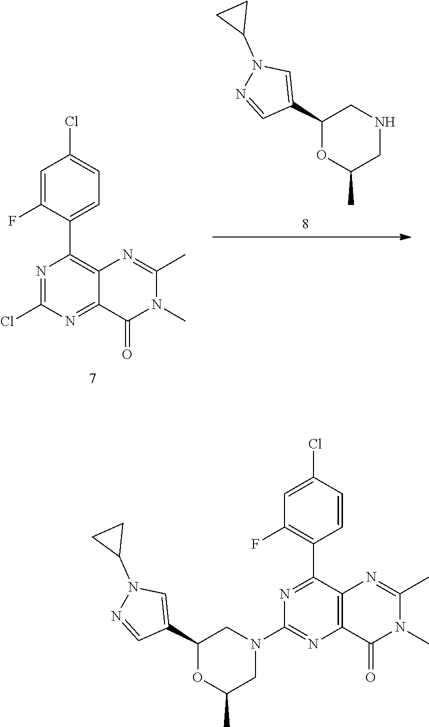 [SMILES: Cc1nc2c(-c3ccc(Cl)cc3F)nc(Cl)nc2c(=O)n1C ; C[C@@H]1CNC[C@H](c2cnn(C3CC3)c2)O1 ; Cc1nc2c(-c3ccc(Cl)cc3F)nc(N3C[C@@H](C)O[C@@H](c4cnn(C5CC5)c4)C3)nc2c(=O)n1C]

Step 1: To a solution of ethyl 5-amino-2,6-dichloro-pyrimidine-4-carboxylate (1.00 eq, 2000 mg, 8.47 mmol), (4-chloro-2-fluoro-phenyl) boronic acid (1.00 eq, 1477 mg, 8.47 mmol) and Cs₂CO₃ (1.20 eq, 3304 mg, 10.2 mmol) in 1,4-Dioxane (20 mL) and Water (2 mL) was added Pd(dppf)Cl₂·CH₂C12 (0.100 eq, 620 mg, 0.847 mmol). The reaction mixture was purged with N₂ for 3 times, then stirred at 20° C. for 12 h. LCMS showed ˜70% of desired product was detected (70%, Rt=0.634 min; [M+H]+=330.0 at 220 nm). The mixture was quenched by 400 mL H2O, extracted with EA (300 mL*3), the combine organic layers was dried over anhydrous Na₂SO₄, filtered and concentrated under reduced pressure to give the residue. The residue was purified by silical gel chromatography (PE:EA=1:0 to 5:1, PE:EA=5:1, the desired product Rf=0.4) to give the ethyl 5-amino-2-chloro-6-(4-chloro-2-fluoro-phenyl)pyrimidine-4-carboxylate (2200 mg, 6.66 mmol, 78.65% yield) as light yellow solid. [M+H]+=330.0; 1H NMR (400 MHz, CHLOROFORM-d) δ=7.50 (t, J=7.9 Hz, 1H), 7.36 (dd, J=1.9, 8.3 Hz, 1H), 7.30 (dd, J=1.9, 9.6 Hz, 1H), 5.87 (br s, 2H), 4.51 (q, J=7.1 Hz, 2H), 1.47 (t, J=7.1 Hz, 3H).

Step 2: A mixture of ethyl 5-amino-2-chloro-6-(4-chloro-2-fluoro-phenyl)pyrimidine-4-carboxylate (1.00 eq. 2200 mg. 6.66 mmol) in Methanol (40 mL). THF (40 mL) and Water (40 mL) was added LiOH·H2O (1.20 eq. 335 mg. 8.00 mmol). The mixture was stirred at 20° C., for 1 h. LCMS (NT-2022-03-050-89-PIA) showed a major peak with desired product was detected (89%, Rt=0.577 min; [M+H]+=302.0 at 220 nm). 1 N HCl (aq.) was added to the reaction mixture until the PH=3˜4. The mixture was extracted with EtOAc (200 mL*3), the organic phases were dried over anhydrous Na₂SO₄ to give 5-amino-2-chloro-6-(4-chloro-2-fluoro-phenyl)pyrimidine-4-carboxylic acid (2000 mg, 6.50 mmol, 97.56% yield) as orange solid (crude). The H NMR confirmed the structure. The crude was used for next step directly. (P1): 89%, Rt=0.577 min: [M+H]+=302.0 at 220 nm ¹H NMR (400 MHz, DMSO-d6) δ=7.65 (dd, J=1.9, 9.9 Hz, 1H), 7.62-7.54 (m, 1H), 7.47 (dd, J=1.9, 8.3 Hz, 1H), 7.03-6.34 (m, 2H)

Step 3: A mixture of 5-amino-2-chloro-6-(4-chloro-2-fluoro-phenyl)pyrimidine-4-carboxylic acid (1.00 eq. 2400 mg. 7.94 mmol) in DMF (200 mL) was added HATU (2.00 eq. 6042 mg. 15.9 mmol). The mixture was stirred at 20° C., for 30 mins. METHYLAMINE HYDROCHLORIDE (5.00 eq. 2682 mg. 39.7 mmol) and DIPEA (5.00 eq. 6.9 mL. 39.7 mmol) was added to the reaction mixture, then the reaction mixture was stirred at 20° C., for 12 h. LCMS showed ˜44% of desired product was detected (44%. Rt=0.613 min: [M+H]⁺=315.0 at 220 nm). The mixture was quenched by 200 mL H2O, extracted with EA (200 mL*3), the combine organic layers was dried over anhydrous Na₂SO₄, filtered and concentrated under reduced pressure to give the residue. The residue was purified by silical gel chromatography (PE:EA=1:0 to 3:1. PE:EA=3:1 the desired product Rf=0.5) to give the 5-amino-2-chloro-6-(4-chloro-2-fluoro-phenyl)-N-methyl-pyrimidine-4-carboxamide (2.20 g. 6.98 mmol. 87.87% yield) as light red solid. Ms=315.0. [M+H]+, ESI+¹H NMR (400 MHz, CHLOROFORM-d) δ=8.01 (br d, J=9.9 Hz, 1H), 7.49 (t, J=7.9 Hz, 1H), 7.36-7.28 (m, 2H), 6.15 (br s, 2H), 3.01 (d, J=5.1 Hz, 3H)

Step 4: A mixture of TRIETHYL ORTHOACETATE (18.9 eq. 5.5 mL. 30.0 mmol) in 5-amino-2-chloro-6-(4-chloro-2-fluoro-phenyl)-N-methyl-pyrimidine-4-carboxamide (1.00 eq. 500 mg, 1.59 mmol) was added TsOH (3.00 eq. 819 mg. 4.76 mmol). The mixture was stirred at 100° C., for 12 h. LCMS showed ˜34% of desired product was detected (34%, Rt=0.594 min: [M+H]⁺=339.0 at 220 nm). The mixture was quenched by 100 mL H₂O, extracted with EA (100 mL*3). The combined organic layers was dried over anhydrous Na₂SO₄, filtered and concentrated under reduced pressure to give the residue. The residue was purified by silical gel chromatography (PE:EA=1:1, the desired product Rf=0.3) to give the 6-chloro-8-(4-chloro-2-fluoro-phenyl)-2,3-dimethyl-pyrimido[5,4-d]pyrimidin-4-one (220 mg. 0.649 mmol. 40.88% yield) as white solid, the residue was used to the next step directly.

Step 5: To a solution of 6-chloro-8-(4-chloro-2-fluoro-phenyl)-2,3-dimethyl-pyrimido[5,4-d]pyrimidin-4-one (1.00 eq. 100 mg. 0.295 mmol) in DMSO (1 mL) was added (2S,6R)-2-(1-cyclopropylpyrazol-4-yl)-6-methyl-morpholine (1.00 eq, 61 mg, 0.295 mmol) and DIPEA (5.00 eq, 0.26 mL, 1.47 mmol). The mixture was stirred at 100° C., for 1 h. LCMS showed ˜85% of desired product was detected (85%, Rt=0.637 min: [M+H]⁺=510.1 at 220 nm). The reaction mixture was quenched by 10 mL H₂O, extracted with EA (20 mL*3), the combined organic layers was dried over anhydrous Na₂SO₄, filtered and concentrated under reduced pressure to give the residue. The residue was purified by Prep-HPLC (Phenomenex luna C18 150*25 mm*10 um, water (FA)-ACN) and lyophilized to gvie the 8-(4-chloro-2-fluoro-phenyl)-6-[(2S,6R)-2-(1-cyclopropylpyrazol-4-yl)-6-methyl-morpholin-4-yl]-2,3-dimethyl-pyrimido[5,4-d]pyrimidin-4-one (147 mg, 0.289 mmol, 98.00% yield) as yellow solid. [M+H]⁺=510.2; purity=99.2% (220 nm); Retention time=0.962 min. ¹H NMR (400 MHz, CHLOROFORM-d) δ=7.60 (t, J=7.8 Hz, 1H), 7.55 (d, J=2.2 Hz, 2H), 7.31-7.27 (m, 1H), 7.23 (dd, J=1.9, 9.6 Hz, 1H), 4.98-4.77 (m, 2H), 4.59 (dd, J=2.5, 10.9 Hz, 1H), 3.81 (ddd, J=2.4, 6.2, 10.5 Hz, 1H), 3.62 (s, 3H), 3.58 (td, J=3.6, 7.2 Hz, 1H), 3.04 (dd, J=11.1, 13.1 Hz, 1H), 2.81 (dd, J=10.9, 13.1 Hz, 1H), 2.52 (s, 3H), 1.33 (d, J=6.1 Hz, 3H), 1.16-1.09 (m, 2H), 1.05-0.98 (m, 2H).

### Example 22: Synthesis of Compound I-157: 2-[(2R)-2-(1-cyclopropyl-2-methyl-pyrazol-2-ium-4-yl)tetrahydropyran-4-yl]-4-(2,4-difluorophenyl)-7-methyl-pyrimido[4,5-d]pyridazin-8-one

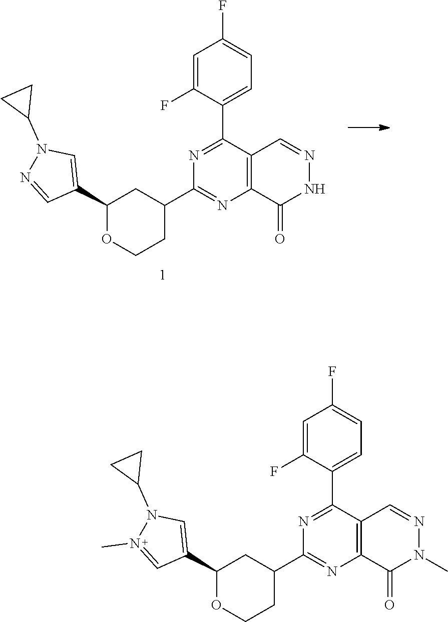

Step 1: A solution of 2-[(2R)-2-(1-cyclopropylpyrazol-4-yl)tetrahydropyran-4-yl]-4-(2,4-difluorophenyl)-7H-pyrimido[4,5-d]pyridazin-8-one (1.00 eq, 50 mg, 0.111 mmol) in DMF (1 mL) was added K₂CO₃ (3.00 eq, 46 mg, 0.333 mmol) and CH₃I (3.00 eq, 47 mg, 0.333 mmol) and stirred at 25° C., for 3 hours. LCMS showed raw material consumed and the major peak showed (M+H)+=465.1, purity=76.49%, uv=220 nm, Ret. Time=0.559 min and the weak peak showed (M+H)+=479.2, purity=7.6%, uv=220 nm, Ret. Time=0.468 min. The reaction was stirred for 16 h at 25° C. LCMS showed raw material consumed and the major peak showed (M+H)+=465.1, purity=43.37%, uv=220 nm, Ret. Time=0.560 min, and the weak peak showed (M+H)+=479.2, purity=33.61%, uv=220 nm, Ret. Time=0.465 min. The reaction solution pour into water (10 mL) and then extracted with ethyl actate (3 mL*3), the organics was washed with 5 mL saturated brine solution. Then the organics separated and dried (Na₂SO₄) before concentration to dryness and the residue purified by prep-HPLC (FA) (3_Phenomenex Luna C18 75*30 mm*3 um, water (FA)-ACN (15˜35)) and freeze-drying to give 2-[(2R)-2-(1-cyclopropyl-2-methyl-pyrazol-2-ium-4-yl)tetrahydropyran-4-yl]-4-(2,4-difluorophenyl)-7-methyl-pyrimido[4,5-d]pyridazin-8-one (0.92 mg, 0.00192 mmol, 1.73% yield) as light yellow solid which was confirmed by 2D NMR. (M+H)⁺=479.2, purity=100%, uv=220 nm, Ret. Time=0.773 min. ¹H NMR (400 MHz, CHLOROFORM-d) δ ppm 1.35 (br s, 2H) 1.43-1.50 (m, 2H) 2.01-2.21 (m, 3H) 2.42-2.55 (m, 1H) 3.58-3.66 (m, 1H) 3.73-3.84 (m, 1H) 3.88-3.97 (m, 4H) 4.23 (br dd, J=10.76, 3.75 Hz, 1H) 4.41 (s, 3H) 4.64 (br d, J=11.51 Hz, 1H) 7.01-7.08 (m, 1H) 7.18-7.24 (m, 1H) 7.77-7.85 (m, 1H) 7.98 (s, 1H) 8.05 (d, J=4.50 Hz, 1H) 8.56 (s, 1H)

### Example 23: Synthesis of Compound I-163: 6-((2S,6R)-2-(1-cyclopropyl-1H-pyrazol-4-yl)-6-methylmorpholino)-8-(2,4-difluorophenyl)-3-methylpyrimido[5,4-d]pyrimidin-4 (3H)-one

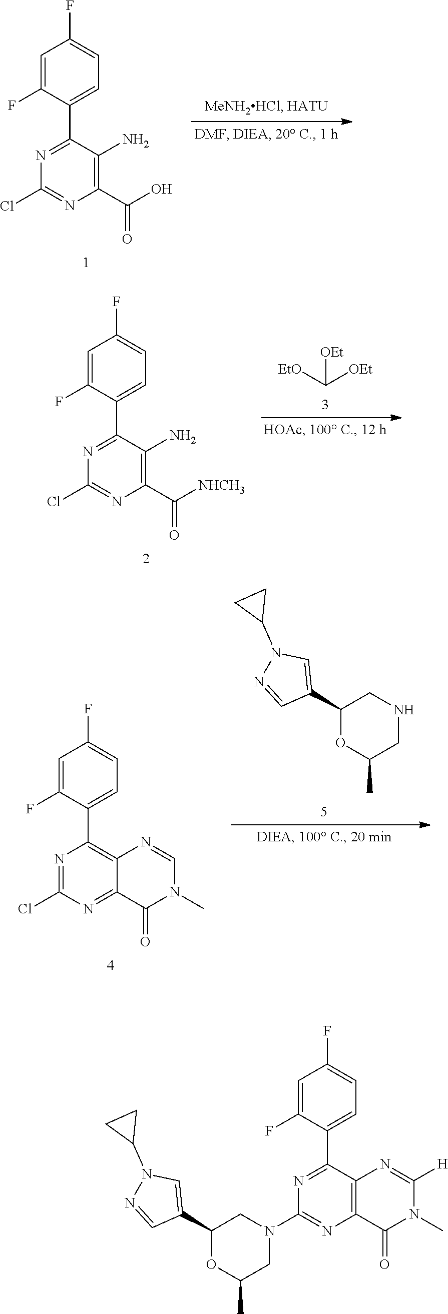 [SMILES: Nc1c(C(=O)O)nc(Cl)nc1-c1ccc(F)cc1F ; CNC(=O)c1nc(Cl)nc(-c2ccc(F)cc2F)c1N ; CCOC(OCC)OCC ; Cn1cnc2c(-c3ccc(F)cc3F)nc(Cl)nc2c1=O ; C[C@@H]1CNC[C@H](c2cnn(C3CC3)c2)O1 ; C[C@@H]1CN(c2nc(-c3ccc(F)cc3F)c3ncn(C)c(=O)c3n2)C[C@H](c2cnn(C3CC3)c2)O1]

Step 1: A mixture of 5-amino-2-chloro-6-(2,4-difluorophenyl)pyrimidine-4-carboxylic acid (1.00 eq, 1000 mg, 3.50 mmol) in DMF (20 mL) was added DIPEA (8.00 eq, 4.9 mL, 28.0 mmol). The mixture was stirred for 5 mins. The mixture was added HATU (2.00 eq, 2662 mg, 7.00 mmol) and MeNH₂·HCl (5.00 eq, 1182 mg, 17.5 mmol). The mixture was stirred at 20° C., for 1 h. LCMS showed that the starting material was consumed completely and the desired mass was detected (17%, Rt: 0.602 min; [M+H]⁺=299.1 at 220 nm). The mixture was diluted with water (30 mL) and extracted with EtOAc (40 mL) twice. The combined organic layers were washed with an aqueous solution with brine (50 mL) and dried over Na₂SO₄. The solvent was filtered and concentrated under reduced pressure. The residue was purified by prep-TLC (SiO₂, PE/EtOAc=2/1: the desired product R (=0.3) to give 5-amino-2-chloro-6-(2,4-difluorophenyl)-N-methylpyrimidine-4-carboxamide (600 mg, 2.01 mmol, 57.38% yield) as solid, checked by HNMR. [M+H]⁺=299.1: ¹H NMR (400 MHz, CDCl₃) δ=8.06-7.95 (m, 1H), 7.57-7.51 (m, 1H), 7.10-6.98 (m, 2H), 6.14 (br s, 2H), 3.02 (d, J=5.2 Hz, 3H)

Step 2: A mixture of 5-amino-2-chloro-6-(2,4-difluorophenyl)-N-methylpyrimidine-4-carboxamide (1.00 eq, 100 mg, 0.335 mmol) in triethoxymethane (18.9 eq, 1.1 mL, 6.33 mmol) was added TsOH (3.00 eq, 173 mg, 1.00 mmol). The mixture was stirred at 100° C., for 12 h. LCMS showed raw material remained and 70% of the desired mass was detected (70%, Rt: 0.603 min: [M+H]⁺=308.9 at 220 nm). The reaction mixture was quenched by addition saturated NaHCO₃ (10 mL) at 0° C., and then extracted with EtOAc (20 mL×2). The combined organic layers were washed with saturated NaCl solution (10 mL), dried over Na₂SO₄, filtered and concentrated under reduced pressure to give a residue. The crude product was purified by column chromatography on silica gel eluted (PE/EtOAc=I/O to 1/2; PE/EtOAc=1/1, the desired product Rf=0.5 showed by 254 nm) to 6-chloro-8-(2,4-difluorophenyl)-3-methylpyrimido[5,4-d]pyrimidin-4 (3H)-one (70 mg. 0.227 mmol, 67.73% yield) as solid, checked by HNMR and LCMS. [M+H]⁺=309.0; purity=94% (220 nm); Retention time=0.518 min. ¹H NMR (400 MHz, CDCl₃) δ=8.13 (s, 1H), 7.72 (dt, J=6.4, 8.3 Hz, 1H), 7.14-6.95 (m, 2H), 3.70 (s, 3H)

Step 3: To a solution of 6-chloro-8-(2,4-difluorophenyl)-3-methylpyrimido[5,4-d]pyrimidin-4 (3H)-one (1.00 eq. 60 mg. 0.194 mmol) and (2S,6R)-2-(1-cyclopropylpyrazol-4-yl)-6-methyl-morpholine (1.00 eq, 40 mg, 0.194 mmol) in DMSO (6 mL) was added DIEA (5.00 eq. 0.16 mL, 0.972 mmol) and the mixture was stirred at 100° C., for 20 min. LCMS showed the starting material was consumed completely and 96% of the desired mass was detected (96%, Rt: 0.602 min: [M+H]⁺=480.4 at 220 nm). The reaction mixture was quenched by addition water (20 mL), and then extracted with EtOAc (20 mL×2). The combined organic layers were washed with saturated NaCl (20 mL), dried over Na₂SO₄, filtered and concentrated under reduced pressure to give a residue. The crude product was purified by column chromatography on silica gel eluted (PE/EtOAc=I/O to 1/2; PE/EtOAc=1/1, the desired product Rf=0.5 showed by 254 nm) to 6-((2S,6R)-2-(1-cyclopropyl-1H-pyrazol-4-yl)-6-methylmorpholino)-8-(2,4-difluorophenyl)-3-methylpyrimido[5,4-d]pyrimidin-4 (3H)-one (21 mg, 0.0436 mmol, 22.42% yield) as yellow solid, checked by LCMS, HPLC, HNMR, FNMR. [M+H]⁺=480.2; purity=99.5% (220 nm); Retention time=0.696 min. ¹H NMR (400 MHz, CDCl₃) δ=7.81 (s, 1H), 7.69-7.59 (m, 1H), 7.55 (br s, 2H), 7.08-7.01 (m, 1H), 7.00-6.93 (m, 1H), 5.04-4.79 (m, 2H), 4.59 (dd, J=2.0, 10.8 Hz, 1H), 3.89-3.73 (m, 1H), 3.66-3.52 (m, 4H), 3.06 (br t, J=12.0 Hz, 1H), 2.89-2.73 (m, 1H), 1.33 (d, J=6.3 Hz, 3H), 1.12 (br d, J=2.9 Hz, 2H), 1.06-0.98 (m, 2H)

### Example 24: Synthesis of Compound I-183 and I-184: 4-(4-chloro-2-fluoro-phenyl)-2-[(2R,6S)-2-cyclopropyl-6-(1-cyclopropylpyrazol-4-yl) morpholin-4-yl]-7-methyl-pyrimido[4,5-d]pyridazin-8-one (1-183) and 4-(4-chloro-2-fluoro-phenyl)-2-[(2S,6R)-2-cyclopropyl-6-(1-cyclopropylpyrazol-4-yl) morpholin-4-yl]-7-methyl-pyrimido[4,5-d]pyridazin-8-one (I-184)

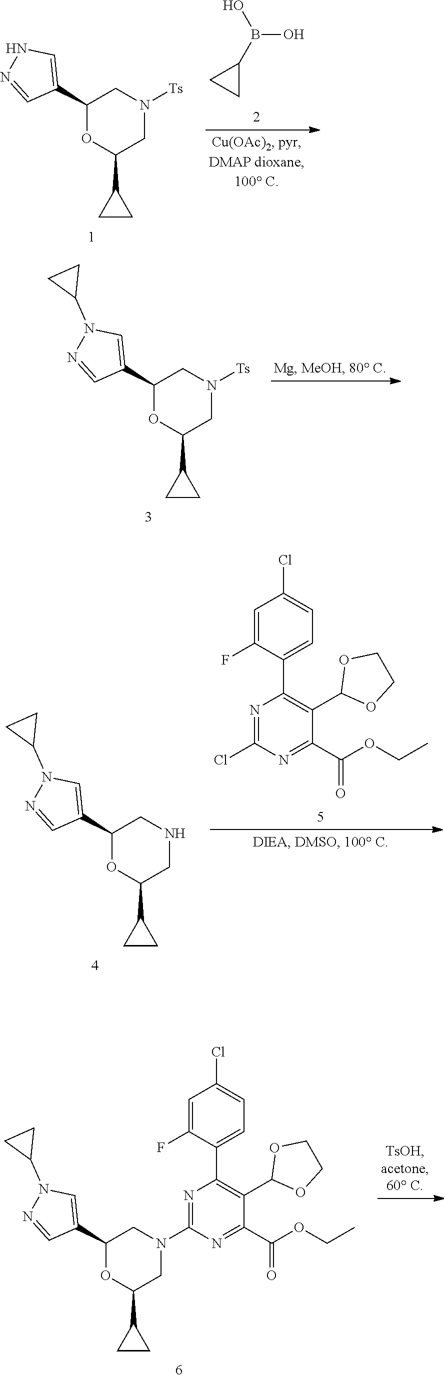

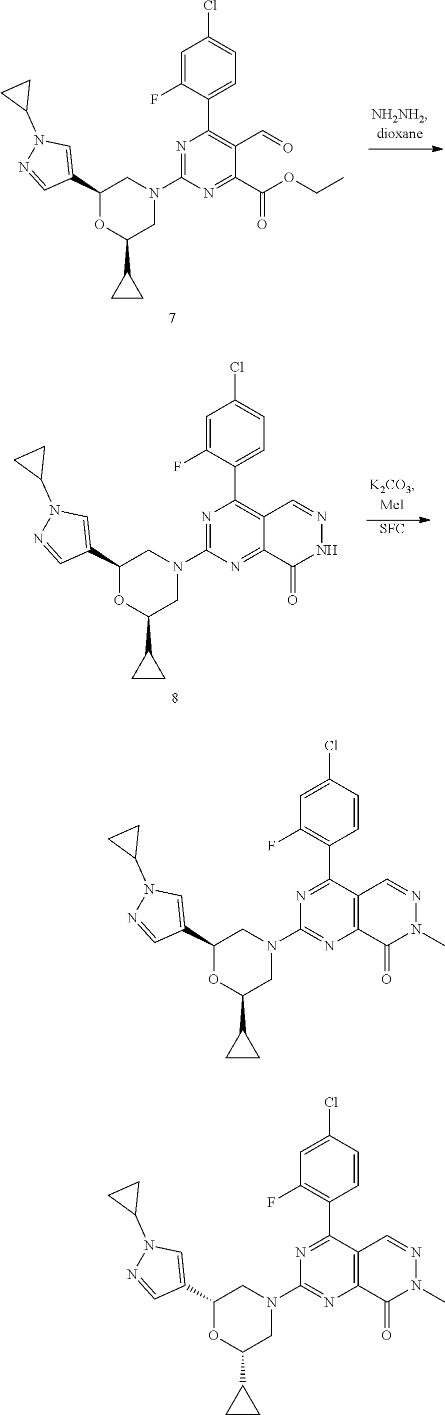

Step 1: A mixture of (2R,6S)-2-cyclopropyl-4-(p-tolylsulfonyl)-6-(1H-pyrazol-4-yl) morpholine (1.00 eq, 850 mg, 2.45 mmol), cyclopropylboronic acid (2.00 eq, 420 mg, 4.89 mmol), DMAP (4.00 eq, 1194 mg, 9.79 mmol), Pyridine (2.50 eq, 0.49 mL, 6.12 mmol) and Cu(OAc) 2 (1.00 eq, 444 mg, 2.45 mmol) in 1,4-Dioxane (60 mL) was stirred at 100° C., for 12 h under 02. LCMS showed ˜94% of desired product was detected (94%, Rt=0.653 min; [M+H]⁺=388.2 at 220 nm). The reaction was quenched by 250 mL H₂O, extracted with EA (150 mL*3), the combined organic layers was dried over anhydrous Na₂SO₄, filtered and concentrated under reduced pressure to give the residue. The residue was purified by flash silica gel chromatography (Eluent of 0˜50% Ethyl acetate/Petroleum ether) (TLC:EA=1:1, Rf=0.4) to give (2R,6S)-2-cyclopropyl-6-(1-cyclopropylpyrazol-4-yl)-4-(p-tolylsulfonyl) morpholine (940 mg, 2.43 mmol, 99.15% yield) as yellow oil, checked by H NMR and LCMS. [M+H]⁺=388.2; purity=100% (220 nm); Retention time=0.654 min. ¹H NMR (400 MHz, CHLOROFORM-d) δ=7.64 (d, J=8.3 Hz, 2H), 7.39 (d, J=5.1 Hz, 2H), 7.35 (d, J=8.0 Hz, 2H), 4.54 (dd, J=2.6, 10.6 Hz, 1H), 3.71 (tdd, J=2.0, 11.3, 19.5 Hz, 2H), 3.52 (tt, J=3.8, 7.3 Hz, 1H), 2.98 (ddd, J=2.5, 8.2, 10.4 Hz, 1H), 2.45 (s, 3H), 2.21 (t, J=11.0 Hz, 2H), 1.06 (br dd, J=1.1, 3.8 Hz, 2H), 1.03-0.95 (m, 2H), 0.84-0.72 (m, 1H), 0.60-0.48 (m, 2H), 0.45-0.37 (m, 1H), 0.34-0.26 (m, 1H)

Step 2: To a mixture of (2R)-2-cyclopropyl-6-(1-cyclopropylpyrazol-4-yl)-4-(p-tolylsulfonyl) morpholine (1.00 eq. 940 mg. 2.43 mmol) in Methanol (20 mL) was added Mg (Chips) (10.0 eq. 582 mg, 24.3 mmol) and Mg (powder) (10.0 eq, 582 mg, 24.3 mmol), then the mixture was stirred at 80° C., for 12 h under N₂ atmosphere to give white solution. LCMS showed 16% of starting material was still remained. Then the mixture was added Mg (Chips) (10.0 eq, 582 mg, 24.3 mmol) and stirred at 80° C. for 12 h under N₂ atmosphere. LCMS showed ˜82% of desired product was detected (82%, Rt=0.494 & 0.539 min: [M+H]⁺=234.1 at 220 nm). The reaction mixture was cooled to room temperature. The mixture was filtered and the filter cake was washed with MeOH (30 mL*3), the combined organic layers was concentrated under reduced pressure to give the (2R)-2-cyclopropyl-6-(1-cyclopropylpyrazol-4-yl) morpholine (900 mg, 1.93 mmol, 79.51% yield) as yellow oil. [M+H]⁺=234.1 at 220 nm, 82%, Rt=0.494 & 0.539 min.

Step 3: To a solution of (2R,6S)-2-cyclopropyl-6-(1-cyclopropylpyrazol-4-yl) morpholine (1.00 eq, 676 mg, 1.45 mmol) in DMSO (15 mL) was added ethyl 2-chloro-6-(4-chloro-2-fluoro-phenyl)-5-(1,3-dioxolan-2-yl)pyrimidine-4-carboxylate (1.00 eq. 850 mg, 1.45 mmol), DIEA (3.00 eq, 0.76 mL, 4.35 mmol) and stirred at 100° C., for 1 h. LCMS showed ˜41% of desired product was detected (41%, Rt=0.713 min: [M+H]⁺=584.3 at 220 nm). The reaction was quenched by 100 mL H₂O, extracted with EA (40 mL*3), the combined organic layers was dried over anhydrous Na₂SO₄, filtered and concentrated under reduced pressure to give the residue. The residue was purified by Flash Column (PE:EA=0˜50%, PE:EA=1:1, the desired product Rf=0.5) to afford ethyl 6-(4-chloro-2-fluoro-phenyl)-2-[(2R,6S)-2-cyclopropyl-6-(1-cyclopropylpyrazol-4-yl) morpholin-4-yl]-5-(1,3-dioxolan-2-yl)pyrimidine-4-carboxylate (600 mg, 1.03 mmol, 70.90% yield) as yellow oil, checked by LCMS and H NMR. [M+H]⁺=584.2; purity=86.86% (220 nm); Retention time=0.670 min. ¹H NMR (400 MHz, CHLOROFORM-d) δ=7.50 (br d, J=3.1 Hz, 2H), 7.39 (t, J=7.8 Hz, 1H), 7.24-7.15 (m, 2H), 5.65 (s, 1H), 4.74 (br d, J=10.3 Hz, 2H), 4.40 (br d, J=7.1 Hz, 3H), 3.87-3.79 (m, 3H), 3.59-3.49 (m, 1H), 2.89 (br d, J=8.1 Hz, 3H), 1.69-1.62 (m, 1H), 1.40 (t, J=7.2 Hz, 3H), 1.10 (br s, 2H), 0.99 (br d, J=6.5 Hz, 3H), 0.66-0.51 (m, 2H), 0.50-0.39 (m, 1H), 0.31 (br dd, J=4.1, 8.6 Hz, 1H)

Step 4: A mixture of ethyl 6-(4-chloro-2-fluoro-phenyl)-2-[(2R,6S)-2-cyclopropyl-6-(1-cyclopropylpyrazol-4-yl) morpholin-4-yl]-5-(1,3-dioxolan-2-yl)pyrimidine-4-carboxylate (1.00 eq, 550 mg, 0.942 mmol) in Acetone (10 mL) was added TsOH (0.200 eq, 32 mg, 0.188 mmol). The mixture was stirred at 60° C., for 1 h. LCMS showed ˜71% of desired product was detected (71%, Rt=0.711 min; [M+H]⁺=540.2 at 220 nm). The reaction was quenched by 80 mL H₂O, extracted with EA (30 mL*3), the combined organic layers was dried over anhydrous Na₂SO₄, filtered and concentrated under reduced pressure to give the residue. The residue was purified by Flash Column (PE:EA=0˜50%, PE:EA=1:1, the desired product Rf=0.5) to afford ethyl 6-(4-chloro-2-fluoro-phenyl)-2-[(2R,6S)-2-cyclopropyl-6-(1-cyclopropylpyrazol-4-yl) morpholin-4-yl]-5-formyl-pyrimidine-4-carboxylate (490 mg, 0.907 mmol, 96.36% yield) as yellow oil, checked by LCMS and H NMR. [M+H]⁺=540.2; purity=84.96% (220 nm); Retention time=0.714 min. ¹H NMR (400 MHz, CHLOROFORM-d) δ=9.65 (d, J=4.3 Hz, 1H), 7.52 (br d, J=4.2 Hz, 2H), 7.49-7.42 (m, 1H), 7.31 (br dd, J=7.6, 18.1 Hz, 1H), 7.24-7.19 (m, 1H), 5.01-4.84 (m, 2H), 4.56-4.40 (m, 3H), 3.70 (s, 2H), 3.55 (br dd, J=3.8, 7.2 Hz, 1H), 3.13-2.89 (m, 4H), 1.13-1.00 (m, 4H), 0.65-0.42 (m, 4H), 0.40-0.26 (m, 1H)

Step 5: A mixture of ethyl 6-(4-chloro-2-fluoro-phenyl)-2-[(2R,6S)-2-cyclopropyl-6-(1-cyclopropylpyrazol-4-yl) morpholin-4-yl]-5-formyl-pyrimidine-4-carboxylate (1.00 eq, 440 mg, 0.815 mmol) in 1,4-Dioxane (9 mL) was added hydrazine; hydrate (2.00 eq, 82 mg, 1.63 mmol). The mixture was stirred at 90° C., for 2 h. LCMS showed ˜72% of desired product was detected (72%, Rt=0.578 min; [M+H]⁺=508.2 at 220 nm). The reaction was quenched by 80 mL H₂O, extracted with EA (30 mL*3), the combined organic layers was dried over anhydrous Na₂SO₄, filtered and concentrated under reduced pressure to give the 4-(4-chloro-2-fluoro-phenyl)-2-[(2R,6S)-2-cyclopropyl-6-(1-cyclopropylpyrazol-4-yl) morpholin-4-yl]-7H-pyrimido[4,5-d]pyridazin-8-one (300 mg, 0.591 mmol, 72.48% yield) as yellow solid, checked by LCMS and H NMR. [M+H]⁺=508.2 at 220 nm, 72%, Rt=0.578 min: ¹H NMR (400 MHz, CHLOROFORM-d) δ=10.46-10.30 (m, 1H), 7.79 (d, J=3.7 Hz, 1H), 7.53 (br s, 3H), 7.43-7.29 (m, 2H), 5.17-4.84 (m, 2H), 4.55-4.40 (m, 1H), 3.12-2.95 (m, 3H), 2.04 (s, 1H), 1.07-0.83 (m, 5H), 0.64-0.45 (m, 4H)

Step 6: A mixture of 4-(4-chloro-2-fluoro-phenyl)-2-[2-cyclopropyl-6-(1-cyclopropylpyrazol-4-yl) morpholin-4-yl]-7H-pyrimido[4,5-d]pyridazin-8-one (1.00 eq. 300 mg. 0.591 mmol) and K₂CO₃ (2.00 eq, 163 mg, 1.18 mmol) in DMF (5 mL) was added Mel (3.00 eq, 0.11 mL, 1.77 mmol). The mixture was stirred at 50° C., for 2 h. LCMS showed ˜18% of desired product was detected (18%, Rt=0.627 min: [M+H]⁺=522.2 at 220 nm). Then the mixture was added Mel (3.00 eq, 0.11 mL, 1.77 mmol) and stirred at 50° C., for 2 h. LCMS showed ˜45% of desired product was detected (45%, Rt=0.637 min: [M+H]⁺=522.2 at 220 nm). The reaction was quenched by 30 mL H₂O, extracted with EA (15 mL*3), the combined organic layers was dried over anhydrous Na₂SO₄, filtered and concentrated under reduced pressure to give the residue. The residue was purified by prep-HPLC (flow: 100 mL/min; gradient: from 1-30% Hexane-EtOH over 15 min; column: Welch Ultimate XB—CN 250*50*10 um) and lyophilized to afford 4-(4-chloro-2-fluoro-phenyl)-2-[2-cyclopropyl-6-(1-cyclopropylpyrazol-4-yl) morpholin-4-yl]-7-methyl-pyrimido[4,5-d]pyridazin-8-one (54 mg, 0.0993 mmol, 16.82% yield). The product was purified by SFC (DAICEL CHIRALPAK AD (250 mm*30 mm, 10 um), Gradient elution: IPA-ACN from 45% to 45%, Flow rate: 70 mL/min) and lyophilized to afford the 4-(4-chloro-2-fluoro-phenyl)-2-[(2R,6S)-2-cyclopropyl-6-(1-cyclopropylpyrazol-4-yl) morpholin-4-yl]-7-methyl-pyrimido[4,5-d]pyridazin-8-one (23 mg. 0.0419 mmol, 7.10% yield) as yellow solid and the 4-(4-chloro-2-fluoro-phenyl)-2-[(2S,6R)-2-cyclopropyl-6-(1-cyclopropylpyrazol-4-yl) morpholin-4-yl]-7-methyl-pyrimido[4,5-d]pyridazin-8-one (14 mg, 0.0248 mmol, 4.20% yield) as yellow solid.

(P1, single enantiomer of unknown absolute configuration): peak 1 in SFC test, [M+H]⁺=522.3; purity=98.15% (220 nm); Retention time=0.681 min. HPLC: Retention time=2.633 min, 97.38% purity at 220 nm. ¹H NMR (400 MHz, CHLOROFORM-d) δ=7.76 (d, J=3.9 Hz, 1H), 7.53 (br s, 3H), 7.42-7.29 (m, 2H), 5.15 (br s, 1H), 4.91 (br d, J=11.5 Hz, 1H), 4.48 (br s, 1H), 3.82 (br s, 3H), 3.57 (br s, 1H), 3.18-2.90 (m, 3H), 1.16-0.95 (m, 5H), 0.67-0.41 (m, 4H)

(P2, single enantiomer of unknown absolute configuration): peak 2 in SFC test, [M+H]⁺=522.3; purity=97.17% (220 nm); Retention time=0.680 min. HPLC: Retention time=2.635 min, 92.81% purity at 220 nm. ¹H NMR (400 MHz, CHLOROFORM-d) ¿=7.76 (d, J=3.9 Hz, 1H), 7.60-7.45 (m, 3H), 7.42-7.27 (m, 2H), 5.23-5.07 (m, 1H), 4.98-4.85 (m, 1H), 4.48 (br s, 1H), 3.83 (br s, 3H), 3.56 (br d, J=1.1 Hz, 1H), 3.18-2.91 (m, 3H), 1.14-0.97 (m, 5H), 0.66-0.41 (m, 4H)

### Example 25: Synthesis of Compound I-188: 8-(4-chloro-2-fluoro-phenyl)-2,3-dimethyl-6-[(2R)-2-(2-methyl-4-pyridyl)tetrahydropyran-4-yl]pyrimido[5, 4-d]pyrimidin-4-one

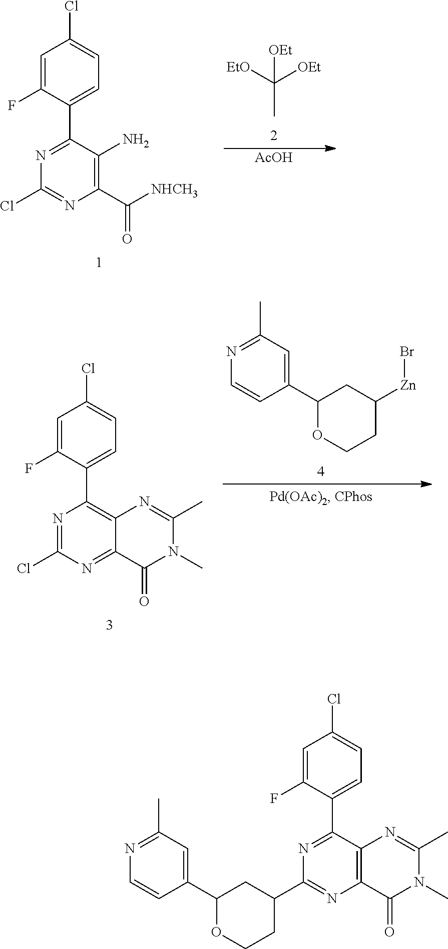

Step 1: A mixture of 5-amino-2-chloro-6-(4-chloro-2-fluoro-phenyl)-N-methyl-pyrimidine-4-carboxamide (1.00 eq, 200 mg, 0.635 mmol) in TRIETHYL ORTHOACETATE (18.9 eq, 2.2 mL, 12.0 mmol) was added AcOH (3.00 eq, 263 mg, 1.90 mmol). The mixture was stirred at 100° C., for 16 h. LCMS showed the starting material was consumed completely and a major peak with desired MS (LCMS: (M+H)+=339.0; purity=95.57% (UV 220 nm); Retention time=0.527 min) was detected. The solution was added 10 mL water and extracted with EtOAc (10 mL*3). The mixture was added silica gel and concentrated under reduced process to give the residue. The residue was purified by flash column (PE:EtOAc=1:1; UV, Rf=0.5) and concentrated under vacuum to give 6-chloro-8-(4-chloro-2-fluoro-phenyl)-2,3-dimethyl-pyrimido[5,4-d]pyrimidin-4-one (180 mg, 0.531 mmol, 83.63% % yield) as white solid. (M+H)+=339.0; purity=100% (UV 220 nm); Retention time=0.876 min. ¹H NMR (400 MHz, CHLOROFORM-d) δ=7.68-7.58 (m, 1H), 7.35-7.27 (m, 2H), 7.23-7.21 (m, 1H), 3.66 (s, 2H), 3.74-3.56 (m, 1H), 2.58 (s, 3H).

Step 2: To a suspension of 6-chloro-8-(4-chloro-2-fluoro-phenyl)-2,3-dimethyl-pyrimido[5,4-d]pyrimidin-4-one (1.00 eq, 180 mg, 0.531 mmol) and CPhos (0.1000 eq, 23 mg, 0.0531 mmol) in THF (3 mL) (99.5%, Extra Dry over Molecular Sieve, Stabilized, Acros) was added PALLADIUM (II) ACETATE (0.0500 eq, 6.0 mg. 0.0265 mmol), followed by bromo-[(2R)-2-(2-methyl-4-pyridyl)tetrahydropyran-4-yl]zinc (1.20 eq, 205 mg. 0.637 mmol) and the mixture was then stirred at 55° C., for 2 h. LCMS showed ˜27.9% of desired mass, the reaction solution was poured into H₂O (10 mL), extracted with EtOAc (10 mL*3), dried over Na₂SO₄ and evaporated under reduced pressure to give the residue, which was then purified with Prep-HPLC (FA) and lyophilized to give 8-(4-chloro-2-fluoro-phenyl)-2,3-dimethyl-6-[(2R)-2-(2-methyl-4-pyridyl)tetrahydropyran-4-yl]pyrimido[5,4-d]pyrimidin-4-one (120 mg, 0.250 mmol, 47.11% yield) as yellow solid. This product was purified by prep-HPLC (FA, Column; phenomenex luna C18 150*25 mm*10 μm, the condition was water (FA)-ACN; Gradient Time (min): 10; Flow Rate (ml/min): 25) and lyophilized to give 8-(4-chloro-2-fluoro-phenyl)-2,3-dimethyl-6-[2-(2-methyl-4-pyridyl)tetrahydropyran-4-yl]pyrimido[5,4-d]pyrimidin-4-one (20 mg, 0.0416 mmol) as white solid. (M+H)+=480.2; purity=99.76% (UV 220 nm); Retention time=0.457 min. ¹H NMR (400 MHz, CHLOROFORM-d) δ=8.49-8.40 (m, 1H), 7.72-7.57 (m, 1H), 7.35-7.27 (m, 2H), 7.26-7.20 (m, 2H), 7.18-7.07 (m, 1H), 4.92-4.81 (m, 1H), 4.57-4.44 (m, 1H), 4.40-4.28 (m, 1H), 4.03-3.96 (m, 1H), 3.89-3.75 (m, 1H), 3.75-3.71 (m, 1H), 3.66 (br s, 3H), 3.65-3.57 (m, 1H), 2.81-2.69 (m, 1H), 2.66-2.53 (m, 6H), 2.49-2.41 (m, 1H), 2.36-2.29 (m, 1H), 2.28-2.17 (m, 1H), 2.14-2.12 (m, 1H), 2.01-1.89 (m, 1H). SFC: 4 peaks, ratio ˜1:1:1:1.

### Example 26: Synthesis of Compound I-193: 4-(4-chloro-2-fluorophenyl)-2-((2S, 6R)-2-(1-cyclopropyl-1H-pyrazol-4-yl)-6-methylmorpholino)-7-ethyl-pyrimido[4, 5-d]pyridazin-8 (7H)-one

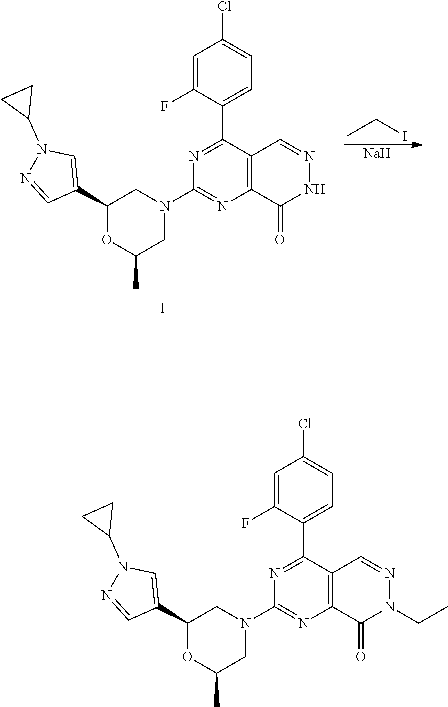

Step 1: A mixture of 4-(4-chloro-2-fluoro-phenyl)-2-[(2S, 6R)-2-(1-cyclopropylpyrazol-4-yl)-6-methyl-morpholin-4-yl]-7H-pyrimido[4, 5-d]pyridazin-8-one (1.00 eq, 100 mg, 0.208 mmol) in THF (5 mL) was added NaH (3.00 eq, 25 mg, 0.623 mmol). The mixture was stirred at 25° C. for 0.5 h, then EtI (3.00 eq, 0.050 mL, 0.623 mmol) was added. The mixture was stirred at 25° C., for 2 h. LCMS showed 64.49% material was remained. Then the solution was heated to 50° C., and stirred for 12 h. LCMS (PJ-2022-01-080-54-P1A1) showed the starting material was consumed completely and a major peak with desired MS was detected. (LCMS: (M+H)+=510.2; purity=43.87% (UV 220 nm); Retention time=0.637 min). The mixture was added 10 mL water and extracted with EtOAc (10 mL*3). The mixture was purified by prep-HPLC (FA, Column: phenomenex luna C18 150*25 mm*10 μm, the condition was water (FA)-ACN; Gradient Time (min): 10; Flow Rate (ml/min): 25) and lyophilized to give 4-(4-chloro-2-fluoro-phenyl)-2-[(2S, 6R)-2-(1-cyclopropylpyrazol-4-yl)-6-methyl-morpholin-4-yl]-7-ethyl-pyrimido[4,5-d]pyridazin-8-one (14 mg, 0.0281 mmol, 13.54% yield) as yellow solid. (M+H)+=510.2; purity=99.49% (UV 220 nm); Retention time=0.831 min. ¹H NMR (400 MHz, DMSO-d6) δ=7.94-7.80 (m, 2H), 7.78-7.63 (m, 2H), 7.60-7.42 (m, 1H), 7.58-7.41 (m, 1H), 4.94-4.63 (m, 2H), 4.60-4.47 (m, 1H), 4.11 (br s, 2H), 3.71 (br d, J=4.6 Hz, 2H), 3.25-3.06 (m, 1H), 2.76 (s, 1H), 1.32-1.16 (m, 6H), 1.08-0.89 (m, 4H). SFC: 100% ee.

### Example 27: Synthesis of Compound I-198: 2-[(2S, 6R)-2-(1-cyclopropylpyrazol-4-yl)-6-methyl-morpholin-4-yl]-7-methyl-4-[6-(trifluoromethyl)-3-pyridyl]pyrimido[4,5-d]pyridazin-8-one

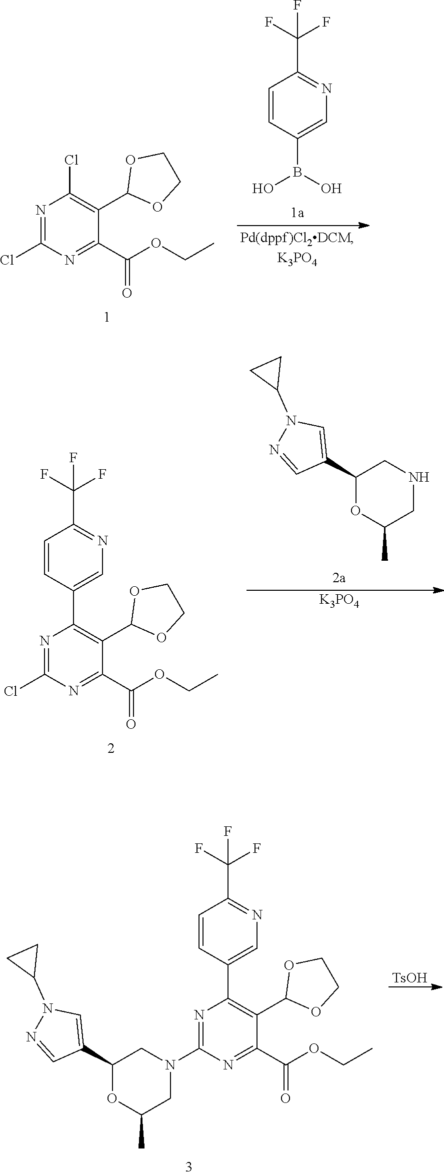

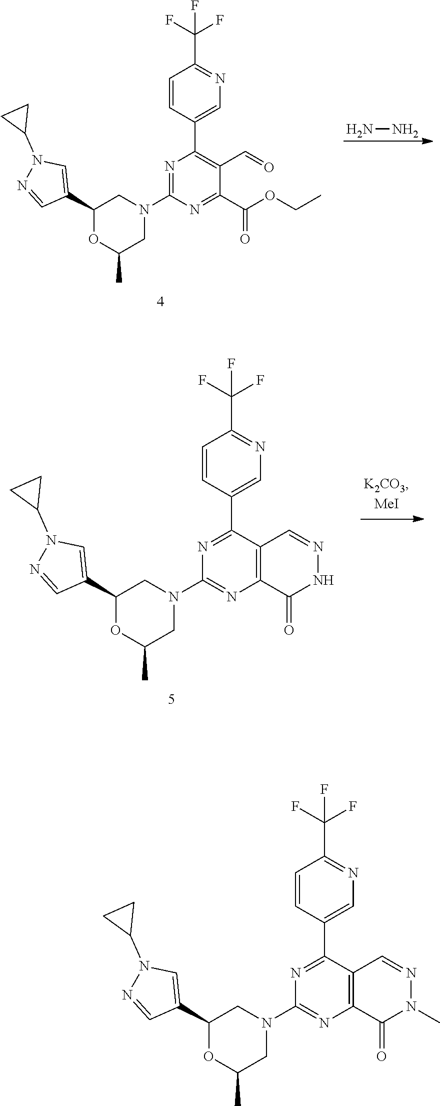

Step 1: A mixture of ethyl 2,6-dichloro-5-(1,3-dioxolan-2-yl)pyrimidine-4-carboxylate (1.00 eq, 500 mg, 1.71 mmol) and [6-(trifluoromethyl)-3-pyridyl]boronic acid (0.950 eq, 309 mg, 1.62 mmol) in 1,4-Dioxane (10 mL) and Water (1 mL) was added K₃PO₄ (2.00 eq, 724 mg, 3.41 mmol) and Pd(dppf)Cl₂·DCM (0.1000 eq, 125 mg, 0.171 mmol). The mixture was degassed with N₂ for 3 times and heated to 60° C., and stirred for 2 h. The reaction mixture was brown solution. LCMS showed a major peak with desired MS (LCMS: (M+H)+=404.1; purity=58.07% (UV 220 nm); Retention time=0.590 min) and 20.36% of ethyl5-(1,3-dioxolan-2-yl)-6-(6-(trifluoromethyl) pyridin-3-yl)-2-(2-(trifluoromethyl) pyridin-4-yl)pyrimidine-4-carboxylate was detected. The crude was purified by flash column (PE:EtOAc=5:1; UV) and concentrated to give ethyl 2-chloro-5-(1, 3-dioxolan-2-yl)-6-[6-(trifluoromethyl)-3-pyridyl]pyrimidine-4-carboxylate (0.37 g, 0.916 mmol, 53.72% yield) as light orange solid. (M+H)+=404.1; purity=97.23% (UV 220 nm); Retention time=0.586 min.

Step 2: A mixture of ethyl 2-chloro-5-(1,3-dioxolan-2-yl)-6-[6-(trifluoromethyl)-3-pyridyl]pyrimidine-4-carboxylate (1.00 eq. 350 mg, 0.867 mmol) and (2S,6R)-2-(1-cyclopropylpyrazol-4-yl)-6-methyl-morpholine (1.20 eq, 215.62 mg. 0.0600 mmol) in 1,4-Dioxane (9 mL) was added K₃PO₄ (2.00 eq. 368 mg, 1.73 mmol). The mixture was heated to 100° C., and stirred for 2 h. LCMS showed 21.96% material was remained and a peak with desired MS (LCMS: (M+H)+=575.3; purity=75.03% (UV 220 nm); Retention time=0.651 min) was detected. Then the solution was stirred at 100° C., for 12 h. LCMS showed the material was no remained and a major peak with desired MS (LCMS: (M+H)+=575.2; purity=77.78% (UV 220 nm); Retention time=0.847 min) was detected. The mixture was concentrated under vacuum to give a crude. The crude was purified by flash column (PE:EtOAc=1:1; UV, Rf=0.3) to give ethyl 2-[(2S,6R)-2-(1-cyclopropylpyrazol-4-yl)-6-methyl-morpholin-4-yl]-5-(1,3-dioxolan-2-yl)-6-[6-(trifluoromethyl)-3-pyridyl]pyrimidine-4-carboxylate (0.52 mg, 0.000887 mmol, 0.1000% yield) as orange solid. (M+H)+=575.4; purity=97.98% (UV 220 nm); Retention time=0.657 min.

Step 3: A mixture of ethyl 2-[(2S, 6R)-2-(1-cyclopropylpyrazol-4-yl)-6-methyl-morpholin-4-yl]-5-(1, 3-dioxolan-2-yl)-6-[6-(trifluoromethyl)-3-pyridyl]pyrimidine-4-carboxylate (1.00 eq. 500 mg, 0.870 mmol) in Acetone (13 mL) was added TsOH (0.200 eq, 30 mg, 0.174 mmol). The mixture was stirred at 60° C., for 1 h. LCMS showed the starting material was consumed completely and a major peak with desired MS (LCMS: (M+H)+=531.3; purity=97.67% (UV 220 nm); Retention time=0.629 min) was detected. The mixture was concentrated under vacuum to give ethyl 2-[(2S, 6R)-2-(1-cyclopropylpyrazol-4-yl)-6-methyl-morpholin-4-yl]-5-formyl-6-[6-(trifluoromethyl)-3-pyridyl]pyrimidine-4-carboxylate (0.48 g, 0.877 mmol, 100.76% yield) as yellow solid (crude). (M+H)+=531.2; purity=96.91% (UV 220 nm); Retention time=0.629 min).

Step 4: A mixture of ethyl 2-[(2S, 6R)-2-(1-cyclopropylpyrazol-4-yl)-6-methyl-morpholin-4-yl]-5-formyl-6-[6-(trifluoromethyl)-3-pyridyl]pyrimidine-4-carboxylate (1.00 eq, 430 mg, 0.811 mmol) in 1, 4-Dioxane (4.5 mL) was added hydrazine; hydrate (2.00 eq, 81 mg, 1.62 mmol). The mixture was stirred at 90° C., for 2 h. LCMS showed a major peak with desired MS (LCMS: (M+H)+=499.2; purity=81.61% (UV 220 nm); Retention time=0.698 min) was detected. The mixture was added silica gel and concentrated under reduced process to give the residue. The residue was purified by flash column (EtOAc; and DCM:MeOH=10:1) and concentrated under vacuum to give 2-[(2S,6R)-2-(1-cyclopropylpyrazol-4-yl)-6-methyl-morpholin-4-v1]-4-[6-(trifluoromethyl)-3-pyridyl]-7H-pyrimido[4,5-d]pyridazin-8-onc (366 mg. 0.629 mmol, 77.54% yield) as yellow solid. (M+H)+=499.2; purity=99.67% (UV 220 nm); Retention time=0.527 min.

Step 5: To a solution of 2-[(2S,6R)-2-(1-cyclopropylpyrazol-4-yl)-6-methyl-morpholin-4-yl]-4-[6-(trifluoromethyl)-3-pyridyl]-7H-pyrimido[4,5-d]pyridazin-8-one (1.00 eq, 150 mg, 0.301 mmol) and K₂CO₃ (2.00 eq, 83 mg, 0.602 mmol) in DMF (7.5 mL) was added Mel (3.00 eq, 0.056 mL, 0.903 mmol). The solution was stirred at 50° C., for 2 h. LCMS showed the material was consumed completely and a major peak with desired ms (LCMS: (M+H)+=513.2; purity=89.62% (UV 220 nm); Retention time=0.737 min) was detected. Then the solution was added 5 mL water and extracted with DCM (5 mL*3) and concentrated under reduce pressure to give a crude. The crude was purified by reversed-phase chromatography (mobile phase: 40% water (FA)-60% ACN) and lyophilized to give 2-[(2S,6R)-2-(1-cyclopropylpyrazol-4-yl)-6-methyl-morpholin-4-yl]-7-methyl-4-[6-(trifluoromethyl)-3-pyridyl]pyrimido[4,5-d]pyridazin-8-one (46 mg, 0.0886 mmol, 29.45% yield) as yellow solid. (M+H)+=513.2; purity=97.90% (UV 220 nm); Retention time=0.565 min. ¹H NMR (400 MHz, CHLOROFORM-d) δ=9.07 (br d, J=10.9 Hz, 1H), 8.32-8.15 (m, 1H), 8.00-7.96 (m, 1H), 7.95-7.86 (m, 1H), 7.63-7.44 (m, 2H), 5.30-4.76 (m, 2H), 4.56 (br t, J=9.9 Hz, 1H), 3.98-3.70 (m, 4H), 3.57 (br d, J=3.5 Hz, 1H), 3.22-3.02 (m, 1H), 2.97-2.80 (m, 1H), 1.33 (br dd, J=5.8, 11.6 Hz, 3H), 1.11 (br s, 2H), 1.05-0.96 (m, 2H). SFC: 96.23% ee.

### Example 28: Synthesis of Compound I-203: 2-((2S,6R)-2-(1-cyclopropyl-1H-pyrazol-4-yl)-6-methylmorpholino)-4-(2-fluoro-4-methylphenyl)-7-methylpyrimido[4,5-d]pyridazin-8 (7H)-one

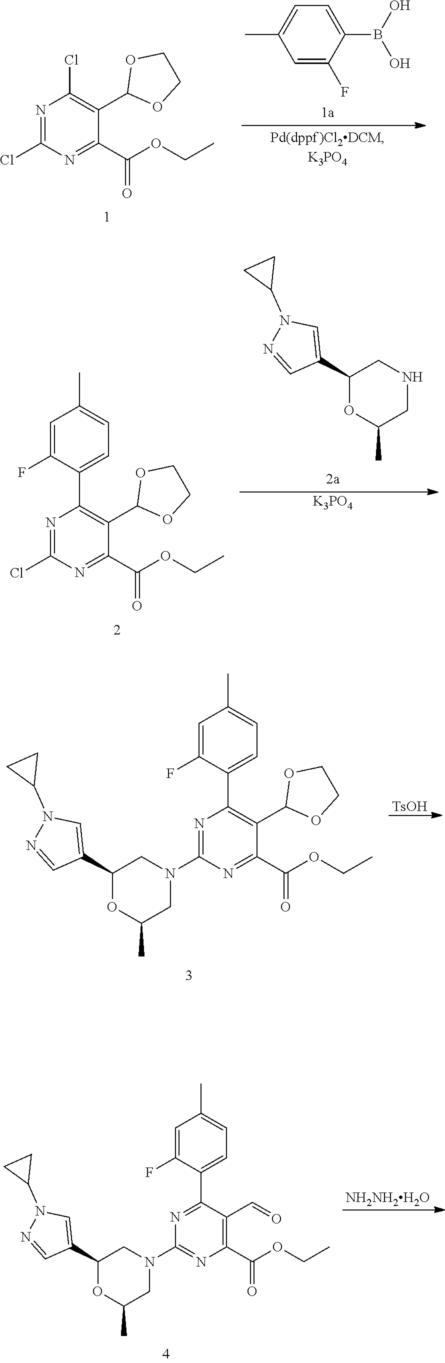

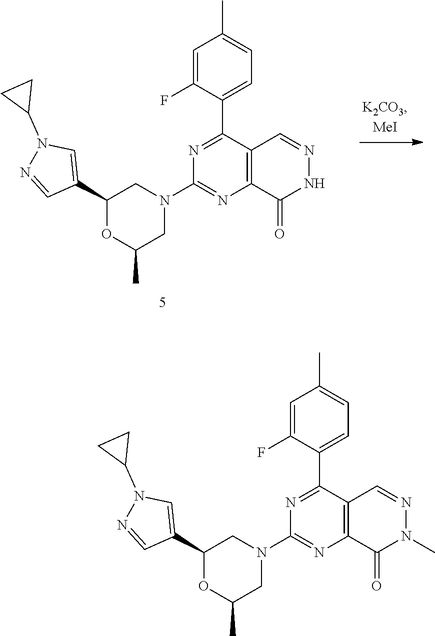 [SMILES: Cc1ccc(-c2nc(N3C[C@@H](C)O[C@@H](c4cnn(C5CC5)c4)C3)nc3c(=O)[nH]ncc23)c(F)c1 ; Cc1ccc(-c2nc(N3C[C@@H](C)O[C@@H](c4cnn(C5CC5)c4)C3)nc3c(=O)n(C)ncc23)c(F)c1]

Step 1: A mixture of ethyl 2,6-dichloro-5-(1,3-dioxolan-2-yl)pyrimidine-4-carboxylate (1.00 eq, 500 mg, 1.71 mmol) and (2-fluoro-4-methyl-phenyl) boronic acid (0.950 eq, 249 mg, 1.62 mmol) in 1,4-Dioxane (10 mL) and Water (1 mL) was added K₃PO₄ (2.00 eq, 724 mg, 3.41 mmol) and Pd(dppf)Cl₂-DCM (0.1000 eq, 125 mg, 0.171 mmol). The mixture was degassed with N₂ for 3 times and heated to 60° C., and stirred for 2 h. The reaction mixture was brown solution. LCMS (PJ-2022-01-080-59-P1A) showed a major peak with desired MS (LCMS: (M+H)+=367.1; purity=66.63% (UV 220 nm); Retention time=0.605 min) was detected. The crude was purified by flash column (PE:EtOAc=5:1; UV) and concentrated to give ethyl 2-chloro-5-(1,3-dioxolan-2-yl)-6-(2-fluoro-4-methyl-phenyl)pyrimidine-4-carboxylate (0.41 g, 1.12 mmol, 65.53% yield) as white solid. (M+H)+=367.1; purity=100% (UV 220 nm); Retention time=0.604 min. ¹H NMR (400 MHz, CHLOROFORM-d) δ=7.49-7.33 (m, 1H), 7.30-7.19 (m, 1H), 7.12-7.04 (m, 1H), 7.03-6.96 (m, 1H), 5.97-5.87 (m, 1H), 4.54-4.36 (m, 2H), 3.94-3.79 (m, 4H), 2.48-2.35 (m, 3H), 2.49-2.32 (m, 3H), 1.45-1.40 (m, 3H)

Step 2: A mixture of ethyl 2-chloro-5-(1,3-dioxolan-2-yl)-6-(2-fluoro-4-methyl-phenyl)pyrimidine-4-carboxylate (1.00 eq, 390 mg, 1.06 mmol) and (2S,6R)-2-(1-cyclopropylpyrazol-4-yl)-6-methyl-morpholine (1.20 eq, 264 mg, 1.28 mmol) in 1,4-Dioxane (10 mL) was added K₃PO₄ (2.00 eq, 451 mg. 2.13 mmol). The mixture was heated to 100° C., and stirred for 1 h. The mixture was yellow suspension. LCMS showed a major peak with desired MS (LCMS: (M+H)+=538.3; purity=94.88% (UV 220 nm); Retention time=0.661 min) was detected. The mixture was concentrated under vacuum to give a crude. The crude was purified by flash column (PE:EtOAc=1:1; UV, Rf=0.3) to give ethyl 2-[(2S,6R)-2-(1-cyclopropylpyrazol-4-yl)-6-methyl-morpholin-4-yl]-5-(1,3-dioxolan-2-yl)-6-(2-fluoro-4-methyl-phenyl)pyrimidine-4-carboxylate (0.54 g. 0.999 mmol, 93.97% yield) as orange solid. (M+H)+=538.3; purity=99.47% (UV 220 nm); Retention time=0.849 min. ¹H NMR (400 MHz, CHLOROFORM-d) δ=7.57-7.44 (m, 2H), 7.36-7.28 (m, 1H), 7.37-7.27 (m, 1H), 7.07-6.90 (m, 2H), 5.74-5.64 (m, 1H), 4.86-4.75 (m, 1H), 4.73-4.63 (m, 1H), 4.57-4.47 (m, 1H), 4.44-4.34 (m, 2H), 3.95-3.69 (m, 5H), 3.61-3.48 (m, 1H), 2.99-2.88 (m, 1H), 2.77-2.63 (m, 1H), 2.47-2.34 (m, 3H), 1.70-1.56 (m, 1H), 1.48-1.36 (m, 3H), 1.32-1.23 (m, 3H), 1.15-1.06 (m, 2H), 1.04-0.92 (m, 2H)

Step 3: A mixture of TsOH (0.200 eq, 33 mg, 0.193 mmol) in Acetone (13 mL) was added ethyl 2-[(2S, 6R)-2-(1-cyclopropylpyrazol-4-yl)-6-methyl-morpholin-4-yl]-5-(1,3-dioxolan-2-yl)-6-(2-fluoro-4-methyl-phenyl)pyrimidine-4-carboxylate (1.00 eq. 520 mg. 0.967 mmol). The mixture was stirred at 60° C., for 1 h. LCMS showed the material was consumed completely and a major peak with desired MS (LCMS: (M+H)+=494.3; purity=96.95% (UV 220 nm); Retention time=0.651 min) was detected. The mixture was concentrated under vacuum to give ethyl 2-[(2S,6R)-2-(1-cyclopropylpyrazol-4-yl)-6-methyl-morpholin-4-yl]-6-(2-fluoro-4-methyl-phenyl)-5-formyl-pyrimidine-4-carboxylate (0.67 mg, 0.00131 mmol, 0.1400% yield) as yellow solid (crude). LCMS: (M+H)+=494.3; purity=96.48% (UV 220 nm); Retention time=0.657 min.

Step 4: A mixture of ethyl 2-[(2S,6R)-2-(1-cyclopropylpyrazol-4-yl)-6-methyl-morpholin-4-yl]-6-(2-fluoro-4-methyl-phenyl)-5-formyl-pyrimidine-4-carboxylate (1.00 eq, 620 mg, 1.26 mmol) in 1,4-Dioxane (6 mL) was added hydrazine; hydrate (2.00 eq, 126 mg. 2.51 mmol). The mixture was stirred at 90° C., for 2 h. LCMS showed a major peak with desired MS (LCMS: (M+H)+=462.2; purity=87.89% (UV 220 nm); Retention time=0.723 min) was detected. The mixture was added silica gel and concentrated under reduced process to give the residue. The residue was purified by flash column (EtOAc; and DCM:MeOH=10:1) and concentrated under vacuum to give 2-[(2S,6R)-2-(1-cyclopropylpyrazol-4-yl)-6-methyl-morpholin-4-yl]-4-(2-fluoro-4-methyl-phenyl)-7H-pyrimido[4,5-d]pyridazin-8-one (375 mg, 0.804 mmol, 63.99% yield) as yellow solid. (M+H)+=462.2; purity=98.93% (UV 220 nm); Retention time=0.553 min). ¹H NMR (400 MHz, CHLOROFORM-d) δ=10.56-10.41 (m, 1H), 7.90-7.81 (m, 1H), 7.61-7.39 (m, 3H), 7.21-6.98 (m, 2H), 5.22-4.81 (m, 2H), 4.63-4.50 (m, 1H), 3.89-3.71 (m, 1H), 3.62-3.50 (m, 1H), 3.22-2.98 (m, 1H), 2.94-2.73 (m, 1H), 2.55-2.36 (m, 3H), 1.40-1.27 (m, 3H), 1.18-1.07 (m, 2H), 1.05-0.92 (m, 2H).

Step 5: To a solution of 2-[(2S,6R)-2-(1-cyclopropylpyrazol-4-yl)-6-methyl-morpholin-4-yl]-4-(2-fluoro-4-methyl-phenyl)-7H-pyrimido[4,5-d]pyridazin-8-one (1.00 eq, 150 mg, 0.325 mmol) and K₂CO₃ (2.00 eq, 90 mg, 0.650 mmol) in DMF (7.5 mL) was added Mel (3.00 eq, 0.061 mL, 0.975 mmol). The solution was stirred at 50° C., for 2 h. LCMS showed the material was consumed completely and a major peak with desired ms (LCMS: (M+H)+=476.2; purity=94.02% (UV 220 nm); Retention time=0.780 min) was detected. Then the solution was diluted with water 5 mL and filtered, the filter cake was concentrated under reduce pressure to give a crude. The crude was purified by reversed-phase chromatography (mobile phase: 60% water (FA)-40% ACN) and lyophilized to give 2-[(2S,6R)-2-(1-cyclopropylpyrazol-4-yl)-6-methyl-morpholin-4-yl]-4-(2-fluoro-4-methyl-phenyl)-7-methyl-pyrimido[4,5-d]pyridazin-8-one (38 mg, 0.0773 mmol, 23.77% yield) as light yellow solid. (M+H)+=476.2; purity=96.69% (UV 220 nm); Retention time=0.596 min. ¹H NMR (400 MHz, CHLOROFORM-d) δ=7.89-7.76 (m, 1H), 7.61-7.38 (m, 3H), 7.21-7.11 (m, 1H), 7.10-7.02 (m, 1H), 5.29-4.79 (m, 2H), 4.67-4.47 (m, 1H), 3.82 (s, 4H), 3.64-3.49 (m, 1H), 3.20-2.77 (m, 2H), 2.53-2.38 (m, 3H), 1.38-1.25 (m, 3H), 1.11 (br s, 2H), 1.04-0.97 (m, 2H). SFC: 100% ee. 60 mg (˜95% purity) was kept in hands. (M+H)+=476.2; purity=96.89% (UV 220 nm); Retention time=0.595 min. ¹H NMR (400 MHz, CHLOROFORM-d) δ=7.81 (d, J=4.0 Hz, 1H), 7.60-7.38 (m, 3H), 7.20-7.03 (m, 2H), 5.28-4.77 (m, 2H), 4.66-4.52 (m, 1H), 3.82 (s, 4H), 3.56 (br s, 1H), 3.20-2.76 (m, 2H), 2.47 (s, 3H), 1.39-1.22 (m, 3H), 1.14-1.08 (m, 2H), 1.04-0.97 (m, 2H).

### Example 28: Synthesis of Compound I-208: 8-cyclohexyl-6-((2S,6R)-2-(1-cyclopropyl-1H-pyrazol-4-yl)-6-methylmorpholino)-2,3-dimethylpyrimido[5,4-d]pyrimidin-4 (3H)-one

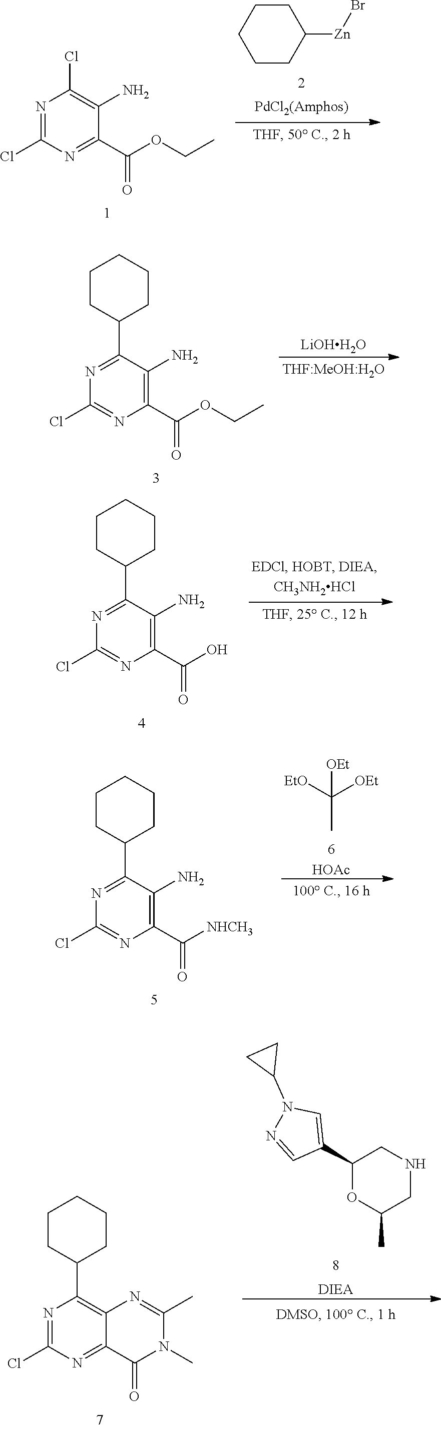

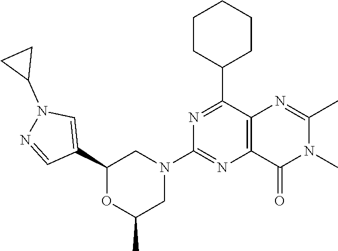 [SMILES: Cc1nc2c(C3CCCCC3)nc(N3C[C@@H](C)O[C@@H](c4cnn(C5CC5)c4)C3)nc2c(=O)n1C]

Step 1: A mixture of ethyl 5-amino-2,6-dichloro-pyrimidine-4-carboxylate (1.00 eq, 500 mg, 2.12 mmol) in THF (5 mL) under N₂ atmosphere and purged with N₂ for three times. Then bromo (cyclohexyl) zinc (1.00 eq, 4.2 mL, 2.12 mmol) was added dropwise at 0° C. under N₂ atmosphere. The mixture was stirred at 50° C., for 2 h. LCMS RT=0.928 min, 284.2=[M+H]⁺, ESI+ showed 39% of desired product. The reaction mixture was partitioned between ethyl acetate (100 mL*2) and water (80 mL). The combined organic layers were dried over Na₂SO₄, filtered and concentrated under reduced pressure to give a residue. The crude product was purified by column chromatography on silica gel chromatography (petroleum ether/ethyl acetate=0˜100%, petroleum ether/ethyl acetate=3/1, the desired product Rf=0.6) to give ethyl 5-amino-2-chloro-6-cyclohexylpyrimidine-4-carboxylate (700 mg, 2.47 mmol, 116.47% yield) as yellow oil, checked by LCMS and H NMR. (M+H)⁺=284.3; purity=81% (220 nm); Retention time=0.863 min. ¹H NMR (400 MHz, CDCl₃) δ=6.01-5.70 (m, 2H), 4.50-4.43 (m, 2H), 2.73-2.64 (m, 1H), 1.94-1.57 (m, 10H), 1.44 (t, J=7.1 Hz, 3H).

Step 2: A mixture of ethyl 5-amino-2-chloro-6-cyclohexyl-pyrimidine-4-carboxylate (1.00 eq, 600 mg, 2.11 mmol) in Methanol (3 mL), THF (3 mL) and Water (3 mL) was added LiOH·H₂O (1.20 eq, 106 mg, 2.54 mmol). The mixture was stirred at 25° C., for 1 h. LCMS (5-95 AB/1.5 min): RT=0.842 min, 256.2=[M+H]⁺, ESI+ showed 90% of desired product. The mixture was added 1 N HCl (aq.) to pH=3-4. The mixture was extracted with ethyl acetate (200 mL*3). The organic phases were dried over anhydrous Na₂SO₄ to give 5-amino-2-chloro-6-cyclohexylpyrimidine-4-carboxylic acid (630 mg, 2.46 mmol, 116.52% yield) as brown red solid, checked by LCMS m and H NMR. (M+H)⁺=256.2; purity=85% (220 nm); Retention time=0.829 min. ¹H NMR (400 MHz, CDCl₃) δ=6.04-5.88 (m, 2H), 2.75-2.66 (m, 1H), 1.97-1.46 (m, 10H).

Step 3: To a solution of 5-amino-2-chloro-6-cyclohexyl-pyrimidine-4-carboxylic acid (1.00 eq, 500 mg, 1.96 mmol) in THF (10 mL) was added EDC·HCl (1.50 eq, 562 mg, 2.93 mmol), HOBt (1.50 eq, 396 mg, 2.93 mmol) and DIEA (3.00 eq, 0.97 mL, 5.87 mmol), stirred at 20° C., for 5 min. Then the mixture was added METHYLAMINE HYDROCHLORIDE (2.00 eq, 264 mg, 3.91 mmol) and stirred at 25° C., for 3 h. LCMS (5-95 AB/1.5 min): RT=0.570 min, 269.1=[M+H⁺], ESI+ showed 30% of desired product. The reaction mixture was partitioned between ethyl acetate (80 mL*2) and water (100 mL). The combined organic layers were dried over Na₂SO₄, filtered and concentrated under reduced pressure to give a residue. The crude product was purified by reversed-phase HPLC (0.1% FA condition) to give 5-amino-2-chloro-6-cyclohexyl-N-methylpyrimidine-4-carboxamide (85 mg. 0.316 mmol, 16.18% yield) as yellow solid, checked by LCMS and H NMR. (M+H)⁺=269.1; purity=88% (220 nm); Retention time=0.864 min. ¹H NMR (400 MHz, CDCl₃) δ=7.95 (br dd, J=2.3, 4.6 Hz, 1H), 6.13-6.05 (m, 2H), 2.98 (d, J=5.1 Hz, 3H), 2.70-2.64 (m, 1H), 1.78-1.58 (m, 10H).

Step 4: A mixture of 5-amino-2-chloro-6-cyclohexyl-N-methyl-pyrimidine-4-carboxamide (1.00 eq. 75 mg, 0.279 mmol) in TRIETHYL ORTHOACETATE (29.2 eq, 1.5 mL, 8.14 mmol) was added AcOH (3.00 eq, 116 mg, 0.837 mmol). The mixture was stirred at 100° C., for 12 h. LCMS (5-95 AB/1.5 min): RT=0.888 min, 293.1=[M+H⁺], ESI+ showed 70% of desired product. The reaction mixture was partitioned between ethyl acetate (50 mL*2) and water (80 mL). The combined organic layers were dried over Na₂SO₄, filtered and concentrated under reduced pressure to give a residue. The crude product was purified by column chromatography on silica gel chromatography (petroleum ether/ethyl acetate=0˜100%, petroleum ether/ethyl acetate=1/1, the desired product Rf=0.6) to give 6-chloro-8-cyclohexyl-2,3-dimethylpyrimido[5,4-d]pyrimidin-4 (3H)-one (65 mg. 0.222 mmol, 79.56% yield) as off-white solid. (M+H)⁺=293.1; purity=100% (220 nm); Retention time=0.915 min. ¹H NMR (400 MHz, CDCl₃) δ=3.83-3.73 (m, 1H), 3.67 (s, 3H), 2.68 (s, 3H), 1.97-1.57 (m, 8H), 1.53-1.43 (m, 2H).

Step 5: To a mixture of 6-chloro-8-cyclohexyl-2,3-dimethyl-pyrimido[5,4-d]pyrimidin-4-one (1.00 eq. 60 mg. 0.205 mmol) and (2S,6R)-2-(1-cyclopropylpyrazol-4-yl)-6-methyl-morpholine (1.00 eq, 42 mg. 0.205 mmol) in DMSO (3 mL) was added DIEA (3.00 eq, 0.10 mL, 0.615 mmol). Then the mixture was stirred at 100° C., for 1 h. LCMS RT=0.905 min, 464.4=[M+H⁺], ESI+ showed 93% of desired product. The reaction mixture was partitioned between ethyl acetate (50 mL*2) and water (60 mL). The combined organic layers were dried over Na₂SO₄, filtered and concentrated under reduced pressure to give a residue. The crude product was purified by prep-TLC (DCM/MeOH=12:1, R_{f}=0.6) twice and lyophilized give 8-cyclohexyl-6-((2S,6R)-2-(1-cyclopropyl-1H-pyrazol-4-yl)-6-methylmorpholino)-2,3-dimethylpyrimido[5,4-d]pyrimidin-4 (3H)-one (41 mg, 0.0871 mmol, 42.49% yield) (SFC showed ee. is ˜100.0%) as yellow solid, checked by LCMS, HPLC, and H NMR. (M+H)⁺=464.3; purity=99% (220 nm); Retention time=0.981 min. ¹H NMR (400 MHz, CDCl₃) δ=7.55 (s, 2H), 4.98-4.80 (m, 2H), 4.57 (dd, J=2.6, 10.8 Hz, 1H), 3.80 (ddd, J=2.5, 6.2, 10.5 Hz, 1H), 3.70-3.63 (m, 1H), 3.61 (s, 4H), 2.99 (dd, J=11.0, 13.3 Hz, 1H), 2.75 (dd, J=10.8, 13.1 Hz, 1H), 2.58 (s, 3H), 1.94-1.69 (m, 6H), 1.60-1.45 (m, 4H), 1.33 (d, J=6.3 Hz, 3H), 1.15-1.10 (m, 2H), 1.04-0.98 (m, 2H).

### Example 29: Synthesis of Compound I-213: 8-(2, 4-difluorophenyl)-2,3-dimethyl-6-[(2R, 6S)-2-methyl-6-(2-methyl-4-pyridyl) morpholin-4-yl]pyrimido[5, 4-d]pyrimidin-4-one

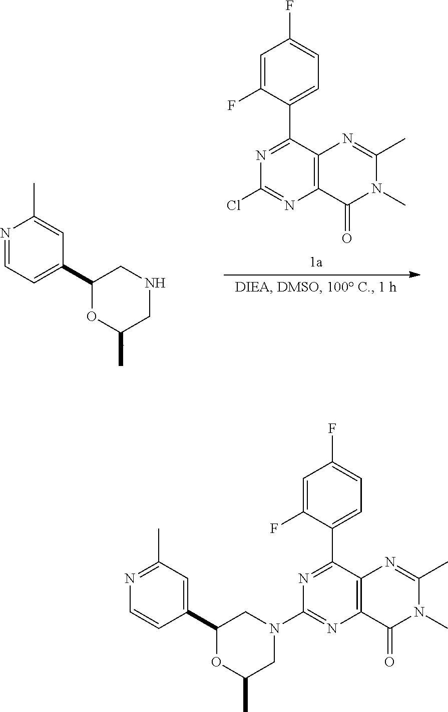

Step 1: A solution of (2R,6S)-2-methyl-6-(2-methyl-4-pyridyl) morpholine (1.10 eq, 26 mg, 0.136 mmol) and 6-chloro-8-(2,4-difluorophenyl)-2,3-dimethyl-pyrimido[5,4-d]pyrimidin-4-one (1.00 eq, 40 mg, 0.124 mmol) in DMSO (1 mL) was added DIEA (5.00 eq, 80 mg, 0.620 mmol), then stirred at 100° C., for 1 hour. LCMS (YG-2022-05-054-5-P1A1) showed 70% of desired mass. The reaction mixture was extracted with ethyl acetate (20 mL*3). The combined organic layers were dried over Na₂SO₄, filtered and concentrated under reduced pressure to give a residue. The residue was purified by prep-HPLC (Column, [Phenomenex luna C18 250*50 mm*10 um]; mobile phase: [ACN] and [H₂O](conditions: [water (0.225% FA)-ACN], B %: 65%-90%; Detector, UV 254 nm. RT: [22 min]) to afford 8-(2,4-difluorophenyl)-2,3-dimethyl-6-[(2R,6S)-2-methyl-6-(2-methyl-4-pyridyl) morpholin-4-yl]pyrimido[5,4-d]pyrimidin-4-one (24 mg, 0.0508 mmol, 40.99% yield) as yellow solid, which confirmed by ¹H NMR, F NMR, LCMS, HPLC, SFC. [M+H]⁺=479.3; purity=100% (220 nm); Retention time=0.809 min. ¹H NMR (400 MHz, CDCl₃) δ=8.50 (d, J=5.1 Hz, 1H), 7.72-7.63 (m, 1H), 7.28 (br s, 1H), 7.21 (br d, J=4.8 Hz, 1H), 7.03 (dt, J=2.1, 8.3 Hz, 1H), 6.96 (dt, J=2.4, 9.4 Hz, 1H), 5.07-4.83 (m, 2H), 4.59 (dd, J=2.4, 10.6 Hz, 1H), 3.93-3.79 (m, 1H), 3.64 (s, 3H), 3.00-2.80 (m, 2H), 2.59 (s, 3H), 2.53 (s, 3H), 1.39 (d, J=6.1 Hz, 3H)

### Example 30: Synthesis of Compound I-218: 2-((2S,6R)-2-(1-cyclopropyl-1H-pyrazol-4-yl)-6-methylmorpholino)-7-methyl-4-(2,4,5-trifluorophenyl)pyrimido[4,5-d]pyridazin-8 (7H)-one

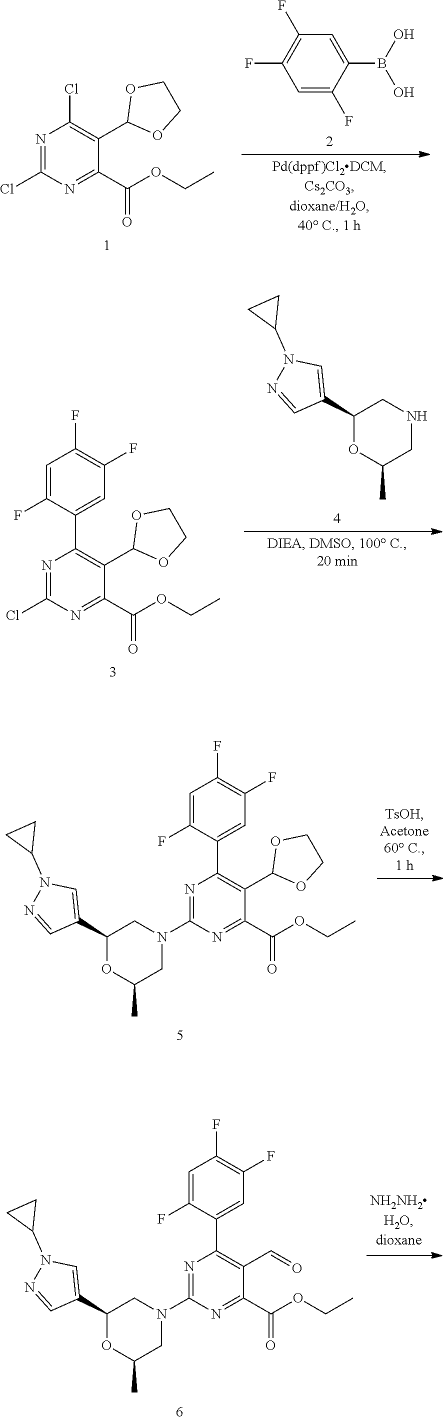 [SMILES: CCOC(=O)c1nc(Cl)nc(Cl)c1C1OCCO1 ; OB(O)c1cc(F)c(F)cc1F ; CCOC(=O)c1nc(Cl)nc(-c2cc(F)c(F)cc2F)c1C1OCCO1 ; C[C@@H]1CNC[C@H](c2cnn(C3CC3)c2)O1 ; CCOC(=O)c1nc(N2C[C@@H](C)O[C@@H](c3cnn(C4CC4)c3)C2)nc(-c2cc(F)c(F)cc2F)c1C1OCCO1 ; CCOC(=O)c1nc(N2C[C@@H](C)O[C@@H](c3cnn(C4CC4)c3)C2)nc(-c2cc(F)c(F)cc2F)c1C=O]

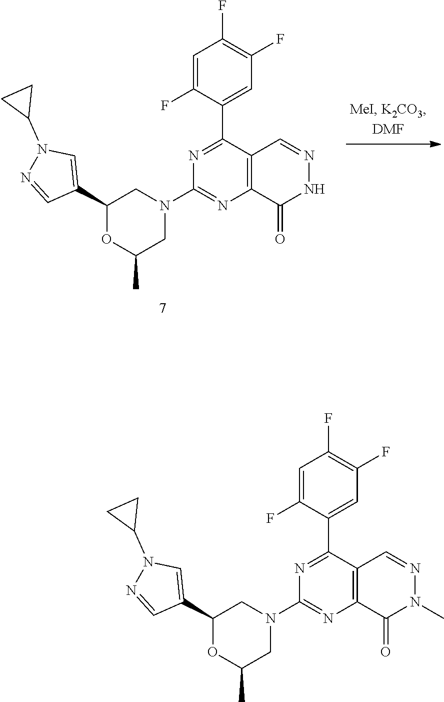 [SMILES: C[C@@H]1CN(c2nc(-c3cc(F)c(F)cc3F)c3cn[nH]c(=O)c3n2)C[C@H](c2cnn(C3CC3)c2)O1 ; C[C@@H]1CN(c2nc(-c3cc(F)c(F)cc3F)c3cnn(C)c(=O)c3n2)C[C@H](c2cnn(C3CC3)c2)O1]

Step 1: A solution of ethyl 2,6-dichloro-5-(1,3-dioxolan-2-yl)pyrimidine-4-carboxylate (1.00 eq, 1000 mg, 3.41 mmol) and (2,4,5-trifluorophenyl) boronic acid (1.00 eq, 600 mg, 3.41 mmol) in 1,4-Dioxane (10 mL) and H₂O (1 mL) was added Cs₂CO₃ (3.00 eq, 3327 mg, 10.2 mmol) and Pd(dppf)Cl₂·DCM (0.1000 eq, 278 mg, 0.341 mmol) at N₂ atmosphere. The mixture was stirred at 40° C. for 1 hour. LCMS showed the starting material was consumed completely and 35% of the desired mass was detected (35%, Rt: 0.662 min; [M+H]+=389.1 at 220 nm). The reaction mixture was poured into H₂O (100 mL) and then extracted with EtOAc (100 mL×3). The combined organic layers were dried over Na₂SO₄, filtered and concentrated under reduced pressure to give a residue. The crude product was purified by silica gel chromatography (PE/EtOAc=I/O to 1/1; PE/EtOAc=3/1, the desired product R_{f}=0.6) to afford ethyl 2-chloro-5-(1,3-dioxolan-2-yl)-6-(2,4,5-trifluorophenyl)pyrimidine-4-carboxylate (440 mg, 1.13 mmol, 33.18% yield) as brown solid, which confirmed by LCMS and HNMR. [M+H]⁺=389.0; purity=98% (220 nm); Retention time=0.642 min. ¹H NMR (400 MHz, CDCl₃) δ=7.43 (ddd, J=6.6, 8.6, 9.7 Hz, 1H), 7.06 (dt, J=6.3, 9.5 Hz, 1H), 5.99 (d, J=0.9 Hz, 1H), 4.46 (q, J=7.2 Hz, 2H), 3.93-3.82 (m, 4H), 1.44 (t, J=7.1 Hz, 3H).

Step 2: To a solution of ethyl 2-chloro-5-(1,3-dioxolan-2-yl)-6-(2,4,5-trifluorophenyl)pyrimidine-4-carboxylate (1.00 eq. 400 mg, 1.03 mmol) and (2S,6R)-2-(1-cyclopropyl-1H-pyrazol-4-yl)-6-methylmorpholine (1.00 eq, 213 mg, 1.03 mmol) in DMSO (10 mL), then the solution was added DIEA (5.00 eq, 0.85 mL, 5.14 mmol) and stirred at 100° C., for 20 min. LCMS showed the starting material was consumed completely and 80% of the desired mass was detected (80%, Rt: 0.683 min: [M+H]+=560.3 at 220 nm). The was diluted with H₂O (40 mL) and extracted with EtOAc (40 mL) twice. The combined organic layers were washed with an aqueous solution with brine (30 mL) three times and dried over Na₂SO₄. The solvent was filtered and concentrated under reduced pressure. The residue was purified by column chromatography (SiO₂, PE/EtOAc=I/O to 0/1; PE/EtOAc=3/1, R_{f}-0.3) to give ethyl 2-((2S,6R)-2-(1-cyclopropyl-1H-pyrazol-4-yl)-6-methylmorpholino)-5-(1,3-dioxolan-2-yl)-6-(2,4,5-trifluorophenyl)pyrimidine-4-carboxylate (550 mg, 0.983 mmol, 95.53% yield) as yellow oil, checked by LCMS and HNMR. [M+H]⁺=460.3; purity=91% (220 nm); Retention time=0.673 min. ¹H NMR (400 MHz, CDCl₃) δ=7.52 (s, 2H), 7.37-7.28 (m, 1H), 7.01 (dt, J=6.6, 9.4 Hz, 1H), 5.70 (s, 1H), 4.85-4.61 (m, 2H), 4.52 (dd, J=2.2, 10.9 Hz, 1H), 4.41 (q, J=7.2 Hz, 2H), 3.91-3.80 (m, 4H), 3.79-3.70 (m, 1H), 3.56 (tt, J=3.8, 7.3 Hz, 1H), 2.96 (dd, J=11.4, 13.0 Hz, 1H), 2.72 (dd, J=11.0, 13.0 Hz, 1H), 1.42 (t, J=7.1 Hz, 3H), 1.32-1.26 (m, 3H), 1.15-1.08 (m, 2H), 1.04-0.97 (m, 2H).

Step 3: A mixture of ethyl 2-((2S,6R)-2-(1-cyclopropyl-1H-pyrazol-4-yl)-6-methylmorpholino)-5-(1,3-dioxolan-2-yl)-6-(2,4,5-trifluorophenyl)pyrimidine-4-carboxylate (1.00 eq, 500 mg, 0.894 mmol) in Acetone (10 mL) was added TsOH (0.200 eq, 31 mg, 0.179 mmol). The mixture was stirred at 60° C., for 1 h. LCMS showed the starting material was consumed completely and the desired mass was detected (75%, Rt=0.662 min: [M+H]+=516.2 at 220 nm). The reaction was quenched by 80 mL H₂O, extracted with EtOAc (30 mL×3). The combine organic layers was dried over anhydrous Na₂SO₄, filtered and concentrated under reduced pressure to give the residue. The residue was purified by Flash Column (PE/EtOAc=0˜50%; PE/EA=1/1, the desired product R_{f}=0.5) to afford ethyl 2-((2S,6R)-2-(1-cyclopropyl-1H-pyrazol-4-yl)-6-methylmorpholino)-5-formyl-6-(2,4,5-trifluorophenyl)pyrimidine-4-carboxylate (450 mg, 0.873 mmol, 97.69% yield) as yellow oil, checked by [M+H]+=516.4; purity=97% (220 nm); Retention time=0.667 min. ¹H NMR (400 MHz, CDCl₃) δ=9.69 (d, J=4.6 Hz, 1H), 7.58-7.50 (m, 2H), 7.46-7.33 (m, 1H), 7.14-7.01 (m, 1H), 5.02-4.76 (m, 2H), 4.62-4.44 (m, 3H), 3.84-3.71 (m, 1H), 3.65-3.53 (m, 1H), 3.15-2.99 (m, 1H), 2.89-2.75 (m, 1H), 1.43 (q, J=7.3 Hz, 3H), 1.32 (br d, J=5.8 Hz, 3H), 1.12 (br s, 2H), 1.03 (br d, J=4.0 Hz, 2H).

Step 4: To a solution of ethyl 2-((2S,6R)-2-(1-cyclopropyl-1H-pyrazol-4-yl)-6-methylmorpholino)-5-formyl-6-(2,4,5-trifluorophenyl)pyrimidine-4-carboxylate (1.00 eq, 350 mg, 0.679 mmol) in 1,4-Dioxane (10 mL) was added Hydrazine hydrate (0.900 eq. 31 mg, 0.611 mmol). The mixture was stirred at 25° C., for 2 h. LCMS showed that 17% the starting material remained. Then the reaction mixture was heated to 90° C., for 6 h. LCMS showed that the desired mass was major (66%, Rt: 0.583 min; [M+H]+=484.3 at 220 nm). The reaction mixture was concentrated under reduced pressure to give a residue. The residue was purified by column chromatography (SiO₂, PE/EtOAc=I/O to 0/1; PE/EtOAc=3/1 the desired product R_{f}-0.5) to give 2-((2S,6R)-2-(1-cyclopropyl-1H-pyrazol-4-yl)-6-methylmorpholino)-4-(2,4,5-trifluorophenyl)pyrimido[4,5-d]pyridazin-8 (7H)-one (280 mg. 0.573 mmol, 84.45% yield) as yellow solid. [M+H]+=484.4; purity=99% (220 nm); Retention time=0.582 min. ¹H NMR (400 MHz, CDCl₃) δ=10.27-10.06 (m, 1H), 7.80 (d, J=4.3 Hz, 1H), 7.59-7.51 (m, 2H), 7.50-7.40 (m, 1H), 7.21-7.12 (m, 1H), 5.20-5.01 (m, 1H), 4.99-4.77 (m, 1H), 4.65-4.51 (m, 1H), 3.88-3.72 (m, 1H), 3.58 (br d, J=1.4 Hz, 1H), 3.23-3.03 (m, 1H), 2.97-2.79 (m, 1H), 1.36 (br d, J=5.5 Hz, 3H), 1.17-1.10 (m, 2H), 1.02 (br d, J=6.3 Hz, 2H).

Step 5: To a mixture of 2-((2S,6R)-2-(1-cyclopropyl-1H-pyrazol-4-yl)-6-methylmorpholino)-4-(2,4,5-trifluorophenyl)pyrimido[4,5-d]pyridazin-8 (7H)-one (1.00 eq, 90 mg. 0.186 mmol) and K₂CO₃ (2.00 eq, 51 mg. 0.372 mmol) in DMF (3 mL) was added Mel (3.00 eq, 79 mg. 0.558 mmol) and the reaction mixture was stirred at 25° C., for 4 h. LCMS showed that 36% of the starting material and 36% of the desired mass (36%, Rt: 0.625 min: [M+H]+=498.2 at 220 nm). The reaction mixture was stirred at 25° C., for 16 h. LCMS showed that 12% of the starting material, 17% the by-product (two Me mass) and 67% of the desired mass (67%, Rt: 0.618 min: [M+H]+=498.3 at 220 nm). The reaction mixture was quenched by addition H₂O (30 mL) at 0° C., and then extracted with EtOAc (30 mL×2). The combined organic layers were washed with brine (30 mL×3), dried over Na₂SO₄, filtered and concentrated under reduced pressure to give a residue. The crude product was purified prep-TLC (SiO₂, DCM/EtOAc=3/1: the desired product R_{f}=0.6, the starting material R_{f}-0.55 showed by 254 nm) and lyophilized to give 2-((2S,6R)-2-(1-cyclopropyl-1H-pyrazol-4-yl)-6-methylmorpholino)-7-methyl-4-(2,4,5-trifluorophenyl)pyrimido[4,5-d]pyridazin-8 (7H)-one (67 mg, 0.133 mmol, 71.55% yield) as yellow solid, checked by [M+H]⁺=498.2; purity=99.6% (220 nm); Retention time=0.762 min. ¹H NMR (400 MHz, CDCl₃) δ=7.76 (d, J=4.4 Hz, 1H), 7.58-7.51 (m, 2H), 7.50-7.41 (m, 1H), 7.20-7.11 (m, 1H), 5.25-5.03 (m, 1H), 5.00-4.74 (m, 1H), 4.67-4.51 (m, 1H), 3.84 (s, 4H), 3.58 (br s, 1H), 3.21-3.01 (m, 1H), 2.97-2.77 (m, 1H), 1.34 (br s, 3H), 1.16-1.08 (m, 2H), 1.06-0.98 (m, 2H).

### Table B. Exemplary Compounds

The compounds disclosed below in Table B were made by a method of the present disclosure or a similar method. The appropriate reagents, starting materials and conditions necessary for synthesizing the compounds of Table B would be apparent to a person of ordinary skill in the art. Compounds designated with “(+/−)” were isolated as a mixture of diastereomers sharing the same relative stereochemistry (ie. cis or trans). Compounds designated with “(rac)” were isolated as a mixture of all possible stereoisomers of the shown compound. Compounds lacking either designation were isolated with the specific stereochemistry shown, such that the specific stereoisomer shown made up at least 90% of the isolated product.

| Ex | | | Method used |
|---|---|---|---|
| # | Structure | Name | to synthesize |
| 1 | 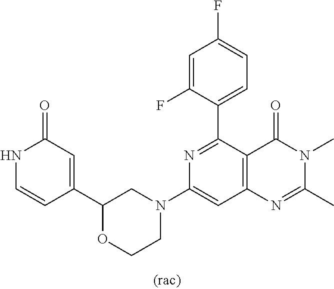 [SMILES: Cc1nc2cc(N3CCOC(c4cc[nH]c(=O)c4)C3)nc(-c3ccc(F)cc3F)c2c(=O)n1C] | 5-(2,4-difluorophenyl)-2,3-dimethyl- 7-(2-(2-oxo-1,2-dihydropyridin-4- yl)morpholino)pyrido[4,3- d]pyrimidin-4(3H)-one | |
| 2 | 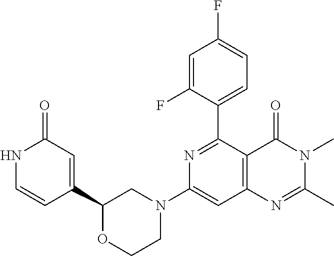 [SMILES: Cc1nc2cc(N3CCO[C@@H](c4cc[nH]c(=O)c4)C3)nc(-c3ccc(F)cc3F)c2c(=O)n1C] | (S)-5-(2,4-difluorophenyl)-2,3- dimethyl-7-(2-(2-oxo-1,2- dihydropyridin-4- yl)morpholino)pyrido[4,3- d]pyrimidin-4(3H)-one | |
| 3 | 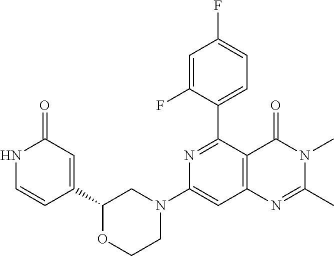 [SMILES: Cc1nc2cc(N3CCO[C@H](c4cc[nH]c(=O)c4)C3)nc(-c3ccc(F)cc3F)c2c(=O)n1C] | (R)-5-(2,4-difluorophenyl)-2,3- dimethyl-7-(2-(2-oxo-1,2- dihydropyridin-4- yl)morpholino)pyrido[4,3- d]pyrimidin-4(3H)-one | |
| 4 | 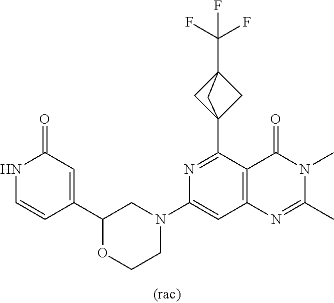 [SMILES: Cc1nc2cc(N3CCOC(c4cc[nH]c(=O)c4)C3)nc(C34CC(C(F)(F)F)(C3)C4)c2c(=O)n1C] | 2,3-dimethyl-7-(2-(2-oxo-1,2- dihydropyridin-4-yl)morpholino)-5- (3- (trifluoromethyl)bicyclo[1.1.1]pentan- 1-yl)pyrido[4,3-d]pyrimidin-4(3H)- one | |
| 5 | 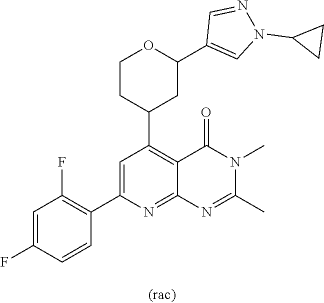 [SMILES: Cc1nc2nc(-c3ccc(F)cc3F)cc(C3CCOC(c4cnn(C5CC5)c4)C3)c2c(=O)n1C] | 5-(2-(1-cyclopropyl-1H-pyrazol-4- yl)tetrahydro-2H-pyran-4-yl)-7-(2,4- difluorophenyl)-2,3- dimethylpyrido[2,3-d]pyrimidin- 4(3H)-one | |
| 6 | 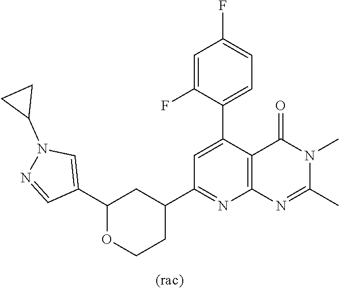 [SMILES: Cc1nc2nc(C3CCOC(c4cnn(C5CC5)c4)C3)cc(-c3ccc(F)cc3F)c2c(=O)n1C] | 7-(2-(1-cyclopropyl-1H-pyrazol-4- yl)tetrahydro-2H-pyran-4-yl)-5-(2- fluoro-4-(trifluoromethyl)phenyl)-2,3- dimethylpyrido[2,3-d]pyrimidin- 4(3H)-one | |

### Example A3: In Vitro Assay Data

### In Vitro Measurement of Triggering Receptor Expressed on Myeloid Cells 2 Activity Using Cellular Phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 μL of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluorescence intensity was measured using a Perkin Elmer Envision plate reader. Intensities were used to generate a standard curve, and % activation was calculated. Curve fitting was performed using Prism v9 software, log (agonist) vs response-variable slope (four parameters), and EC50s were calculated from the curve fit.

The results presented in Table D have been generated with the in vitro assay described above. This assay may be used to test any of the compounds described herein to assess and characterize a compound's ability to act as an agonist of TREM2.

Compounds designated as “A” demonstrated an EC50 of ≤0.05 μM. Compounds designated as “B” demonstrated an EC50>0.05 UM and ≤0.5 μM. Compounds designated as “C” demonstrated an EC50>0.5 μM and ≤3.0 μM. Compounds designated as “D” demonstrated an EC50>3.0 μM and ≤100 μM. Compounds designated as “-” had not been tested as of the filing of the present application, but can be tested using the methods described herein.

TABLE D
hTREM2 EC50 Data (HEK293 Cells)

| Ex # | hTREM2 EC50 μM |
|---|---|
| 1 | B |
| 2 | B |
| 3 | D |
| 4 | A |
| 5 | C |
| 6 | B |

TABLE D-2
hTREM2 EC50 Data (HEK293 Cells)

| I# | hTREM2 EC50 μM |
|---|---|
| I-40 | A |
| I-42 | C |
| I-45 | B |
| I-48 | A |
| I-53 | B |
| I-58 | A |
| I-63 | B |
| I-69 | B |
| I-74 | A |
| I-79 | A |
| I-84 | B |
| I-90 | A |
| I-95 | A |
| I-100 | B |
| I-105 | B |
| I-110 | A |
| I-115 | C |
| I-120 | B |
| I-125 | B |
| I-130 | A |
| I-132 | B |
| I-134 | A |
| I-136 | A |
| I-141 | A |
| I-146 | B |
| I-152 | A |
| I-157 | C |
| I-163 | A |
| I-168 | B |
| I-173 | C |
| I-178 | A |
| I-183 | A |
| I-184 | A |
| I-188 | A |
| I-193 | A |
| I-198 | A |
| I-203 | A |
| I-208 | A |
| I-213 | A |
| I-218 | A |

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula I 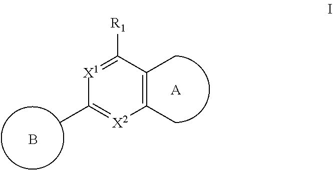
or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CR¹³, CH or N;
X² is CR¹⁴, CH or N;
Ring A together with the 6-membered ring system to which it is fused forms a bicyclic ring system of formula 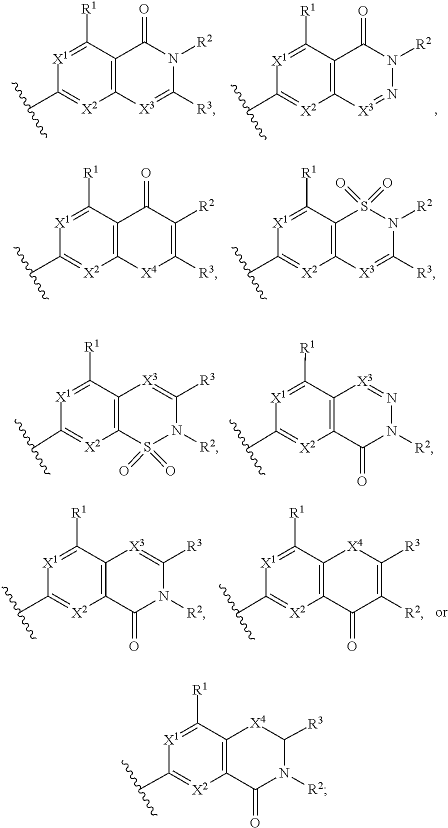
X³ is CR¹⁵, CH or N;
X⁴ is O, NR⁴, C(R⁴)₂, CHR⁴, SO₂, or C═O;
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
each R⁴ is independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
Ring B is 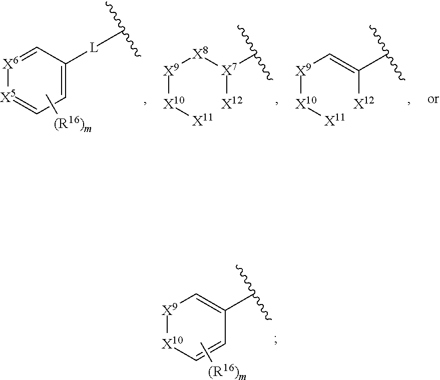
L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆ haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³, R¹⁴ and R¹⁵ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
each R is independently hydrogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

2. The compound of claim 1, wherein R¹ is optionally substituted C₃₋₆cycloalkyl, optionally substituted spiro[3.3]heptanyl, optionally substituted spiro[5.2]octanyl, optionally substituted 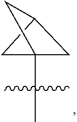 [SMILES: *C12CC(C1)C2]
optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted-OCH₂—(C₃₋₄cycloalkyl).

3. The compound of claim 1, wherein R¹ is optionally substituted phenyl.

4. The compound of claim 1, wherein R¹ is:
(A) —CH₂CH₂CF₃, 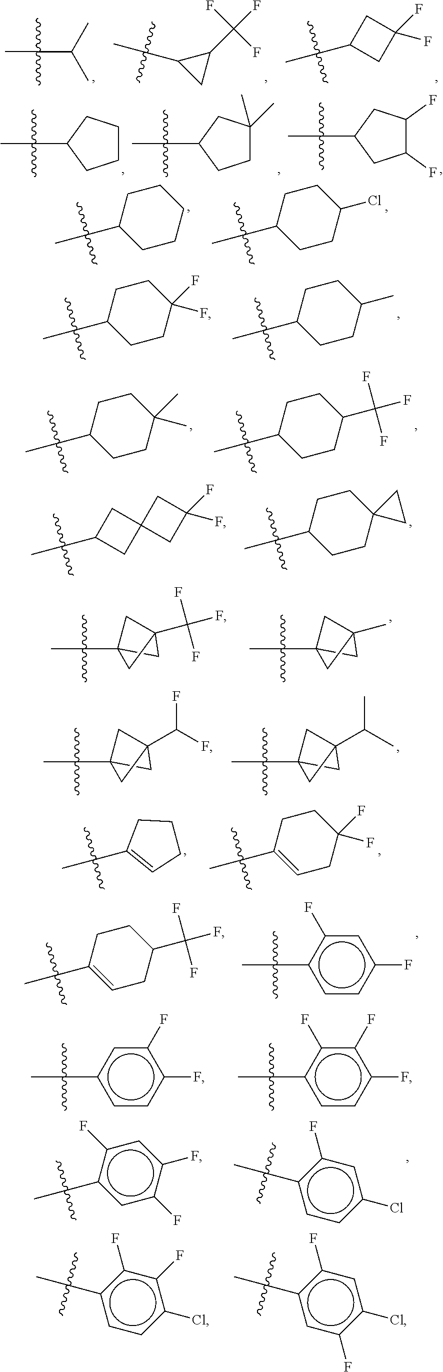 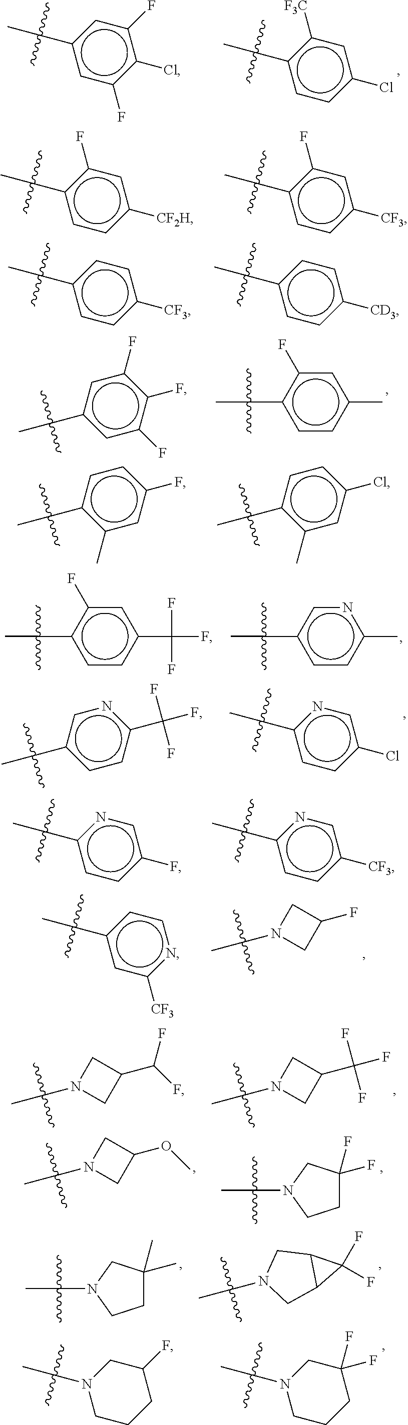 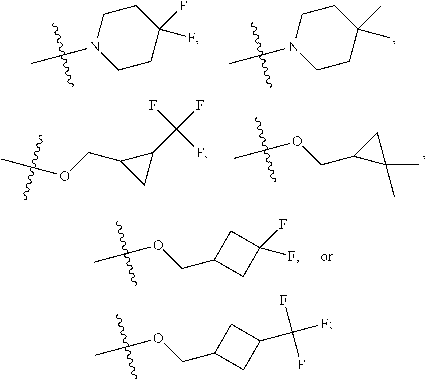
or
(B) a substituent selected from:

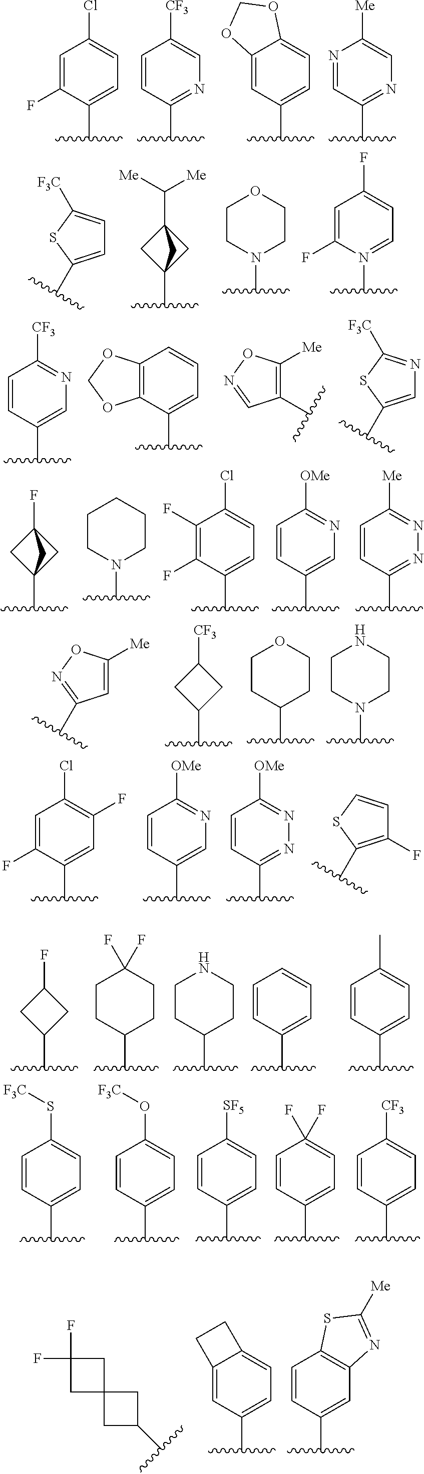

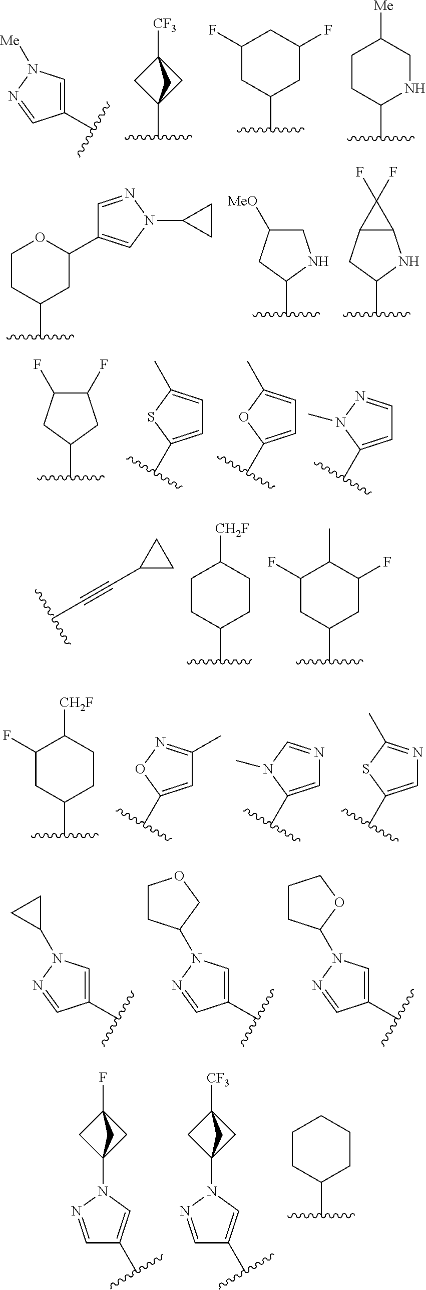

5. The compound of any one of claims 1-4, wherein Ring A together with the 6-membered ring system to which it is fused forms a bicyclic ring system of formula:

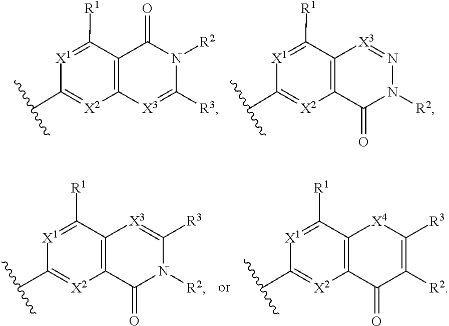

6. The compound of any one of claims 1-5, wherein X¹ is CH or N.

7. The compound of any one of claims 1-6, wherein X² is CH or N.

8. The compound of any one of claims 1-7, wherein X³ is CH or N.

9. The compound of any one of claims 1-8, wherein X⁴ is NR⁴.

10. The compound of any one of claims 1-9, wherein Ring B is

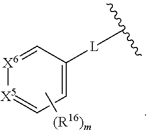

11. The compound of any one of claims 1-10, wherein L is a bond.

12. The compound of any one of claims 1-11, wherein Ring B is

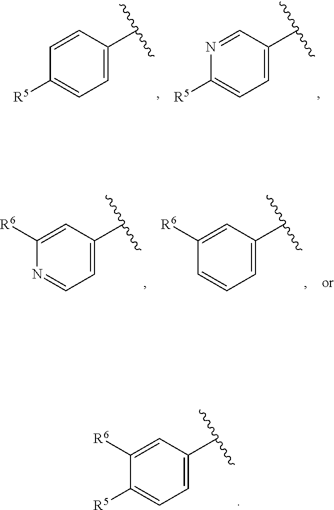

13. The compound of any one of claims 1-9, wherein Ring B is

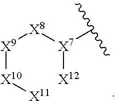

14. The compound of any one of claims 1-9, wherein Ring B is selected from:

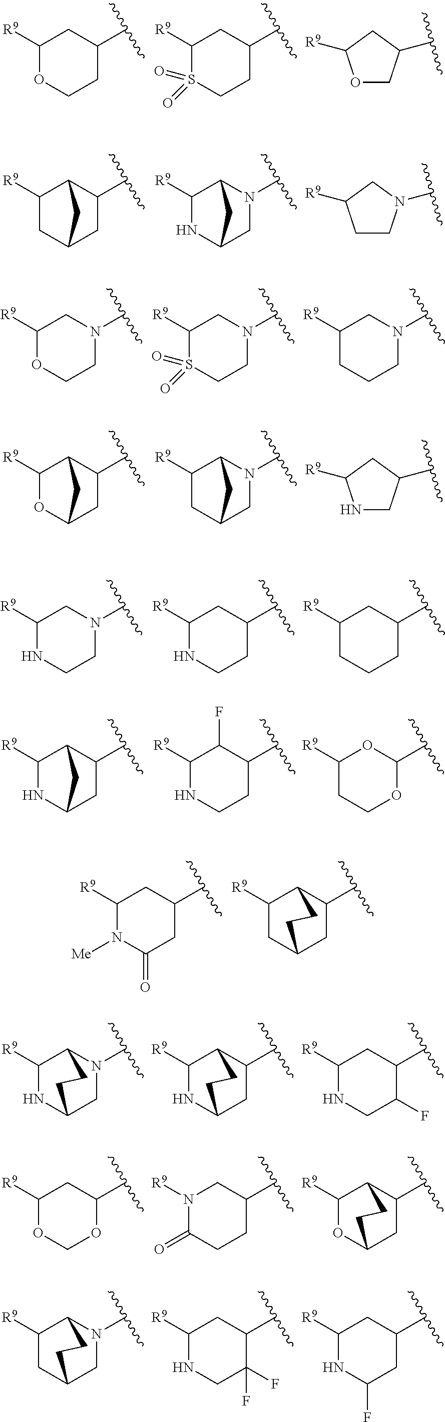

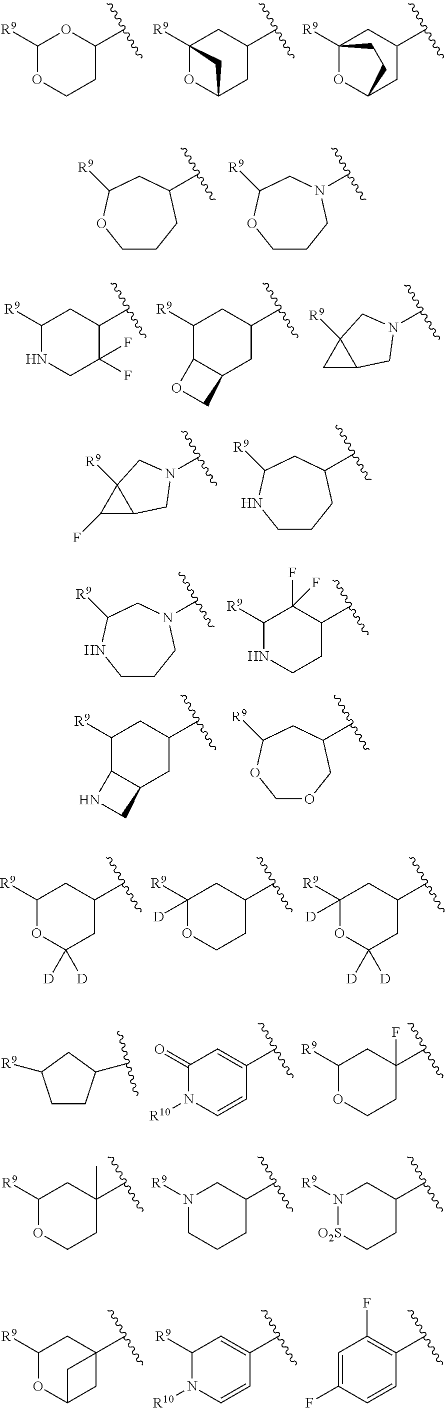

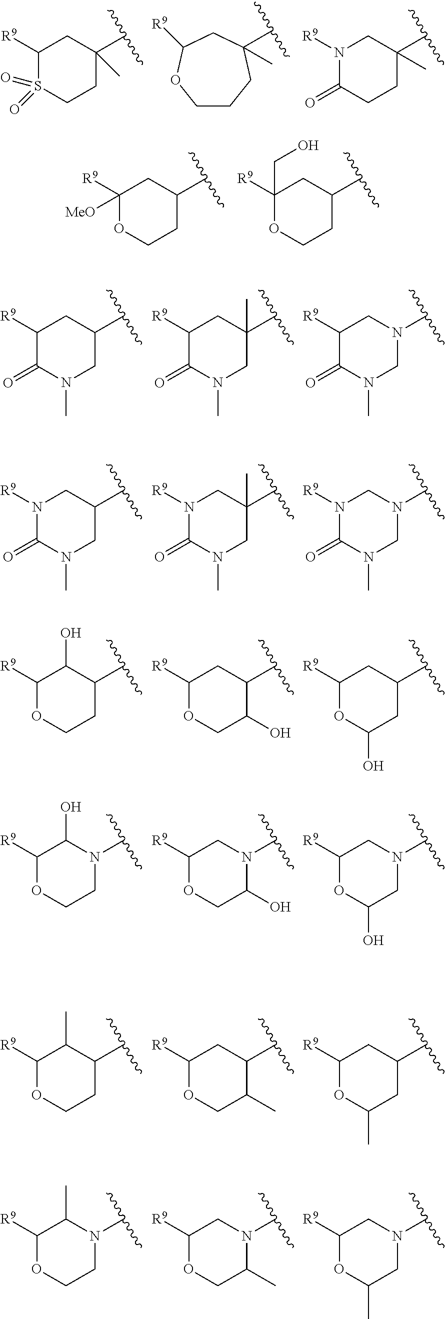

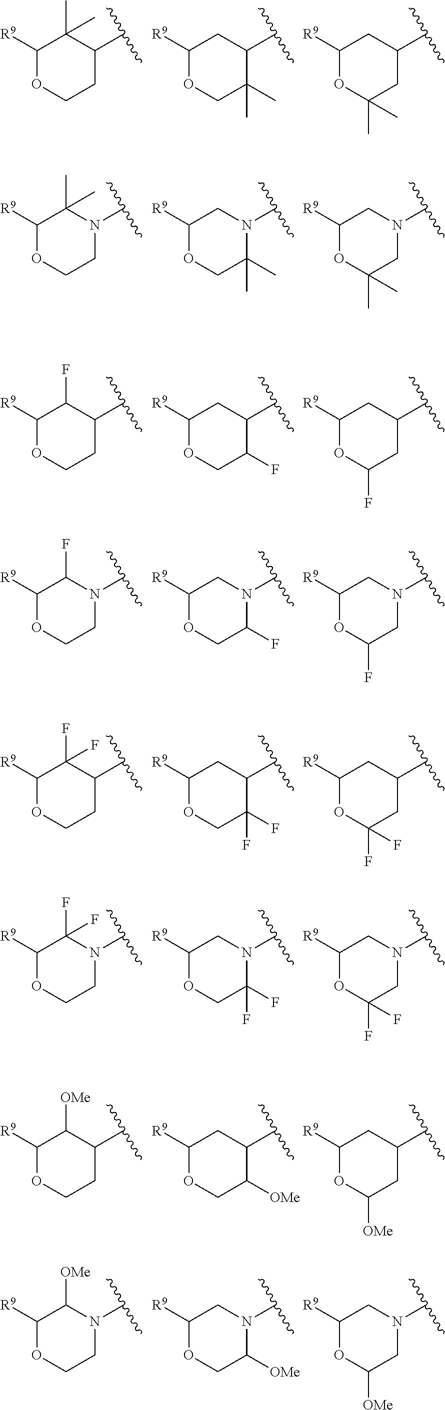

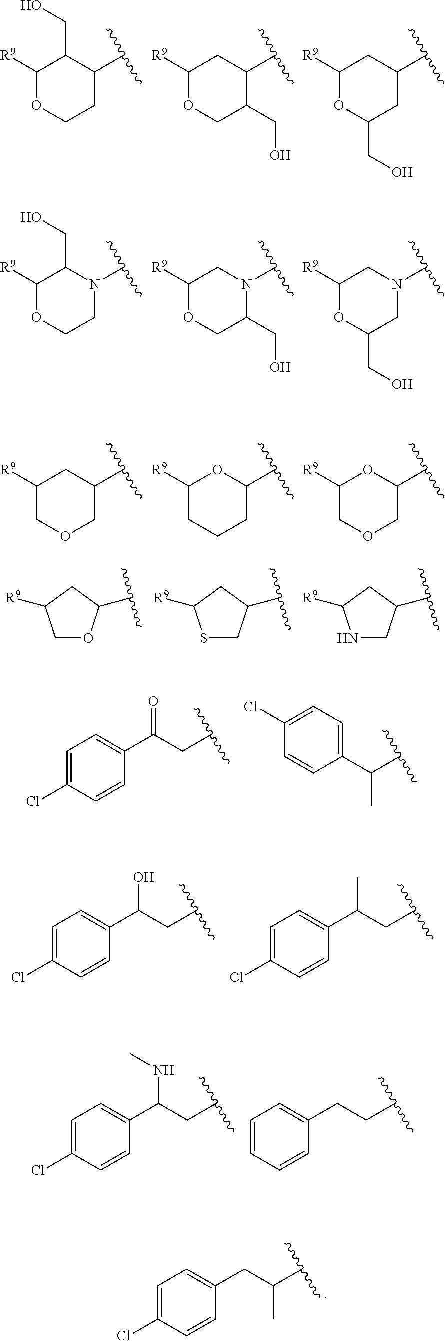

15. The compound of any one of claims 1-9, wherein Ring B is

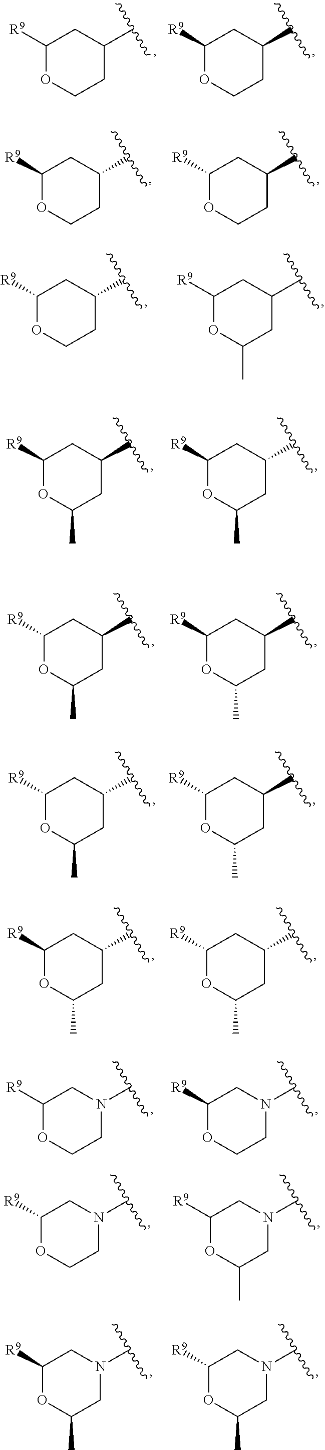

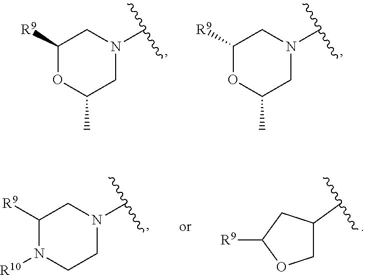

16. The compound of any one of claims 1-9, 13 and 14, wherein R⁹ is selected from:

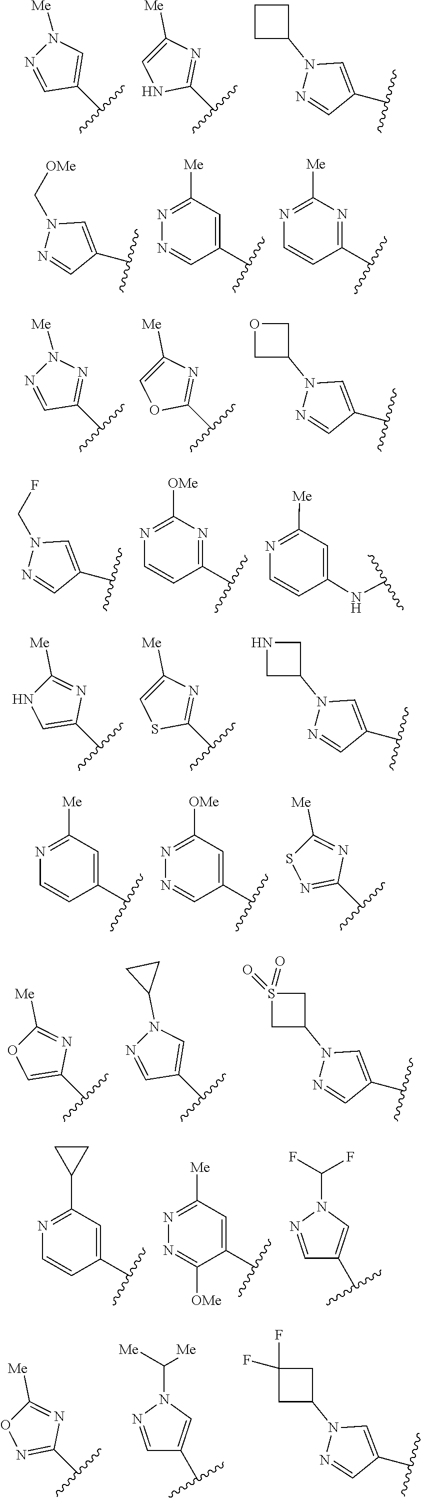

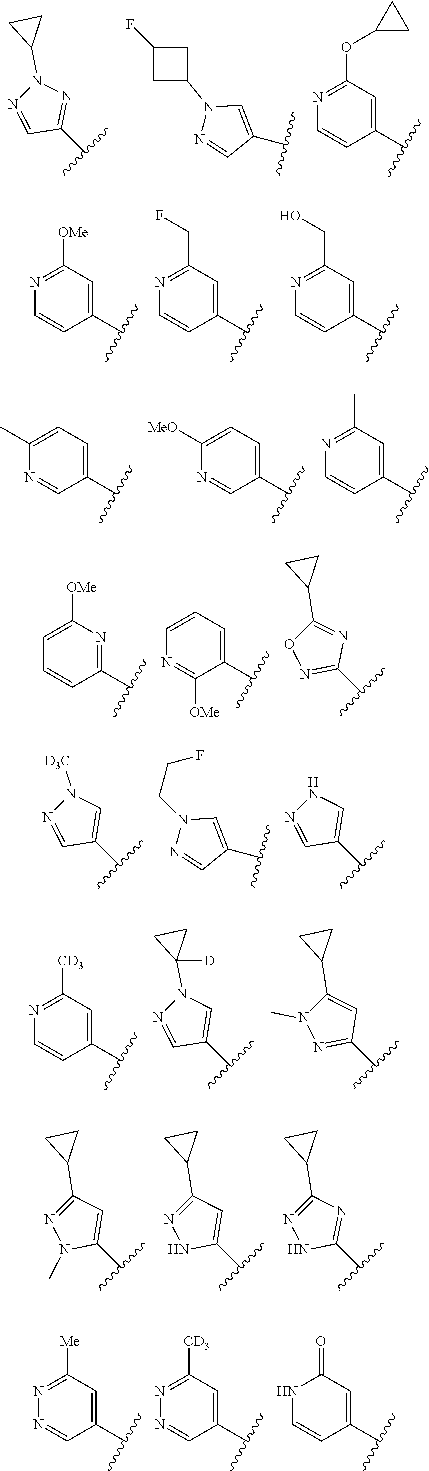

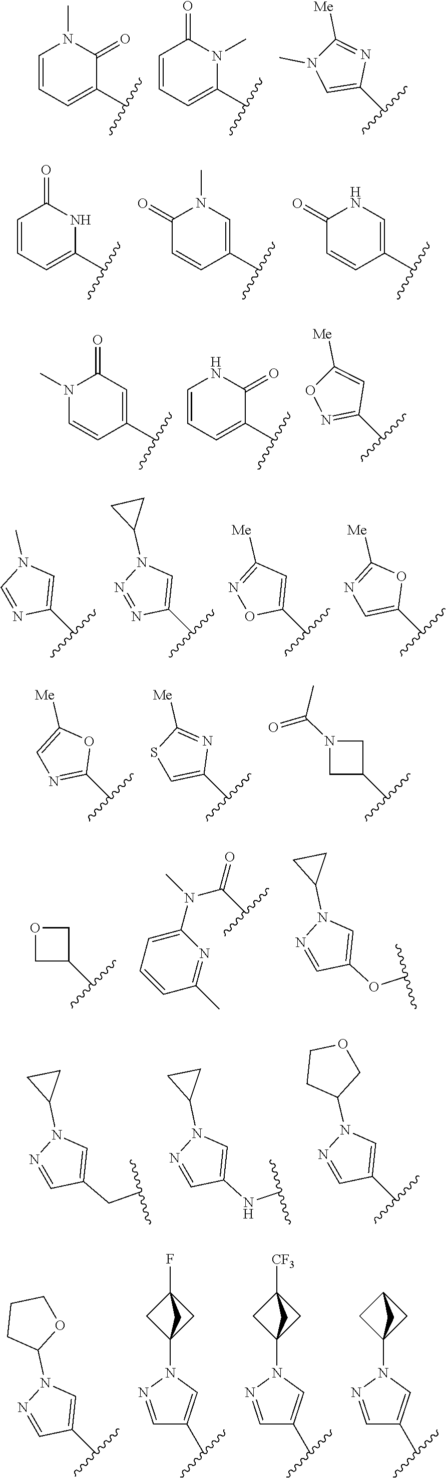

17. The compound of any one of claims 1-9, 13 and 14, wherein R⁹ is

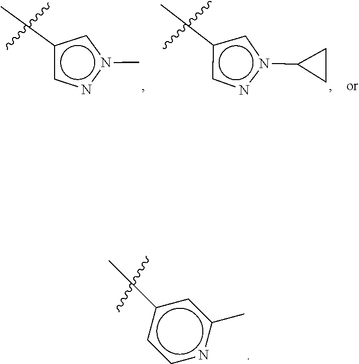

18. The compound of any one of claims 1-17, wherein the compound is a compound of Formula IIa, IIb, IIb′, IIb″, IIc, IIc′, IIc″, IIc′″, IIc″″, IIIa, IVa, Va, VIa, VIIa, VIIIa, or IXa.

19. A compound of Table A of Table A-2, or a pharmaceutically acceptable salt thereof.

20. A pharmaceutical composition comprising the compound according to any one of claims 1-19, or a tautomer thereof, or a pharmaceutically acceptable salt of said compound or said tautomer, and a pharmaceutically acceptable excipient.

21. A compound according to any one of claims 1-19, or a tautomer thereof, or a pharmaceutically acceptable salt of said compound or said tautomer, or the pharmaceutical composition according to claim 20 for use as a medicament.

22. A compound according to any one of claims 1-19, or a tautomer thereof, or a pharmaceutically acceptable salt of said compound or said tautomer, or the pharmaceutical composition according to claim 20 for use in treating or preventing a condition associated with a loss of function of human TREM2.

23. A compound according to any one of claims 1-19, or a tautomer thereof, or a pharmaceutically acceptable salt of said compound or said tautomer, or the pharmaceutical composition according to claim 20 for use in treating or preventing Parkinson's disease, rheumatoid arthritis, Alzheimer's disease, Nasu-Hakola disease, frontotemporal dementia, multiple sclerosis, prion disease, or stroke.

24. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing a condition associated with a loss of function of human TREM2.

25. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing Parkinson's disease, rheumatoid arthritis, Alzheimer's disease, Nasu-Hakola disease, frontotemporal dementia, multiple sclerosis, prion disease, or stroke.

26. A method of treating or preventing a condition associated with a loss of function of human TREM2 in a subject in need thereof, the method comprising administering to the subject a therapeutically effective amount of the compound according to any one of claims 1-19, or a tautomer thereof, or a pharmaceutically acceptable salt of said compound or said tautomer.

27. A method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound according to any one of claims 1-19, or a tautomer thereof, or a pharmaceutically acceptable salt of said compound or said tautomer.
